(19) United States
(12) Patent Application Publication
(10) Pub. No.: US 2003/0134278 A1
(43) Pub. Date: Jul. 17, 2003

(54) Chromosome inheritance modifiers and their uses

(76) Inventors: Gary H. Karpen, San Diego, CA (US); Kenneth W. Dobie, Del Mar, CA (US); Kevin R. Cook, Bloomington, IN (US); Terence D. Murphy, Baltimore, MD (US)

Correspondence Address: SCHWEGMAN, LUNDBERG, WOESSNER & KLUTH, P.A., P.O. BOX 2938, MINNEAPOLIS, MN, 55402, US

(21) Appl. No.: 09/949,029

(22) Filed: Sep. 7, 2001

Related U.S. Application Data
(60) Provisional application No. 60/231,178, filed on Sep. 7, 2000.

(51) Int. Cl. C12Q 1/68; C12Q 1/00; A61K 31/00

(52) U.S. Cl. 435/6; 435/4; 514/1

(57) ABSTRACT

The invention provides a method to identify agents and polynucleotides that modulate chromosomal inheritance. The invention also provides polynucleotides isolated according to the method as well as orthologous polynucleotides and expression cassettes and vectors containing the polynucleotides.

### STATEMENT OF GOVERNMENT FUNDING

At least a part of the invention described in this patent application was funded under a grant from the National Institutes of Health, grant no. RO1-GM54549

### BACKGROUND OF THE INVENTION

Accurate chromosome inheritance is a dynamic and multifactorial process (Rieder and Salmon, Trends. Cell Biol., 8:310 (1998)). Early in mitotic prophase chromosomes are condensed and sister chromatids are held together at centric heterochromatin. As mitosis progresses the chromosome arms and centromeres associate with microtubules radiating from centrosomes and chromosomes congress to the metaphase plate due to the action of antipoleward forces and motor proteins. The spindle assembly checkpoint apparatus monitors this process and sister chromatids segregate to opposite poles only after all the chromosomes have aligned at the plate. The kinetochore, a specialized proteinaceous structure, is a central focus for checkpoint proteins as well as proteins required for spindle attachment, chromosome congression and segregation (Dobie et al., Curr. Opin. Genet. Dev., 9:206-217 (1999)). While cytokinesis marks the end of mitosis, the chromosomes still have to undergo decondensation and DNA replication before chromosome division can be repeated. A further level of complexity is added in germ cells where homologous chromosomes pair and segregate in meiosis I and sister chromatids remain associated until meiosis II.

Errors in the above processes can result in aneuploidy which is associated with birth defects such as Down syndrome and most types of tumors (Hook, Aneuploidy: Etiology & Mechanisms, ed. Dellarco et al., New York, Plenum Press (1985); Mitelman, Catalog of Chromosome Aberrations in Cancer, 5th Ed., New York: Wiley (1994)). Studies performed in diverse organisms have been crucial in the identification of genes involved in chromosome inheritance (Pluta et al., Science, 270:1591-1594 (1995)). However, due to the complexity of chromosome architecture and inheritance we are only beginning to scratch the surface in our understanding of the gene products required for chromosome inheritance. A more complete understanding will require the identification and characterization of novel components of chromosome architecture and a deeper understanding of how chromosome movements are governed and orchestrated with the cell cycle. Knowledge of how these processes operate will be essential if we are to understand the relationship between aneuploidy and birth defects or cancer progression, and to diagnose and treat these conditions.

The fruit fly Drosophila melanogaster is a model system for higher eukaryotic chromosome inheritance. This genetically amenable organism displays diverse types of chromosome cycles and cell divisions. For example, there are multiple rapid divisions without cellularization during early embryonic development, somatic and germ-line mitosis, meiosis I and II and sex-specific patterns of meiosis; chromosome segregation has to be accomplished appropriately through these different types of division to ensure viability and normal function of the organism. Because of this complexity, the centromeres share many structural similarities (e.g., large amount of DNA, kinetochore structure, heterochromatic location and attachment to several microtubules) with mammalian cells which also undergo a gamut of division types. Therefore information derived from studies on chromosome inheritance in Drosophila is relevant to human chromosome inheritance and the causes of aneuploidy.

Therefore, there is a need for the identification and analysis of genes and proteins involved in chromosome inheritance. There is a further need to develop a cellular model to study effects of pharmaceutical agents upon chromosomal inheritance. A further need is the use the Drosophila genome as a starting point for such a cellular model.

### SUMMARY OF THE INVENTION

The invention is directed to a method to identify agents, including pharmaceutical agents, that modulate chromosome inheritance. An additional aspect of the invention is a method to diagnose a patient who has, or is at risk for developing, an indication associated with altered chromosome inheritance. A therapeutic method to treat a patient who has, or is at risk for developing, an indication associated with altered chromosome inheritance is also provided. The invention is further directed to one or more polynucleotide(s) at least encoding one or more polypeptide(s) that affect chromosome inheritance. The invention is also directed to polypeptides that affect chromosome inheritance. Another aspect of the invention is a method for identifying a polynucleotide that encodes a polypeptide that affects chromosome inheritance.

The method to identify agents that modulate chromosomal inheritance according to the invention involves the use of a sensitized minichromosome that functions as a marker of chromosomal inheritance. In particular, the method of the invention includes screening a candidate agent to determine whether the agent modulates chromosome inheritance. The agent may be a pharmaceutical compound, a peptide, a viral agent, a polynucleotide and the like. This method involves obtaining a normal or germ cell line containing a sensitized minichromosome, such as the J21A minichromosome for the Drosophila genome, or a minichromosome marker (hereinafter, modified cells). The minichromosome will be compatible with the cell line into which it is inserted. The candidate agent and such modified cells are contacted together, the modified cells are allowed to combine and/or divide, and the chromosome inheritance pattern of the minichromosome in progeny cells is determined. An alteration in the minichromosome inheritance pattern indicates that the candidate compound modulates chromosome inheritance. This method is useful to screen for candidate agents that favorably affect chromosome inheritance, for example, to screen for pharmaceutical compounds that may be useful to treat cancer. This method can also be useful to screen for candidate agents that unfavorably affect chromosome inheritance, for example, to determine that the pharmaceutical compound identified as a candidate for another purpose is a mutagenic compound.

The invention is also directed to a method for identifying a polynucleotide of the invention. This method involves determining the inheritance of a sensitized minichromosome in progeny cells following mutagenesis and division of the parent cell. The inheritance of the minichromosome in the progeny cells may additionally be compared to inheritance of the minichromosome in a non-mutagenized cell, wherein an alteration in inheritance of the minichromosome indicates that a mutated polynucleotide affects chromosome inheritance. The polynucleotide can be mutated by various techniques such as, for example, insertion of a genetic construct such as a P element or virus. Alternatively, chemical mutagenesis such as by a chemical, pharmaceutical composition, peptide, polypeptide and the like may be used to mutate a gene of interest. The minichromosome can be, for example, the J21A minichromosome or any of the sensitized minichromosomes described in references described in the “Detailed Description of the Invention.” As mentioned above, these sensitized minichromosomes may also be used in the modified cell line for candidate compound screening. The mutated polynucleotide and the marker can be localized to the same cell, for example, by selective crossing of cell line germ cells, such as from Drosophila. Altered inheritance may be determined, for example, by the monosome transmission assay as described by Cook et al., Genetics, 145:737-747 (1997), and the mutated polynucleotide is characterized, for example by sequencing following inverse PCR. The sequence data can be analyzed, for example, using the Berkeley Drosophila Genome Project (BDGP) WU-BLAST 2.0 and National Center for Biotechnology Information (NCBI) Advanced BLAST servers.

The polynucleotide(s) and polypeptide(s) discovered according to the invention affect chromosome inheritance. Such polynucleotide(s) and polypeptide(s) may be from any organism from which a cell containing a sensitized minichromosome may be obtained and screened. Such cells include but are not limited to, mammalian, insect, yeast and the like. Such cells include human cells. As described herein below, polynucleotides of the invention may be identified by screening lines of appropriate cells, such as Drosophila, which have mutations in their genome, for altered chromosome inheritance. The majority of the Drosophila lines presented herein have mutations in novel loci, and many of those loci have human homologs. This collection of loci includes novel genes involved in inheritance at several levels of control, such as centromere structure and function, chromosome movement (motor proteins), chromosome architecture (sister chromatid cohesion, condensation and replication) or cell-cycle regulation (checkpoint proteins or the APC). These genes equate with and/or incorporate the polynucleotides of the invention. The polynucleotides include those having the nucleotide sequences listed in SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145, 147-149 and described in Tables 4 and 5. The polynucleotides of the invention also include homologs of the indicated nucleic acid sequences and those described in Tables 4 and 5, i.e., the corresponding polynucleotides in organisms other than Drosophila as well as fragments thereof. Thus, the invention includes an isolated polynucleotide comprising a nucleic acid sequence encoding a polypeptide having at least 70% identity to a polypeptide encoded by one or more of the Drosophila sequences. Additionally, the invention includes an isolated polynucleotide comprising a nucleic acid sequence encoding a polypeptide having a substantially similar function to a polypeptide encoded by one or more of the Drosophila sequences. Databases such GenBank may be employed to identify sequences related to the Drosophila sequences. Alternatively, recombinant DNA techniques such as hybridization or PCR may be employed to identify sequences related to the Drosophila sequences.

The invention also provides polypeptides encoded by the polynucleotides of the invention. The polypeptides are involved in the control of chromosome segregation, including arrangement and direction during cell division. The polypeptides are characterized by their amino acid given in SEQ ID NO: 2, 4, 6, 8, 10, 12, 14, 16, 18, 20, 22, 24, 26, 28, 30, 32, 34, 36, 38, 40, 44-46, 48, 50, 52, 54, 56, 58, 60, 62, 64, 66, 68, 70, 72, 74, 76, 78, 80, 82, 84, 87, 88, 90, 93, 94, 96, 98, 100, 102, 104, 106, 108, 111, 112, 115, 116, 118, 120, 122, 124, 126, 128, 130, 132, 134, 138-140, 142, 144 and 146 and described in Tables 4 and 5, and by the polynucleotide sequences that code for the corresponding polypeptides. The invention also includes the isolated polypeptides, polypeptides having at least about 70% identity to the polypeptides having the sequences given in SEQ ID NO: 2, 4, 6, 8, 10, 12, 14, 16, 18, 20, 22, 24, 26, 28, 30, 32, 34, 36, 38, 40, 44-46, 48, 50, 52, 54, 56, 58, 60, 62, 64, 66, 68, 70, 72, 74, 76, 78, 80, 82, 84, 87, 88, 90, 93, 94, 96, 98, 100, 102, 104, 106, 108, 111, 112, 115, 116, 118, 120, 122, 124, 126, 128, 130, 132, 134, 138-140, 142, 144 and 146 and described in Tables 4 and 5, as well as fragments and substitutions thereof. Additionally, the invention includes polypeptides having a substantially similar function to the polypeptides having the sequences given in SEQ ID NO: 2, 4, 6, 8, 10, 12, 14, 16, 18, 20, 22, 24, 26, 28, 30, 32, 34, 36, 38, 40, 44-46, 48, 50, 52, 54, 56, 58, 60, 62, 64, 66, 68, 70, 72, 74, 76, 78, 80, 82, 84, 87, 88, 90, 93, 94, 96, 98, 100, 102, 104, 106, 108, 111, 112, 115, 116, 118, 120, 122, 124, 126, 128, 130, 132, 134, 138-140, 142, 144 and 146. The polypeptide fragments may include functional domains, such as binding sites, for example DNA binding. The polypeptides may also include substitutions that include conservative amino acid substitutions as well as non-natural amino acid substitutions. Such substitutions may be made according to the strategy outlined in Proteins-Structure and Molecular Properties, 2d ed., T. E. Creighton, W. H., Freeman and Company, New York (1993); Wold, F., Posttranslational Protein Modifications: Perspectives and Prospects, Posttranslational Covalent Modification of Proteins, 193:1-12 B. C. Johnson, Ed., Academic Press, New York; Seifter et al., Analysis for protein modifications and nonprotein cofactors, Methods in Enzymol, 182:626-646 (1990) and Rattan et al., Protein Synthesis: Posttranslational Modifications and Aging, Ann. N.Y. Acad. Sci., 663:48-62 (1992).

The invention also provides anti-sense polynucleotides corresponding to the polynucleotides identified as involved in chromosome inheritance. Also provided are expression cassettes, e.g., recombinant vectors, and host cells comprising polynucleotides of the invention.

An additional aspect of the invention is a method for diagnosing a patient who has, or is at risk for developing, an indication associated with altered chromosome inheritance. This method involves determining the presence of a mutation in a polynucleotide, wherein a mutation in the polynucleotide indicates that the patient has, or is at risk for, an indication associated with altered chromosome inheritance. This method is useful, for example, during genetic counseling.

A therapeutic method to treat a patient who has, or is at risk for developing, an indication associated with altered chromosome inheritance is also provided. For example, a patient who has, or is at risk for developing, an indication associated with altered chromosome inheritance can be treated with a compound that reduces the effects of the indication. This treatment could include, for example, gene therapy, antisense therapy, or pharmacological therapy.

### BRIEF DESCRIPTION OF THE DRAWINGS

FIG. 1 shows the dominant interaction between a P element-induced mutation and a sensitized minichromosome. Inheritance of J21A was used as a sensitized assay to detect dominant mutations that affect chromosome inheritance. J21A is only 580 kb and exhibits moderate instability in a monosome transmission assay; it is transmitted to only 27% of the progeny, in comparison to the 50% transmission exhibited by larger, monosomic minichromosomes and 100% transmission for the disomic autosomes and sex chromosomes.

FIG. 2 illustrates a screen for sensitized chromosome inheritance mutations using P element mutagenesis. (A) A schematic of the Drosophila genome. SUPor-P (Roseman et al., Genetics, 141:1061-1074 (1995)) was mobilized from the CyO chromosome using TMS,Sb 2,3ry⁺. (B) An outline of the multiple generations in the screen. (1) CyOP[y⁺] males containing SUPor-P were crossed with TMS,Sb 2,3ry⁺ virgin females containing the transposase activity. (2) A pilot study demonstrated there was no difference in SUPor-P mobilization frequency between males or females. Therefore we mobilized the SUPor-P from males because CyOP[y⁺];TMS,Sb 2,3ry⁺ males were more convenient to collect than CyOP[y⁺];TMS,Sb 2,3ry⁺ virgin females and y;ry virgin females were relatively plentiful. (3,4) New SUPor-P insertions were collected by selecting for P[y⁺] and against the CyO and TMS chromosomes. (4) X chromosome insertions were recovered by collecting non-virgin females (see materials and methods). This was possible because the non-virgin females remated with y;ry;J21A,ry⁺ males and produced offspring with the appropriate phenotype (5). (3,4) J21A was crossed into the SUPor-P-induced mutant background. (6) Three virgin y⁺;ry⁺ (and therefore containing P[y⁺] and J21A) females were collected for each SUPor-P line and three individual transmission tests were performed by outcrossing each female to y;ry males in individual vials. (7) The average transmission rate was calculated from the three vials. If a line exhibited <22% or >37% ry⁺ transmission then it was retained and retested. (8,9) The retests were essentially a repeat of steps 3 and 6, only with 10-15 vials per line instead of only three. (10) Seventy-eight lines retested with significantly interesting transmission rates. These were established as balanced stocks and subjected to further genetic and molecular analyses.

FIG. 3 shows P element insertion locations. (A) The ORFs of 19 Drosophila loci are presented. Exons are depicted as boxes; the 5′ UTRs are dark boxes. P elements are represented by triangles and the orientation is indicated by an arrow (5′ to 3′). Loci with two P insertions at an identical position (oaf, sca and eIF-4E) are indicated by a “2” next to the P insertion site. The ORFs are to scale. (B) A map of eight P insertions within a novel 3 kb locus. The P insertion sites and predicted ORF were established by aligning two ESTs and the P insertion flanking sequences with the genomic clone AC019974 (Table 3). The lines are Scim12¹ (51%), Scim12² (21%), Scim12³ (18%), Scim12⁴ (17%), Scim12⁵ (40%), Scim12⁶ (40%), Scim12⁷ (39%) and Scim12⁸ (19%) [left to right].

FIG. 4 illustrates mitotic chromosome defects in known loci. Wild type metaphase (A), anaphase (B) and interphase (C) figures are presented. The metaphase X, 2 and 3 chromosomes are indicated in panel (A) and the two small dots in the center are the 4 chromosomes. Figures depicting the predominant defects in the mutant lines are presented; rfc4^{Scim13} metaphase (D), and anaphase (E); Gap1^{Scim16.2} metaphase (F) and anaphase (G); eIF-4E^{Scim15.1} metaphase (H) and interphase (I); Rab5^{Scim5} metaphase (colcemid treated) [J]. See text for details and interpretations.

FIG. 5 illustrates mitotic chromosome defects in novel loci. Representative figures depicting the predominant defects are presented for mutant lines. Scim25 metaphases (A, B) and interphase nucleus (B); Scim9 metaphases (C, D); Scim31 metaphase (E) and anaphase (F); Scim24 metaphases (G, H); Scim1 metaphases (I, J); Scim12⁶ metaphase (colcemid treated) [K]. See text for details and interpretations.

FIG. 6 shows a model representing processes involved in chromosome inheritance and associated genes recovered in the screen.

### DETAILED DESCRIPTION OF THE INVENTION

The present invention is founded upon the development of a sensitive minichromosome that acts as a marker of chromosome inheritance for the corresponding cell line. The cell line may be a germ or non-germ cell line that is capable of cell division. The sensitive minichromosome and cell line will be compatible. A cell line carrying the sensitive minichromosome can be challenged with a candidate such as a pharmaceutical agent, peptide, virus and the like. If the challenge causes an alteration in the control mechanisms of chromosome inheritance, an alteration of the inheritance pattern of the sensitive minichromosome will appear in the progeny of the cell line. The alteration then indicates that the candidate favorably affects chromosome inheritance, and would be a desirable anticancer or antiviral agent. Alternatively, the alteration indicates that the candidate causes mutagenesis and would be an undesirable agent for pharmaceutical use. Examples of such minichromosomes and cell lines include the J21A minichromosome from Drosophila as well as the cell lines and minichromosomes characterized in the following references: Au et al., Cytogenet. Cell. Genet., 86:194-203 (1999); Buchowicz, Acta Biochim. Pol., 44(1):13 (1997)(Review); Kapler, Curr. Opin. Genet. Dev., 3(5):730-5 (1993); Crooke et al., Res. Microbiol., 142(2-3):127-30 (1991); Shirakata et al., Virology, 263(1):42-54 (1999); Martino et al., Structure Fold Des., 7(8):1009-22 (1999); Guiducci et al., Hum. Mol. Genet., 8(8):1417-24 (1999). The screen also allows identification of genes and proteins encoded by those genes that are involved in the control and direction of chromosomal inheritance. The Drosophila genome and minichromosome J21A provide a demonstration of the methods and biological materials of the invention.

Drosophila has a minichromosome Dp(1 ;f)1187 (Dp 1187) that may be useful for the study of chromosome inheritance. Dp1187 is derived from the X chromosome and is not required for viability (Murphy and Karpen, Cell, 82:599-609 (1995b); Williams et al., Nature Genetics, 18:30-37 (1998)). It is only 1.3 Mb, it is transmitted normally through mitosis and meiosis, and it binds known kinetochore proteins, demonstrating that it contains a fully functional centromere. The relatively small size of the minichromosome has enabled detailed restriction mapping of the entire minichromosome using pulsed-field gel electrophoresis and Southern analysis (Le et al., Genetics, 141:283-303 (1995); Sun et al., Cell, 91:1007-1019 (1997)). Gamma irradiation mutagenesis, in combination with the above techniques, has enabled the identification of a 420 kb region within Dp1187 that is essential for normal chromosome transmission (Murphy and Karpen, Cell, 82:599-609 (1995b); Sun et al., Cell, 91:1007-1019 (1997)). Irradiation mutagenesis of Dp1187 generated the 580 kb J21A derivative (Murphy and Karpen, Cell, 82:599-609 (1995b); Sun et al., Cell, 91:1007-1019 (1997)). J21A contains only 290 kb of centric heterochromatin, corresponding to two-thirds of the cis-acting DNA sequences required for normal inheritance, and is inherited only half as well as larger derivatives. Previous studies demonstrated that J21A transmission is affected by a heterozygous mutant background for genes required for inheritance while the inheritance of normal chromosomes is unaffected (Murphy and Karpen, Cell, 81, 139-148 (1995a); Cook et al., Genetics, 145:737-747 (1997). This demonstrated that J21A is sensitized for detecting proteins involved in inheritance. The small size of J21A per se likely predisposes sensitivity in a mutant background in several ways including sensitivity to spindle components (Murphy and Karpen, Cell, 81:139-148 (1995a); Cook et al., Genetics, 145:737-747 (1997)), sister chromatid cohesion (Lopez et al. in press) and overall chromosome architecture.

I. Definitions

An “agent” can be a chemical, drug, pharmaceutical composition, polypeptide and the like that modulates chromosomal inheritance.

A “detectable marker” includes any trait that may be screened or selected for, such as expression of a fluorescent protein, drug resistance or the like.

The term “modulate” or “modulates” means an increase or decrease in the occurrence of an event. For example, an agent that modulates chromosomal inheritance in a cell will either increase or decrease chromosomal inheritance in progeny of cells treated with the agent.

The terms “polypeptide,” “protein,” “peptide” are used interchangeably herein.

The term “polynucleotide” or “nucleic acid sequence” are used interchangeably herein and mean an isolated nucleic acid segment. The term encompasses nucleic acid sequences that may be either RNA or DNA.

A “sensitized minichromosome” is a nucleic acid construct that undergoes chromosomal segregation during cell division. Examples of sensitized minichromosomes include, but are not limited to, Dp1187 and J212A. Sensitized minichromosomes of the invention also include nucleic acid constructs having a minimal functional centromere.

The term “substantially similar” refers to nucleotide and amino acid sequences that represent equivalents of the instant inventive sequences. For example, altered nucleotide sequences which simply reflect the degeneracy of the genetic code but nonetheless encode amino acid sequences that are identical to the inventive amino acid sequences are substantially similar to the inventive sequences. In addition, amino acid sequences that are substantially similar to the instant sequences are those wherein overall amino acid identity is 95% or greater to the instant sequences. Modifications to the instant invention that result in equivalent nucleotide or amino acid sequences is well within the routine skill in the art. Moreover, the skilled artisan recognizes that equivalent nucleotide sequences encompassed by this invention can also be defined by their ability to hybridize, under stringent conditions (0.1×SSC, 0.1% SDS, 65° C.), with the nucleotide sequences that are within the literal scope of the instant claims.

II. A Method to Screen for at Least one Agent that Modulates Chromosomal Inheritance

The invention provides a method to screen for an agent that modulates chromosomal inheritance. The method involves contacting a cell that contains a sensitized minichromosome with a candidate agent and determining if the candidate agent increases or decreases inheritance of the minichromosome in progeny of the treated cell.

Sensitized minichromosome: Sensitized minichromosomes for use in the method include the minichromosome Dp1187 and the J21A derivative described herein. Additionally, sensitized minichromosomes may be produced through recombinant methods. These methods are well known in the art and are described within Sambrook et al., Molecular Cloning: A Laboratory Manual (2d ed., Cold Spring Harbor Laboratory Press, Plainview, N.Y.) (1989). Such minichromosomes may be exemplified by those having the 420 Kb region of Dp1187 or the 290 Kb region of J21A cloned into a vector backbone to form a recombinant minichromosome that is heritable. The recombinant minichromosomes may also contain a minimal element that provides for inheritance of the minichromosome. Methods for isolation of minimal elements required for chromosomal segregation and which confer inheritance on a vector sequence are within the skill of the art in light of the disclosure herein. Sensitized minichromosomes may also include genes that encode selection markers or marker genes. Such selection markers include those that confer resistance to a chemical, such as a drug. Such markers and methods are well know in the art. Sensitized minichromosomes may also include marker genes that express a detectable product. Examples of such gene products include fluorescent proteins, such as green fluorescent protein, red fluorescent protein, yellow fluorescent protein, cyan fluorescent protein and the like.

Cells for use in the method: Any cell may be used within the assay method that is compatible with a sensitized minichromosome. Such cells may be germ-line or non-germ line cells. Additionally, cells may be obtained from a multitude of organisms, such as mammals, insects, yeast and the like. Examples of cells in common use include 3T3, BHK21, MDCK, HeLa, PtK1, L6 PC12 and SP2 cells. Additional cells may be obtained from the American Type Culture Collection. Hay et al., eds., American Type Culture Collection Catalogue of Cell Lines and Hybridomas, 6th ed. Rockville, Md.: American Type Culture Collection, 1988. These cells can be grown under any condition that allows them to divide. Cell and tissue culture conditions are well known in the art. Ham, Proc. Natl. Acad. Sci. USA, 53:288 (1965); Loo et al., Science, 236:200 (1987); Sato et al., eds. Growth of Cells in Hormonally Defined Media. Cold Spring Harbor, N.Y.: Cold Spring Harbor Laboratory (1982).

Methods for detecting inheritance of the minichromosome: Many methods may be used within the method to detect inheritance of a sensitized minichromosome. Such methods include, but are not limited to, fluorescent in situ hybridization (FISH), drug resistance, fluorescence and the like. The detection methods may involve lysis of the cell or may involve analysis of a whole cell or cells. In one embodiment of the method, cells may be contacted with a candidate agent and then the inheritance of a sensitized minichromosome may be determined through lysis of the cells and hybridization with a probe that is specific to the minichromosome. Probes may be prepared that are labeled in a variety of ways that include fluorescence, radiolabel, antibody label or many other art recognized methods. Detection methods in such cases include, but are not limited to, use of fluorescent microscopy, autoradiography, phosphorimaging, and the like. In another embodiment, the sensitized minichromosome expresses a fluorescent gene product, such as green fluorescent protein (GFP), yellow fluorescent protein (YFP), cyan fluorescent protein (CFP) and the like. Inheritance of the minichromosome may be determined through detecting fluorescence of the gene product in progeny of the treated cell through use of fluorescent microscopy or fluorescence activated cell sorting (FACS). In another embodiment of the invention, drug resistance may be used to determine inheritance of the sensitized minichromosome. This may be done by treating a cell containing a sensitized minichromosome that confers drug resistance with a candidate agent. A portion of the progeny of the treated cell are then plated on a plate containing a selective drug and on a plate lacking the selective drug. Inheritance of the drug may be determined by comparing the number of colonies on the plate lacking the selective drug compared to the number of colonies on the plate containing the drug. One of skill in the art will recognize that the invention encompasses a multitude of art recognized methodologies that can be used to detect a minichromosome that may be used according to the method.

Agents: Agents include chemical, biological, or physical agents. It is contemplated that the inventive method may be used to identify agents useful for treatment of disease or afflictions related to abnormal chromosomal inheritance. Examples of chemical agents include, but are not limited to, pharmaceuticals and pharmaceutical compositions. Biological agents are exemplified by gene therapy agents, therapeutic polypeptides, anti-sense constructs and the like. Physical agents include light, ionizing radiation, electromagnetic radiation and the like. It is also contemplated that the inventive method may be used as a screen for agents, such as chemicals, pharmaceuticals, and other therapies to ensure that the agents do not adversely affect chromosomal inheritance.

The above described methods are illustrative of the many ways in which inheritance of a sensitized minichromosome may be determined and are not meant to be limiting in any way.

III. A Method for Diagnosing a Patient who has, or who is at Risk of Developing an Indication Associated with Altered Chromosome Inheritance

The invention provides a method for determining if a patient has a mutated gene that may predispose them or their progeny to development of genetic disease. Such information is useful for purposes of genetic counseling. The method involves screening a patient for deleterious mutations occurring in genes involved with chromosomal inheritance. Such genes are described herein and may also be identified according to the methods described herein.

Methods for identifying mutations in nucleic acid sequences are well known in the art. Briefly, a nucleic acid sample can be obtained from a patient through collection and extraction of a tissue or bodily fluid sample, such as blood. The collected nucleic acid may then be probed to detect the presence of a mutation. Examples of methods to detect mutations in isolated nucleic acids include, sequencing, digestion with restriction enzymes, polymerase chain reaction, nucleic acid hybridization and the like.

The invention describes nucleic acid sequences, polypeptides, and methods for identifying additional genes involved with chromosomal inheritance that may be used in conjunction with the diagnostic method. For example, the nucleic acid sequences disclosed herein, and orthologs thereof, may be used as probes to screen patients for mutations in genes involved with inheritance. Alternatively, the nucleic acid sequence of the genes and orthologs identified herein may be compared to the sequence of nucleic acid isolated from a patient to determine if the patient has an alteration in a gene involved with chromosomal inheritance.

IV. A Method to Treat a Patient who has, or is at Risk for Developing an Indication Associated with Altered Chromosomal Inheritance

The invention provides a method to treat a patient having an affliction associated with altered chromosomal inheritance or to lessen the risk of onset of an affliction associated with altered chromosomal inheritance. The method involves administering an agent that affects inheritance of a chromosome to the patient in need thereof. Such an agent may be identified according to the methods disclosed herein. Agents of the invention include chemicals, pharmaceutical compositions, gene therapy agents and the like.

Gene therapy agents: In one embodiment of the invention, a gene therapy agent able to express a polypeptide involved in chromosomal inheritance is administered to a patient identified as having reduced expression of the polypeptide in the form of a vector. Vectors include, but are not limited to, a plasmid, a phagemid, a raus sarcoma virus (RSV) vector or an adenoviral vector. In addition, a variety of viral vectors, such as retroviral vectors, herpes simplex virus (U.S. Pat. No. 5,288,641), cytomegalovirus, and the like may be employed. Recombinant adeno-associated virus (AAV) and AAV vectors may also be employed, such as those described in U.S. Pat. No. 5,139,941. Techniques for preparing replication-defective infective viruses are well known in the art, as exemplified by Ghosh-Choudhury and Graham, Biochem. Biophys. Res. Comm., 147:964 (1987); McGrory et al., Virology, 163:614 (1988); and Gluzman et al., Eukaryotic Viral Vectors, Gluzman ed., pp. 187-192, Cold Spring Harbor Press, Cold Spring Harbor, N.Y. (1982). Plasmid vectors may also be used. Tripathy et al., Proc. Natl. Acad. Sci. USA, 93:10876 (1996).

A replication-defective adenovirus that may be used in the practice of the present invention. An example of a replication-defective adenovirus is one that lacks the early gene region E1 or the early gene regions E1 and E3. The DNA of interest, such as a promoter and a gene of the present invention, may be inserted into the region of the deleted E1 and E3 regions of the adenoviral genome. In this way, the entire sequence is capable of being packaged into virions that can transfer the inserted DNA into an injectable host cell.

The vector of the present invention may be dispersed in a pharmaceutically acceptable solution. Such solutions include neutral saline solutions buffered with phosphate, lactate, Tris, and the like. Vectors may be purified through use of buoyant density gradients, such as cesium chloride gradient centrifugation, through use of gel filtration chromatography or filter sterilization.

Formulations of compounds: In cases where compounds such as the polypeptides of the invention or those pharmaceutical compounds that modulate the action of the polypeptides of the invention are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts.

Pharmaceutically acceptable salts are obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids also are made. The compounds may be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained. The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions that can be used to deliver the compounds of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 mM, preferably, about 1 to 50 mM, most preferably, about 2 to about 30 mM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

V. A Method to Identify Polynucleotides Involved with Chromosome Inheritance

The invention provides a method to identify polynucleotides involved with chromosome inheritance determined through use of a sensitized minichromosome.

In one embodiment, the method involves mutagenizing a cell that contains a sensitized minichromosome and determining if inheritance of the minichromosome is affected by mutagenesis. If inheritance of the minichromosome is increased or decreased following mutagenesis, the mutagenized polynucleotide producing the alteration can be identified through use of an art recognized method. In another embodiment of the method, a sensitized minichromosome is introduced into a mutagenized cell and inheritance of the minichromosome in the progeny of the mutagenized cell is compared to the inheritance of the minichromosome in a non-mutagenized control cell. If the inheritance of the minichromosome in the mutagenized cell is increased or decreased relative to inheritance in the non-mutagenized control cell, the mutagenized polynucleotide producing the alteration is identified according to art recognized methods. In yet another embodiment of the method, a nucleic acid construct, such as a plasmid containing a gene of interest or a genomic or cDNA library, may be mutagenized in vitro and then introduced into a modified cell that contains a sensitized minichromosome. Inheritance of the minichromosome in the progeny of the modified cell is then determined as described above. Use of such a method allows for the identification of mutants that dominantly interfere with cellular machinery involved with chromosomal inheritance. Methods to mutagenize nucleic acids in vitro are well known in the art. (Greenfield et al., Biochim. Biophys. Acta., 407:365 (1985); Botstein and Shortle, Science, 229:1193 (1985)).

Examples and descriptions of cells and minichromosomes suitable for use according to the method are described herein (Section II).

Cells may be mutagenized according to many methods well known in the art. These methods include, but are not limited to, use of chemical mutagenesis, ultraviolet light, radiation, viral infection and the like. Such methods are further explained and described in the examples section included herein.

Methods to identify mutated polynucleotides: Methods to identify mutated polynucleotides are well known in the art. For example, one can introduce a library, such as a cDNA or genomic library, into mutated cells that display altered inheritance of a sensitized minichromosome and then select for cells that display a reverted phenotype based on minichromosome inheritance. The complementing polynucleic acid clone can then be recovered and sequenced to identify the polynucleotide responsible for the reverted phenotype. Another method for isolating a polynucleotide that is involved with chromosomal inheritance is to use an integrating virus to mutagenize the modified cell and to then isolate the polynucleotide of interest based on localization of the virus sequence. This viral sequence can be isolated through use of standard techniques, such as polymerase chain reaction, hybridization with probes that recognize the viral sequence, and other like methods. Such methods are well known in the art and are included within the scope of the invention. Once a polynucleotide is identified, a corresponding functional polynucleotide can be introduced into the mutagenized cell to compliment the inheritance phenotype and confirm the identity of the polynucleotide as one involved in chromosomal inheritance. Other methods for identifying polynucleotides are disclosed within the examples.

VI. Polynucleotides and Constructs Containing the Polynucleotides as well as Polypeptides of the Invention

The invention provides isolated polynucleotides involved with chromosomal inheritance as well as expression cassettes and vectors containing the polynucleotides. Accordingly, the invention also provides polypeptides involved with chromosomal inheritance.

Polynucleotides and polypeptides: The polynucleotides of the invention include those listed in SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 and 147-149. The invention also provides polynucleotides having 70% or greater sequence identity to the polynucleotides listed in SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 and 147-149. In another embodiment, the invention provides polynucleotides having 80% or greater sequence identity to the polynucleotides listed in SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 and 147-149. In yet another embodiment, the invention provides polynucleotides having 90% or greater sequence identity to the polynucleotides listed in SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 and 147-149. The invention also provides polynucleotides that encode polypeptides having substantially similar function to a polypeptide encoded by a polynucleotide listed in SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 and 147-149. Such polynucleotides include orthologous polynucleotides isolated from other organisms, such as humans.

The polynucleotides of the invention include polynucleotides having mutations in these sequences that encode the same amino acids due to the degeneracy of the genetic code. For example, the amino acid threonine is encoded by ACU, ACC, ACA and ACG. It is intended that the invention includes all variations of the polynucleotides of SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 and 147-149 that encode the same amino acids. Such mutations are known in the art (Watson et al., Molecular Biology of the Gene, Benjamin Cummings, 1987). Mutations also include alteration of a polynucleotide to encode for conservative amino acid substitutions.

Conservative amino acid substitutions include groupings based on side chains. Members in each group can be substituted for one another. For example, a group of amino acids having aliphatic side chains is glycine, alanine, valine, leucine, and isoleucine. These may be substituted for one another. A group of amino acids having aliphatic-hydroxyl side chains is serine and threonine. A group of amino acids having amide-containing side chains is asparagine and glutamine. A group of amino acids having aromatic side chains is phenylalanine, tyrosine, and tryptophan. A group of amino acids having basic side chains is lysine, arginine, and histidine. A group of amino acids having sulfur-containing side chains is cysteine and methionine. For example, replacement of a leucine with an isoleucine or valine, an aspartate with a glutamate, a threonine with a serine, or a similar replacement of an amino acid with a structurally related amino acid may be accomplished to produce a mutant polypeptide of the invention.

Expression cassettes and vectors: A polynucleotide of the invention can be inserted into an expression cassette or a recombinant expression vector. An expression cassette refers to a DNA sequence capable of directing expression of a particular nucleotide sequence in an appropriate host cell, comprising a promoter operably linked to the polynucleotide of interest. The expression cassette may also comprise a termination sequence operably linked to the polynucleotide of interest. A recombinant expression vector generally refers to a plasmid, virus or other vehicle known in the art that has been manipulated by insertion or incorporation of a polynucleotide. For example, a recombinant expression vector of the invention includes a polynucleotide encoding a polypeptide that affects chromosomal inheritance. The expression vector typically contains an origin of replication, a promoter, as well as genes which allow phenotypic selection of a cell transformed with the vector. Vectors suitable for use in the present invention include, but are not limited to, the T7-based expression vector for expression in bacteria (Rosenberg et al., Gene, 56:125 (1987)), the pMSXND expression vector for expression in mammalian cells (Lee and Nathans, J. Biol. Chem., 263:3521 (1988)) and baculovirus-derived vectors for expression in insect cells. The polynucleotides of the invention can also be expressed in plant cells using vectors such as cauliflower mosaic virus (CaMV) and tobacco mosaic virus (TMV). The construction of expression vectors and the expression of genes in transfected cells involves the use of molecular cloning techniques that are well known in the art. (Sambrook et al., Molecular Cloning—A Laboratory Manual, Cold Spring Harbor Laboratory, Cold Spring Harbor, N.Y., 1989; and Current Protocols in Molecular Biology, M. Ausubel et al., eds., (Current Protocols, a joint venture between Greene Publishing Associates, Inc. and John Wiley & Sons, Inc., most recent Supplement)). These methods include in vitro recombinant DNA techniques, synthetic techniques and in vivo recombination. (Maniatis, et al., Molecular Cloning A Laboratory Manual, Cold Spring Harbor Laboratory, N.Y., 1989).

An insect cell based expression system may also be used to express the polynucleotides of the invention. In one such system, Autographa californica nuclear polyhedrosis virus (AcNPV) is used as a vector to express foreign polynucleotides. The virus grows in Spodoptera frugiperda cells. The polynucleotide encoding a polypeptide of the invention may be cloned into non-essential regions (for example, the polyhedrin gene) of the virus and placed under control of an AcNPV promoter (for example the polyhedrin promoter). Successful insertion of the sequences coding for a polypeptide of the invention will result in inactivation of the polyhedrin gene and production of non-occluded recombinant virus (i.e., virus lacking the proteinaceous coat coded for by the polyhedrin gene). These recombinant viruses can then be used to infect S. frugiperda cells in which the inserted gene is expressed. (Smith et al., J. Viol., 46:584 (1983); Smith, U.S. Pat. No. 4,215,051).

The vectors of the invention can be used to transform a host cell by methods well known in the art such as viral infection, electroporation, CaCl₂ or PEG transformation. By transform or transformation is meant a permanent or transient genetic change induced in a cell following incorporation of a new polynucleotide (i.e., nucleic acid exogenous to the cell). A permanent genetic change may be achieved by insertion of the polynucleotide into the genome of the cell through mechanism such as viral integration or homologous recombination. These methods may be used in many cell types that include, but are not limited to, mammalian, insect, plant, bacterial, yeast and the like.

Mammalian cell systems which utilize recombinant viruses or viral elements to direct expression of an operably linked polynucleotide may be engineered. For example, when using adenovirus expression vectors, a polynucleotide of the invention may be ligated to an adenovirus transcription/translation control complex e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a polypeptide of the invention in infected hosts (Logan & Shenk, Proc. Natl Acad. Sci. USA, 81:3655-3659 (1984)). Alternatively, the vaccinia virus 7.5K promoter may be used. (Mackett et al., Proc. Natl. Acad. Sci. USA, 79:7415-7419 (1982); Mackett et al., J. Virol., 49:857-864 (1984); Panicali et al., Proc. Natl. Acad. Sci. USA, 79:4927-4931 (1982)). Vectors based on bovine papilloma virus may also be used which have the ability to replicate as extrachromosomal elements. (Sarver et al., Mol. Cell. Biol., 1:486 (1981)). These vectors are capable of a very high level of expression. Alternatively, a retrovirus can be modified for use as a vector capable of introducing and directing the expression of a polynucleotide of the invention in host cells. (Cone & Mulligan, Proc. Natl. Acad. Sci. USA, 81:6349-6353 (1984)). The herpes virus can also be used a vector. The use of herpes simplex virus vectors is well known in the art and has been described. (Glorioso et al., Annu. Rev. Microbiol., 49:675-710 (1995); U.S. Pat. No. 6,106,826).

Antisense constructs and expression cassettes and vectors able to produce an antisense message are also provided by the invention. These antisense constructs can be according to methods well known in the art and described herein. (Sambrook et al., Molecular Cloning: A Laboratory Manual, Cold Spring Harbor Laboratory, Cold Spring Harbor, N.Y. (1989); Current Protocols in Molecular Biology, M. Ausubel et al., eds., (Current Protocols, a joint venture between Greene Publishing Associates, Inc. and John Wiley & Sons, Inc., most recent Supplement); Burden-Gulley and Brady-Kalnay, J. Cell Biol., 144:1323-1336 (1999)). Briefly, a polynucleotide of the invention may be placed into an expression vector such that the polynucleotide is in reverse orientation relative to the promoter causing transcription of an antisense message. These antisense messages can be used to inhibit the expression of a selected gene through inhibition resulting from duplex formation between the antisense and sense message.

The invention is described with reference to various specific and preferred embodiments and techniques. It should be understood, however, that many variations and modifications may be made while remaining within the scope of the invention.

### EXAMPLES

### Materials and Methods

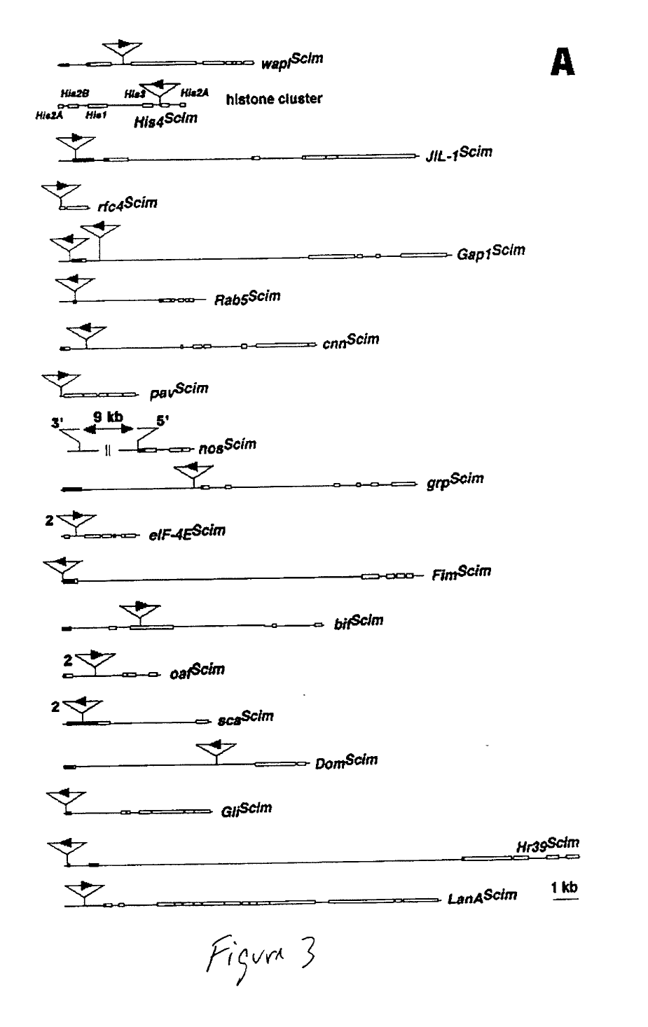

Drosophila stocks and culture: The SM1 and TM3 balancer chromosome and y;ry stocks are described by Cook et al., Genetics, 145:737-747 (1997)). The strain containing the SUPor-P (suppressor-P) element on the CyO balancer chromosome is described by Roseman et al., Genetics, 141:1061-1074 (1995). The P element was mobilized using P[ry⁺2-3](99B) transposase on the TMS balancer chromosome (Robertson et al., Genetics, 118:461-470 (1988)) [FIG. 2A]. The genotypes of the GFP balancer chromosome lines are w⁺; In(2LR)noc^{4L}sco^{rv9R}, b¹/CyO, P{w^{+mC}=ActGFP}JMR1 for the 2 chromosome, w⁺; Sb ¹/TM3, P{w^{+mC}=ActGFP}JMR2, Ser¹ for the 3 chromosome and FM7i, P{w^{+mC}=ActGFP}JMR3/C(1)DX, f¹ for the X chromosome (see http://flybase.bio.indiana.edu/.bin/fbquery/). Flies were grown on standard corn meal/agar media at 25° C.

Recovery of insertions on the X chromosome: The mobilization-generating crosses were performed in vials as a precaution against recovering multiple lines from the same insertion event. This involved setting up >10,000 vials which made the collection of virgin females containing new mobilization events impractical. Eleven individual loci on the X chromosome (Tables 2, 3) were recovered by collecting y⁺;ry non-virgin females and crossing in J21A (FIG. 2B). Males carrying the P element and J21A (y⁺;ry⁺) were selected and outcrossed to y;ry virgin females. Incorporating this extra generation enabled selection of y⁺;ry⁺ virgin females in the next generation that had the new P insertion and J21A which could be transmission tested in the normal fashion. Insertions in the Y chromosome were not tested for transmission defects because the transmission tests were performed in females (FIG. 2B). However, about one-hundred and seventy lines were established that exhibit variegated expression of the yellow (y⁺) marker on the P element. These insertions represent a collection of insertions within heterochromatin, some of which are on the Y chromosome (K. W. Dobie, C. Yan and G. H. Karpen, unpublished data).

Monosome transmission assay: The monosome transmission assay is described by Cook et al., Genetics, 145:737-747 (1997)). A one-tailed students t-test demonstrated that lines exhibiting an average of <22% or >37% transmission are usually significantly different (p<0.05) from the normal 27% transmission for J21A (data not shown). If a line met the above transmission criteria using up to three vials per line, the transmission test was repeated with 10-15 vials to make the result more significant (FIG. 2B). A stock was made if a line still exhibited <22% or >37% transmission; 78 lines met this criteria.

Inverse PCR: Genomic DNA preparation, digests and ligations were performed using standard methods (Gloor et al., Genetics, 135:81-95 (1993); Spralding et al., Genetics, 153:135-177 (1999)). All lines were digested independently using three restriction enzymes (HpaII or HhaI or HaeIII) to give the greatest chance of generating 5′ and/or 3′ flanking DNA. Primers tgaaccactcggaaccatttgagcga (KWD2) (SEQ ID NO: 147) and cgatcgggaccaccttatgttatttcatcat (GK36) (SEQ ID NO: 148) were used to amplify off the 5′ end of SUPorP while primers ccagattggcgggcattcacataagt (KWD4) (SEQ ID NO: 149) and GK36 were used to amplify off the 3′ end. Amplified DNA bands were cut from agarose gels and reamplified before sequencing using ABI377 automated sequencers (Perkin Elmer).

Blast search strategy: Sequence data was analyzed using the Berkeley Drosophila Genome Project (BDGP) WU-BLAST 2.0 and National Center for Biotechnology Information (NCBI) Advanced BLAST servers. Initial searches were performed using a blastn search of the BDGP non-redundant (nr) DNA database. This provided a rich source of hits on large genomic clones (20-350 kb), known Drosophila genes, expressed sequence tags (ESTs) and P insertions from other screens (Enhancer-Promoter [EP: RØRTH 1996] or lethal P lines [Spralding et al., Genetics, 153:135-177 (1999)]). At least one large clone was obtained for every line that was generated from inverse PCR sequence data. This facilitated searches in BDGP using 5 kb of sequence surrounding the insertion site (2.5 kb either side) to identify neighboring genes, ESTs and other P elements. These 5 kb blocks and ESTs were also used to search for homologs in other species by performing a blastx search of the NCBI nr database. Hits on Drosophila ESTs demonstrate that the P insertion is close to or within an expressed sequence and homology with DNA flanking other lethal P insertions demonstrate that the insertion is close to or within a gene that is essential for viability. Protein accession numbers for similar human genes for Drosophila wap1, grp, Gli, cnn, pav, eIF-4E, Gap1 and JIL-1 were directly available from FlyBase reports (http://flybase.bio.indiana.edu/) while the Online Mendelian Inheritance in Man (OMIM) database (within the FlyBase reports) was used for Fim, Rab5, Hr39, His4, Sca, LanA. ESTs were identified for 80% of the novel loci. Blastx searches in NCBI using EST sequences from the novel loci were performed to identify predicted gene products (denoted by “GC” followed by a number). Similar human sequences for the novel loci were determined using the Genome Annotation Database of Drosophila (GadFly: http://flybase.bio.indiana.edu/).

Stage of lethality and cytological analysis of mitotic defects: Embryo collections were performed on apple juice plates supplemented with yeast paste to encourage egg laying. The stage of lethality was determined using standard procedures and by normalizing to inter se crosses using control non-lethal +/P, +/SM1 and +/TM3 lines. A line was classified as lethal if it exhibited <5% of the expected number of P/P flies and semilethal if it exhibited between 5% and 50% of the expected number of P/P flies (Ashburner, Drosophila: A Laboratory handbook, Cold Spring Harbor Laboratory Press, Cold Spring Harbor, N.Y.: 421 (1989)). Homozygous lethal and semilethal lines were crossed with GFP balancer chromosome lines which enabled discrimination of P/GFP and P/P larvae using a Zeiss Axiophot fluorescence microscope fitted with an FITC filter. Larval neuroblast squashes were prepared using a standard method (Ashburner, Drosophila: A Laboratory handbook, Cold Spring Harbor Lavoratory Press, Cold Spring Harbor, N.Y.: 8-9 (1989)) with some modifications. Neuroblasts were fixed in 45% acetic acid followed by 60% acetic acid for 45 sec each. Squashes were performed in 60% acetic acid and chromosomes were stained in 1 μg/ml DAPI. Citrate swelling was not used as it can result in artificial sister chromatid separation. Chromosome defects were examined at 63× magnification with 1.25× optivar on a Zeiss Axiophot fluorescence microscope and classified independently by two investigators.

### RESULTS

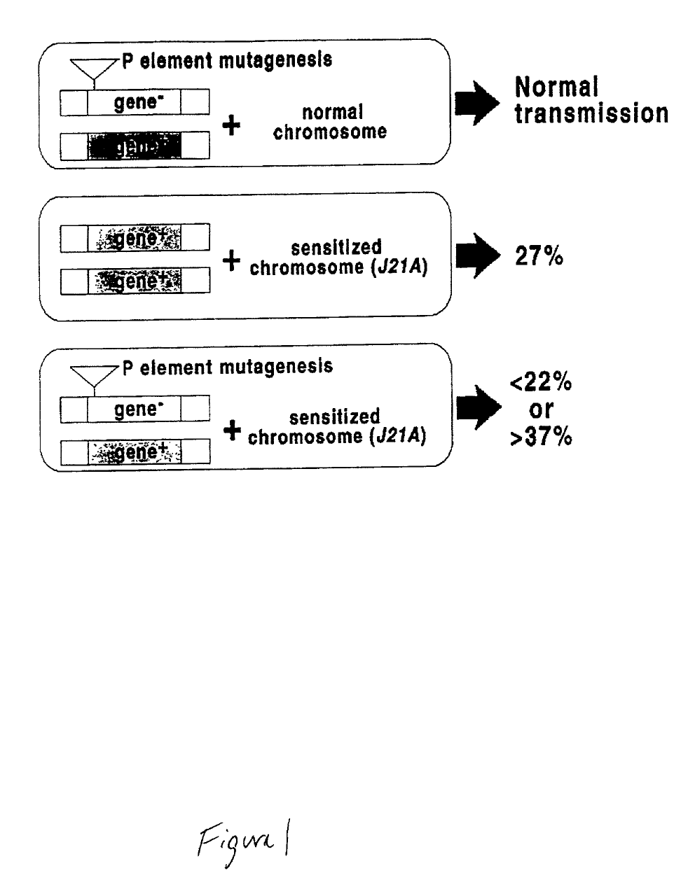

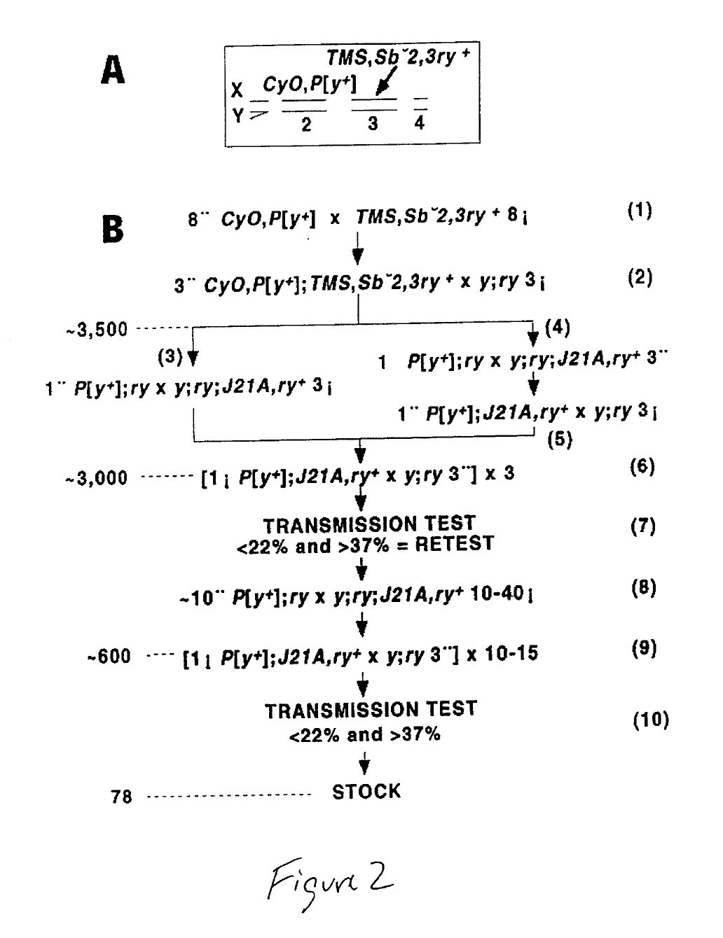

A screen was designed to search for new genes involved in chromosome inheritance by identifying mutations that affect inheritance of a sensitized minichromosome, such as J21A. A screen using inheritance of a sensitized minichromosome, such as J21A, as a dosage-sensitive substrate enabled the recovery of mutations that would otherwise be undetectable as heterozygotes and/or lethal as homozygotes (FIG. 1). P element mutagenesis was used for this screen due to the ease with which a “transposon-tagged” gene can be cloned using inverse PCR amplification of the flanking DNA. (Gloor et al., Genetics, 135:81-95 (1993); Spradling et al., Genetics, 153:135-177 (1999)). Use of P element mutagenesis greatly facilitated cloning and subsequent molecular-genetic analysis. However, other methods of mutagenesis may be readily used to mutagenize genes involved with chromosome inheritance. Examples of such methods include use of chemicals, ultraviolet light, transposable elements, viruses, as well as many other methods known in the art. Through use of the inventive method, seventy-eight sensitized chromosome inheritance modifier (Scim) lines were isolated that exhibited significantly altered levels of J21A inheritance. Analysis of DNA sequences flanking the P elements combined with the complete euchromatic sequence of Drosophila (Adams et al., Science, 287:2185-2195 (2000)) identified several known genes, many of which have chromosome inheritance-related functions. This result demonstrated that the method was able to identify genes related to chromosomal inheritance. The majority of lines represent mutations in novel Drosophila loci, many of which have human homologs, and most have been localized to a specific region of the Drosophila genomic sequence. This collection includes several novel genes involved in inheritance at several levels of control, such as centromere structure and function, chromosome movement (motor proteins), chromosome architecture (sister chromatid cohesion, condensation and replication) or cell-cycle regulation (checkpoint proteins or the APC).

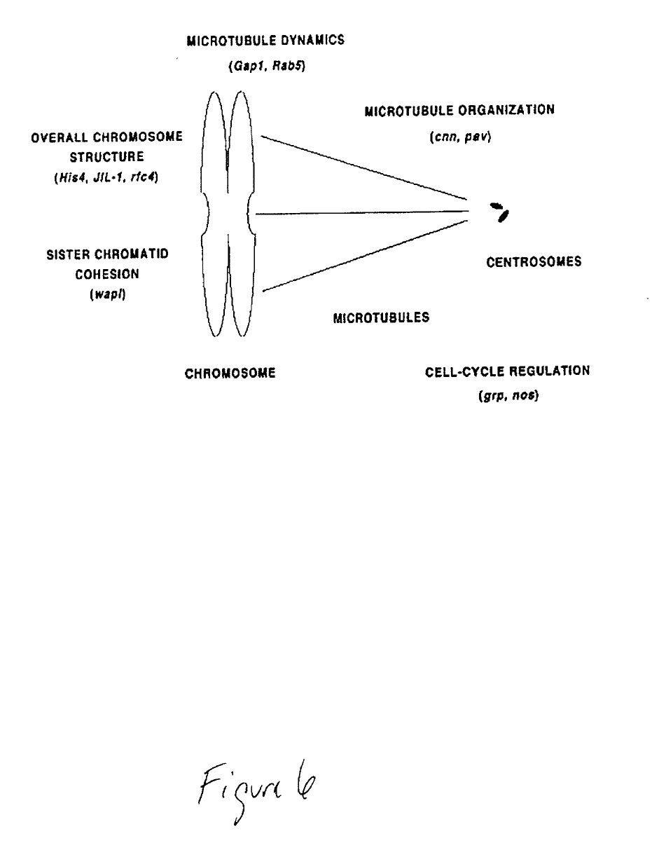

Analyses demonstrated that inheritance of the J21A minichromosome derivative is sensitive to mutations in genes important for inheritance. Of the about three-thousand lines of Drosophila that were screened, seventy-eight lines exhibited significantly altered levels of chromosome inheritance. In those lines of Drosophila that displayed altered chromosome inheritance, the polynucleotide that was mutated was identified and characterized. Through use of the inventive method, seventy-eight lines were recovered that exhibit altered J21A inheritance; sixty-nine lines exhibit significantly decreased transmission and nine lines exhibit significantly increased transmission. The use of P elements as the mutagenic agent and inverse PCR enabled the generation and isolation of genomic DNA flanking 90% of the P element insertion sites. The completion of the euchromatic Drosophila genome sequence (Adams et al., Science, 287:2185-2195 (2000)) and analysis of the flanking sequences allowed the collection to be divided into two groups. First, P insertions within, or close to, eighteen known Drosophila genes were identified. Mutagenized genes were involved in overall chromosome architecture/organization (His4 and JIL-1), DNA replication (rfc4), sister chromatid cohesion (wap1), microtubule dynamics (Gap1 and Rab5), spindle organization (cnn and pav), and cell cycle regulation (nos and grp) (FIG. 6). Four of these genes (cnn, pav, wap1 and grp) have published abnormal metaphase phenotypes associated with null mutations. It is unlikely that so many loci with chromosome-related functions would be recovered by chance. This result demonstrates that the collection is enriched for genes that promote inheritance. Second, forty-six lines representing thirty-four individual loci at known locations in the genome representing mutations in novel loci were identified. Based on the precedent set by the known loci, it is thought that >50% of the insertions in novel loci (>17 genes) will also have direct roles in chromosome inheritance at several levels of control. Eighteen percent of the lines are lethal or semilethal when homozygous for the P element and exhibit dramatic and distinctive mitotic chromosome defects, demonstrating that these loci play vital and different roles in inheritance. Cytological studies demonstrate that J21A binds the outer kinetochore protein ZW10 (Williams et al., Nature Genetics 18:30-37 (1998)), MEI-S332, another protein that binds the centromere region (Lopez et al. in press) and CID, the functional orthologue of CENP-A, a centromere-specific histone H3-like protein (M. Blower and G. H. Karpen, unpublished results), demonstrating that J21A contains a functional kinetochore.

The small size of J21A per se likely predisposes sensitivity in a mutant background in several ways. First, J21A inheritance is particularly sensitive to reduced levels of kinesin-like proteins (KLPs) that function in spindle organization and cytokinesis. The Drosophila KLP family includes no distributive disjunction (nod), non-claret disjunction (ncd) and kinesin-like protein 3A (klp3A) (Adams et al., Genes Dev., 12:1483-1494 (1998)) and all three genes have very dramatic dominant effects on J21A inheritance (Murphy and Karpen, Cell, 81:139-148 (1995); Cook et al., Genetics, 145:737-747 (1997)). The small size of J21A and/or a limited amount of centric heterochromatin likely renders it susceptible to falling off a compromised spindle. Centrosomes are not present in female meiosis I, and such anastral spindle formation appears to initiate from the chromosomes rather than the poles (Hawley and Theurkauf, Trends Genet., 9:310-317 (1993); Karpen and Endow, Meiosis: Chromosome Behavior and Spindle Dynamics, in Frontiers in Biology, eds. Endow and Glover, Oxford University Press (1998)). Effects on the sensitized minichromosome in females were screened for, and the small size of J21A may make it particularly susceptible to heterozygosity for mutations in spindle components. Second, the lack of substantial amounts of centric heterochromatin likely compromises heterochromatin-specific functions such as cohesion (Lopez et al. in press) and pairing (Demburg et al., Cell, 86:135-146 (1996); Karpen et al., Science, 273:118-122 (1996)). Third, J21A inheritance may be sensitive to the dose of proteins involved in overall chromosome structure and DNA replication because the small size renders it susceptible to stochastic factors that influence chromosome architecture such as limited origins of replication. The unusual properties of J21A enabled the recovery of mutations with diverse functions including spindle dynamics and organization, overall chromosome architecture (e.g., chromatin structure, sister chromatid cohesion, DNA replication) and broader functions such as cell-cycle regulation (FIG. 6).

The Sensitized Screen Identifies Known Genes Involved in Chromosome Architecture

Mutations in wap1 result in an increase in X chromosome nondisjunction during female meiosis and partial separation of all sister chromatids at heterochromatic regions in mitotic chromosomes (Verni et al., Genetics, 154:1693-1710 (2000)). In addition, wap1 is a dominant suppressor of PEV, the heterochromatin-induced gene silencing of normally euchromatic genes (Wakimoto, Cell, 93:321-324 (1998)). These phenotypes imply a role for WAPL in achiasmate chromosome segregation during meiosis, which is heterochromatin-dependent (Karpen et al., Science, 273:118-122 (1996); Demburg et al., Cell, 86:135-146 (1996)), and pairing between the heterochromatic portions of all the sister chromatids during mitosis. It is thought that inheritance of J21A is more sensitive to a mutation in wap1 than the X, 2 and 3 chromosomes which have intact centromeres and large amounts of heterochromatin. The collection of mutations likely contains other genes with roles in heterochromatin biology. Thus, a useful secondary screen would be to test whether the disclosed P insertions enhance or suppress PEV which involves heterochromatic-dependent gene regulation. In addition, it will be useful to determine the cytological reasons for J21A loss in wap1 mutants, which may allow the determination of which heterochromatic functions are related to inheritance.

A P insertion associated with one of the histone H4 (His4) genes was recovered. There are five classes of major histone genes that are grouped as a unit (His2A, His2B, His1, His3, and His4) and, in Drosophila, the histone unit is repeated ˜100 fold to achieve sufficient expression for the enormous task of packaging the genome (Kedes, Annu. Rev. Biochem. 48:837-870 (1979)). The P insertion in His4^{Scim} appears to be close to a copy of His4 at the edge of the histone cluster (data not shown) which may represent a differentially expressed or alternative form of H4. Genetic (Smith et al., Mol. Cell Biol., 16:1017-1026 (1996)) and molecular (Meluh et al., Cell, 94:607-613 (1998)) analyses have demonstrated that histone H4 interacts with Cse4p, the Saccharomyces cerevisiae centromere-specific histone H3-like protein, and that this interaction is required for the formation of centromeric chromatin and faithful chromosome inheritance. Inheritance of J21A would be particularly sensitive to mutations in genes required for centromere formation because it is missing one-third of the functional centromere. Further analysis will utilize a minichromosome deletion series (Williams et al., Nature Genetics, 18:30-37 (1998); Murphy and Karpen, Cell, 81: 139-148 (1995a); Cook et al., Genetics, 145:737-747 (1997)) to determine whether this mutation interacts directly with the centromere.

JIL-1 is localized on chromosomes throughout the cell cycle in Drosophila, to the gene-rich interband regions of larval polytene chromosomes, and is present approximately twice as much on the hypertranscribed male X chromosome compared to autosomes (Jin et al., Mol. Cell 4:129-135 (1999)). The phosphorylation properties and characteristic localization pattern suggest that JIL-1 is a chromosomal kinase involved in regulating the chromatin structure of regions of the genome that are actively transcribed. A mutation in JIL-1 could affect J21A inheritance by either affecting the regulation of a gene or genes required for inheritance or by affecting overall chromatin structure and thereby interfering with inheritance. J21A inheritance may be particularly sensitive to affects on chromatin structure because it has a greatly reduced amount of heterochromatin.

Similarly, the null mutation in rfc4^{Scim} may compromise the assembly of the RFC complex and result in a block at S-phase. In heterozygotes, J21A maintenance may be more sensitive to the dose of replication factors because it is much smaller than the other chromosomes and 50% comprises heterochromatin, which replicates late in S phase. Incomplete replication of J21A would reduce J21A's ability to be transmitted intact during mitosis. Analysis of chromosome morphology in homozygous larvae from rfc4^{Scim} demonstrated dramatic and characteristic chromosome defects associated with this line that are consistent with aberrant replication. The recovery of rfc4 demonstrates the benefit of a sensitized screen to uncover essential loci that have little or no effect on endogenous chromosomes as heterozygous mutations, and this mutation will be an important tool in future analyses of replication in Drosophila. This mutation will also provide an important tool in homologous genes that are found in other organisms that include mammals, such as humans.

The Sensitized Screen Identifies Known Genes Involved in Spindle Organization/function

CNN is required for localization of the other centrosomal proteins such as tubulin, CP60 and CP190 for the assembly of functional centrosomes that are required for mitotic spindle organization. The cnn^{Scim} P insertion may reduce the levels of CNN to a phenocritical level, such that mitotic spindles are sufficient to organize full sized chromosomes but are compromised to a degree that results in loss of J21A. Megraw et al., Development, 126:2829-2839 (1999) describe that mitotic spindle defects in cnn mutants occur in a cumulative fashion and that some mitotic spindles look completely normal. Furthermore, CP190 and tubulin are present at low levels at these centrosomes. This indicates that functional centrosomes can still form even in a cnn mutant background. Ultimately the embryos die at around cycle 12 before cellularization can occur. cnn^{Scim} is not lethal when homozygous for the P element implying that it could be a hypomorphic mutation. The description that the effects of a cnn mutant background are cumulative (Megraw et al., Development, 126:2829-2839 (1999)), in conjunction with a heterozygous hypomorphic P insertion, may explain why J21A is lost in the P insertion background while the other chromosomes are not.

PAV is a member of the kinesin-like protein (KLP) superfamily of microtubule motor proteins that are required for centrosome organization, spindle assembly and chromosome movement (Moore and Endow, Bioessays, 18:207-219 (1996)). Inheritance of J21A appears to be particularly sensitive to reduced levels of the KLPs nod, ncd and klp3A (Murphy and Karpen, Cell, 81:139-148 (1995a); Cook et al., Genetics, 145:737-747 (1997)). J21A inheritance may be compromised in these mutant backgrounds because J21A does not contain all the cis-acting sequences required for normal inheritance. For example, a partially-defective spindle may enhance loss of a partially-defective centromere because it binds fewer microtubules, in comparison to a normal centromere. Another possibility is that J21A inheritance may be particularly compromised due to the greatly reduced size and an incapacity to bind chromokinesins that interact all along chromosome arms, and are thought to mediate antipoleward forces (Murphy and Karpen, Cell, 81:139-148 (1995a); Afshar et al., Cell, 81:129-138 (1995)).

The Sensitized Screen Identified known Genes involved in Neural Development or with Actin-Related functions.

At least four P insertions (two in oaf, and two in sca ) in genes with potential roles in neural development in Drosophila (Bergstrom et al., Genetics, 139:1331-1346 (1995); Lee et al., Genetics, 150:663-673 (1998)) were recovered. There is a strong precedent for problems in neural development being a secondary consequence of defects in early chromosome inheritance. Several mutations have been described in Drosophila which affect PNS development (Kania et al., Genetics, 139:1663-1678 (1995); Salzberg et al., Genetics, 147:1723-1741 (1997)) that result from defects in processes essential for chromosome inheritance including chromatid decatenation (barr: Bhat et al., Cell, 87:1103-1114 (1996)), spindle formation (pav: Adams et al., Genes Dev. 12:1483-1494 (1998)) and cytokinesis (pav: Adams et al., Genes Dev., 12:1483-1494 (1998); pb1: Propopenko et al., Genes Dev., 13:2301-2314 (1999)). Thus, while some of the insertions are in genes that have documented roles in PNS development, they may have primary roles in inheritance. Analysis of mitotic chromosomes from lines with null mutations (imprecise excisions) is necessary to test this hypothesis.

Mutations in two genes (bif and fim) that function in the actin cytoskeleton were also recovered. BIF colocalizes with actin as early as cycle 10 in preblastoderm embryos in defined cytoplasmic domains (Bahri et al., Mol. Cell Biol., 17:5521-5529 (1997)). The colocalization of BIF with actin at early stages of embryogenesis may be significant for chromosome inheritance (see below). Yeast fimbrin (SAC6) is lethal when overexpressed and cells exhibit an abnormal distribution of actin with defects in cytoskeletal organization (Adams et al., Nature, 354:404-408 (1991)). Drosophila embryos undergo 13 rapid cell divisions (syncytial divisions) without cellularization. The organization of the actin cytoskeleton is essential for correct distribution of syncytial nuclei during this period (Foe et al., The development of Drosophila Melanogaster, Eds. Bate and Martinez-Arias, Cold Spring Harbor Laboratory Press, Cold Spring Harbor, N.Y. (1993)). Mutations in proteins that interact with actin may affect the architecture of the actin cytoskeleton during early embryogenesis and have an impact on chromosome inheritance.

The Sensitized P Element Screen to Identify Dominant Mutations that Affect Chromosome Inheritance

The J21A minichromosome is transmitted to only 27% of the progeny in a monosome transmission assay. It was predicted that some heterozygous mutations in genes important for chromosome inheritance would affect J21A transmission, but would not affect inheritance of the sex chromosomes or autosomes (FIG. 1). Indeed, previous studies have shown that J21A transmission is more sensitive than the sex chromosomes or autosomes to heterozygous mutations in genes known to be important for mitosis and meiosis (Murphy and Karpen, Cell, 82:599-609 (1995); Cook et al., Genetics, 145:737-747 (1997)). The SUPor-P element was used to generate the mutations because the presence of two Suppressor of Hairy Wing [Su(Hw)] binding sites enhance its mutagenic properties (Roseman et al., Genetics, 141:1061-1074 (1995)).

SUPor-P was mobilized off the CyO 2 chromosome and about three-thousand five-hundred mobilizations were recovered with the P element inserted in a different chromosome. This strategy enabled the targeting of the entire Drosophila genome (X, Y, 2, 3 and 4 chromosomes) with P element insertions (FIG. 2A). Approximately five-hundred lines were not tested due to insertions in the Y chromosome (transmission tests were performed in females) or flies dying in the food. Each of the three-thousand remaining lines were tested for dominant effects (increases or decreases) on J21A transmission (FIG. 2B). Statistical analyses indicated that lines exhibiting J21A transmission to <22% or >37% of progeny are potentially interesting and warrant further analyses (see Materials and Methods). Seventy-eight lines were recovered with altered J21A transmission, which were named “Scim”, for Sensitized chromosome inheritance modifiers (Table 1). Sixty-nine lines exhibited significantly reduced transmission of J21A, ranging from 9% to 21%. In addition, nine lines were recovered that significantly increased J21A transmission. These ranged from 38% to as high as 51% (completely normal) transmission. The lines that exhibited increased transmission could represent an interesting class of mutations in cell-cycle regulatory genes or genes involved in the repression of proteins involved in inheritance (see Discussion). Fourteen lines were lethal or semilethal when homozygous for the P element. Thus, 18% (14 out of 78) of the collection affect genes that are important for viability and strongly influence minichromosome inheritance.

P Insertions in Known Genes Involved in Chromosome Inheritance

P element mutagenesis was utilized to facilitate molecular analysis of the mutated loci. Inverse PCR was used to generate P element flanking DNA sequence and we capitalized on the recent maturation of Drosophila genome sequencing projects (Adams et al., Science, 287:2185-2195 (2000)) to position 90% (70 out of 78) of the lines in the genome. This approach enabled division of the collection into P insertions associated with known (Table 2) or novel (Table 3) loci.

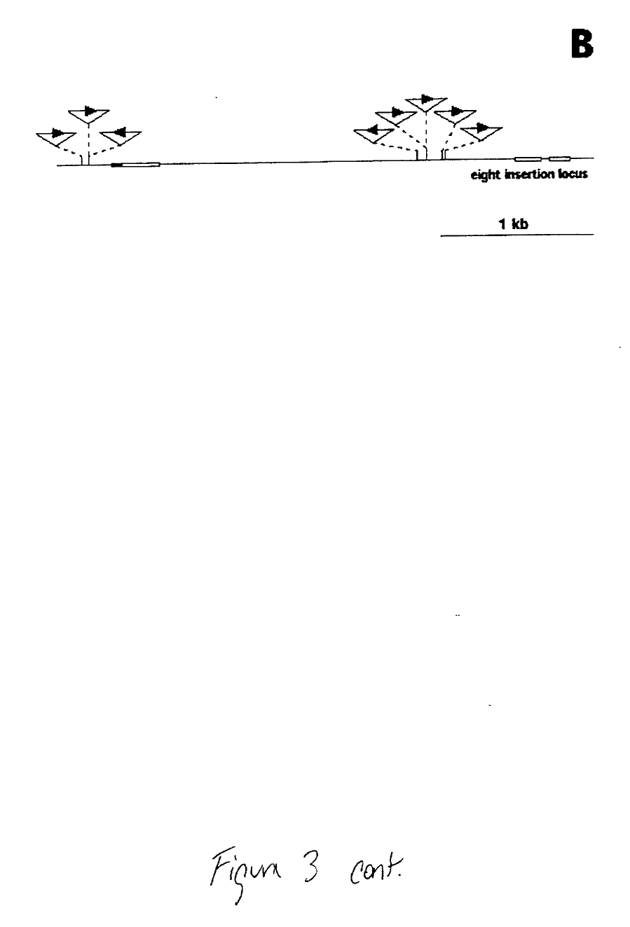

Twenty-two P insertions were recovered within or close to the open reading frame (ORF) of 18 known Drosophila genes (Table 2; FIG. 3A). 78% (14 out of 18) of the known Drosophila loci have homologous sequences in humans. The recovery of a gene in the screen suggests that the normal product is dose limiting (the mutations are dominant) and that it may be important for chromosome inheritance. The P insertion was positioned relative to the ORF for all the known loci and demonstrated that the majority of the P insertions have inserted within or close to the 5′ untranslated region (UTR) [FIG. 3A]. The preference for P elements to insert close to the start of transcription of genes has been documented previously (Spradling et al., Proc. Natl. Acad. Sci. USA, 92:10824-10830 (1995); Liao et al., Proc. Natl. Acad. Sci. USA, 97:3347-3351 (2000) and is confirmed by this study. In some cases the P element could have hopped in and out of a locus in another region of the genome that has the bona fide effect on J21A transmission; however, it is likely that the deviant J21A transmission phenotype is associated with P element-induced mutations in most or all of these loci. Precise excision analysis may be performed to test for reversion of the transmission and viability defects with any lines.

P insertions were identified that are associated with four genes that are known to play a role in chromosome architecture and function: wings-apart like (wap1), histone H4 (His4), JIL-1 and replication factor complex-4 (rfc4) [Table 2]. The P insertion in wap1^{Scim} is within the second intron in wap1 (FIG. 3A). Mutations in wap1 result in partial separation of all sister chromatids in heterochromatic regions of mitotic chromosomes (Verni et al., Genetics, 154:1693-1710 (2000)). The P insertion in His4^{Scim} is ˜50 bp 5′ of the start of transcription of His4 within the histone gene cluster (FIG. 3A) that encodes a fundamental structural subunit of chromatin (Kedes, Annu. Rev. Biochem., 48:837-870 (1979)). JIL-1^{Scim} is a P insertion within the 5′ UTR of JIL-1 (FIG. 3A). JIL-1 can phosphorylate histone H3 in vitro and has been described as a chromosomal kinase involved in regulating the chromatin structure of actively transcribed regions of the genome (Jin et al., Mol. Cell 4:129-135 (1999)). rfc4^{Scim} is a homozygous lethal P insertion within the first exon of rfc4 (FIG. 3A and see below). It is thought that P insertions disrupting wap1, His4, JIL-1 and rfc4 affect chromosome inheritance because the gene products have general roles in maintaining chromosome architecture, which may impact processes such as condensation, cohesion, centromere function or transcription.

Three P insertions were recovered in two loci involved in GTP metabolism. Two independent P insertions are associated with GTPase-activating protein (Gap1); Gap1^{Scim-a} is homozygous viable while Gap1^{Scim-b} is semilethal when homozygous (Table 2). Gap1^{Scim-a} is ˜480 bp 5′ of the start of transcription while Gap1^{Scim-b} is within the first intron (FIG. 3A). While Gap1 has been shown to be involved in Sevenless signaling (Gaul et al., Cell, 68:1007-1019 (1992)), this function is linked to the hydrolysis of GTP, a process that is also essential for the binding of kinetochores to microtubules and chromosome movement during prometaphase (Severin et al., Nature, 388:888-891 (1997)). Rab5^{Scim} is homozygous lethal and the P insertion is within the 5′ UTR of the small GTPase Rab-protein 5 (Rab5) [Table 2; FIG. 3A]. The activated GTP-bound form of Rab5 has a role in the motility of endosomes along microtubules both in vivo and in vitro by interacting with an as yet unidentified kinesin-like motor (Nielsen et al., Nature Cell Biol., 1:376-382 (1999)). The Gap1^{Scim-a}, Gap1^{Scim-b} and Rab5^{Scim} mutations may affect chromosome inheritance due to perturbation of microtubule dynamics (see below).

Insertions were also recovered in two loci, centrosomin (cnn) and pavarotti (pav), that are required for spindle organization (Table 2). The P insertion in cnn^{Scim} is within the first intron of cnn (FIG. 3A). CNN is required for the assembly of functional centrosomes that are in turn required for mitotic spindle organization during early embyogenesis (Megraw et al., Development, 126:2829-2839 (1999)). Mutations in cnn result in dramatic defects in embryonic nuclear division; mitotic spindles are often clumped together and unevenly distributed in the embryo cortex. The P insertion in pav^{Scim} is ˜120 bp 5′ of the start of pav transcription (FIG. 3A). PAV is involved in the organization of the central spindle at telophase and this organization appears to influence the localization of architectural proteins (e.g., Peanut, Actin and Anillin) required for cytokinesis and at least one regulatory protein (Polo kinase) that may have a role in signaling between the centromere, the spindle midzone and the centrosomes (Adams et al., Genes Dev., 12:1483-1494 (1998); Logarinho et al., J. Cell Sci., 111:2897-2909 (1998)). The recovery of genes involved in spindle dynamics and organization is significant because it demonstrates an enrichment for loci with direct roles in chromosome inheritance.

Two independent P insertions are associated with cell cycle regulatory genes. Analysis of genomic sequence flanking nos^{Scim} demonstrated that the 5′ and 3′ parts of the P element appear separated by 9 kb of genomic DNA and that the 5′ region of the P element is ˜260 bp 5′ of the start of transcription for nanos (nos) [Table 2; FIG. 3A]. No evidence was found for an ORF around the 3′ region of the P element. One explanation for this unusual arrangement is that the P element underwent an imprecise excision that separated the 5′ and 3′ ends. While nos has classically been demonstrated to be involved in establishing polarity in the Drosophila embryo (Wang and Lehmann, Cell, 66:637-647 (1991)), it is also involved in the downregulation of mitosis and transcription in the Drosophila germline (Deshpande et al., Cell, 99:271-281 (1999)). Failure to attenuate the cell cycle during early syncytial divisions may promote the loss of the sensitized minichromosome. Second, a line with two P insertions was identified, one within the first intron of grapes (grp) [Table 2; FIG. 3A] and the other within a multiple insertion locus at 23A1-B2 (Scim12⁴, Table 3, FIG. 3b and see below). grp is homologous to chk1/rad27, a DNA checkpoint gene in Schizosaccharomyces pombe. Flies mutant for grp exhibit abnormal metaphases and the protein appears to be involved in DNA replication/damage checkpoint regulation (Fogarty et al., Curr. Biol., 7:418-426 (1997)) via a role in centrosome formation (Sibon et al., Nature Cell Biol., 2:90-95 (2000)). Separation of the two insertions by recombination will allow for the determination of whether one or both of these loci is responsible for the transmission defect.

Two homozygous lethal P insertions were recovered within the first intron of eukaryotic initiation factor 4E (eIF-4E) [Table 2; FIG. 3A]. EIF-4E is required for translation initiation (Hernandez et al., Mol. Gen. Genet., 253:624-633 (1997)) and it is likely that reduced levels of EIF-4E could affect levels of a protein or proteins that are directly involved in inheritance. Mutations in genes were also recovered that likely represent a class of functions that play indirect roles in inheritance including Fimbrin (Fim), bifocal (bif), out at first (oaf) and scabrous (sca) [Table 2; FIG. 3A]. The functions of these loci and how they might impact minichromosome inheritance is discussed herein.

Scim31 is a P insertion within the first intron of Domina (Dom) [Table 3; FIG. 3A]. Dom has been described as a suppressor of position effect variegation (PEV) (M. Strödicke, S. Karberg and G. Korge, unpublished data), implying that it may have a role in chromatin structure and could therefore impact chromosome inheritance. However the P insertion is relatively far from the start of transcription for Dom (˜6 kb 3′, FIG. 3A) when compared with the other insertions and ORFs described here, and sequence analysis has identified novel ESTs that span the insertion site. Therefore the inheritance defect may be due to a disruption in Dom and/or the novel locus represented by the ESTs.

A small subset of insertions were recovered in genes with no obvious role in inheritance including Gliotactin (Gli), Hormone receptor-like in 39 (Hr39) and laminin A (LanA) [Table 2; FIG. 3A]. This latter group could uncover previously unknown functions for these proteins. Finally, three P insertions are associated with mobile genetic elements (mdg3, gypsy, YOYO) and therefore have not been positioned precisely within the genome (Table 2). For example, it has been estimated that the mdg3 element is present at 15-17 sites on different chromosomes (Ilyin et al., Chromosoma, 81:27-53 (1980)). Presumably the transmission defects in Scim's 1, 2 and 3 and the lethal phenotype in Scim1 are due to disruptions in neighboring loci.

In sum, of the eighteen known genes, ten genes (cnn, pav, wap1, His4, JIL-1, rfc4, Gap1, Rab5, nos, grp) have direct roles in chromosome inheritance (56%) and a further five genes (Fim, bif, oaf, sca, eIF-4E) may have an indirect role (28%).

Homozygous Lethal P Insertions in Known Loci Exhibit Mitotic Chromosome Defects

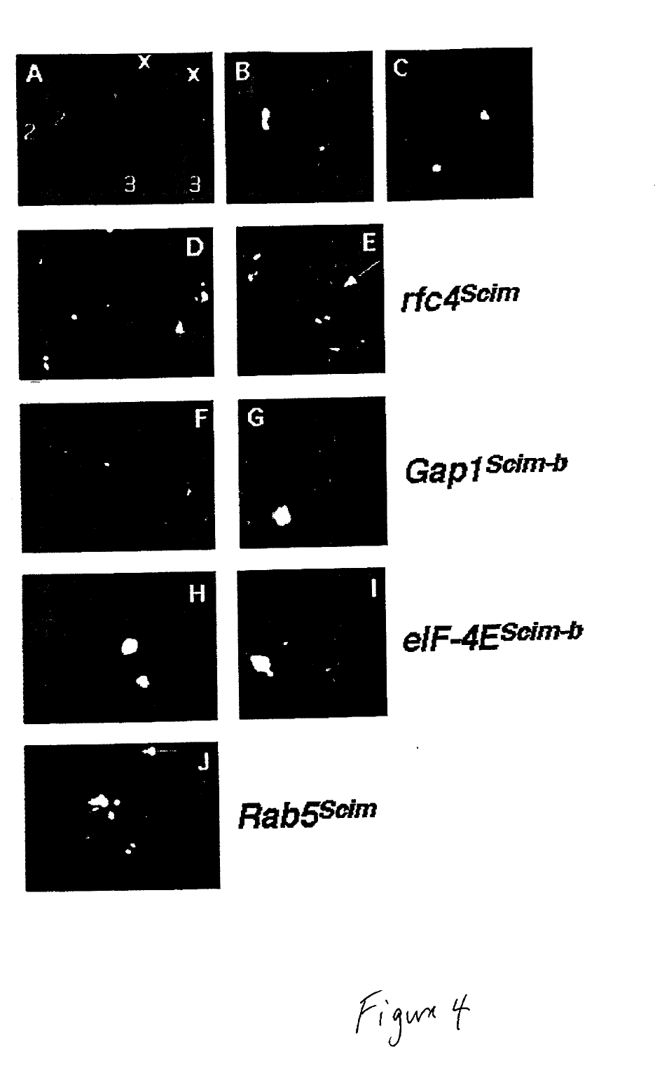

Insertions were recovered in four genes with previously documented abnormal mitotic phenotypes associated with null mutations (wap1: Verni et al., Genetics, 154:1693-1710 (2000), cnn: Megraw et al., Development, 126:2829-2839 (1999), pav: Adams et al., Genes Dev., 12:1483-1494 (1998), grp: Fogarty et al., Curr. Biol. 7:418-426 (1997); Sibon et al., Nature Cell Biol., 2:90-95 (2000)). The insertions associated with cnn, wap1 and grp are not lethal when homozygous for the P insertion and likely represent hypomorphic alleles (Table 2). The analysis of mitotic phenotypes was extended to the lethal insertions in known loci. Analysis of mitotic chromosomes prepared from larval neuroblasts demonstrated a range of dramatic defects associated with all four homozygous larval lethal lines (FIG. 4). The mitotic chromosome phenotypes described below have not been described previously for these known loci.

Harrison et al., Genetics, 139:1701-1709 (1995) describe the cloning of rfc4 (rfc40) in Drosophila and demonstrate that the gene encodes a 40-kDa protein suggesting that rfc4 is the gene for one of the small subunits of the Drosophila RFC complex. The RFC complex is required for loading proliferating cell nuclear antigen (PCNA) onto DNA which in turn tethers the polymerase to the DNA template during synthesis (Mossi et al., J. Biol. Chem., 272:1769-1776 (1997)). Analysis of mitotic chromosomes prepared from rfc4^{Scim} neuroblasts demonstrated fragmented metaphase and anaphase figures (FIG. 4D, E). While individual chromosomes can easily be identified in control metaphase figures (FIG. 4A), the individual chromosomes in rfc4^{Scim} are difficult to identify and some regions of the chromosome arms exhibit what appears to be aberrant condensation (FIG. 4D, E arrows). The lethal insertion in Gap1^{Scim-b} results in precocious sister chromatid separation and aberrant anaphase figures (FIG. 4F, G). Given that Gap1 is involved in spindle formation (see above), it is thought that precocious sister chromatid separation in homozygous mutants may be due to an inability of the chromosomes to segregate to the poles correctly at anaphase. The phenotypes associated with rfc4^{Scim} and Gap1^{Scim-b} are satisfying because they represent what one might predict from mutations in these genes. This suggests that it is possible to make predictions about gene function from the chromosome phenotypes associated with some of the novel loci (see below).

It was very difficult to find any mitotic figures in neuroblast squashes prepared from the eIF-4E and Rab5 lines indicating that the mitotic index is extremely low. The most obvious phenotype associated with the insertions in eIF-4E was fragmented interphase nuclei that were 2-4 times the size of wild type nuclei (FIG. 41). Again, in the rare mitotic figures, the individual chromosome morphology is disrupted and the chromosomes appear decondensed (FIG. 4H). No mitotic figures were found in six slides prepared from the insertion in Rab5. Colcemid treatment enabled the identification of a few mitotic figures, all of which were grossly disrupted, exhibiting chromosome fragmentation (FIG. 4J, arrow). The extreme phenotypes associated with eIF-4E and Rab5 are thought to reflect the general functions of these loci; the affects on chromosome architecture could be due to an indirect role in chromosome inheritance or due to a general affect on cellular health.

Homozygous P-induced mutations in the collection are concluded to result in characteristic defects in autosome and sex chromosome inheritance, and the effect of the mutations is not limited to minichromosome inheritance. Further, novel mitotic chromosome defects are characterized that are associated with homozygous lethal P-induced mutations in known loci.

The Majority of the Collection Comprises P Insertions in Novel Loci

The insertion sites for a further forty-six lines representing thirty-four independent loci have also been identified (Table 3). No known Drosophila genes have been identified that are associated with these lines. This result has been determined after extensive analysis of the P insertion sites. Based on the precedent set by the insertions in known loci, a significant number (>50%) of these lines likely represent mutations in novel genes with roles in chromosome inheritance.

Eight independent insertions at 23A1-B2 were recovered which surprisingly includes four low and four high transmitting lines (Scim12¹-Scim12⁸; Table 3). Analysis of inverse PCR sequence enabled the identification of a large genomic clone (AC019974) and two ESTs which positioned the P insertions relative to a putative ORF (FIG. 3B). The eight insertions are grouped as two clusters with ˜2.5 kb separating them; three insertions are ˜100 bp 5′ of the CAAT and TATA boxes while five lines are between the predicted first and second exons. A conceptual translation of the locus does not contain any signature motifs and database searches suggest that the locus is novel. An epitope-tagged cDNA expressed in S2 embryonic tissue culture cells localizes to the nucleus but is not found on metaphase chromosomes (K. W. Dobie, C. D. Kennedy and G. H. Karpen, unpublished data).

Four independent loci were recovered with two P insertions associated with them (Table 3). Scim8¹ and Scim8² have inserted in the same orientation on the X chromosome and a novel EST is associated with the insertion site. Scim13¹ and Scim13² have inserted in opposite orientations at the same site and are associated with novel ESTs. Surprisingly, they exhibit very different primary J21A transmission rates (19% vs. 39% respectively; Table 3) which may be due to the opposite orientation of the insertions. Scim14¹ and Scim14² have insertions in opposite orientations at the same site; this region is rich with P elements from other screens including a lethal P element line. Given that hypomorphic insertions were recovered in known loci, the homozygous viable P insertions in Scim14¹ and Scim14² may be associated with a locus important for both chromosome inheritance and viability. Scim15¹ and Scim15² are intriguing because they exhibit the lowest (9%) and third lowest (14%) J21A transmission rates recovered from the screen (Table 3). The P insertions are in the same orientation at the same site in 30D 1-2 and are associated with novel ESTs. No known genes or homologs surrounding the P insertion site in the four loci described above were detected. This supports the thought that the above P insertions may represent mutations in four novel loci that affect chromosome inheritance.

The remaining twenty-eight lines are single P insertions that have been localized to a specific region of the genome sequence and likely represent mutations in novel loci (Table 3). ESTs were identified that are associated with 80% (37 out of 46) of the novel lines and 40% (15 out of 37) of these have homologous human sequences (Table 3). Further analysis will be facilitated by the genomic clones, ESTs and other P insertions surrounding these loci. Eight lines were not localized to a specific region of the genome because sequence data from the flanking regions was not generated, potentially due to deletions or rearrangements in the P element sequence, or the absence of relevant restriction sites in the flanking DNA.

Homozygous Lethal P Insertions in Novel Loci Exhibit Mitotic Chromosome Defects

The analysis of mitotic chromosomes in larval neuroblasts was extended to those novel loci that are homozygous lethal. Again, characteristic mitotic defects associated with all of these loci were observed.

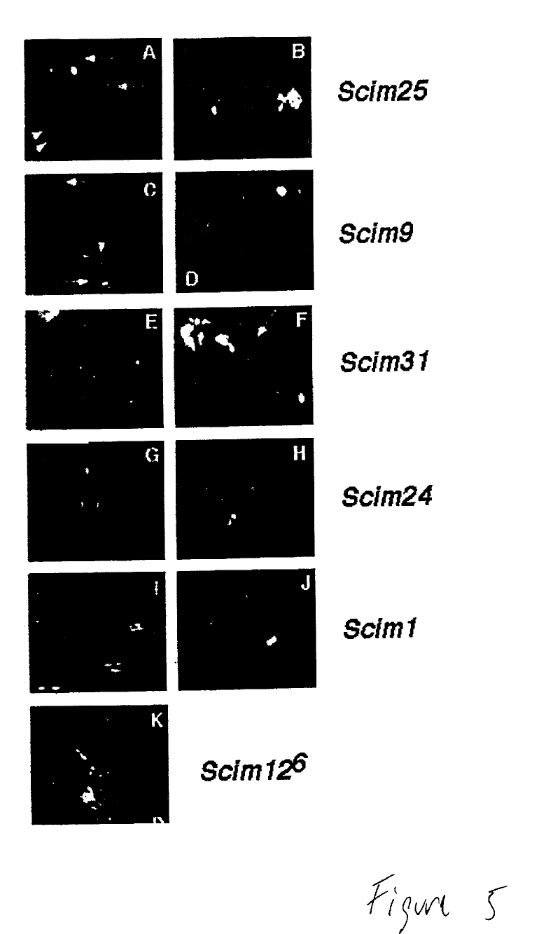

Two novel loci exhibit similar but distinctive degrees of precocious sister chromatid separation. Scim25 has a P insertion associated with a novel locus at 51A1-2 (Table 3). This line exhibits a very low mitotic index and partial loss of sister chromatid cohesion in some mitotic figures (FIG. 5A). The chromosomes appear to lose a degree of cohesion at heterochromatic regions, but the sister chromatids do not completely separate; instead they remain attached by some chromatin (FIG. 5A, arrowsheads). The 4 chromosomes appear as “dumbbells” due to the partial loss of cohesion and the sister chromatids of the Y chromosome are partially separated (FIG. 5A, arrows). This phenotype is very similar to that described for wap1 (Verni et al., Genetics, 154:1693-1710 (2000)), suggesting that the locus disrupted in Scim25 may have a function in maintaining heterochromatin architecture and sister chromatid cohesion/separation. Further, interphase nuclei appear disintegrated and some mitotic figures are clumped together (FIG. 5B). These may represent downstream phenotypes that are induced by precocious loss of cohesion. The P insertion in Scim9 is associated with a novel locus at 10C1-2 (Table 3). Although the mitotic index appears normal, some metaphase figures exhibit partial sister chromatid separation. In FIG. 5C the sister chromatids in one of the 2 chromosomes and the Y chromosome are partially separated (arrows) and one of the 4 chromosomes appears larger than the other, as though the sister chromatids are starting to separate (FIG. 5C, arrowhead). Partial loss of cohesion represents an intermediate phenotype, and many of the metaphase figures exhibit complete sister chromatid separation (FIG. 5D). This phenotype is similar to that observed for Gap1^{Scim-b} (see above) which could suggest a role for Scim9 in microtubule dynamics. A second possibility is that a mutation in a locus required to hold sister chromatids together might also result in a similar phenotype.

Scim31 has a homozygous lethal P insertion within the first intron of Dom (Table 3; FIG. 3). The insertion within this locus results in a unique phenotype; although the mitotic index appears normal, a large number of the mitotic figures exhibit polyploidy (FIG. 5E). Some anaphase figures exhibit missegregation of chromatids, which likely represent early stages in the progression to polyploidy (FIG. 5F, arrow). In this example, only seven sister chromatids, rather than the expected eight chromatids, are present at the lower right pole. A high degree of aneuploidy is also observed, which would be expected to accompany this type of segregation defect (data not shown). Interestingly, Scim31 is one of the high transmitting lines and, as mentioned earlier, novel ESTs associated with the P insertion within the Dom ORF have been identified.

The homozygous lethal P insertion in Scim24 results in a lower than normal mitotic index and some mitotic figures exhibit aneuploidy and/or decondensed chromosomes (FIG. 5G, H). Further, many of the nuclei appear disintegrated, similar to that depicted in Scim25. The P insertion in Scim1 is associated with a mdg3 retrotransposon and the insertion is homozygous lethal (Table 2). Mitotic chromosomes exhibit several defects including disintegrated chromosome arms, decondensed centric heterochromatin and sister chromatid separation (FIG. 51). Further, a high proportion of mitotic figures are so hypocondensed that it is difficult to distinguish individual chromosomes (FIG. 5J). Finally, Scim12⁵ and Scim12⁶ are lethal insertions within the multiple insertion locus at 23A1-B2 (Table 3). Again, colcemid treatment was required to find any mitotic figures, all of which exhibit aberrant metaphases and sister chromatid separation (FIG. 5K).

The Sensitized Screen Recovered Mutations in Genes with Diverse Biological Roles

It was not immediately clear why mutations in gli, Hr39 and lamA were recovered in a screen for chromosome inheritance mutations. Briefly, gliotactin is a transmembrane protein involved in the establishment of the blood/nerve barrier (Auld et al., Cell, 81:757-767 (1995)); Hr39 (also know as DHR39 or FTZ-F1beta) is a member of the Drosophila nuclear hormone receptor family (Horner et al., Dev. Biol., 168:490-502 (1995)); Laminin A is localized to the basement membrane and has been shown to be involved in growth cone guidance of axons (Garcia-Alonso et al., Development, 122: 2611-2621 (1996)). It is possible that some mutations reflect the random noise that accompanies most screens; for example these insertions may have resulted from “hit-and-run” events, which result in mutations at loci unlinked to the final resting site of the P element. Alternatively, these loci may have as yet undescribed functions in inheritance.

Most of the isolated lines (88%) exhibit significantly reduced levels of transmission. This would be expected because P element mutagenesis should result in reduced levels of gene expression. In most cases this will perturb a particular function that is involved in inheritance and result in reduced J21A transmission. Some genes may dominantly increase transmission, however, for example, mutations in genes that encode repressor functions may result in misexpression of a protein required for proper spindle attachment to the kinetochore. Mutations of this sort may rescue J21A transmission by allowing more spindles to attach to the compromised centromere. Mutations in cell cycle regulatory proteins may also result in high transmission. Mutations in a regulator of the metaphase to anaphase checkpoint might result in a delay of the cell cycle and enable time for more faithful inheritance of J21A. Therefore this small subset of the collection (six individual loci) represent a very interesting class of genes and warrant further analysis.

The Majority of the Collection Represents P Insertions in Novel Loci

The identification of P insertions in known genes demonstrates some of the cellular functions that can be expected to be represented in the rest of the collection. It is estimated that >50% of the thirty-four novel loci will have roles in some of the functions already discussed (FIG. 6) as well as other essential inheritance functions such as kinetochore structure and microtubule capture and chromosome congression. Indeed, it has been demonstrated that all of the six independent homozygous lethal or semilethal mutations in novel loci exhibit dramatic mitotic chromosome defects. The identification of genomic clones, ESTs and other P insertions for many of these loci will greatly facilitate further analysis. Broad genetic screens performed in Drosophila have had an enormous impact on the field that they were designed to investigate, and also on other fields and in other organisms (Sandler et al., Genetics, 60:525-558 (1968); Baker and Carpenter, Genetics, 71 :255-286 (1972); Nüsslein-Volhard et al., Roux's Arch. Dev. Biol., 193:267-282 (1984); Kania et al., Genetics, 139:1663-1678 (1995); Salzberg et al., Genetics, 147:1723-1741 (1997); Sekelsky et al., Genetics, 152:529-542 (1999)). The tools are now in place to capitalize on this collection. The screening method of the invention enables the analysis of novel gene products that are required in multicellular eukaryotes for spindle formation, cell-cycle regulation, chromosome structure and centromere structure and function. At least two of the genes identified in the screen may have relevance to a human genetic disorder (wap1^{Scim} and Scim25). Patients with Roberts syndrome (RS) exhibit growth retardation, craniofacial malformations and tetraphocomelia (Van den berg and Francke, Am. J. Med. Genet., 47:1104-1123 (1993)). Mitotic cells from affected individuals exhibit chromosomes with a “railroad-track appearance” that look very similar to the wap1 mutant phenotype in Drosophila (Verni et al., Genetics, 154:1693-1710 (2000)). In sum, discoveries from this screen will impact on the understanding of how chromosomes and the cellular machinery are orchestrated to promote chromosome inheritance in muticellular eukaryotes and should inform us of the causes and consequences of human disorders associated with aneuploidy, such as birth defects and cancer.

TABLE 1
Dominant modifiers ofJ21A inheritance

| | # of | transmission | # homozygous |
|---|---|---|---|
| | lines | (%) | lethal* |
| Decreased | 69 | 9-21 | 11 (16%) |
| transmission | | | |
| Increased | 9 | 38-51 | 3 (33%) |
| transmission | | | |
| TOTAL | 78 | | 14 (18%) |

TABLE 2
Dominant modifiers of J21A inheritance in known loci

| | | | | |
|---|---|---|---|---|
| | T.T. | | | |
| Line | (%) | Location | Stage of Lethality | Human Accession # |

Insertions at known loci

| | | | | |
|---|---|---|---|---|
| Fim^{Scim} | 19 | 16A1-2 | — | P13797 |
| bif^{Scim} | 16 | 10D1-2 | — | — |
| wapl^{Scim} | 19 | 2D6 | — | BAA13391 |
| *grp^{Scim} | 17 | 36A6-7 | — | NP_001265 |
| Rab5^{Scim} | 21 | 22E1-2 | 1^{st} instar | NP_004153 |
| oaf^{Scim-a} | 20 | 22F3 | — | — |
| oaf^{Scim-b} | 20 | 22F3 | — | — |
| Gli^{Scim} | 19 | 35D4 | — | NP_000046 |
| Hr39^{Scim} | 18 | 39C1-3 | — | NP_004950 |
| His4^{Scim} | 20 | 39D | — | HSHU4 |
| Sca^{Scim-a} | 17 | 49D1-3 | — | NP_000499 |
| Sca^{Scim-b} | 20 | 49D1-3 | — | NP_000499 |
| cnn^{Scim} | 17 | 50A3-6 | — | AAC31665 |
| pav^{Scim} | 18 | 64B2-7 | embryonic | NP_004847 |
| rfc4^{Scim} | 21 | 64A10 | 2^{nd} instar/pupal | — |
| LanA^{Scim} | 14 | 65A10-11 | — | P25391 |
| eIF- | 13 | 67B1-2 | 3^{rd} instar | NP_001959 |
| 4E^{Scim-a} | | | | |
| eIF- | 17 | 67B1-2 | 3^{rd} instar | NP_001959 |
| 4E^{Scim-b} | | | | |
| Gap1^{Scim-a} | 18 | 67D2-3 | — | CAA61580 |
| Gap1^{Scim-b} | 10 | 67D2-3 | embryonic/1^{st} | CAA61580 |
| | | | instar** | |
| JIL-1^{Scim} | 19 | 68A4-5 | — | AAC31171 |
| nos^{Scim} | 17 | 91F4-5 | embryonic** | — |

Insertions at mobile elements

| | | | |
|---|---|---|---|
| Scim1 | 10 | 2 | 3^{rd}instar** |
| (mdg3) | | | |
| Scim2 | 20 | 2 | — |
| (gypsy) | | | |
| Scim3 | 20 | 2 | — |
| (YOYO) | | | |

T.T.= transmission test data.
*There are two P insertions in this line; one within grp (FIG. 3A) and one within a novel locus at 23A3-4 (Scim12⁴ , Table 3, FIG. 3B).
**semilethal. See Methods and Materials for derivation of Human Accession #.

TABLE 3
Dominant modifiers of J21A inheritance in novel loci

| Line | T.T. (%) | Location | Stage of Lethality | Clone accession # | Human accession # |
|---|---|---|---|---|---|
| Scim4 | 20 | X | — | AC012823 | a |
| Scim5 | 21 | X | — | AC019800 | a |
| Scim6 | 20 | 8C4-5 | — | AC014159 | b |
| Scim7 | 18 | 9B7-8 | — | AC013173 | a |
| Scim8¹ | 19 | 11B16-17 | — | AC019992 | b |
| Scim8² | 20 | 11B16-17 | — | AC019992 | b |
| Scim9 | 17 | 10C1-2 | 3^{rd} instar^{**} | AC017852 | a |
| Scim10 | 21 | 6D1-2 | — | AC013845 | BAA34480 |
| Scim11 | 25 | 19F2-3 | — | AC019797 | a |
| Scim12¹ | 51 | 23A3-4 | — | AC019974 | b |
| Scim12² | 21 | 23A3-4 | — | AC019974 | b |
| Scim12³ | 18 | 23A3-4 | — | AC019974 | b |
| Scim12⁴ | 17 | 23A3-4 | — | AC019974 | b |
| Scim12⁵ | 40 | 23A3-4 | embryonic/larval | AC019974 | b |
| Scim12⁶ | 40 | 23A3-4 | embryonic/larval | AC019974 | b |
| Scim12⁷ | 39 | 23A3-4 | — | AC019974 | b |
| Scim12⁸ | 19 | 23A3-4 | — | AC019974 | b |
| Scim13¹ | 19 | 23B1-2 | — | AC019901 | b |
| Scim13² | 39 | 23B1-2 | — | AC019901 | b |
| Scim14¹ | 19 | 28B1-2 | — | AC020004 | a |
| Scim14² | 21 | 28B1-2 | — | AC020004 | a |
| Scim15¹ | 9 | 30D1-2 | — | AC020324 | b |
| Scim15² | 14 | 30D1-2 | — | AC020324 | b |
| Scim16 | 21 | 31F1-2 | — | AC020157 | AAB07777 |
| Scim17 | 16 | 33B3-4 | — | AC019795 | b |
| Scim18 | 18 | 38C5-6 | — | AC017171 | b |
| Scim19 | 17 | 39A3-B1 | — | AC018212 | NP_003866 |
| Scim20 | 21 | 42A8-B3 | — | AC015089 | NP_002653 |
| Scim21 | 20 | 42B1-3 | — | AC013962 | b |
| Scim22 | 38 | 42C8-9 | — | AC014497 | AAC79152 |
| Scim23 | 19 | 44B | — | AC020344 | Q92539 |
| Scim24 | 19 | 47C3-4 | 3^{rd} instar | AC017793 | NP_005350 |
| Scim25 | 15 | 51A1-2 | 2^{nd} instar | AC015180 | NP_005307 |
| Scim26 | 14 | 50E6-51A2 | — | AC012771 | AAC32592 |
| Scim27 | 21 | 54B4-5 | — | AC020084 | AAC63061 |
| Scim28 | 43 | 57B2-3 | — | AC020202 | a |
| Scim29 | 14 | 58E4-F4 | — | AC020206 | b |
| Scim30 | 19 | 84D9-E2 | — | AC013928 | b |
| Scim31 | 45 | 86B1-2 | 3^{rd} instar | AC017117/Dom | b |
| Scim32¹ | 18 | 87C-D | — | AC017336 | NP_002386 |
| Scim32² | 14 | 87C-D | — | AC017336 | NP_002386 |
| Scim33 | 18 | 91A4-A6 | — | AC014473 | NP_005168 |
| Scim34 | 10 | 91F6-11 | — | AC015189 | AAA61314 |
| Scim35 | 22 | 92E-93A | — | AC014084 | b |
| Scim36 | 14 | 97D6-E6 | — | AC014839 | 226753 |
| Scim37 | 18 | 98C1-2 | — | AC019593 | a |

T.T. = transmission test data; high transmitters are in bold. We have been unable to localize one of the high transmitters and it is not represented in the table. Scim30⁵ and Scim30⁶ are also lethal over the SM1, SM5 and CyO balancer chromosomes. Scim29 and Scim53 exhibited a bimodal distribution of J21A inheritance and were retained as stocks even though they were outside of the standard <22% or >37% cutoff.
^{**}semilethal.
a = no Drosophila ESTs,
b = there are Drosophila ESTs but no significant homology was found. See Materials and Methods for a description of Human Accession No's.

TABLE 4

NA and AA sequences for novel loci

Scim4
AE003424 (insertion @188565)
Nearest ORFs are CG12497 @164549 to 167398 and CG13758 @216214 to
219666.
>>CG12497|FBgn0029621|cDNA sequence
ATGCTGGCAGATGATGAGTCGCTGCAGGGCATCAACGATTCCGAGTGGC
AGCTCATGGGTGATGACATTGACGACGGCCTACTGGACGATGTCGATGA
GACACTGAAGCCCATGGAGACCAAGTCCGAGGAGGAAGACTTGCCCAC
TGGCAACTGGTTCAGCCAGAGTGTCCATCGCGTTCGCCGTTCCATAAACC
GTTTATTTGGTTCCGACGACAATCAGGAACGGGGACGACGACAACAGCG
TGAGCGGTCGCAAAGGAATCGCGATGCGATTAATCGGCAAAAAGAACT
GCGCCGCAGACAAAAGGAGGACCACAACCGCTGGAAGCAAATGCGAAT
GGAGCGACAACTGGAGAAACAGCGCTTGGTCAAACGGACCAATCATGTT
GTCTTCAACCGCGCCACCGATCCTCGCAAGCGGGCATCGGACCTTTACG
ACGAGAACGAGGCATCCGGCTATCACGAGGAGGATACAACTCTCTATCG
TACCTACTTCGTCGTTAACGAACCTTATGACAACGAATACAGAGATCGA
GAAAGCGTACAGTTCCAGAACCTGCAAAAACTTCTGGACGATGATCTGC
GCAACTTCTTCCACAGCAACTACGAAGGTAACGATGACGAGGAGCAGG
AAATTCGCAGCACACTGGAGCGCGTTGAAATAGAGCTGCCCACTTCGGT
CAACGACTTTGGAAGTAAGTTGCAGCAGCAACTGAATGTCTATAATCGT
ATCGAAAACTTGAGCGCCGCTACCGATGGCGTATTTTCCTTCACTGAATC
TAGTGATATCGAGGAAGAGGCAATCGATGTTACATTGCCCCAGGAAGAG
GTTGAGGGCTCTGGTAGCGATGACTCCAGCTGTCGTGGAGACGCCACCT
TCACCTGTCCCCGGAGCGGAAAAACCATTTGCGATGAAATGCGCTGCGA
TAGAGAGATCCAATGTCCCGATGGCGAGGACGAAGAGTACTGCAACTAT
CCAAATGTTTGCACTGAAGATCAGTTCAAGTGCGACGATAAGTGTCTGG
AGCTCAAAAAACGCTGCGATGGAAGTATCGATTGTCTGGATCAGACCGA
CGAGGCTGGCTGCATTAATGCGCCAGAACCAGAACCAGAGCCTGAACCA
GAGCCAGAGCCTGAACCGGAATCTGAACCAGAGGCCGAACCCGAACCC
GAACCTGAGCCTGAGCCTGAGTCTGAACCAGAACAAGAACCTGAACCCC
AAGTCCCGGAAGCCAATGGTAAGTTCTATTGA
>CG12497|FBgn0029621
MLADDESLQGINDSEWQLMGDDIDDGLLDDVDETLKPMETKSEEEDLPTGN
WFSQSVHRVRRSINRLFGSDDNQERGRRQQRERSQRNRDAINRQKELRRRQ
KEDHNRWKQMRMERQLEKQRLVKRTNHVVFNRATDPRKRASDLYDENEA
SGYHEEDTTLYRTYFVVNEPYDNEYRDRESVQFQNLQKLLDDDLRNFFHSN
YEGNDDEEQEIRSTLERVEIELPTSVNDFGSKLQQQLNVYNRIENLSAATDG
VFSFTESSDIEEEAIDVTLPQEEVEGSGSDDSSCRGDATFTCPRSGKTICDEMR
CDREIQCPDGEDEEYCNYPNVCTEDQFKCDDKCLELKKRCDGSIDCLDQTD
EAGCINAPEPEPEPEPEPEPEPESEPEAEPEPEPEPEPESEPEQEPEPQVPEANG
KFY
>>CG13758|FBgn0029622|cDNA sequence
ATGACCCTCCTGTCGAACATTCTCGACTGCGGAGGCTGTATTTCCGCCCA
GCGCTTCACCCGCCTGCTGCGCCAGTCCGGCTCATCAGGACCATCCCCAT
CTGCACCGACGGCCGGAACATTTGAATCAAAATCCATGCTGGAGCCAAC
ATCCTCGCACAGCCTGGCGACCGGACGCGTGCCACTACTGCACGATTTC
GATGCCTCGACAACGGAATCGCCGGGAACGTATGTCCTCGACGGTGTCG
CCAGGGTGGCCCAATTGGCCCTGGAGCCCACCGTCATGGACGCACTGCC
CGATTCGGACACGGAACAGGTTCTCGGACTCTACTGCAATTGGACCTGG
GACACATTGCTCTGCTGGCCACCCACTCCGGCTGGAGTCCTTGCACGGAT
GAATTGTCCTGGCGGCTTTCATGGCGTAGATACGCGCAAATTCGCCATCC
GAAAGTGTGAGCTGGATGGTCGATGGGGCAGCAGGCCAAATGCCACGG
AGGTGAATCCGCCGGGATGGACGGACTACGGGCCGTGTTACAAGCCGG
AGATTATCCGTCTCATGCAGCAGATGGGCAGCAAGGACTTCGATGCCTA
CATAGACATTGCCAGGAGGACTCGAACCCTGGAGATCGTGGGCCTCTGC
CTCTCCCTGTTCGCCCTTATAGTTTCCCTGCTGATCTTCTGCACATTTCGC
TCGCTGCGAAACAATCGCACCAAGATCCACAAGAATCTTTTCGTCGCCA
TGGTGCTGCAGGTGATCATTCGCCTGACCTTGTATCTCGACCAATTCCGG
CGGGGAAACAAGGAGGCGGCCACCAACACGAGTCTCTCTGTCATTGAGA
ACACGCCCTATTTGTGCGAAGCATCCTATGTACTTCTGGAGTACGCTCGT
ACCGCCATGTTCATGTGGATGTTCATCGAGGGCCTTTACCTGCACAACAT
GGTCACCGTGGCCGTTTTCCAGGGCAGCTTTCCCCTCAAGTTCTTCTCGC
GACTCGGCTGGTGTGTGCCCATTCTGATGACCACCGTGTGGGCGAGATG
CACGGTCATGTATATGGACACCTCGCTGGGCGAATGCTTGTGGAACTAT
AATCTCACGCCCTACTACTGGATCCTCGAGGGGCCACGACTAGCGGTCA
TACTGCTAAACTTCTGTTTCCTGGTGAACATTATCCGAGTGCTGGTAATG
AAGCTGCGTCAATCGCAGGCCAGCGATATAGAACAGACTCGCAAGGCA
GTTAGAGCGGCTATAGTCCTACTACCACTTTTGGGTATAACCAATCTCCT
GCACCAGCTGGCTCCTCTGAAAACGGCCACGAACTTCGCGGTCTGGTCG
TATGGCACCCACTTTCTCACCTCGTTTCAGGGATTTTTTATAGCGCTAATT
TACTGCTTTCTAAATGGCGAGGTTCGTGCCGTGCTACTAAAGAGTCTGGC
CACCCAGCTGTCGGTGCGAGGTCATCCGGAATGGGCGCCGAAAAGGGC
ATCTATGTACTCGGGTGCTTATAACACGGCGCCGGATACGGATGCAGTG
CAGCCTGCAGGAGATCCATCGGCCACTGGAAAGCGAATATCACCGCCGA
ATAAAAGGCTGAATGGAAGAAAGCCGAGCAGTGCCAGCATTGTGATGA
TTCACGAGCCTCAACAGCGCCAGCGACTGATGCCCCGGCTGCAAAACAA
GGCGCGGGAAAAGGGCAAGGACCGGGTGGAGAAGACGGATGCGGAAG
CGGAGCCGGATCCGACCATCTCCCACATTCACAGCAAGGAGGCGGGCAG
CGCGAGATCGCGAACTCGCGGCTCCAAGTGGATAATGGGCATCTGCTTC
CGGGGTCAAAAGGTACTAAGAGTACCGTCAGCGTCATCCGTGCCACCCG
AGTCAGTTGTATTTGAGTTGTCAGAGCAGTAG
>CG13758|FBgn0029622
MTLLSNILDCGGCISAQRFTRLLRQSGSSGPSPSAPTAGTFESKSMLEPTSSHS
LATGRVPLLHDFDASTTESPGTYVLDGVARVAQLALEPTVMDALPDSDTEQ
VLGLYCNWTWDTLLCWPPTPAGVLARMNCPGGFHGVDTRKFAIRKCELDG
RWGSRPNATEVNPPGWTDYGPCYKLPEIIRLMQQMGSKDFDAYIDIARRTRT
LEIVGLCLSLFALIVSLLIFCTFRSLRNNRTKIHKNLFVAMVLQVIIRLTLYLDQ
FRRGNKEAATNTSLSVIENTPYLCEASYVLLEYARTAMFMWMFIEGLYLHN
MVTVAVFQGSFPLKFFSRLGWCVPILMTTVWARCTVMYMDTSLGECLWN
YNLTPYYWILEGPRLAVILLNFCFLVNIIRVLVMKLRQSQASDIEQTRKAVRA
AIVLLPLLGITNLLHQLAPLKTATNFAVWSYGTHFLTSFQGFFIALIYCFLNGE
VRAVLLKSLATQLSVRGHPEWAPKRASMYSGAYNTAPDTDAVQPAGDPSA
TGKRISPPNKRLNGRKPSSASIVMIHEPQQRQRLMPRLQNKAREKGKDRVEK
TDAEAEPDPTISHIHSKEAGSARSRTRGSKWIMGICFRGQKVLRVPSASSVPP
ESVVFELSEQ
Scim5
AE003506 (insertion @187490), nearest ORF (CG15816) @188338 (800 bp away).
>>CG15816|FBgn0030866|cDNA sequence
ATGTTGCAAGCCGCTAGCAGCACAACAACAGCACCAGTGGGAAATACA
GCAGACACAGGAAACAGTGAAAGCCCGATAATAGCGACGCCGGAGGAG
AAATCCCAAAGACGACGCTCCACATTCTATGTACCATTGGTAATAGAAG
ACGAAGAGGAGACCAAAAAGGATACGCCCGCAGATCATCTGGTCCAAA
AGTCCTCGAGCAATACGAGCCTAAGTAGCAATAGCAATTCCCTAACGAA
TTCCGAAACAAAATCATCGAAAAGCTATAGTCTGCGCAAATCGAGTTCG
GTGAAAAGCGGCGTGGCCAAGGTTAGTGCTCTCTTCGAGCGGAAAACTC
CATCGAAAATGTCGCCACCTTGCGGCTTCAATTGGAGCATCAGTGGCAG
CGAAAATACGGCCCAATACTCCGATACCGATGATGATGAGGAGAACTCC
ACGGAGGCACGTCATCGCGAACAGCTGCTCAAGACCCTGCCCAGCGGTA
ATAATAATTCCACCACCGCATCCCCATCGAAACTGAAACGATATGGCAT
CGTACTGAACGTCATCAGTTTGAATGGCAGCGATAACGAGCAGTCCTCG
TTGGGTAGTAATGGTAGCAGCATGCCATCCATGCCATCAATGCCAAACG
GCCAGAACATACCAAATGCGGCTGCGCCCAGGACTCATTTCAATGAGGA
GAACGACATTGTCCTGGCCACGCCCCCGCCGCCCAAACAGCAGGCACTA
TCCGCCGCTCATGAGTCTAACGACTACGATGATGACTCAGAAATAAGTC
GCATGCAGACGAACACCTCGACGCCCATAAAGCTAATGAAATCGCGATC
GCGAACCAATATACTAGCCGTACCGCTGCCATCGGTGGAGCGTGGTTTG
GCCACAACAAATACGACGCCCAATAATAATAATATCAATGGTAATAGTA
ATGGTAGTACCAGCAATACGACCACTACGACAACGACGACGACGTTGAT
TACGCTTCGTGCAAAATCGAAGACCCTGCCGCAAAATCTATCGCCCTCA
GTTGTTTTACGCGAGGCAGCCGCACTGGATGAGCTCGAGAAGAAGCGGG
AGAAGTATCAGGAGAAGCAGGAGAAGCGGGAAAAGCTGCAGGAGAAA
CAGCGTCAGCTGTTCGGCGGCAGTACGGCCAGTCAGATAGCGGGCTCTT
CGCCCTACAAACTGCAGAACAGCTGTTCGGCCACCTCGATACTAACGCA
CAGTTTTCCGCCGAAGAACCTTTTTCTACTTAAGTCCACGCCCAAACTGT
CAACGGATATAGCCGCGGCCACGCCCCCAAATACATCGGCAATCTGTTC
GCCGCCCAAGAAATCGCTGAGCTTCATTCGACGTGCCCACTCCACCAAG
GTGGCACGCAGCAATTCGCTGCTTAAACCAAATCAGGCTGGAATCCTAG
GATCGGGCAGTGGATCCAACGGACTCGGAGTCCATCAGGGCGTCATGCA
GGGAGCATTGTCCATCAGCTGTGCTGGGGACAATTCCAGCAACAATGGC
AGTTGGGGCAAACACTTCTACCAGCCCTACGATGTGTGTCCCTTGAGTCT
GGACGAGCTCAATTGCTATTTCCAGGCGGATCAGTGCGAGAAACTGATC
TGCGAACGATTCAAGATCAGGGATCTGGCCATACACATGGCATCCGCAT
CCGCAATTGGAGCGGATCTCTCTGTGACCACAGAGAATGAGACAACGGC
AACGGCGGACGACGATGCGGGACATCATTCGGGTAGGTCGATTCACCCC
CCCCCCCCAAAAAACGAAAATTCTTTGTCCCTGCTCAATGGGCAGCAAG
TACTACATACATAA
>CG15816|FBgn0030866
MLQAASSTTTAPVGNTADTGNSESPIIATPEEKSQRRRSTFYVPLVIEDEEET
KKDTPADHLVQKSSSNTSLSSNSNSLTNSETKSSKSYSLRKSSSVKSGVAKV
SALFERKTPSKMSPPCGFNWSISGSENTAQYSDTDDDEENSTEARHREQLLK
TLPSGNNNSTTASPSKLKRYGIVLNVISLNGSDNEQSSLGSNGSSMPSMPSMP
NGQNIPNAAAPRTHFNEENDIVLATPPPPKQQALSAAHESNDYDDDSEISRM
QTNTSTPIKLMKSRSRTNILAVPLPSVERGLATTNTTPNNNNINGNSNGSTSN
TTTTTTTTTLITLRAKSKTLPQNLSPSVVLREAAALDELEKKREKYQEKQEK
REKLQEKQRQLFGGSTASQIAGSSPYKLQNSCSATSILTHSFPPKNLFLLKSTP
KLSTDIAAATPPNTSAICSPPKKSLSFIRRAHSTKVARSNSLLKPNQAGILGSG
SGSNGLGVHQGVMQGALSISCAGDNSSNNGSWGKHFYQPYDVCPLSLDEL
NCYFQADQCEKLICERFKIRDLAIHMASASAIGADLSVTTENETTATADDDA
GHHSGRSIHPPPPKNENSLSLLNGQQVLHT
Scim6
AE003446 (insertion @55800), nearest ORF (CG6999) @55741 (60 bp away)
>>CG6999|FBgn0030085|cDNA sequence
AAAACGAAAGCTACAATGAAAAAAATAATTAATACATCAAAGCCAAAG
CGCAAGTCCACTTCCATGAAAGTGGAGGAGACTAAGCTAGACGAGGCG
CGCTGGGGTAAGCCGCAGACAAAGGAAGGTGAGTCTGCAAATGGGATA
GCAAATCCCTCAAATGACGATAAAAAGGAGCTGGCCAATTTCAAAGCCA
CCTTCAATTCCTGGGCCCCCGAGAAGAAACGCGAGAAGATGCACAAGGT
AGGCGTCATCTTAATATCCAACATACCCAAGGACATGGACGGGGACTGC
CTGAAGGAAATCATGAACTTGCACAGCGTCGTCGGCAGAGTTTACGTGC
AGCCGGAAACGCTGTCAAGTTTCAAGACAAAGAAGAACATGCGTAAGG
GCTGGGTGGAGTTCATTTCGAAAAGTGGGGCCAAAAAAATCGCTCTAGA
GCTGAACAATAAGCCTATAACCGATGGCAAGTCGTCCCGATTCCGTGGC
TTGCTGTGGAAAATGAAGTTCCTGCCACGCTTCAAGTGGTACTATCTAAC
CGATCGCATGGACTACGAGCTGGCGGTTTGCAAAGTTCGCGTATGGTCG
CAGGCCCGCAAGCGGGCCACCTTCTGGTACGATCCCGACCAGATGGAGT
ATTTCAAGAAGCAAGTGAAGAAGATGAAGAAGATGAAGAAGGTCAAGG
AAGCGGAGATGGCTACCAGGAATGCGGAGATGGCTGCCAAGAAAGCGG
AGATGGCTGCCAAGAAATTGAAGAAGTCTGCCTGAGTTCACGCTAGACC
TTTGCTTCCAATGTCTACCTGACTGCAAATATACTTCAATAAAGTAAATC
AAATC
>CG6999|FBgn0030085
MKVEETKLDEARWGKPQTKEGESANGIANPSNDDKKELANFKATFNSWAP
EKKREKMHKVGVILISNIPKDMDGDCLKEIMNLHSVVGRVYVQPETLSSFKT
KKNMRKGWVEFISKSGAKKIALELNNKPITDGKSSRFRGLLWKMKFLPRFK
WYYLTDRMDYELAVCKVRVWSQARKRATFWYDPDQMEYFKKQVKKMK
KMKKVKEAEMATRNAEMAAKKAEMAAKKLKKSA
Scim7
AE003574 (insertion @132480), nearest ORF (CG13238) @122949 (10 kb away)
>>CG13238|FBgn0031198|cDNA sequence
ATGCGGCCCATCATCATTACTGTTTTGTCCGGGCCACAGGTGTACATCGT
ACAGGTGCACTGTCGTAGCAAAAACATACCTGACGTCTACATCCTGACC
GTTACCCAGATGCTCCAGTACGTGACCGACCCAAAGGAGCTTCGCGATG
TCAGCCAAATTGAGTCGTGGAAGTGCGACAAGAGCGTGTCTGTAGCCCC
CAAGCCCTGCAATATCTGGCAGACGTGTGCGCTGCCCTTCAAGATTCCC
GAACAGAATCTGACGGATACGCGCTATATGGAGACCTGTCGGGAATGCC
CTAATGTGTATCCCTGGCTGGGCGATGCAGGCGGTACGGGAATCGCGGG
TCGCGATAACTATATCTTTGCCGGTGGCGAAAATCCAGAGGAAGAAGAC
TCTGCGAAGTAG
>CG13238|FBgn0031198
MRPIIITVLSGPQVYIVQVHCRSKNIPDVYILTVTQMLQYVTDPKELRDVSQIE
SWKCDKSVSVAPKPCNIWQTCALPFKIPEQNLTDTRYMETCRECPNVYPWL
GDAGGTGIAGRDNYIFAGGENPEEEDSAK
Scim8¹
Scim8²
AE003490 (insertion @120150), nearest ORF (CG4004) @120304 (200 bp away)
>>CG4004|FBgn0030418|cDNA sequence
TCGAATCGAGCGTGAAAACGTGCAATAAAACCAAAGTTAACAAAAACA
AAAAAAAAAAAACCAGACTACTTAATGTCCCAGATGGGCGGCACATGCT
TGTACGATGAGCCCGAAATCATGGAGGAGTTCATCAGCTGTTATCAGTA
TTTCACCGCCCTGTGGGACAGCAGCAGTCCCGATTATCTATCGAAACAG
AAAAAGGAGCCCGGCTATCAGGAGCTATTGAAGATACTGCGACGCGTTA
ATAGCAACTGTTCGATTCAGGATGTTAAGCGAAAGATAAACTCGCTGCG
TTGCTGCTATCGTCGTGAATTCAAAAAGGTACAGGAATCGGTCAATGGC
TACCAGACGCGTCTCTGGTGGTTTCATCTGATGGATTTCCTCAAGCCGGT
ACTCAACATACAATCGCCGGCCAGGGTGAAATCCGAGAACGTGGACGAT
AGTCTCGACGAGACCAGCATTCAGGATGTTGACATTATGTCTGATGCCTT
TCCACACGAAGAGGATATGCTACGTCTTGATGCCGTGGGTGATGGCGAT
GTTGAACCGGAACCCGAGCCTGATAACGATCCCGAATTGGATAACATGG
ATGATCATGTTGATGATTATCGTAACAATTCATCGGCTGGGAGCATTAAG
AACAATGGCTATCAGCAGCACACCGTATCTTCGCACCAGCAGCATAACG
GTGAATCGCAGACTTCGGATAAATCCGGACGTCGCATCCGTAACCGACG
AAGACGCAGTAGCAATGACACCGATTACGTTGAAGCGGCGAGAAAGCG
TAGAAATGTGGAGACTTCGAATAGAGATAGAGACTGGCATAGAGAGCG
GGATAGGGAGCGAGACAGAAAGCATGAAAGCGACAGCGAGTACGAGTG
CGAGCTGA
>CG4004|FBgn0030418
MEEFISCYQYFTALWDSSSPDYLSKQKKEPGYQELLKILRRVNSNCSIQDVK
RKINSLRCCYRREFKKVQESVNGYQTRLWWFHLMDFLKPVLNIQSPARVKS
ENVDDSLDETSIQDVDIMSDAFPHEEDMLRLDAVGDGDVEPEPEPDNDPEL
DNMDDHVDDYRNNSSAGSIKINNGYQQHTVSSHQQHNGESQTSDKSGRRIR
NRRRRSSNDTDYVEAARKRRNVETSNRDRDWHRERDRERDRKHESDSEYE
CEL
Scim9
AE003422 (insertion @182395), nearest ORF (CG3587) @174339 (8 kb away)
>>EG:39E1.2|FBgn0023521|cDNA sequence
GCAAGCACATATCTAAATCTAGCTCGAAACCAGATGGATGCTCATCTTG
CACACTGTCACCAGTGTTGGTAACCGAGTGCATTGTGAGCGGAACGTTC
CGACACCTACTTTGTTTATTTATTGTTATTAATTAGGAAGCATGCCCCTC
GTGGTGATTACGGGCCTGCCAGCCAGCGGAAAGAGCACACGTGCCCGCC
AGCTACGGGATCATTTCGTGGAGCGCGGCAGGAAGGTGCATCTAATCAG
CGAAAACGAGGCAGTGCCCAAGGCGGGTTTTGGAAAGAATTCCCATACA
GGTGATTCGCAGAAGGAGAAGGTGGTACGTAGCGATCTTAAGTCGGAAG
CCTCGCGTCACCTTAACCAGGAGGATCTGGTCATCTTGGACGCCGGGAA
CTACATCAAAGGCTACCGCTACGAATTGTACTGCATGTCCAAGGTGTCA
AGGACCACCCAGTGCACTGTGTTTACCTGCATACCCCAGGAGGAGGCGT
GGACCTTTAATAGCCAAAGAACGGCGCCGGATGAACTGCCTGGCGACAG
TGAAAGAGTTCAGCCGGTGGACAACTCGGATGTTCCCTACACCAGAGAG
ACTTTTGATGCTCTGTGCCAGCGCTACGAGGAGCCGCAGAGCAACAACC
GTTGGGACAGTCCGCTGGTGGTAGTCTTGCCCAAGGACACGCTCGACAT
GGAGGCCATCTACAAGGCCTTGTACGAGTCCCAGCCACTGCCACCCAAC
CAGAGTACTTATAATGCACCGCTGGGAACAACCAACTACCTGTTCGAAC
TGGACAAAATCGTGCAGGCGATCATCAAGGAGATCCTCGGCGCCGTCAA
GATCAAGGCCTTCGGCCAGCTGCGCATCCCAGGGAGCAGAAATCCCGTG
AAGGTCGCCACTTCGATGAATGCCCTCCAGCTGAACCGCCTGCGCCAGA
AGTTCATCACGAGCACGTGCCACGCCAGCCAGACGTCACCCACTCCGCT
GGAGCAGGTGCCGCACTTGTTCGTGCAGTTCATCAATGCCAACACGATC
GGCTGCTAG
>EG:39E1.2|FBgn0023521
MPLVVITGLPASGKSTRARQLRDHFVERGRKVHLISENEAVPKAGFGKNSHT
GDSQKEKVVRSDLKSEASRHLNQEDLVILDAGNYIKGYRYELYCMSKVSRT
TQCTVFTCIPQEEAWTFNSQRTAPDELPGDSERVQPVDNSDVPYTRETFDAL
CQRYEEPQSNNRWDSPLVVVLPKDTLDMEAIYKALYESQPLPPNQSTYNAP
LGTTNYLFELDKIVQAIIKEILGAVKIKAFGQLRIPGSRNPVKVATSMNALQL
NRLRQKFITSTCHASQTSPTPLEQVPHLFVQFINANTIGC
Scim 10
AE003438 (insertion @216460), between two OREs (CG14439 @213729 and
CG14438 @219903)
>>CG14439|FBgn0029898|cDNA sequence
ATGATACCTATTCTGGAGAAACTCAGCGGGTTCTACAACACCTACGTCTT
GGCCGTACTCACCATTGGTTATATCCTGGGCGAATTGGGTCACTATCTGA
TCGGAGTGACCTCCAAGCAGACGGCCATTGAGTTGGACTACGGTGATCA
TGCCTGCCAGCAGAACACCTCGATGTTCAATCGCCACGAGTTGCCCACC
CAGTGCTCGGCGGTTATGAATGAGACCAGCTGCTATGCCCTTGATTTCAA
CGGCACTGGCTATTGCGAGTGGAACTACAATGGACTGGGCATCGACTAC
CAGATCCTGGCCGGACCCACCTTCATCCTGATTTTCACCATCGCCGGCGT
ATTTATGGGCTTCGCAGCGGACAAGTACAATCGCGTCAACATGCTGACT
GTGTGCACAGTGATCTTCGGCATTGCCATGATTCTGCAGGGCACCGTTAA
GGAATACTGGCAGCTGGTAATTTTGCGTATGATCATGGCAGCCGGCGAG
TCGGGTTGCAATCCCTTGGCCACGGGCATTATGTCCGATATCTTTCCGGA
GGATAAGAGAGCACTAGTCATGGCCATCTTCAACTGGGGAATTTATGGA
GGATATGGAATCGCCTTCCCCGTGGGTCGCTACATCACCAAGCTGAATTT
CTGGAATCTGGGATGGCGCGTTTGCTACTTGGGCGCCGGTGTCCTTACCG
TAATTATGGCCGCACTGACCGGAACCACTTTGCGGGAGCCGGAGCGCAA
GGCCATCGGTGAGGGTGACCGCCAGACGTCTAGCGGCAAACCAGTGAG
CCTGTGGCAAGTTATCAAGAATCCGGCAATGATCATGTTGATGATTGCC
GCGTCCATCCGTCACTGCGGTGGCATGACCTTTGCCTACAACGCCGATCT
CTACTACAACACGTACTTCCCCGACGTGGACTTGGGCTGGTGGCTCTTTG
GGGTCACCATTGGCATTGGCAGCGTGGGTGTGGTCGTCGGTGGCATTGT
GTCGGACAAGATTGTCGCCAAGATGGGCATTCGATCACGCGCCTTTGTA
TTGGCTGTTAGCCAGCTAATTGCCACACTACCAGCCTTCGGATCGGTCTA
CTTTGACCCGCTGTGGGCCATGATCACGCTGGGCCTGAGTTATTTCTTCG
CCGAGATGTGGTTCGGTATTGTCTTTGCCATTGTTGTGGAGATTGTTCCG
CTGCGCGTTCGCTCCTCGACCATTGGCGTCTTTCTGTTTGTGATGAACAA
CATTGGCGGCAACCTGCCCATCCTGGTGGATCCGGTGGCCAAGATCCTG
GGCTATCGCGGTTCGATCATGATCTTCTACGCTGGATTCTACGGCATCAG
TTCTATTCTCTTCTTCATCACCTGTTTCCTGCTGGAAGGCAAGCCTGATG
AGGTGGGACAGCCGGAGTCGCCGAAGAGCCATCCGGATGCCGTGCTCA
ATGCTCGCCACATGCACGGACACGACAACTCCGTGTTCTCCGTGGACGA
GACCTTGCCCTCCAACGGACGTCCTGCCCAACTTCCGCAGCATCTGCAG
ATGTCCAGCAATGGATACGACAAGTCCCAGATTTCTCCGCCACGACAAA
ATGGCGCGGAGAGCAGTAGACTATAG
>CG14439|FBgn0029898
MIPILEKLSGFYNTYVLAVLTIGYILGELGHYLIGVTSKQTAIELDYGDHACQ
QNTSMFNRHELPTQCSAVMNETSCYALDFNGTGYCEWNYNGLGIDYQILA
GPTFILIFTIAGVFMGFAADKYNRVNMLTVCTVIFGIAMILQGTVKEYWQLVI
LRMIMAAGESGCNPLATGIMSDIFPEDKRALVMAIFNWGIYGGYGIAFPVGR
YITKLNFWNLGWRVCYLGAGVLTVIMAALTGTTLREPERKAIGEGDRQTSS
GKPVSLWQVIKNPAMIMLMIAASIRHCGGMTFAYNADLYYNTYFPDVDLG
WWLFGVTIGIGSVGVVVGGIVSDKIVAKMGIRSRAFVLAVSQLIATLPAFGS
VYFDPLWAMITLGLSYFFAEMWFGIVFAIVVEIVPLRVRSSTIGVFLFVMNNI
GGNLPILVDPVAKILGYRGSIMIFYAGFYGISSILFFITCFLLEGKPDEVGQPES
PKSHPDAVLNARHMHGHDNSVFSVDETLPSNGRPAQLPQHLQMSSNGYDK
SQISPPRQNGAESSRL
>>CG14438|FBgn0029899|cDNA sequence
ATGGAGGATAGCGAGGACGACGTGGTGGTGGTGAGCTGCGATACCTCGA
TGAAGGAGAAGGTAAAGGCCAAGCTGGTGGAGATCCGTAAGTTTGTGCC
CTTTATCCGGCGTGTGCGAATAGACTTCCAGGATACTTTGTCCAAGGTTC
AGGGTCATCGTCTGGATGCCCTGGTTAACCTGCTGGATCGCGAGGACGT
ATCGATGAGCTCTCTTAACAAGATCGAGGTGATCATTGATAAGCTAAGG
ACGCGCTTCAATCCGAGGATCGAAATTGACACTGGCGAAATCATTGATA
TCACTGAAAACACTGACGCCAAGGCATCGGATGAGGGGCAGCGGTCAC
CTGCAGAACCACGTGCCGCCCTTCAAGCTATAGTTCAAGATACGAAAAC
ACCAACCATTCCAGAACCAACATCACCAGCGGCGCTTAAGCATTCCTCC
CTTCGTGGCAGTCGTGGATTTCTGGCTGTCATGCAGAAGGCCTTAATTGA
AGAGAAGAAGCAGCGAGCTAGCGAACAGAAAACTGATAAAGAAACTAA
CGGTGTAACGCAGCTAGAGACAAACTTCTCGCGGCGATCTTATACAACA
TCGTCACAGTCAACCTGCCGTTCTTCAGAAATATCGGTAAGAGCAGAAA
ACCCAGATTTTAAGCGACGAAGCACATCGCTTGTGCAGCATGCTCCTCT
ACAGGAGGCCTCCCCAGGGCAATCCAAAAAAGACTTGCCCATATCCTTG
TCGGTACAGGGTCTACCAGCTTTGGTCAGTGCCAGCACTGCAAGTCCAG
CAAATACGCTTGAGGAGGCCCGCAAGAAGCTGGCGGCCTTGAAATATGG
ACTAGGAACAACGGTACCAAGCATGCCTCCACTGGCCTCCAATATAAAT
GATCCACGCGGTAGAAAAGGAATAAACCTGCCTGAAACTAACAACAAT
AAAGACAACGACTTGGGTATAGCGCTGCAATCCCCGCCGCCTATGCGGA
CTCCCTCGCCTATTCCGCCGCCACCAAGGATGAAGGCCGGTACGTGGGC
CTCATTTTCAAATGTTCCCCAGGAAAGCGCATTTACAGGCCAGCATGCTG
TGCAGCGCAACTCGGTACCTCCGGGAGATTCTCGAGCCTTTGGGGATGC
TTTGGCACATGAACCAAGGTCCTTCTATGGCACTGATTCCCGAGAACCCC
GAGACCCTCGTATCTGGAAGAGCAAGACTTCCCAACAGCAGCAACATCA
GCAGGCGCCACAGGCACAAATTCCTCCGTATTCCAGTGACCCGCGTCGT
TCTATAAGCACTTACAGCGGTTTCGAAGAGGGCGGATTTCGCGGCGGTC
ACAACAAACGGGGCTTTGGACGACACAATGACGTGCCACGCACATATGG
GGAACACCGCAAAGCCAAGGCCCGTGCTGAGGCGGAGGCCAAGGCTAA
GGCTGAGGCGGAGGCCAAGGCTAAGGCTGCGGCGGAGGCCAAGGCTAA
GGCTGCGGCGGAGGTACGCCAATTAGAAACGGAAGTTTCGCGGGAGAT
GGAAGCCCAAGAAAAAAATAAACAGCAAAAGGAAAAGCCGGAGGAGA
GCGAGGCGGAGAAGTCGACGATCGCAGTGACTCAGGTTCCGGAATTGGA
CACCTCCTACCGCAACGTTAATCTGGGGGTGCTAAACAAGAAGCTAGAC
TTTCGAATACCGAAGAAAACCCTCCCACCGGCAACAACAATAACCTCAA
CAAGTCCAGTCAATGGTAATGGGGAGAATCCAAGCTGCCCCTCAAATTC
CCCCACAAGCAAAAGCTGTGATGCCAACCAGGACAAAGATACTTATAAG
AATAAAGATAGGTATTTAAATAAGGCTAAGGCTAAAGACAAGGTAGAT
AAGGGCAATGAGGTGTCGGAGAACAATCTGGATAAGTCTGAGAAGCTTG
AAAAATCGCAGGATAAGAAGGCAAATGACAAGGAGAACAAGTCCGACA
AAAAGGAGAAGAAGAGACTGAACAGGGAGCCTGAAAAGAAATCAAAG
GTTGAGAACCCCCTCGAGATTGTGGACTCGAATAGCGTGGTCAGTGAGG
AAAGCTCGGAAAATACAGACAATGTGGAAAATGAACCGCCTCTTAGCGA
GACTAACGCGTCTCCAGTTCCAGAGCTAGCTACCAGCACTCAGGACAGT
CAACAGGACCAGTCAGTGAGTGAAGAGTTGGACATCCTfGCCAAAAACC
GTAGAATGTCCGGAACTAGAATAAAGACTCCCATTTCGTCTACTGGAAA
CCCTGCACTAAAGCGACGGGCAGATGATGATGTTGAGGACAAGTTGGAA
AATCCGACTAAAAAGAACTGCGCAAAGTGGGAGGCAAAGCCTGACAAG
GAAAAATCGGAGGATGATACCATCGACAAGATTAAATCCATGAAAGTA
ACTAAATTCGCTGATGTCGAGATGAAGGTTACAGAAGAAAGCCAGAGTG
CTGAGGAGGAGGAGATTACTGAGCAGAAAGAGAGTACTGAGGAGGAGG
AAGGTACTGAGCATAAAAAGAGTACTGAAGAGAAGGATAAGCCGCCAA
AAATCTCCAAGATAAAAATTGTCCTTACTCCCATTGCCCATACAACACA
AGTGGTTCGTCCTAATGATGGCTTCAAGAACAATCAAGAAAAGATCTTG
GACAACATGGCAACTGATGAGCACGATGATGAGGAGGTCCCCGGGCCC
CCAGCTCAATTCCTACGCCGGATTATGCAGCGTCGGAACTCCTTGGCTCC
TACGTATATGAAGCCGATGGTGGACAAGGATAAGATTGCTTCTTCCAGT
TTTACCTACGAGGATCTGCCGGAGCAGAAGCGCGGAAATCAGAACGCCC
GAAACCTGGCCATCATTTTCGAAAAAACTAGTGACAACTGCAGCGTGTC
CACTCAAAACATTATTAATGGCAAACGTCGCACTCGTGGATGTGAGACC
TCTTTTAACGAGACCCAATTGAGCCGAAACATCTTTGGCATGGGCCAGA
TAAACAGGTCGCGGCCAAAGGCCACCCGAGGGCAAGCTATTCACAAGG
AAACAGAGGATGATGTGGAGATGAAGCCTAAGAAGGCTCGATTGGAAG
CACAGGAGATAAATGGGGTCAGTGTTACGCCTGATGAACAGCAGGTAGA
GAACAACGTGGAAGTCACCCAAAAGGAGGTGGAAGCAATATCCTCAGA
GCCACTTCTCTCTTCTGAGGTTGAACCGACACGAAAGCCTCGCACGAAA
CCGCGAAAAAACGAGCTGGACAAGCTAAACGACGACATTGCGCAAATG
TATTACGGGGAGGAAGTGATGCGTGCCACCAGTCGCAGGGCTTGTACCC
GTCGATCGCGCACGTCCTCGCACACGCGCACCAGTAGCCAGCATTCCAG
GACGTCCTCTGTATCGCGAACCGATAGCATATCCACCGTATCGGATATTA
GTTCCATAATCGTCAGGAACACGGCGCGAAGGGGTAGAGGCATCAGATC
ATCCGAAAATGGCATCAACCGTGCCACGTTTAATGCATCCTTGAATGCA
AAAAAACCAAAGTTGTGCCGTGTTAGAATAAAGCGATGTGCTGCATTGA
TGGAGATGATAAAGGACCAGGAAAAGGAGGAACAGGAGAAGAAGGAG
CAGAAGAATAAAGAACCGAAGAAGAAGAAAGTGGGTGTGCAAAAAAA
GCCATTGAAAAGTAAGCCGAAAAGAGAGAATAGCGTTATTCTTAACACA
AATCCCGAATGGCACTCCATTTCGAAGGCTGTTATCAAGTGTGTCGTCTG
CTCGAAGTGGGTTCGCAGGAGCCCACTCTCTCATTATATGATGTGCCATA
AGGAGCACTATGCCGCCCGATTGCCACCCGATGTGCTTAAAGAGCTGCG
GGCCGGGCGCGGAAATCGACCGGATTACTGGGTTTCGCAACGCGGCGGC
TACACATTGCACTTCACTTGCCCGTTCTGCCAGAAGCCACTGCTACTCTG
CCAAAAAGGCATGATCGAGCACTTGATCGGCCATATGGGCGAGTCTCGT
TTTTACTGCTCCAACTGTAATATGCCACAGAACCGCCTCAGTAGGCTGCT
GGACCACACCGCATCCTGTGGGCCAGGTGCGAAGCCTTTAAGTAGCAAA
ACCGTCTGCCTACCGATGAGTGTTCACGTGTGCCACATCTGCCAGTTTAT
GCAGTACAGCAAGGAAAATATGGACCGGCATCTTACTGTTCAGCATGGC
CTAACGAAGGAGGAACTAGAAAGTGTGGAGCGCGAGGAGTTGATGCTC
TGCGACACAACAGACGTACCATATGCAGATTCGAATAAGGATGGCAGCG
CCCGAGAAAGAGAAGAGCAGGATGACCAGAACATGACCCAGGCTAACG
AAGGGTCGGAAGTGCCGGAAATACCGCCGCCTCCGCCAGAAATTGAGCC
CTTGTTTGTGGTCAATGAGTGTCTAATGACCTCTGAAATGGACACGGACA
TGGAAGAAGTCTTGGAACAGCCCGTTCAACATATGAGCTTAATGGTAGA
CGAAAAGCCTGTGACGCTACTCAGTGGGGCCACAGAACAGCTGGAGCCT
AGTGTCCCTGATCCCGAGCCTGTTGTTCCATCTGCACAAGATGATGGCAA
AGATGTAAATGAAGATGAAGACGTAGACGTGGAGGCAGTAGTGGATTC
CCTTCAGTCACACACTGACCAGACGGCTACTTCTATGTTAGCAGAAGTC
AGTCTAGCCGAATTGGCTGGGGATGTACTTGATGGTATTGGCAGCGACG
CGTCCGACTATGAGATGGATGATAATTCAGAGCAAGTGGATACAACTAA
CAAAAACGGCTACGGTGATGATGACGATGATGCGCTTACCGACGATTGG
GTGGATCTGGAGACTGCCAAGCGCAATTCCAAGTCCGCCAAGAGCATTT
TTAGAGTGTTCAATCGCTTCTGCTCGCGTTTAAACAAATTACCCCGATCC
AGCAGAGCAGTGCCCTCGAATGGGAGTGAAAACAGCGATGGCAGCGAC
AACAACGACGACGACGGCGATAATCCTGATCCCAGCGAGCTAATGCCAA
CAATGCAACCATTGGAGCCGGAGCCAGAGATGGGGGATTCATCCACATC
TACAGGTGCTAAGTCGCTATCCGAACGGGTGGAGAATGTGGGCTTTCAA
AAGCCCTCTTCAGACGAGGATCAAAATCGCGTGGCAGCATCCTATTACT
GCGTGCAGCCGGGTTGCACTTTCCTCTTTTCCAATGAGCTGGAAGGCCTC
GAGAATCATTTTGCGTTAGAGCACCCTCTTGTTCGATGGAGCGGCAAAT
GTGGCATGTGCCGTCAGAAAATCACGGCAACGGAAACGAATCTCAGAAT
TTCTGAAGAGTTGCGCCACATGAGGGACGTGCACATGAAGGACATATCC
ACCCTGCCTCCTCCTCAGTCATCTGCGGTTGAAAGCCCAGCCGTTATTGA
ATCCTGCCTGAATCAGCGTGAACCAGTACCTGAATCAGAACCTGATCCC
GTTCCTGAGCTTCCCAAGCTGCGTGTTCGACGCTTCACTGGGGATCGCCT
TGTTGTGGATTCACAAGCGGAAAAGAGCCAACCGGTAGCAATAGTTGTC
AGTGATGATGATAATCCGCGAAATGGGATGCTAAGGGACTTGCTGGCGG
CGGATCCACGGCCACCCAATCAGCAGTTGGACCTCCAAGCCGCTGGACT
GGGCGAGTTCCTTTGCGCCAAGCCCGATTCACCGTCAACAGAACCGGTC
AAGCAGACGCCCGTAATTGTTGGCTATTCGAGTGGCTTGGGCTTGAAAA
TCGGCCAGGTCCTTAGCAGAACTCAGATTTCAGCTAACTCACGGCTATC
GCCAGTCGTTAACGATCCCCTGCCAGAGAAGTCTTCTGCTCCTGCTGCCG
TTGAAGAGAATCGTAATCGATTCAGGTGCATGGCCACCAACTGCAATTT
TGTTGCTCACAAGCTCATGTTCATGCGGGAGCACATGAAGTTTCACAGCT
ACAGTTTCAGCAGCACCGGTCACCTGAACTGCGCGTACTGCTCCCATGT
GGCAGTCGATGTGGATGATTACTTGCGCCACGGAGTGATCATTCACGAC
CTGGCACCACGCTCCGAACTGGAGAGTTCAACTGGACCACCATCTGTTA
CCCAGAAAATCCGGGATATGCTCAGCCAGCGGGAAAATGGTCGTGTTCC
ACCACCAACTCCTCAAGTCACTCTGTCTGATGTGGTCCTGGGTCTTTTAG
AATGCACCGGATACAGCGAGGATAAACTGTACGCCTGTCCCCAAAAGGG
CTGCATCGTGCGGCTGACAGATGAGCAGCTTGTAAACCATTTGCGCTAC
CACATTCGTAGCACTCATCAGGGCAGCGAGTTGGTGAAATGCAAGTTTT
GCACCAAGGCGATGCATCCGCCGGCACTTCGTACGCATCTGCAGCAGTA
CCACGCCCGGCACAGCATCTTCTGCGGCATTTGCTTAGCCACATCGGTCA
ACCAGCGCATAATGATGTATCACATGAGCACGGTGCACTCCAAGGCCTA
CGGCCGGCCTAACGCGCGGCTGGCGTTTGTGTCACTGCCCGTGAAGATC
GACGCGAGTAAGAAGAACGTAGAAAGCGAGTTCTACGTGGCCGTCGTG
GAACAGCCCTTTGGCAACCTCCAGATGCAGGATTTCCAGCGCAAGCTGT
TCGATGAAATGGACCGTCGGCGTTCGGGAACAAAGACGTACTTCCGCAG
CTCCGAGGTGCATATCCTGCCAACGCAGCCAACATTCCAGCGACCGCTA
TACTGTACGGAGTGCCCCTTCTCCACCACGTCAAGGGTTAACATGCAGA
TGCACCTCTATGAGCACAAGGATGAGACCATTCGGGAAGCCTCCAATT
GGCGGACTTGATAGTTCCAGCAACCTCTTCGGTATTAACTGTTTCGGCGA
GTACGTTGGTGGCACCGCCGAGGCCAGGCAAAGATTCAGAAAAACCATC
TACTTCCGGACAAAGTGGTGATGCAGCGACGGAGCAGCTGAATCCAGAT
GTTCCTGGAACCCACAAGCCCATCAAGCCACCGTTACGCTATGTGCCCC
CGGACCAACGCTACCGCTGTGGCTTCCTCCGATGTAGCGTCCTTTGTTTT
TCGGAATCTGCGGTGCGCAAACACATGCAGGCTAACCACAAATACTCGG
AGGTGGTAAGGTGCCCGCACTGCAAGAACTGCCAGGGTCAGTTTGGAGT
AGATAAGTACTTTGACCATCTTGCAATGCATAAGCGGCACATCTTCCAAT
GCGGCGCTTGCTCACGTCACAATAGCAGGCGTGTCATCGAGCGGCACAT
ACAGGAACGTCACAATATTCAAGATGTGGACATGATCGTACACCGCCAT
AATGACAGCAACAAAACGACCGAAGCCCGCTGGCTGAAGGCGCCTAAA
TTGGCACGTCATTCGCTAATGGAGTACACGTGTAACCTGTGCCTCAAGTA
CTTTCCAACGACCGTGCAGATCATGGCCCATGCGGCGTCCGTTCACAAA
CGCAACTACCAGTACCACTGTCCGTACTGTGAATTTGGTGGAAACCTCG
CCACCGCGCTCATTGAACACATCCTTCGCGAGCACCCGGAAAGGGAAGT
GCAGCCTGTGCAAATCTACCAGCGCATCGTGTGTAAGAACAAGCAGACG
CTAGGCTTCTACTGCACCACCTGTCACGAGGTGGCCAGCAGCTTCCAGA
AGATCGCTATGCACTGCGACAAGGAGCATAAGTCGCGCAATCCGGTGCA
ATGTCCCCACTGCATTTTCGGGCATTTGGCCGAACGCCAGGTTGTCTTAC
ACATACAAGAGAAGCATCCCCATGAACGCGGACTGGCAATGGTGCAGTT
CGAACGCGTGCTTAATGACATCCCGAACAGCATAAGCTGGGAGATAGGT
CGGCCCATCGAAGTGGAGCCTGAGAAGGAGATCCCGAACAATGGGGAG
AGTGCATTCCTGCCGCTAAGCCAGAGACAGGTTGTAACGGAAGTGGTGG
ACCTGCTGGATTCAGACGACGAGGCGGACGAGTACGGTGAACAAGATG
ACGCGAAAATCGTGGAGTTCGCCTGCACACACTGCGACGGGACAAACAC
CAACTTGCCGGACCTACGCTCCCAGCACTGGGCCCGCGAACATCCCGAC
CAGCCCTTCTATTTCCGCGTTCAGCCGATGCTGCTCTGCTCCGAGTGCAA
GAGATTTAGGGGCAATGCAAAGGCACTTCGCGAGCACCTGCGTGCGACA
CACTCTATCCGGAGCATAGTGGCTGCGGACATTCGTCGACCGATGGAGT
GCGCTTACTGCGACTACCGCTATAAAAACAGGCACGATCTTGCGAAACA
CATCAGTGAGATAGGTCACCTGCCCAATGACCTGAAGCACGTAACAGAT
GATGAAATTGATGCCCTGATGCTGCTCAGTGCCAGTGGAAGTGGTGGGG
CTGTTAACGAATACTACCAGTGCGGATTGTGCAGTGTGGTTATGCCAAC
GAAGGAGACAATTGTCCAGCACGGCCAAGTGGAACACTGCAAGCCCGA
CGAGCGTTTCTGCTTCCGGCAGCTAGTGTCGCCAGTGATATACCATTGTT
CCTTCTGCATGTTCAACTCGACCGATGAGCTGACTACGCTGCGCCATATG
GTGGACCACTACAGCCGCTTCCTGGTCTGCCATTTCTGCACACGCTCTCA
GCCGGGTGGTTTCGATGAGTACATCCAGCACTGCTATACCTACCACCGG
GACGATATCAAATCCTTCCGGGACGTGCACACGTTTAGCGATCTGAAGA
GGTACCTTAGTCAGGTGCATTACCAATTCCAGAATGGGTTGATTATCACA
AAAAGCAGTCTCCGTTATACACGTTACAAATCCGACAAATGTATGCTTG
AGCTAGACGCTGAGCTAATGGCCAAGGCCCAGCGGCCACCCATTCCGCG
TCTGCATATCAGACTCAAGTCGACCGGCGTTCAGATGCAGAGCCCCGAG
GGGGCTGATGTGGAGAAACCTGTGTCGTTGTTGCGGATCACAAAGCGAC
GAAAAACGCTTAATCCTGGCGAATTGCTCCGCTCATTCCGCGAGGAGAA
TGAGGTACAGCCACAGCCACCGGCCTCTTCAACATCGTCGGGGACGGCT
CCTTCTCCTGCGGCAGGTTCTGTGTTCAACCTGTTCAAGCGCCGCAACAG
TCTCGTTGTCCGCCCAGCAACCAGCAACTTGGATCAACACTAA
>CG14438|FBgn0029899
MEDSEDDVVVVSCDTSMKEKVKAKLVEIRKFVPFIRRVRIDFQDTLSKVQG
HRLDALVNLLDREDVSMSSLNKAEVIIDKIRTRFNPRIEIDTGEIIDITENTDAK
ASDEGQRSPAEPRAALQAIVQDTKTPTIPEPTSPAALKHSSLRGSRGFLAVM
QKALIEEKKQRASEQKTDKETNGVTQLETNFSRRSYTTSSQSTCRSSEISVRA
ENPDFKRRSTSLVQHAPLQEASPGQSKKDLPISLSVQGLPALVSASTASPANT
LEEARKKLAALKYGLGYfVPSMPPLASNTNDPRGRKGINLPETNNNKDNDL
GIALQSPPPMRTPSPIPPPPRMKAGTWASFSNVPQESAFTGQHAVQRNSVPP
GDSRAFGDALAHEPRSFYGTDSREPRDPRIWKSKTSQQQQHQQAPQAQIPP
YSSDPRRSISTYSGFEEGGFRGGHNKRGFGRHNDVPRTYGEHRKAKARAEA
EAKAKAEAEAKAKAAAEAKAKAAAEVRQLETEVSREMEAQEKNKQQKEK
PEESEAEKSTIAVTQVPELDTSYRNVNLGVLNKKLDFRIPKKTLPPATTTTSTS
PVNGNGENPSCPSNSPTSKSCDANQDKDTYKNKDRYLNKAKAKDKVDKG
NEVSENNLDKSEKLEKSQDKAANDKINKSDKKEKKRLNREPEKKSKVENP
LEIVDSNSVVSEESSENTDNVENEPPLSETNASPVPELATSTQDSQQDQSVSE
ELDILAKNRRMSGTRIKTPISSTGNPALKRRADDDVEDKLENPTKKNCAKW
EAKLPDKEKSEDDTIDKIKSMKVTKFADVEMKVTEESQSAEEEEITEQKESTE
EEEGTEHKKSTEEKDKPPKISKIKIVLTPIAHTTQVVRPNDGFKQEKILDN
MATDEHDDEEVPGPPAQFLRRIMQRRNSLAPTYMKPMVDKDKIASSSFTYE
DLPEQKRGNQNARNLAIIFEKTSDNCSVSTQNIINGKRRTRGCETSFNETQLS
RNIFGMGQINRSRPKATRGQAIHKITEDDVEMKPKKARLEAQEINGVSVTP
DEQQVENNVEVTQKEVEAISSEPLLSSEVEPTRKPRTKPRKNELDKLNDDIA
QMYYGEEVMRATSRRACTRRSRTSSHTRTSSQHSRTSSVSRTDSISTVSDISS
IIVRNTARRGRGIRSSENGINRATFNASLNAKKPKLCRVRIKRCAALMEMIKD
QEKEEQEKKEQKNKEPKKKVGVQKKPLKSKPKRENSVILNTNPEWHSISK
AVIKCVVCSKWVRRSPLSHYMMCHKEHYAARLPPDVLKELRAGRGNRPDY
WVSQRGGYTLHFTCPFCQKPLLLCQKGMIEHLIGHMGESRFYCSNCNMPQN
RLSRLLDHTASCGPGAKPLSSKTVCLPMSVHVCHICQFMQYSKENMDRHLT
VQHGLTKBELESVEREELMLCDTTDVPYADSNKDGSAREREEQDDQNMTQ
ANEGSEVPEIPPPPPEIEPLFVVNECLMTSEMDTDMEEVLEQPVQHMSLMVD
EKPVTLLSGATEQLEPSVPDPEPVVPSAQDDGKDVNEDEDVDVEAVVDSLQ
SHTDQTATSMLAEVSLAELAGDVLDGIGSDASDYEMDDNSEQVDTTNKNG
YGDDDDDALTDDWVDLETAKRNSKSAKSIFRVFNRFCSRLNKLPRSSRAVP
SNGSENSDGSDNNDDDGDNPDPSELMPTMQPLFPEPEMGDSSTSTGAKSLS
ERVENVGFQRPSSDEDQNRVAASYYCVQPGCTFLFSNELEGLENHFALEHP
LVRWSGKCGMCRQKITATETNLRISEELRHMRDVHMKDISTLPPPQSSAVES
PAVIESCLNQREPVPESEPDPVPELPKIRVRPYTGDRLVVDSQAEKSQPVAIV
VSDDDNPRNGMLRDLLAADPRPPNQQLDLQAAGLGEFLCAKPDSPSTEPVK
QTPVIVGYSSGLGLMGQVLSRTQISANSRLSPVVNDPLPEKSSAPAAVEENR
NRFRCMATNCNFVAHKIMFMREHMKFHSYSFSSTGHLNCAYCSHVAVDV
DDYLRHGVIIHDLAPRSELESSTGPPSVTQKIRDMLSQRENGRVPPPTPQVTL
SDVVLGLLBCTGYSEDKLYACPQKGCIVRLTDEQLVNHLRYHIRSTHQGSBL
VKCKFCTKAMHPPALRTHLQQYHARHSIFCGICLATSVNQRIMMYHMSTVH
SKAYGRPNARLAFVSLPVKIDASKKNVESEFYVAVVEQPFGNLQMQDFQRK
LFDEMDRRRSGTKTYFRSSEVHILPTQPTFQRPLYCTECPFSTTSRVNMQMH
LYEHKDETIREASKLADLIVPATSSVLTVSASTLVAPPRPGKDSEKPSTSGQS
GDAATEQLNPDVPGTHRPIRPPLRYVPPDQRYRCGFLRCSVLCFSESAVRKH
MQANHKYSEVVRCPHCKNCQGQFGVDKYFDHLAMHKRHIFQCGACSRHN
SRRVIERHIQERHNIQDVDMIVHRHNDSNKTTEARWLKAPKLARHSLMEYT
CNLCLKYFPTTVQIMAHAASVHKRNYQYHCPYCEFGGNLATALIEHILRELIP
EREVQPVQIYQRIVCKNKQTLGFYCTTCHEVASSFQKIAMHCDKEHKSRNP
VQCPHCIFGHLAERQVVLHIQEKHPHERGLAMVQFERVLNDIPNSISWEIGRP
IEVEPEKEIPNNGESAFLPLSQRQVVTEVVDLLDSDDEADEYGEQDDAKIVEF
ACTHCDGTNTNLPDLRSQHWAREHPDQPFYFRVQPMLLCSECKRFRGNAK
ALREHLRATHSIRSIVAADIRRPMECAYCDYRYKNRHDLAKHISEIGHLPND
LKHVTDDEIDALMLLSASGSGGAVNEYYQCGLCSVVMPTKETIVQHGQVE
HCKPDERFCFRQLVSPVIYHCSFCMFNSTDELTTLRHMVDHYSRFLVCHFCT
RSQPGGFDEYIQHCYTYHRDDIKSFRDVHTFSDLKRYLSQVHYQFQNGLIIT
KSSLRYTRYKSDKCMLELDAELMAKAQRPPIPRLHIRLKSTGVQMQSPEGA
DVEKPVSLLRITKRRKTLNPGELLRSFREENEVQPQPPASSTSSGTAPSPAAG
SVFNLFKZRRNSLVVRPATSNLDQH
Scim11
AE003568 (insertion @237885), nearest ORF (CG1494) @228917 (9 kb away)
>>CG1494|FBgn0031169|cDNA sequence
ATAAGGTGGGACCAGGACCACGGGGTGCTGACCCAAACACCATGGTAC
ATACTGATCTTAGTGCTGTTCTGCTACAACTGCGCCGCCGTTGCCTTTGC
CATAATGGTGGCTGCCTTTTTCCGGAACGCTCTCAACGCCGTTCGGGTGT
TGACAATCCTGTGGATAATGTCCTACGTGCCCACCTTCATTCTGTCGAAC
AACTTGGAGGGCAATATTCACGCCCTGCGCTACGTGTCGTATGCGCTGC
CAAATGTGGTGGCAACTCTGGTGATTGAATTTCTCATCGAACGGGAGTC
GATCGTCCATATCACGTGGGAGGACTCTGGGTACAGACTCAACTATGAC
GGCGGCCACATAACGGTAACCGCGAGCTCCTGGATCTTCATGCTGAATG
CTTTGGTTTACTGTGCAATTGGTCTCTATGTGGACATGTGGCGGGGTGGC
GACCGATCGGGTAAGAAGATGAAGAAACCCAACACGAATGCCAGTGTA
CAAGAAGATCCATACCACGAACGGGGGGACAGTTTCACTCATCAGGGTC
AGGCCATTGGCGTTAACTCAACGAAAATCTATGAGGTGGAACCCTCACA
TCGGCGCTTCAAGCTAAAGATCAAGAAGCTGTGCAAGCGATTTGCGACA
AACGATCGTCCGGCATTAAATCTCTTCTCGTGGAATGTATACGAGAACG
AGGTCACCGTTCTGATGGGTCACAATGGCTGTGGCAAGAGTACACTGCT
CAAAATACTAGCCGGCTTGGTGGAGCCCAGTCGGGGCACTGTGATGATA
TCCAGCCACAATATACAGACCGAAAGGAAGGCGGCCTCAATGGAGCTG
GGCATCGCATTTGGCCATGACATGCTTCTCACCGGCTTCACAGTCATTGA
TTACTTACGATTCATTTGCCGAGTTAAGGGATTGCACAATAACATCGAGA
TCGATGGTCAGTCCAACTACTTTCTTAACGTCCTGCAAATCGGAGGCCTA
AAGACCAAACGAATCCGCACCCTCACTGATCGCGATTTGTGCCTGGTTA
GCATCTGCTGTGCCTTTGTCGGTAATAGTCCCATAATCCTCATAGACGAC
GTTCACTCCGATCTGGACAAGCGCACGCAGTCGCTGGTCTGGAACCTGA
TTAACGAGGAAAAGTCCAAGCGCACCATTATCCTGGTGTCCAACTCGCC
GGCTCTGGCCGAAAACATTGCCGATCGCATGGCCATTATGTCCAACGGG
GAGCTCAAGTGTACCGGAACGAAACCGTFITCTAAAGAATATGTACGGAC
ATGGCTATCGATTGACCTGCGTTAAGGGGAAGAACTACAAAAGGGATGA
ACTGTTCGGCATGATGAACAGCTATATGCCCAACATGAGCATCGAGAGG
GATATTGGGTACAAGGTCACCTTTGTGCTGGAGAACAAGTTCGAGGATC
AGTTCCCTATGCTAATCGATGATCTGGAGGAGAATATGCAGCAGCTGGG
TGTGGTCAGTTTTCGGATTCGGGACACGTCGATGGAGGAAATCTTCCTGC
GATTTGGATGCGAAGACAATGACCAAAGTGGCGCTTTTCAATCGCACGA
AAACGCGCAAGTCCTGCTGGAGGAGTACTATTCCACACTGGCTGAGGCC
AATGAAAAAGGTCGAAGGACTGGCTGGAAGCTGTTTTTTTTGCATGGCA
GGGCGGTGATCTACAAACGTTGGATTGCGGCCCACCGACACTGGATCGT
ATTGATTTTTGAGGTTCTGGCCATGGCCCTGGTCGCGGTGTGCACATTCT
CCAGCATTTTCATCTACGGCAAGAACTATGAGTTGGAACCGCTGACCTTT
AACCTCAGCCAGCTGCACACTGTGGACGCCTTCGTGGAGCTCTTTTCCGA
AGAGGAGGATGTCAAGGATATGCACGCCTATTACACGGAGCTGCTCTAT
TGGTACGACGCTCATGTGGCGACGCTGACAAAAAACCGTCATAACGCAT
ACGCCCTGTTGACCCAAAACCAATTCACCGCCCACGTCAACTCGCGCTA
CATTTTCGGAGCCACGTTCGATCAAAAGATCGTCACCGCCTGGTTTAATA
ATATACCACTGCACTCTGCACCCTATGCCTTGAATGTTGTCCACAATGCG
GTAGCCAGGCACTTGTTCGACGAGGAGGCCACCATTGATGTGACCCTGG
CGCCGCTGCCGTTCCGGACGGCCATTAACACCTTTCCGCCTAGCAGCCAT
ACATTTGGTGGCTGTTTAGCATTTGGCATTTGCTTCGTGCTGACATTTATT
TGGCCAGCATTCGCGATTTACATGATCACCGAGCGTGGAAGCTTGCTGA
AGAAACAACAGTTTTTGGCCGGAGTCAGGGTGTGCAGCTACTGGACGTT
TACGGTGTTATATGACTTGCTCTTCCTGCTGATCTTCTGCGCGTGCGTTGT
GGTCATGGTGGCATTATACGAGAATCCGAACCACGACGTTATGCTATAC
GGTTACATATCGGTCACATTGATGCTGGGAGGATTCTGGGTGATCCTGCT
TGCGTATTTAATGGCGAGCCTGTGCCGGAACCCGTGCTATGGATTTTTGT
GGCTATGCGGGATTAACAGTATCGGCCTCGTCTGCTTCTCGCAATTCTAT
AGAACTCATCCAGAATCTATGCTCCTCGAGCCGACCTTTATGGCCATGTA
CACGGTGGCCACAGTTATATGCAAGCTTTTCATGATCTACGAATTCAAGC
TAATCTGCATGGATCCCGTCGTGAATTTTACCTCCGTCGAGGTATTCAAA
TCGGAGTGCTTGAGCATCACGGGAGCAAACAACTCCGGCAAGACTACGC
TGCTCAAGGTGGTGGTGAATGAGACAAAGATGAACGCTGGACAGCTCTG
GATCCATGACTACTCGGTGAACACCCACCGTGTCCAGTGCTACCGGATG
GTGGGCTACTGTCCGCAAAAAGACAGCCTTCCGTCGGAGTTTACCCCGC
GTGAATTGCTGTACATTCACGCCATGCTTCAGGGCCACAGGCACCGCAT
AGGCCGCGAATTGTCGGAGGCACTGCTCCGTCTGGTGGGACTCACCCCT
TGCTGGAATCGGTCAGTGCGCATGTGCACCACAGGTCAAATCCGGCGAT
TATATTTTGCCTACGCCGTGCTGGGATCCCCGGATCTCATCTGTGTGGAC
GGTGTACCAGCTGGACTGGATCCGACCGGGAAGCGAATCATCCTGATGA
TGACCTCCACCATGCAGGCGATGGGGTCCAGTTTCTTGTACACTATGCTC
ACAGGTCTGGACGCCGAGCGACTGTCCCTGCGCACGCCACTTCTTTTAG
AGGGCCAACTCTGGATGATTCGGCCCATGGACACAGAGACCGAGAACTA
TAAGAGTGGCTACCAGCTGGAGGTACGATTCAAGAGGAAGGTCAATCCT
AATGTCAGCATGTCCCGGGCCACCTGGAACCTAATCAACCACTTTCCCAT
GTCACCAAACAAGAAGTTCAGTGCCTTCATGGAGATCAAGTTTCCCGAT
GCCGTGCTCACAATTGAAAGAGATGACTCGATGGTATTTGTATTGCCGTT
GGGCACGACCACCTTCTCGGAGATATTTCTTACACTGCGCAAAGATGCC
TTCGAAATGAACATAGAGGACTACTTTATCACACGCAACATGCTCGTGG
GCTTCCAGATATITACCTATGATCAACATCAGGACAATCCATAA
>CG1494|FBgn0031169
MVAAFFRNALNAVRVLTILWIMSYVPTFILSNNLEGNIHALRYVSYALPNVV
ATLVIEFLIERESIVHITWEDSGYRLNYDGGHITVTASSWIFMLNALVYCAIGL
YVDMWRGGDRSGKKMKKKNTNASVQEDPYHERGDSFTHQGQAIGVNSTK
IYEVEPSHRRFKLKIKKLCKRFATNDRPALNLFSWNVYENEVTVLMGHNGC
GKSTLLKILAGLVEPSRGTVMISSHNIQTERKAASMELGIAFGHDMLLTGFTV
IDYLRFICRVKGLHNNIEIDGQSNYFLNVLQIGGLKTKRIRTLTDRDLCLVSIC
CAFVGNSPIILIDDVHSDLDKRTQSLVWNLINEEKSKRTIILVSNSPALAENIA
DRMAIMSNGELKCTGTRPFLKNMYGHGYRLTCVKGKNYKRDELFGMMNS
YMPNMSIERDIGYKVTFVLENKFEDQFPMLIDDLEENMQQLGVVSFRIRDTS
MEEIFLRFGCEDNDQSGAFQSHENAQVLLEEYYSTLAEANEKGRRTGWKLF
FLHGRAVIYKRWIAAHRHWIVLIFEVLAMALVAVCTFSSIFIYGKNYELEPLT
FNLSQLHTVDAFVELFSEEEDVKDMHAYYTELLYWYDAHVATLTKNRHNA
YALLTQNQFTAHVNSRYIFGATFDQKIVTAWENNIPLHSAPYALNVVHNAV
ARHLFDEEATIDVTLAPLPFRTAINTFPPSSHTFGGCLAFGICFVLTFIWPAFAI
YMITERGSLLKKQQFLAGVRVCSYWTFTVLYDLLFLLIFCACVVVMVALYE
NPNHDVMLYGYISVTLMLGGFWVILLAYLMASLCRNPCYGFLWLCGINSIG
LVCFSQFYRTHPESMLLEPTFMAMYTVATVICKLFMIYEFKLICMDPVVNFT
SVEVFKSECLSITGANNSGKTTLLKWVNETKMNAGQLWIHDYSVNTHRVQ
CYRMVGYCPQKDSLPSEFTPRELLYIHAMLQGHRHRIGRELSEALLRLVGLT
PCWNRSVRMCTTGQIRRLYFAYAVLGSPDLICVDGVPAGLDPTGKRIILMM
TSTMQAMGSSFLYTMLTGLDAERLSLRTPLLLEGQLWMIRPMDTETENYKS
GYQLEVRFKRKVNPNVSMSRATWNLINHFPMSPNKKFSAFMEIKFPDAVLTI
ERDDSMVFVLPLGTTTFSEIFLTLRKDAFEMNIEDYFITRNMLVGFQIFTYDQ
HQDNP
Scim12¹
Scim12²
Scim12³
Scim12⁴
Scim12⁵
Scim12⁶
Scim12⁷
Scim12⁸
AE003582 (insertion @76200), nearest ORF (CG9894) @72208.
>>CG9894|FBgn0031453|cDNA sequence
CAGTGTGTTTGTGTGCTTCGTTCGGTGCGGTTCTCTCTGTCTCTCTCTCGC
CTTCCCCGAGTATTTTGCGCTGGTTTTTTGTCAACAACAAGACAATCCAC
AAAACCAACCCGAATTGTTCTCTATATAACGCAGAAACTAAATAGTTCC
GGAAAACCTCAAAGAAACCAATTCAAATATGTCGGCTGCTACGGAACAA
CAGAACAACGGCGATGTGGCCGTGGAGAAGGTGGCGGCAGATGATGTG
TCTGCTGTCAAGGACGATCTCAAGGCGAAGGCGGCCGCCGAGGATAAG
GCCGCTGCTGCCGATGCCGCCGGCGACGCGGCCGACAACGGTACGTCAA
AGGACGGCGAGGATGCCGCCGATGCCGCCGCCGCTGCCCCCGCAAAGG
AATCCGTGAAAGGCACCAAGAGGCCAGCAGAAGCCAAATCCGCAGAAT
CAAAGAAGGCCAAGAAGGCCGCGGCCGCCGATGGAGATTCCGATGAGG
AAGAGGCTCTGGAGGAAATCATCGAGGGCGACAGTGAAATCGAGAGCG
ACGAGTACGACATCCCCTACGATGGTGAGGAGGATGACATTGAATGTGA
TGATGATGATGATGATAATGATGACGGTTCCGGCTCGGACGATCAGGCG
TAATAATAATGTAGTCAAAAATACAAACAAAAACAAACAAAAATTTAA
ATTAATAATAAATAAAAGTTACAAGCAAAAAAAAAAAAAAAAC
>CG9894|FBgn0031453
MSAATEQQNNGDVAVEKVAADDVSAVKDDLKAKAAAEDKAAAADAAGD
AADNGTSKDGEDAADAAAAAPAKESVKGTKRPAEAKSAESKKAKKAAAA
DGDSDEEEALEEIIEGDSEIESDEYDIPYDGEEDDIECDDDDDDNDDGSGSDD
QA
Scim13¹
Scim13²
AE003582 (insertion @96627), nearest ORF (within CG9892) @89868
>>CG9892|FBgn0031449|cDNA sequence
TACATATATATTCTTGGCCAGAGATATACATGGTATATATGGTCTCGGTT
CTTCTGCGCGCGTGTTACAAATCAAAAAGTTTGCATATTflTCGAAATTA
TAAATAAAATCGTTCGTTTCATCGTTTCAATCGCCGGTCAACAATCGAGT
GCCAGCTGTGTTTTTTTGCCACTTCGAGAACGATTCCAGAGTGCTTTTCG
CCAAATTTGATATGTGTAAATAATGTGCGAGCAGAGCCAATAAATATAT
TCCGATAAGCTTCCGAAATAAATCAGCGTTCAAACGTTTAAACGTTTTGT
AAACAGCACGGTGGAACACCAAGAGTACACACAAAATGGATAGCAGCA
AGTTGTTGAAGAATGTCTACGGCATCGACATTCACTTCGAAGATCTCGTC
TACCAGGTCAACGTACCCAAAAAGCCAGAGAAGAAGTCCGTGCTGAAG
GGCATCAAGGGTACGTTCAAGTCGGGCGAACTGACCGCCATAATGGGCC
CCTCGGGGGCGGGCAAATCTAGTCTTATGAACATCCTCACCGGTCTGAC
CAAATCCGGCGTCAGCGGGAAGATCGAGATCGGGAAGGCGCGCAAACT
GTGCGGCTACATTATGCAGGACGATCACTTCTTTCCCTACTTCACCGTCG
AGGAGACCATGCTGATGGCGGCCACACTTAAAATCTCCAATCAGTGCGT
CAGTCTGAAGGAAAAGCGAACTCTGATCGACTATCTGCTGAACTCGCTG
AAGCTGACGAAGACGCGGCAGACGAAGTGCTCCAACCTGAGTGGCGGC
CAGAAGAAGCGCCTATCCATCGCCCTGGAACTGATAGACAATCCAGCTG
TGCTATTTTTAGACGAGCCCACAACCGGATTGGACAGCTCCTCCTCCTTC
GACACCATCCAGCTGCTGCGCGGCCTGGCCAACGAGGGACGTACCATCG
TGTGCACCATCCACCAGCCGTCGACGAACATCTACAATCTCTTTAACCTG
GTCTACGTGCTAAGCGCGGGTCGATGCACCTACCAGGGCACGCCCCAGA
ACACGGTCATGTTTCTCAGCAGCGTGGGCCTGGAGTGCCCGCCCTACCA
CAATCCCGCCGACTTCCTGCTGGAATGCGCGAACGGGGACTACGGCGAT
CAGACGGAGGCTCTGGCGGAAGCGGCCAAGGACATACGCTGGAGATAC
GATCAGCAGTTGATGCAGGGCGAGGATGCCGATGCGCCCAGCGAGACG
CAGGTGGCCAAGTTCAATGAATCTCAGTCACCGGGGCAGGTCCAGGTGC
AGGTGCAGAAGATCGAGATCCAGAACATGGAGTCGTCGAAGGATCTGA
CCAAGCACACCTATCCGCCCACGGAATACATGCGACTGTGGCTGCTCAT
CGGCCGGTGTCATCTTCAGTTCTTCAGGGATTGGACTCTTACCTACCTGA
AGCTGGGCATTCATGTGCTCTGTTCCATTTTGATTGGCTTGTTCTTCGGCG
ATTCGGGCAGCAATGCCACCAAGCAAATTTCCAATGTCGGCATGATCAT
GATCCATTGCGTATATCTCTGGTACACCACCATTATGCCGGGCATATTGA
GATATCCCGCCGAAATAGAGATCATCAGAAAGGAGACCTTCAACAACTG
GTACAAATTGCGAACCTATTACCTTGCCACCATCATCACATCCACACCAG
TCCATATCATCTTCTCGACGGTGTATATAACGATAGGATATCTGATGACC
GATCAGCCCGTGGAAATGGATCGATTTGTTAAGTACCTACTAAGTGCGG
TGGTGGTCACGATCTGTGCGGATGGTCTGGGCGTCTTTCTGGGCACCGTG
CTGAATCCAGTGAATGGAACTTTCGTTGGCGCCGTTTCGACGTCATGTAT
GCTAATGTTCTCCGGCTTCCTCATCCTGCTGAATCACATTCCGGCTGCCA
TGCGATTCATGGCCTATATATCGCCACTTCGCTACGCCCTCGAAAACATG
GTGATCTCGCTGTACGGCAATCAGCGTGGCCAGTTGATCTGCCCGCCCA
CGGAGTTCTATTGCCACTTCAAGAACGCTGTGACTGTGCTGCGACAATTT
GGTATGGAGGACGGCGACTTTGGTCACAACATTCTCATGATCCTCATCC
AAATAGCGATATTCAAGGTTCTGTCCTACTTTACGCTGAAGCACAAGAT
CAAGACGAACTGA
>CG9892|FBgn0031449
MDSSKLLKNVYGIDIHFEDLVYQVNVPKKPEKKSVLKGIKGTFKSGELTAIM
GPSGAGKSSLMNILTGLTKSGVSGKIEIGKARKLCGYIMQDDHFFPYFTVEET
MLMAATLKISNQCVSLKEKRTLIDYLLNSLKLTKTRQTKCSNLSGGQKKRLS
IALELIDNPAVLFLDEPTTGLDSSSSFDTIQLLRGLANEGRTIVCTIHQPSTNIY
NLFNLVYVLSAGRCTYQGTPQNTVMFLSSVGLECPPYHNPADFLLECANGD
YGDQTEALAEAAKDIRWRYDQQLMQGEDADAPSETQVAKFNESQSPGQVQ
VQVQKIEIQNMESSKDLTKHTYPPTEYMRLWLLIGRCHLQFFRDWTLTYLK
LGIHVLCSILIGLFFGDSGSNATKQISNVGMIMIHCVYLWYTTIMPGILIYPAE
IEIIRKETFNNWYKLRTYYLATIITSTPVHIIFSTVYITIGYLMTDQPVEMDRFV
KYLLSAVVVTICADGLGVFLGTVLNPVNGTFVGAVSTSCMLMFSGFLILLNH
IPAAMRFMAYISPLRYALENMVISLYGNQRGQLICPPTEFYCHFKNAVTVLR
QFGMEDGDFGHNILMILIQIAIFKVLSYFTLKHKIKTN
Scim14¹
Scim14²
AE003618 (insertion @24110), nearest ORF (CG13791) @25704
>>CG13791|FBgn0031923|cDNA sequence
ATGAGTTCCTACAGGACATTGGTGGATCATGGCCATCCGATTATAGTGG
GAAGCAGTGAAATATCGCTGGCCCCGAGTTCGGCAGCCAGTTCGCCCAA
GCCCCTACACCGGATGATCAAGTACTGGCGCAACAGTTCCGGATAAAAATT
CCGGGTCTCCGCAAAAGCGAGAGTTTCGCCGAGTATCGTCGCCATTCAT
CCAACTCGGCCACAATTTCGGGAGGATCAGGGGGCAGATCGAGCACTTC
GAGTGCCAGGCAATTGCAATACCAGCGACTGGAGATGGAAAGCTGCGA
GAATATAGATATGCTGACAGAACCACTAAGGTAA
>CG13791|FBgn0031923
MSSYRTLVDHGHPIIVGSSEISLAPSSAASSPKPLHRMIKYWRNSSGKIPGLRK
SESFAEYRRHSSNSATISGGSGGRSSTSSARQLQYQRLEMESCENIDMLTEPL
R
Scim15¹
Scim15²
AE003626 (insertion @73500), nearest ORF (CG4026) @73530
>>CG4026|FBgn0032147|cDNA sequence
AGCCGCTAGACCACGTAACGCCACGATTTTCGCCGGATCCACCGATTCG
ATTCGATTCGCCGCGATCGTCAGTGCCTATATATACAGTTCCCAACGGAG
CCGAGCGATAAAGATAAATGTGCAAAAACAAAGCGCACTTAGATAAAG
ATAGCGAAGTTCTCCCATGTGGAAGGCACAGTGCAAGTGAAGTGAAACG
AGAACGCAGTTTTGAATAGGAAATACGAAAGTACTCACATATATAGAGA
ACCCGAGACTTGGAGTCAGAATGCAAATGTGGCGAGCATAAAGTCGCAA
AGCGTGAAAATCTACGATATATACGAGTATAGTCGATTCCAAGTGTCAG
CCAAGTGAAACCCAGTGTGCAGCCGAAACCAAACCGAATGACTATGACT
TCTACGGTGCTCCAACGGCCCATTCAAGCCAAGCCAGAGAAGAAGGCCT
CCTCCAAATCGACCAGCTCCTCGAGAAGCCGCTCCACGATGGCCTGGTC
CAATGAGAAGCTGCGCTTCTCCTGCATCGACAACATCGGACTCAAGCAG
CTATGGAAGCTGATTGCCCTGGACACGAGTGCTTCATCCAAGCAGCGCA
GTGCCATGATGTTGGAAGTGGAGCAACAGCAGCAACAGCAGCAGCAGC
AGCAATCGAACAACAATAACGAGCGGATACCCAACGAGAACTGCGACT
ATTTGAGTCTACAGAGATCGGGCCAGGCGCCGAAGAATCACATCCAGGC
GCAGGATCCGGCTCAGATGTCCCTGCTCAAGTTFCTTGGCCATTGTAAGTA
CCCCATGTTGTTAG
>CG4026|FBgn0032147
MTMTSTVLQRPIQAKPEKKASSKSTSSSRSRSTMAWSNEKLRFSCIDNIGLK
QLWKLIALDTSASSKQRSAMMLEVEQQQQQQQQQQSNNNNERIPNENCDY
LSLQRSGQAPKNHIQAQDPAQMSLLKFLAIVSTPCC
Scim16
AE003628 (insertion @237450), between two ORF (CG13143 @234496 and
CG6187 @240204)
>>CG13143|FBgn0032255|cDNA sequence
ATGCAGAATTCTCCGGCTCCGTGTGCCTGGTACTTGCCCTGGTCCCTGGC
CGCCCAGCAGCACCAGCAAAAGATGCTGCAAATGCAGTCGCCGTTTCTG
GACAAGATGGGCGCCACATCGGTGGGCGGCATCTTCGCTGGCCAGCCGC
AGATGCAGCAACAATTGTCGCCCAATACGGCAGCAGCACCGCCGGCAA
ACTATCAGCAGCCCGCTTTGCATCCAAGCGCCGCACCAGGCGCACCACA
CTTCCACATGGGATCCCCGTATAGCCATCTGGCACCGCAGCTCCTCAACG
CCGGACAGCTGAACCAGAACGCACTGATGCACTCCGCCATGTTCTCTTC
CCTGCCACTTGGTGCGTACTATGCACCCGCCGCCGGCGCAGGTCACTCG
GCCTTTGGTGGCGTTCCCCTGACCACGGCTGCCCAGCAATCTCTATTGGC
CGCCACCGGAGGAGCAACTGCTGGCCATTTGGCCAACCAGCAGACGACG
GCTCAAGTGCCCGTCCAGGTGCCCGTGCAAATGGCCCAACGGACAGCTC
CGGCCGCCTGCTCCATGGTCCAGCCACTTAACTGCCTGCCGCACCAGGA
ACTGAATCACCTGTCGTCCATCAATCTCAACCTGCTGCGCAGTCCGGCGC
CTCCGCTCCCAGCCATTCAGGTCTTGCCAAGTGCCGAGGTGCCGATTAAT
AAGAAGGTGAGTTGCAGTTTGCTTAGTACTTGTAATGATAGGCACTATTC
GTACTTGAGCGAAGGCTAG
>CG13143|FBgn0032255
MQNSPAPCAWYLPWSLAAQQHQQRMLQMQSPFLDKMGATSVGGIFAGQP
QMQQQLSPNTAAAPPANYQQPALHPSAAPGAPHFHMGSPYSHLAPQLLNA
GQLNQNALMHSAMFSSLPLGAYYAPAAGAGHSAFGGVPLTTAAQQSLLAA
TGGATAGHLANQQTTAQVPVQVPVQMAQRTAPAACSMVQPLNCLPHQEL
NHLSSINLNLLRSPAPPLPAIQVLPSAEVPINKKVSCSLLSTCNDRHYSYLSEG
>>CG6187|FBgn0032256|cDNA sequence
TTTCGGCATAAAAACGTAATTTTCATGCGGTTTTTGCGGCAATTTAGGGA
CGTTTTTCGTTTGGCAAGTGGTGTTTGTGTTATGAATTAAAGTAACATTT
AACTCATTCAATTGAATCATCGCATAAAGCAGAGTGTTTTTGTGTTTGAA
ACTGAAATCTGCGCACGTGTTGACTAACTTGTTGTTATTATTATAGCGTT
GCTTAGATATTCTAGTAAATTGGCCGCAAATCAAAAACTATAAACAATT
CTCGTGGCTGTTGAAAATGGAGAGTTCCAAACTCTTGAGAAATGCACAA
ACCCAGCATGGAGATGCCTCATCCGTGGACGTGGAAAACATATTCCTGC
ACCGCCATATGCTATACACAAATCCCACTTCGGATGGCAATCTCCATGAT
CGGGAAGATTCCCCCGAATGCGTGTGGTGTCCAGACGACAAGGATGGTA
GTCCAGCTGAAAGCAAAGATCCACCGGTGTGGACGGATTGGAAATGTGC
CATCAAGAGCATGTGGAAACAGAAACATGAGCCAATGAAGGCGACGGA
AGAAGAGCATGTCATTATCCTCCAGTTGGATAAGTTCCAAGATGCTGAT
CCGGATGAAATCAGGGTGTATCAAGAAGCAGTACCCAAGGGAATATCCA
TATCAGAGGAAAAGTCTGAAACGCAATCAAAAGAAGTTTTGTCGGAAAA
GCGAAAAGCTAGCAGCACAGATGACGAAGGGCATGTGAAAAAAGTAAA
ATTAGAGGCCAATAGTCTAAAAACGAAGCGTCCTGGATTCAGCGATGAA
AGATACGACGAAACATCGTATTATTTTGAGAATGGTCTGCGGAAGGTGT
ATCCTTATTTTTTCACATTCACCACGTTCGCCAAAGGACGTTGGGTTGAT
GAGAAAATTCTGGATGTATTTGTCCGCGAATTTCGAGCCGCACCGCCGG
AGGAATATGAACGCAGCCTGGAAGCGGGAAAATTGACTGTTAACTCTGA
GAAAGTGCCCAAGGACTATAAAATCAAGCACAATGATCTGCTGGCCAAT
GTGGTGCATAGACACGAAGTCCCTGTTACTTCACAGCCCATCAAGATTG
TGTACATGGACAAGGATATTGTGGTTGTGAATAAGCCGGCATCGATACC
GGTACATCCTTGTGGAAGATATAGACACAACACGGTAGTTTTCATCCTG
GCCAAGGAGCACAATCTGAAGAACCTGCGAACCATTCACAGATTGGATC
GTCTCACATCTGGCCTACTTTTGTTTGGACGGACTGCTGAAAAAGCCCGC
GAATTGGAGCTGCAAATCCGAACTCGCCAAGTGCAAAAGGAATACGTTT
GCCGCGTTGAAGGACGCTTTCCAGATGGCATAGTTGAATGCAATGAGAA
AATCGACGTCGTGAGCTACAAAATAGGTGTTTGTAAGGTTTCACCGAAG
GGCAAGGACTGTAAGACCACATTTAAAAGAATCGGCGAGGTGGGCAGT
GATAGTATTGTTTTGTGCAAACCACTAACAGGACGAATGCACCAAATAC
GAGTGCATTTACAATTCTTGGGCTATCCCATTTCAAATGATCCTTTGTAC
AACCATGAAGTTTTCGGTCCATTGAAAGGAAGAGGAGGAGATATCGGTG
GAATAACCGAGGAACAGTTGATCAGTAACCTAATTAGCATTCATAATGC
AGAAAACTGGTTGGGCTTGGAAGGAGATCAAATCGTATCAGGGGAAAT
AAAAGACGTAGCAGCAAGTACTTCAGTAGTAGAGGCTCCCTCAGTAGTT
CAGGCTCCTATTAATAGTGAAACTGAAAAGCCTGTGATTTCAAAAAACC
TTGAACCAAGTAACGATACAACTTCGGATCCGCAATGTTCTGAATGCAA
GATAAACTACAGAGACCCCGGCACAAAGGATCTCATAATGTACTTGCAT
GCATGGAAATACAAGGTCAGTTTCAAACTATACATTTTTAAAGATTTAAT
TTCAAATTAAATCTTATTTTCAGGGCGTTGGTTGGGA
>CG6187|FBgn0032256
MESSKLLRNAQTQHGDASSVDVENIFLHRHMLYTNPTSDGNLHDREDSPEC
VWCPDDKDGSPAESKDPPVWTDWKCAIKSMWKQKHEPMKATEEEHVIILQ
LDKFQDADPDEIRVYQEAVPKGISISEEKSETQSKEVLSEKRKASSTDDEGHV
KKVKLEANSLKTKRPGFSDERYDETSYYFENGLRKVYPYFFTFTFfFAKGRW
VDEKILDVFVREFRAAPPEEYERSLEAGKLTVNSEKVPKDYKIKHNDLLANV
VHRHEVPVTSQPIKIVYMDKDIVVVNRPASIPVHPCGRYRHNTVVFILAKEH
NLKNLRTIHRLDRLTSGLLLFGRTAEKARELELQIRTRQVQKEYVCRVEGRF
PDGIVECNEKIDVVSYKIGVCKVSPKGKDCKTTFKRIGEVGSDSIVLCKPLTG
RMLIQIRVHLQFLGYPISNDPLYNHEVFGPLKGRGGDIGGITEEQLISNLISIHN
AENWLGLEGDQIVSGEIKDVAASTSVVEAPSVVQAPINSETEKPVISKNLEPS
NDTTSDPQCSECKINYRDPGTKDLIMYLHAWKYKVSFKLYIFKDLISN
Scim17
AE003634 (insertion @146760), nearest ORF (CG17745) @142269 (4 kb away)
>>CG17745|FBgn0032386|cDNA sequence
ATGGCTCCCAAGATCGTCGAGATCTCCGCTCCTCCGGCCAACCATTCAG
ACCCAAGATATATGAGCCAGTGCTATGTTGTGACTGCTGCCCGCTGTGC
ACCTGTGCCCAGGGATGTGGACGTGGATGTGAATGTGGACGAGGATGTG
GATGAGGAGATGAGCCTGGCTAAAAACCGAGCAGATGAGCAGGCGAAA
TGGACTTTTAAATGTTGCCATTTGTTGCGTGAAAACGCAACCGGTAGCCA
AAAAACGTTCAGCATTGCGATTTCTTGGGCTGCGGAGGGTTTCGGAATT
GCCACGTTTCCCGGGATTGCCTCTGATCTTGGACTGTGGGCTCCACTCAG
CTATTAG
>CG17745|FBgn0032386
MAPKIVEISAPPANHSDPRYMSQCYVVTAARCAPVPRDVDVDVNVDEDVD
EEMSLAKNRADEQAKWTFKCCHLLRENATGSQKTFSIAISWAAEGFGIATFP
GIASDLGLWAPLSY
Scim18
AE003666 (insertion @157060), nearest ORF (CG16798) @152043
>>CG16798|FBgn0032856|cDNA sequence
GTTCTTGTGTCGGAACATTCGGTACCAAAACTTCGGACGCTGCGGCTTTC
GTACTATTTATGATTTTTTGTGTTGTGACAAATGCGATTTATTTGCGGAC
AAAAGTGGCTTTTGGCAATCAGCTGGTATTGTTCTGCGAGAGCCGTACC
AAATAGTGCATAACATAAAATAAAACAAAACGAGTACTGGAAAAAAAA
AAGTATCTAAAGTCAAACATTTGGGTCCCCTGGCAACACTTGCATTTTCC
CTCACGACCAATCGCCCAATATAACTCCCGGCTGACACACATTTGATGA
GAACAAACAGCAAACTTAAAAAAATCTACCGAAAATAATGTCAACGAA
ATCAATGGCAGCCCACGGCAGCTGCAACATGTTGCTGCTGTGTCTTCTGC
TCCTGCTGCCGTCGGTCTCCCCCGTCCGCTTGCCCAAAAGCAGCAGCAA
CAATGCAACAGCAGCAACAACAGCAGCGACCGCATCAACCGAATCAAC
CGCAACAGCAGCAACAACTGCTGCAATCCGCAATGCCAATGCCAAGGCT
GGTAGCAAATATGAGATACGCGGCGTTGCCGGTGAACCAAATTACAAAT
CGGTGAATCTGACCTGGGAAGTTGAATTCGTGCCGTCGGCCCATGACAC
AGATTCGAGCCCCAACTCCAACTCCAGAGCGGACCAGGTGAACGCGACA
AATATGAGCGGCGATGTGGAACCGCCCCGGGATCTGGCATTCCAGATAT
TCTACTGTGAGATGCAGAACTACGGCCCACAGCGGTGTCGCGTCAAATT
GGTGAATGGCACCACCGCCGAGGTGTCCCAGGAGGAGAATGAGAAGGC
GACGGATCAGCAGGAGAAGCATGAACCCTCAGGGTCCCAGGTGCACCA
CTTTGTTGCTGCCGTGGACAACTTGCGCATGGCCACCAAATACAGTTTCC
ACGTTCGCCCGGCTGCTCAGAAGCGCCTCCAGGCGGGCGGAACTCGCAG
CTCCAATGCCCGGGCAGACTTTCACGATGAAAACAACGAGATCGAGAGT
GGATCCGGACATCTGGCGGGCCAGAGCATCGTCATACCCACCAAGGGCT
TCACCGCACATGCCACCCAGTGTTTGCCGCATGCCTCAGAGATCGAGGT
GGAGACGGGTCCGTACTTCGGAGGACGCATCGTCGTGGATGGAGGAAAC
TGTGGGATCAAGGGCGATGCCAGCGATGCGGCGGACAAGTACACGATG
AGGATCGATCACAAAGAGTGTGGAAGCTTGGTGAAACCGGAGACCAAC
ACGGTGGAGACCTTCATCACGGTACAGGAAAACCTTGGCATATTTACCC
ACAGCACAAGACGCTTTGTGGTGGTCTGCAGCTACCACTCAGGCATGCA
GACGGTCCGAGCAAGCTTCACTGTACCTGGAAAGAACGGGGTGGCCGCC
GCCTACGAGCCCAACGACCCCTTTGAGCCAGACGAGGATCAACGCCTGG
GCAGGGAACTCCGACCGATGCGCTACGTCAACAAGACGGAGCTGGTGCT
TCGCGAACCGGACTCCCAGCGGGAGTCCCAATCCGATTCGGAGTCCGTG
GAACAGGCTGCGGTGGTGGAACAGGCCCCGACGCCCACCACCGAGCAG
GCTTCTCAGCCCAGAGGTCAGGGCAGAGCTCTGAACCTCAACGAGGTCA
ACAGTTTGGCCGATGAGCCGGCGGAGGAACATCACTTGGAGCCTGTGGT
GGGCACCAAGTACGCCAAACTGGTTGTCGACCAGAGCCACAGTTCCTGG
ATGCCGTTGGAGGTGGGCTCGCCATCAGGTGGTAGCGACGAGAATGAAG
CCGTTCTGCGTTATATTGGCTCCCATCTTAGCAGCGTGCTGGTAACCGTC
TCGCTATCTGTGATAATCATCAGCATTITGCATCGTTCTGCTGCAGCGCCA
GCGGATCCGCTCTCCGCCCCGCAGCCCATCCCCCTGCCTGGCCGCCCACC
TGCCGCACAAAACGTTGCCGCGTGCACTGCAGCAGCAGCAGTACCAGTG
CACCTTGTAG
>CG16798|FBgn0032856
MAAHGSCNMLLLCLLLLLPSVSPVRLPKSSSNNATAATTAATASTESTATAA
TTAAIRNANAIKAGSKYEIRGVAGEPNYKSVNLTWEVEFVPSAHDTDSSPNS
NSRADQVNATNMSGDVEPPRDLAFQIFYCEMQNYGPQRCRVKLVNGTTAE
VSQEENEKATDQQEKHEPSGSQVHHFVAAVDNLRMATKYSFHVRPAAQKR
LQAGGTRSSNARADFHDENNEIESGSGHLAGQSIVIPTKGFTAHATQCLPHA
SEIEVETGPYFGGRIVVDGGNCGIKGDASDAADKYTMRIDHKECGSLVKPET
NTVETFITVQENLGIFTHSTRRFVVVCSYHSGMQTVRASFTVPGKNGVAAA
YEPNDPFEPDEDQRLGRELRPMRYVNKTELVLREPDSQRESQSDSESVEQA
AVVEQAPTPTTEQASQPRGQGRALNLNEVNSLADEPAEEHHLEPVVGTKYA
KLVVDQSHSSWMPLEVGSPSGGSDENEAVLRYIGSHLSSVLVTVSLSVIIISIC
IVLLQRQRIRSPPRSPSPCLAAHLPHKTLPRALQQQQYQCTL
Scim19
AE003669 (insertion @167790), nearest ORF (CG9241) @168642, CG9242 spans
this region also.
>>CG9241|FBgn0032929|cDNA sequence
AGCCCGCCAAAACAGATATGTTATTGCGCTTATTTAGAAAACCAAGAAA
AAACACGAGAACACGTGAAAATACAAATCTACCCAAATGAAATGGGTC
CTGCTCAGAAATCCGGAACAGATATTAGTATCGATGATGAGGAGGAAAT
ACTGGCTCTGGAAAAACTACTGGGTGCAGCAGAAAACGAAAATACAAA
ATCTGCAGAGTCAGAAAAAGCAAAACCCACCGCACCCATTTGGTGCCA
AAACTACGAGAAGACAACAGTTTTGCTAATGCCTTCACCTTCGAGAAGA
TCGTGAAACCGGAAAAGCAGAAGAATGCTGCTATCATTAAGGAACCAG
AGCTGGACTCGTCCGACGACGAGGAGGTAAAGAACTTCCTGGAACGAA
AGTACAATGAGTACGGCAGTGATATAAACAAGAGACTGAAGCAGCAGC
AGGAGAACGCCTACGAGTCCAAGGTGGCGAGGGAGGTGGATCAGGAGC
TTAAGAAGTCTATCCACGTGGTTACATCCACCCCGCAACCCCTGAAAAA
TCCGCATAATCCTATTAAACGGCAATCGGCGGTGAGCACCACGTTTCAA
CGTCCTCCGCCAGTCGCTGCCGCCGTGGCATCTACATCCCAGTCAAGTGC
TCCCGTATCTGCTGTTTTTACGGATCCAGTCTTCGGACTGCGCATGATCA
ATCCGCTAGTCTCCAGCTCACTGCTGCAGGAGCGCATGACGGGCAGGAA
ACCTGTGCCCTTCTCAGGCGTTGCGTATCACATCGAGCGAGGCGATTTGG
CCAAAGATTGGGTCATTGCTGGCGCGCTGGTTTCCAAAAATCCTGTAAA
AAACACCAAGAAGGGTGATCCCTACTCCACGTGGAAACTATCCGATCTA
CGGGGAGAGGTTAAAACGATCTCACTTTTCCTTTTTAAAGAGGCCCACA
AATCCCTGTGGAAAACAGCGGAGGGTCTGTGCTTGGCTGTGTTGAATCC
AACTATTTTCGAGAGGAGAGCGGGAAGCTCCGATGTGGCCTGCCTATCC
ATCGATAGCTCCCAGAAAGTCATGATCCTGGGTCAATCCAAAGATTTGG
GCACATGTCGGGCCACCAAAAAAAATGGGGACAAGTGCACTTCGGTGGT
TAACCTAACCGACTGTGAYATTGCATTTTTCATGTAAAGCAGGAATATG
GCAAGATGTCCCGACGTTCTGAACTGCAATCGGCGACCGCAGGTCGTGG
TATCAATGAACTAAGAAACAAGGTTTTGGGCAAAAACGAGGTATTTTAC
GGCGGCCAAACATTTACTGCAGTTCCCGCAAGAAAAAGTGCCAAGTTAA
TCACCAAGGAACGTGATCGTCTGAGTATGCTGGCTGGCTATGATGTTTCC
CCCTTCGCCCATACCGCTAACCACACCTCAAAGCCCAAAACAGCCGAAC
CCACTAAAATTCCATATGCAGAACGTGGCGGTCCTGTTTCCCGTTTGGCT
GGTGGTGTGGAAGCGTCTAGGAAACAGAGAGTCCAAGATCTAGAGCGG
TTGCGTCTGCTTAAAGAGGAAAATGAGCGCTTTGAAAAAAAGAAGCAGG
CGGAGGGCCATGTCTTGGGAAGTGATAACAAAAAAGAATCTGAAGCAG
GCACACCCGCTGTCAGTATGCCCACTACACCTGTTCCAGATAAATTCAA
AAATCGAGGCTTCTCCTTTGATGCCAGTTTAACGCCCAAGCTTTCCGGTA
GCGAGAACTTTTCCTTTGAAATCAATGTAGGATCTCGCCAGGCACAAAA
TGCTAAGCTGAAAGCAGCTGCCCTGCTGAAGAAGAAGCCACTGGAGAA
GATCAACCCCAACTCCACACGAGGCAGTGAAAGTGGGAAGAGAAGAGC
CATCGATGAACTCAACGAGAAGTTCTCTAGCAGCGCCAAGCGACAAAAA
ATTGATGAGGACGATCGGGAGTTAATGCGCAAATCAAGAATCGAAAAA
ATAATGGCAGCCACCTCATCGCATACGAATCTCGTGGAAATGCGAGAGC
GCGAAGCGCAGGAAGAGTACTTTAACAAGCTTGAACGCAAGGAAGCGA
TGGAAGAGAAGATGCTGACCACATACAAGATGCCATGCAAGGCCGTCAT
CTGCCAGGTGTGCAAGTACACAGCCTTTTCCGCTTCCGATCGCTGCAAGG
AGCAGAAGCACCCCTTAAAGGTGGTCGATGCTGAAAAGCGATTCTTTCA
GTGCAAAGACTGCGGAAATCGAACTACTACCGTATTCAAGUGCCCAAA
CAGAGCTGTAAGAATTGCAAGGGGTCGCGATGGCAAAGGACGGCTATG
ATACGGGAGAAAAAGATACTGACTGGTAGAGAAACTCTATCCGTGAGA
GGAGACGAGGAAACCTTTATGGGCTGCCTAGCAGGCAGTGCTAATCTCA
ACTTGCTGGTACCCGATGAAGAGTGA
>CG9241|FBgn0032929
MINPLVSSSLLQERMTGRKPVPFSGVAYHIERGDLAKDWVIAGALVSKNPV
KNTKKGDPYSTWKLSDLRGEVKTISLFLFKEAHKSLWKTAEGLCLAVLNPTI
FERRAGSSDVACLSIDSSQKVMILGQSKDLGTCRATKKNGDKCTSVVNLTD
CDYCIFHVKQEYGKMSRRSELQSATAGRGINELRNKVLGKNEVFYGGQTFT
AVPARKSAKLITKERDRLSMLAGYDVSPFAHTANHTSKPKTAEPTMPYAER
GGPVSRLAGGVEASRKQRVQDLERLRLLKEENERFEKKKQAEGHVLGSDN
KKESEAGTPAVSMPTTPVPDKFKNRGFSFDASLTPKLSGSENFSFEINVGSRQ
AQNAKLKAAALLKKKPLEKTNPNSTRGSESGKRRAIDELNEKFSSSARRQKI
DEDDRELMRKSRIEKIMAATSSHTNLVEMREREAQEEYFNKLERKEAMEEK
MLTTYKMPCKAVICQVCKYTAFSASDRCKEQKHPLKVVDAEKRFFQCKDC
GNRTTTVFKLPKQSCKNCKGSRWQRTAMIREKKILTGRETLSVRGDEETFM
GCLAGSANLNLLVPDEE
>>CG9242|FBgn0032928|cDNA sequence
ATATGGTCATCCGCTCGTCAATAAGTCATCTTTCGGCTTTAATTCGCGAA
AAAACTGCAGGAAATCCAAAAGGAAAGTCCCTGGAAGCGGCCATAATA
ACGCAGCCGTGAAAATCACAGGGATTTCATCGCCAGCTGTGTCGAGCAG
CCCTGGATACGCGGAAAAGAAGCTGCAGCAGCCGAAGTTTTGAGTG
TGTGCGTGAGGAAGGAAAACGGGGGACCGCAAACAACGGATCGCGAAT
TTCGTCTTAAGACAAAGTCTTGCGCTGCTTGTCACGGTATTCCACGGCCT
TGCCGACGGACTTCCCGGTTCTGGAAAACCGCAGCCAGGCTAAAACGAG
AGAAGTGCTGCAACGATAAAGAAATGAACTCAAACATTTTTCTGGGCAC
AGCAGAGAATGGCCTGCGGCATGATAAGATTGTTATACTTGATGCGGGA
GCACAGTACGGCAAGGTTATCGACCGTAAGGTACGCGAACTCTTCGTTG
AGACGGATATCCTTCCTCTGGATACGCCAGCTGCCACGATACGCAACAA
TGGCTATCGAGGCATCATCATCTCCGGCGGACCCAACTCAGTCTACGCT
GAGGATGCGCCCAGCTATGATCCCGATCTGTTCAAGCTAAAAATACCTA
TGCTGGGCATCTGCTACGGCATGCAGCTAATCAACAAAGAGTTCGGGGG
CACAGTGCTCAAGAAGGATGTTCGAGAGGATGGCCAACAAAATATCGA
GATCGAGACCTCGTGCCCGCTCTTTAGTCGCCTCAGTCGCACACAGTCCG
TGCTGTTAACCCACGGAGATAGCGTTGAGAGGGTAGGCGAGAATCTGAA
GATTGGTGGCTGGTCTACAAACCGCATTGTGACAGCCATTTACAATGAA
GTACTACGCATCTACGGCGTACAGTTCCATCCTGAGGTGGACCTCACTAT
CAATGGCAAACAGATGCTATCGAACTTCCTGTACGAAATCTGCGAACTG
ACACCTAACTTTACCATGGGTAGTCGAAAGGAGGAGTGCATACGCTATA
TCCGTGAGAAAGTGGGCAACAATAAGGTGTTGCTCCTGGTCAGCGGCGG
CGTGGATTCGAGTGTCTGTGCAGCTTTGCTCCGCCGTGCTTTGTACCCTC
ATCAGATAATTGCCGTGCATGTAGATAATGGTTTCATGCGCAAGAAGGA
AAGTGAAAAGGTGGAGCGTTCACTGCGCGATATTGGCATTGATTTAATC
GTCCGAAAAGAAGGCTACACGTTCCTTAAAGGCACCACGCAGGTCAAGA
GGCCCGGACAGTACTCCGTGGTGGAAACGCCGATGTTATGTCAGACATA
CAATCCGGAGGAAAAACGCAAGATAATTGGTGATATATTCGTCAAGGTG
ACCAATGATGTAGTAGCCGAATTGAAACTAAAGCCCGAAGAAGTTATGT
TGGCCCAGGGAACCCTCCGACCAGATCTGATCGAGTCCGCCTCTAGCAT
GGTGAGCACGAATGCAGAAACAATCAAAACGCACCACAATGACACGGA
TCTGATCAGAGAGCTTCGTAACGCAGGACGTGTGGTTGAGCCCCTTTGC
GACTTTCATAAGGATGAAGTGCGCGACCTTGGCAATGATCTTGGCCTGC
CTCAAGAGCTTGTGGAGAGGCAACCCTTTCCGGGTCCTGGCCTGGCAAT
CCGCGTCCTTTGCGCTGAGGAGGCATACATGGAAAAGGACTACTCAGAA
ACTCAGGTTATTATCCGCGTGATTGTAGACTACAAGAATAAACTGCAGA
AGAACCATGCTTFGATCAACCGCGTAACGGCGGCCACGAGCGAGGCGG
AACAGAAAGACCTTATGCGTATCTCATCGAACTCGCAGATCCAGGCAAC
TTTGCTGCCCATCCGATCAGTGGGCGTGCAAGGTGATAAACGGTCATAT
AGCTACGTAGTAGGCCTATCCACGAGCCAGGAGCCCAACTGGCAGGATC
TTCTCTTCCTCGCCAAAATCATACCGCGAATTCTGCACAACGTGAACAGG
GTGTGCTATATCTTCGGCGAACCCGTACAGTATCTAGTGACGGATATTAC
GCACACCACACTGAATACTGTAGTTCTTTCGCAGCTGAGGCAAGCCGAT
GATATTGCCAATGAAATCATAATGCAAGCTGGACTATACCGGAAGATCT
CGCAGATGCCTGTTGTTCTCATACCCGTGCACTTTGACCGCGATCCCATT
AACCGCACACCCTCGTGCAGAAGGTCGGTAGTGCTGCGTCCGTTCATAA
CGAACGACTTTATGACTGGTGTGCCGGCTGAGCCCGGATCCGTGCAAAT
GCCTTTGCAAGTCCTAAATCAAATTGTACGCGATATATCCAAGCTGGAT
GGAATCTCGAGGGTGCTGTACGACTTGACAGCCAAGCCGCCGGGCACCA
CCGAATGGGAATGA
>CG9242|FBgn0032928
MNSNIFLGTAENGLRHDKIVILDAGAQYGKVIDRKVRELFVETDILPLDTPAA
TIRNNGYRGIIISGGPNSVYAEDAPSYDPDLFKLMPMLGICYGMQLINKEFGG
TVLKKDVREDGQQNIEIETSCPLFSRLSRTQSVLLTHGDSVERVGENLMGG
WSTNRIVTAIYNEVLRIYGVQFHPEVDLTINGKQMLSNFLYELCELTPNFTMG
SRKEECIRYIREKVGNNKVLLLVSGGVDSSVCAALLRRALYPHQIIAVHVDN
GFMRKKESEKVERSLRDIGIDLIVRKEGYTFLKGTTQVKRPGQYSVVETPML
CQTYNPEEKRKIIGDIFVKVTNDVVAELKLKPEEVMLAQGTLRPDLIESASS
MVSTNAETIKTHHNDIDLIRELRNAGRVVEPLCDFHKDEVRDLGNDLGLPQ
ELVERQPFPGPGLAIRVLCAEEAYMEKDYSETQVIIRVIVDYKNKLQKHALI
NRVTAATSEAEQKDLMRISSNSQIQATLLPIRSVGVQGDKRSYSYVVGLSTS
QEPNWQDLLFLAMIPRILHNVNRVCYIFGEPVQYLVTDITHTTLNTVVLSQL
RQADDIANEIIMQAGLYRMSQMPVVLIPVHFDRDPINRTPSCRRSVVLRPFIT
NDFMTGVPAEPGSVQMPLQVLNQIVRDISKLDGISRVLYDLTAKPPGTTEWE
Scim20 (the 3′and 5′P element sequences are separated by ˜24 kb)
3′Search AE003784 (insertion @11445), nearest ORF (CG12110) positioned from 1392
to 14629
5′Search AE003784 (insertion @36320), three ORFs are in this region
CG8276 3′end 1 kb away, CG8330 3′end 6 kb away, CG8325 5′end 6 kb away
>>CG12110|FBgn0033075|cDNA sequence
TITTGCTAGGCGTGGAGTAAGATGAACGCGAACAGAAACTTTTGAATTT
TGAAGTAAAATTTAAATTTAAGTGAAAGTGTTAAGTCTGCCATACGAAA
GCATTTAAATGAAGTAATACATATGTATAAATGTACATATATACACTTAA
CCCACTGCTGAGGTCTCCAGCTTTCAGTGCCAGTTTGGAGTCCACGACGG
AGAAGTTAAGCCACAACTTCTGGCATCAGATTAAGAGCTAAACCTATTT
CAGCAGTAGCCGCAAGCATTTGAACACCCCACTGACGATGATACCGGC
CGGCGCACTATGGCGGCTACGTTAACAGAGGCTACGATGATTTAGACAG
TTCCTACTACTTTGCCCAGTACGAGGCGATGGCAGATGCCGGCACCGTT
GGAGGCGCCTTGCCGCCCTACGCACTTACCAACTCGGACGAAGAACATG
GCAGCGGGGAAGAGGACGCGTCGGAGGAGAACTCCAACAATGAAGAGG
GAGAGGGAGTGTTCCGGGACTGCACAGACGAAGCGGTTGTCGAGCATCA
TAACCGCTGCCTGCCAGAATTTCAGTTCTCTCTAGTCGATTCTGAGTACG
ATGAGACCCTCGCTTTTCCTGATTCTGTGACCATTCTATCCAACGTGGGC
GACAAGCCGGTGCTGGTGGAGCGCAAGGAGACGGACGATGATGAGGAG
GAGTTCGACGACGAGGAAAACAACAGTGTAGTCCTGAGACACGAAATA
CCATTTACTAGCATATACGGGGCGAGCGTCAAGTTCAACTCGTTCCAGC
GCAAGGTTTTCATCCCGGGCCGTGAGATTCATGTTCGGATCGTCGATACG
GAGCGTAGCGTCACTACACATCTGCTAAACCCCAATCTGTACACAATCG
AGCTGACCCACGGTCCCTTCAAGTGGACGATCAAGCGGCGATACAAGCA
CTTTAACTCGTTGCACCAGCAGCTCAGCTTTTTCCGCACCTCGCTCAACA
TTCCTTTTCCCAGTCGCAGTCACAAGGAGAAGCGTACCACTTTGAAAGC
CACAGCCAGAGAGATGGCTGACGAGTCCACTCTAAAGGACCTTCCTTCT
CACACCAAGGTCAAACAAACTAGCACTCCGCTGAGGGCTGAAGGCAGA
AGCAGTAAAATCGCGGGCAGTAACGCCAACAATGCCATGGCTATGATCA
GTCCCAATCACAGCTCCATTCTGGCGGGTCTAACACCACGACGCATTCA
AAAGAAGCGCAAAAAAAAGAAGAAACGGAAGCTGCCGCGATTCCCAAA
CCGTCCTGAGAGTCTGGTCACCGTAGAGAATCTGAGCGTCAGAATAAAA
CAGCTGGAGGACTACTTGTACAACCTGCTGAACATCAGCTTGTACCGAT
CTCACCATGAAACGCTAAACTTCGTTGAAGTGTCTAATGTGTCCTTTGTT
CCGGGAATGGGAATTAAGGGCAAGGAAGGCGTGATTTTAAAGCGAACT
GGATCAACGAGACCAGGGCAAGCAGGATGCAATTTTTTTGGGTGCTTTC
AAAAGAACTGCTGTGTGCGCTGCAACTACTTTTGCTCCGACGTAGTTTGC
GGCACGTGGCGGAACCGATGGTTTTTCGTAAAAGAGACCTGCTTCGGCT
ACATCCGTCCAACAGACGGAAGCATCCGGGCAGTGATCCTCTTTGATCA
GGGCTTCGACGTTTCCACGGGTATCTATCAGACGGGCATGCGCAAGGGC
TTGCAGGTACTGACGAACAACCGTCACATTGTGCTCAAGTGCTGGACAC
GGCGTAAGTGTAAAGAGTGGATGCAATACCTCAAGAACACGGCCAACTC
GTATGCGCGCGACTTCACCCTGCCCAATCCGCACATGTCCTTCGCTCCGA
TGCGCGCCAACACTCATGCCACGTGTCCCGAGATATACATGAAGCGACC
CGCACTCGACGGAGACTACTGGCGATTGGACAAGATCCTGTTGCGCAAG
GCCGAACAGGGAGTGCGCGTCTTTGTGCTGCTCTACAAGGAGGTTGAAA
TGGCACTTGGCATAAACAGCTACTACAGCAAGTCCACGCTGGCCAAGCA
TGAAAACATCAAGGTCATGCGTCATCCGGACCATGCTAGAGGAGGTATT
CTGCTTTGGGCACATCACGAAAAGATCGTCGTAATCGACCAAACCTATG
CGTTTATGGGAGGTATTGATTTGTGCTATGGACGTTGGGATGATCACCAC
CATCGGCTAACGGATCTGGGTAGCATATCTACGTCATCTTTTTCTGGCAG
CACGCGTCGAACGCCAAGTTTGTACTTCACCAAAGACGACACGGACTCA
GCTTTCGGATCACGTAAGTCCTCGCGAAACGCTCACTACGATACCTCCGC
CAAGGAAAGGCCACCGTCCCCACCCCCGGATGAGCCCAATACTAGCATA
GAGTTGAAAACTCTTAAGCCTGGTGATCGACTGCTTATACCGTCTACGCT
CGTTTCGAGTCCGGGTGAAACTCCCGCAGAATCGGGAATCGCTTTAGAA
GGGATGAAACTCAACACCCCTGAAATGGAGCGTAAGAACGTACTCGATC
GCCTGAAGAACAACGCGATGAAGGGCGCCCGTATGGGCAAGGACTTTAT
GCACCGTCTAACAGCTACTGAGACGGAGGAAAAATCTGCGGAGGTGTAC
ACTATCGAGTCCGAGGAAGCTACGGACCACGAAGTCAACCTTAACATGG
CTTCAGGTGGGCAGGAAGTGGCGATTACCACTAGCAGTACACAAATACT
CAGTGAGTTCTGCGGCCAGGCCAAGTACTGGTTCGGCAAGGATTACTCC
AACTTTATACTTAAAGACTGGATGAACCTAAACTCGCCGTTCGTGGATAT
CATAGATCGAACAACAACACCGCGGATGCCATGGCACGACGTGGGTCTG
TGTGTGGTGGGTACTTCCGCTAGGGATGTGGCCCGCCACTTCATTCAGCG
CTGGAATGCCATGAAGCTGGAGAAACTACGCGATAACACGAGATTCCCC
TATTTGATGCCAAAAAGCTATCACCAAGTGAGGCTCAATCCGAACATTC
AGCAAAACCGTCAGCAACGGGTCACGTGCCAGCTACTTGGAAGCGTCTC
TGCCTGGAGCTGCGGCTTTATAGAGGCGGATCTTGTGGAGCAAAGCATC
CACGATGCCTACATCCAGACGATCACCAAGGCGCAGCACTACGTGTACA
TCGAAAACCAATTTTTTATCACTATGCAGTTAGGCATGGGTGTGCCAGGT
GCTTATAACAATGTGCGGAATCAAATCGGGGAAACACTCTTTAAACGGA
TCGTTAGAGCGCACAAGTATGAAACCAAAATACTTATCCTGATTCTAGC
AGATCTAATGTTCAGCTCTTCTAGGGAACGGAAGCCTTTCCGAGTTTATG
TGATFTATGCCGCTCCTACCGGGCTTTGAGGGTGATGTCGGTGGCAGTACT
GGGATAGCAGTCAGAGCAATTACACACTGGAACTATGCGTCCATTTCCA
GGGGACGCACATCAATTTTGACCCGCCTGCAGGAGGCGGGTATTGCCAA
TCCGGAAAACTATATCTCATTCCACAGCCTGCGCAACCATTCTTTTTTGA
ATAACACACCCATAACAGAGTTGATATATGTCCACTCAAAGCTCTTGAT
AGCCGACGATCGCGTTGTAATCTGCGGTTCGGCAAACATTAACGATCGC
TCTATGATCGGAAAGCGGGACTCCGAGATAGCGGCTATTCTAATGGACG
AGGAGTTCGAGGACGGACGCATGAATGGCAAGAAGTATCCGAGCGGAG
TGTTTGCCGGTCGCCTTCGAAAATACCTTTTTAAAGAACACTTAGGCCTC
CTGGAAAGCGAAGGTTCCAGTCGGTCTGACCTGGACATTAACGATCCTG
TTTGTGAGAAGTTTTTGGCACGGCACCTGGCGTAGGATTTCAATGCAGAA
CACAGAGATTTACGACGAGGTGTTTAAGTGCATCCCCACTGACTTTGTA
AAAACCTTTGCCAGCCTTCGCAAATACCAGGAGGAGCCGCCTCTTGCCA
AAACCGCCCCTGATCTAGCTGCCAACAGAGCCAACGACATTCAGGGTTA
CTTGGTCGACCTGCCATTGGAATTTCTGAACAAGGAGGTTCTCACGCCGC
CTGGAACTAGTAAGGAGGGCCTAATCCCTACCTCTGTATGGACATAGTC
TGTCAAAAGTGTCTAAGATTTTAGAAAGCTTAAAAACCACTTACCATTTA
CCACCCACCAAAAGCACTATCTTTAACGATGCCAATGTCAAGTCAAACA
TTTTGTAAATAGTGTATAATAGCCGTAGATAACTCTAGATACTTTCAAGT
ACATGTAGCTATTCCTTACCAATAGTTAATTTATTTTACAATGTTTGTCTA
TGTCCTCAAGTAGTTTTAAGATTTTTGTTATTATTTTGTATGATGTTAAAC
AGTATTTTAGACCGATTTACACAAGTTTATTAAAGTGATATGAAGTGCAA
ATGAAGAACTGCAACAT
>>CG12110|FBgn0033075|cDNA sequence
CGTGGAGTAAGATGAACGCGAACAGAAACTTTTGAATTTTGAAGTAAAA
TTTAAATTTAAGTGAAAGGTTCGAAGTAATTGTTAATTGAAAAATAAAT
CAAATGCAGTTTAGCCTGATCTGAGGAAAGAAAGAACGAGTGCTAAGCT
CAATGAACTTTCACTCTCCGCTCTCTCCCTATACATCGCGCTTCCAGCGA
GAAATCTCTGCTGATCGTTCTCATTTCCACGTTCGCTTGGCGTTTTGATCA
GTTTCGAATTTGACTTATAGCGACGCTGGTCGGAGCTCTCTCGGCAAACA
AAAACCGTGACAAGCAAAGATTTGAGCAAAGATTTGCCCAGAAGGGGT
CTTGCTCGACACCAATAATAAAAATGCCGCGATAGAAGTGTGTGTGCCA
TTGACCAACATTTTAATATTTTTAAATTGTTTCTTGTGTGCTCACGAAACG
TGTTCATGTGGCGCCTCAATTGATTTGATCTTATTTCACCAATTATCAAA
GTGTTAAGTCTGCCATACGAAAGCATTTAAATGAAGTAATACATATGTA
TAAATGTACATATATACACTTAACCCACTGCTGAGGTCTCCAGCTTTCAG
TGCCAGTTTGGAGTCCACGACGGAGAAGTTAAGCCACAACTCTGGCAT
CAGATTAAGAGCTAAACCTATTTCAGCAGTAGCCGCAAGCATTTGAACA
CCCCACTGACGATGTTTACCGGCCGGCGCACTATGGCGGCTACGTTAAC
AGAGGCTACGATGATTTAGACAGTTCCTACTACTTTGCCCAGTACGAGG
CGATGGCAGATGCCGGCACCGTTGGAGGCGCCTTGCCGCCCTACGCACT
TACCAACTCGGACGAAGAACATGGCAGCGGGGAAGAGGACGCGTCGGA
GGAGAACTCCAACAATGAAGAGGGAGAGGGAGTGTTCCGGGACTGCAC
AGACGAAGCGGTTGTCGAGCATCATAACCGCTGCCTGCCAGAATTCAG
TTCTCTCTAGTCGATTCTGAGTACGATGAGACCCTCGCTTTTCCTGATTCT
GTGACCATTCTATCCAACGTGGGCGACAAGCCGGTGCTGGTGGAGCGCA
AGGAGACGGACGATGATGAGGAGGAGTTCGACGACGAGGAAAACAACA
GTGTAGTCCTGAGACACGAAATACCATTTACTAGCATATACGGGCCGAG
CGTCAAGTTCAACTCGTTCCAGCGCAAGGTTTITCATCCCGGGCCGTGAG
ATTCATGTTCGGATCGTCGATACGGAGCGTAGCGTCACTACACATCTGCT
AAACCCCAATCTAGATTTACGACGAGGTGTTTAAGTGCATCCCCACTGA
CTTTGTAAAAACCTTTGCCAGCCTTCGCAAATACCAGGAGGAGCCGCCT
CTTGCCAAAACCGCCCCTGATCTAGCTGCCAACAGAGCCAACGACATTC
AGGTACTCTTCCCAATTAATATTTAA
>>CG12110|FBgn0033075|cDNA sequence
CTAGGCGTGGAGTAAGATGAACGCGAACAGAAACTTTTGAATTTTGAAG
TAAAATTTAAATTTTAAGTGAAAGCTTTCAGTGCCAGTTTGGAGTCCACGA
CGGAGAAGTTAAGCCACAACTTCTGGCATCAGATTAAGAGCTAAACCTA
TTTCAGCAGTAGCCGCAAGCATGTGAGTGCTTTAAATTCATAAAAACAC
ATTAAATTGAACACCCCACTGACGATGTTTACCGGCCGGCGCACTATGG
CGGCTACGTTAACAGAGGCTACGATGATTTAGACAGTTCCTACTACTTTG
CCCAGTACGAGGCGATGGCAGATGCCGGCACCGTTGGAGGCGCCTTGCC
GCCCTACGCACTTACCAACTCGGACGAAGAACATGGCAGCGGGGAAGA
GGACGCGTCGGAGGAGAACTCCAACAATGAAGAGGGAGAGGGAGTGTT
CCGGGACTGCACAGACGAAGCGGTTGTCGAGCATCATAACCGCTGCCTG
CCAGAATTTCAGTTCTCTCTAGTCGATTCTGAGTACGATGAGACCCTCGC
TTTTCCTGATTCTGTGACCATTCTATCCAACGTGGGCGACAAGCCGGTGC
TGGTGGAGCGCAAGGAGACGGACGATGATGAGGAGGAGTTCGACGACG
AGGAAAACAACAGTGTAGTCCTGAGACACGAAATACCATTTACTAGCAT
ATACGGGCCGAGCGTCAAGTTCAACTCGTTCCAGCGCAAGGTTTTCATC
CCGGGCCGTGAGATTCATGTTCGGATCGTCGATACGGAGCGTAGCGTCA
CTACACATCTGCTAAACCCCAATCTGTACACAATCGAGCTGACCCACGG
TCCCTTCAAGTGGACGATCAAGCGGCGATACAAGCACTTTAACTCGTTG
CACCAGCAGCTCAGCTTTTTCCGCACCTCGCTCAACATTCCTTTTTCCCAG
TCGCAGTCACAAGGAGAAGCGTACCACTTTGAAAGCCACAGCCAGAGA
GATGGCTGACGAGTCCACTCTAAAGGACCTTCCTTCTCACACCAAGGTC
AAACAAACTAGCACTCCGCTGAGGGCTGAAGGCAGAAGCAGTAAAATC
GCGGGCAGTAACGCCAACAATGCCATGGCTATGATCAGTCCCAATCACA
GCTCCATTCTGGCGGGTCTAACACCACGACGCATTCAAAAGAAGCGCAA
AAAAAAGAAGAAACGGAAGCTGCCGCGATTCCCAAACCGTCCTGAGAG
TCTGGTCACCGTAGAGAATCTGAGCGTCAGAATAAAACAGCTGGAGGAC
TACTTGTACAACCTGCTGAACATCAGCTTGTACCGATCTCACCATGAAAC
GCTAAACTTCGTTGAAGTGTCTAATGTGTCCITTGTTCCGGGAATGGGAA
TTAAGGGCAAGGAAGGCGTGATTTTAAAGCGAACTGGATCAACGAGACC
AGGGCAAGCAGGATGCAATTTTTTTGGGTGCTTTCAAAAGAACTGCTGT
GTGCGCTGCAACTACTTTTGCTCCGACGTAGTTTGCGGCACGTGGCGGA
ACCGATGGTTTTTCGTAAAAGAGACCTGCTTCGGCTACATCCGTCCAACA
GACGGAAGCATCCGGGCAGTGATCCTCTTTGATCAGGGCTTCGACGTTT
CCACGGGTATCTATCAGACGGGCATGCGCAAGGGCTTGCAGGTACTGAC
GAACAACCGTCACATTGTGCTCAAGTGCTGGACACGGCGTAAGTGTAAA
GAGTGGATGCAATACCTCAAGAACACGGCCAACTCGTATGCGCGCGACT
TCACCCTGCCCAATCCGCACATGTCCTTCGCTCCGATGCGCGCCAACACT
CATGCCACGTGTCCCGAGATATACATGAAGCGACCCGCACTCGACGGAG
ACTACTGGCGATTGGACAAGATCCTGTTGCGCAAGGCCGAACAGGGAGT
GCGCGTCTTTGTGCTGCTCTACAAGGAGGTTGAAATGGCACTTGGCATA
AACAGCTACTACAGCAAGTCCACGCTGGCCAAGCATGAAAACATCAAG
GTCATGCGTCATCCGGACCATGCTAGAGGAGGTATTCTGCTTTGGGCAC
ATCACGAAAAGATCGTCGTAATCGACCAAACCTATGCGTTTATGGGAGG
TATTGATTTGTGCTATGGACGTTGGGATGATCACCACCATCGGCTAACGG
ATCTGGGTAGCATATCTACGTCATCTTTTTCTGGCAGCACGCGTCGAACG
CCAAGTTTGTACTTCACCAAAGACGACACGGACTCAGCTTFTCGGATCAC
GTAAGTCCTCGCGAAACGCTCACTACGATACCTCCGCCAAGGAAAGGCC
ACCGTCCCCACCCCCGGATGAGCCCAATACTAGCATAGAGTTGAAAACT
CTTAAGCCTGGTGATCGACTGCTTATACCGTCTACGCTCGTTTCGAGTCC
GGGTGAAACTCCCGCAGAATCGGGAATCGCTTTAGAAGGGATGAAACTC
AACACCCCTGAAATGGAGCGTAAGAACGTACTCGATCGCCTGAAGAACA
ACGCGATGAAGGGCGCCCGTATGGGCAAGGACTTTATGCACCGTCTAAC
AGCTACTGAGACGGAGGAAAAATCTGCGGAGGTGTACACTATCGAGTCC
GAGGAAGCTACGGACCACGAAGTCAACCTTAACATGGCTTCAGGTGGGC
AGGAAGTGGCGATTACCACTAGCAGTACACAAATACTCAGTGAGTTCTG
CGGCCAGGCCAAGTACTGGTTCGGCAAGGATTACTCCAACTTTATACTT
AAAGACTGGATGAACCTAAACTCGCCGTTCGTGGATATCATAGATCGAA
CAACAACACCGCGGATGCCATGGCACGACGTGGGTCTGTGTGTGGTGGG
TACTTCCGCTAGGGATGTGGCCCGCCACTTCATTCAGCGCTGGAATGCCA
TGAAGCTGGAGAAACTACGCGATAACACGAGATTCCCCTATTTGATGCC
AAAAAGCTATCACCAAGTGAGGCTCAATCCGAACATTCAGCAAAACCGT
CAGCAACGGGTCACGTGCCAGCTACTTCGAAGCGTCTCTGCCTGGAGCT
GCGGCTTTATAGAGGCGGATCTTGTGGAGCAAAGCATCCACGATGCCTA
CATCCAGACGATCACCAAGGCGCAGCACTACGTGTACATCGAAAACCAA
TTTTTTATCACTATGCAGTTAGGCATGGGTGTGCCAGGTGCTTATAACAA
TGTGCGGAATCAAATCGGGGAAACACTCTTTAAACGGATCGTTAGAGCG
CACAAGTATGAAACCAAAATACTTATCCTGATTCTAGCAGATCTAATGTT
CAGCTCTTCTAGGGAACGGAAGCCTTTCCGAGTTTATGTGATTATGCCGC
TCCTACCGGGCTTTGAGGGTGATGTCGGTGGCAGTACTGGGATAGCAGT
CAGAGCAATTACACACTGGAACTATGCGTCCATTTCCAGGGGACGCACA
TCAATTTTGACCCGCCTGCAGGAGGCGGGTATTGCCAATCCGGAAAACT
ATATCTCATTCCACAGCCTGCGCAACCATTCTTTTTTGAATAACACACCC
ATAACAGAGTTGATATATGTCCACTCAAAGCTCTTGATAGCCGACGATC
GCGTTGTAATCTGCGGTTCGGCAAACATTAACGATCGCTCTATGATCGG
AAAGCGGGACTCCGAGATAGCGGCTATTCTAATGGACGAGGAGTTCGAG
GACGGACGCATGAATGGCAAGAAGTATCCGAGCGGAGTGTTTGCCGGTC
GCCTTCGAAAATACCTTTTTAAAGAACACTTAGGCCTCCTGGAAAGCGA
AGGTTCCAGTCGGTCTGACCTGGACATTAACGATCCTGTTTGTGAGAAGT
TTTGGCACGGCACCTGGCGTAGGATTTCAATGCAGAACACAGAGATTTA
CGACGAGGTGTTTAAGTGCATCCCCACTGACTTTGTAAAAACCTTTGCCA
GCCTTCGCAAATACCAGGAGGAGCCGCCTCTTGCCAAAACCGCCCCTGA
TCTAGCTGCCAACAGAGCCAACGACATTCAGGGTTACTTGGTCGACCTG
CCATTGGAATTTCTGAACAAGGAGGTTCTCACGCCGCCTGGAACTAGTA
AGGAGGGCCTAATCCCTACCTCTGTATGGACATAGTCTGTCAAAAGTGT
CTAAGATTTTAGAAAGCTTAAAAACCACTTACCATTTACCACCCACCAA
AAGCACTATCTTTAACGATGCCAATGTCAAGTCAAACATTTTGTAAATAG
TGTATAATAGCCGTAGATAACTCTAGATACTTTCAAGTACATGTAGCTAT
TCCTTACCAATAGTTAATTTATTTTACAATGTTTGTCTATGTCCTCAAGTA
GTTTTTAAGATTTTTGTTATTATTTTGTATGATGTTAAACAGTATTTTAGAC
CGATTTACACAAGTTTATTAAAGTGATATGAAGTGCAAATGAAGAACTG
CAACAT
>CG12110|FBgn0033075
MADAGTVGGALPPYALTNSDEEHGSGEEDASEENSNNEEGEGVFRDCTDE
AVVEHHNRCLPEFQFSLVDSEYDETLAFPDSVTILSNVGDKPVLVERKTDD
DEEEFDDEENNSVVLRHEIPFTSIYGPSVKFNSFQRKVFIPGREIHVRIVDTER
SVTTHLLNPNLYTIELTHGPFKWTIKRRYKHFNSLHQQLSFFRTSLNIPFPSRS
HKEKRTTLKATAREMADESTLKDLPSHTKVKQTSTPLRAEGRSSKIAGSNA
NNAMAMISPNHSSILAGLTPRRLQKKRKKKKKRKLPRFPNRPESLVTVENLS
VRIKQLEDYLYNLLNISLYRSHHETLNFVEVSNVSFVPGMGIKGKEGVILKRT
GSTRPGQAGCNFFGCFQKNCCVRCNYFCSDVVCGTWRNRWFFVKETCFGY
IRPTDGSIRAVILFDQGFDVSTGIYQTGMRKGLQVLTNNRHIVLKCWTRRKC
KEWMQYLKNTANSYARDFTLPNPHMSFAPMRANTHATCPEIYMKRPALDG
DYWRLDKILLRKAEQGVRVFVLLYKEVEMALGINSYYSKSTLAKHENIKVM
RHPDHARGGILLWAHHEKIVVIDQTYAFMGGIDLCYGRWDDHHHRLTDLG
SISTSSFSGSTRRTPSLYFTKDDTDSAFGSRKSSRNAHYDTSAKERPPSPPPDE
PNTSIELKTLKPGDRLLIPSTLVSSPGETPAESGIALEGMKLNTPEMERKNVLD
RLKNNAMKGARMGKDFMHRLTATETEEKSAEVYTIESEEATDHEVNLNMA
SGGQEVAITTSSTQILSEFCGQAKYWFGKDYSNFILKDWMNLNSPFVDIIDRT
TTPRMPWHDVGLCVVGTSARDVARHFIQRWNAMKLEKLRDNTRFPYLMP
KSYHQVRLNPNIQQNRQQRVTCQLLRSVSAWSCGFIEADLVEQSIHDAYIQT
ITKAQHYVYIENQFFITMQLGMGVPGAYNNVRNQIGETLFKRIVRAHKYETK
ILILILADLMFSSSRERKPFRVYVIMPLLPGFEGDVGGSTGIAVRAITHWNYAS
ISRGRTSILTRLQEAGIANPENYISFHSLRNHSFLNNTPITELIYVHSKLLIADDR
VVICGSANNDRSMIGRRDSEIAAILMDEEFEDGRMNGKKYPSGVFAGRLRK
YLFKLHLGLLESEGSSRSDLDINDPVCEKFWHGTWRRISMQNTEIYDEVFKCI
PTDFVKTFASLRKYQEEPPLAKTAPDLAANRANDIQGYLVDLPLEFLNKEVL
TPPGTSKFGLIPTSVWT
>CG12110|FBgn0033075
MADAGTVGGALPPYALTNSDEEHGSGEEDASEENSNNEEGEGVFRDCTDE
AVVEHHNRCLPEFQFSLVDSEYDETLAFPDSVTILSNVGDKPVLVERKTDD
DEEEFDDEENNSVVLRHEIPFTSIYGPSVKFNSFQRKVFIPGREIHVRIVDTER
SVTTHLLNPNLDLRRGV
>CG12110|FBgn0033075
MADAGTVGGALPPYALTNSDEEHGSGEEDASEENSNNEEGEGVFRDCTDE
AVVEHHNRCLPEFQFSLVDSEYDETLAFPDSVTILSNVGDKPVLVERKETDD
DEEEFDDEENNSVVLRHEIPFTSIYGPSVKFNSFQRKVFIPGREIHVRIVDTER
SVTTHLLNPNLYTIELTHGPFKWTIKRRYKHFNSLHQQLSFFRTSLNIPFPSRS
HKEKRTTLKATAREMADESTLKDLPSHTKVKQTSTPLRAEGRSSKIAGSNA
NNAMAMISPNHSSILAGLTPRRIQKKRKKKKKRKLPRFPNRPESLVTWNLS
VRIKQLEDYLYNLLNISLYRSHHETLNFVEVSNVSFVPGMGIKEGVILKRT
GSTRPGQAGCNFFGCFQKNCCVRCNYFCSDVVCGTWRNRWFFVKETCFGY
IRPTDGSIRAVILFDQGFDVSTGIYQTGMRKGLQVLTNNRHIVLKCWTRRKC
KEWMQYLKNTANSYARDFTLPNPHMSFAPMRANTHATCPEIYMKRPALDG
DYWRLDKILLRKAEQGVRVFVLLYKEVEMALGINSYYSKSTLAKHENKVM
RHPDHARGGILLWAHHEKIVVIDQTYAFMGGIDLCYGRWDDHHHRLTDLG
SISTSSFSGSTRRTPSLYFTKDDTDSAFGSRKSSRNAHYDTSAKERPPSPPPDE
PNTSIELKTLKPGDRLLIPSTLVSSPGETPAESGIALEGMKLNTPEMERKNVLD
RLKNNAMKGARMGKDFMHRLTATETEEKSAEVYTIESEEATDHEVNLNMA
SGGQEVAITTSSTQILSEFCGQAKYWFGKDYSNFILKDWMNLNSPFVDIIDRT
TTPRMPWIIDVGLCVVGTSARDVARHFIQRWNAMKLEKLRDNTRFPYLMP
KSYHQVRLNPNIQQNRQQRVTCQLLRSVSAWSCGFIEADLVEQSIHDAYIQT
ITKAQHYVYIENQFFITMQLGMGVPGAYNNVRNQIGETLFKRIVRAHKYETK
ILILILADLMFSSSRERKPFRVYVIMPLLPGFEGDVGGSTGIAVRAITHWNYAS
ISRGRTSILTRLQEAGIANPENYISFHSLRNHSFLNNTPITELIYVHSLLIADDR
VVICGSANINDRSMIGKRDSEIAAILMDEEFEDGRMNGKKYPSGVFAGRLRK
YLFKEHLGLLESEGSSRSDLDINDPVCEKFWHGTWRRISMQNTEIYDEVFKCI
PTDFVKTFASLRKYQEEPPLAKTAPDLAANRANDIQGYLVDLPLEFLNKEVL
TPPGTSKEGLIPTSVWT
>>bin3|FBgn0033073|cDNA sequence
ATTCGGACTTCAAGCAAGAGTCCGCTTTGCCGAGATATAAAATTAATAA
CGAGATCGAGTACCAGCTGCACACAGTGGAAATGAGAAAAGACCGACG
GCAAAACAATAGAACACCCGATTAGTCGTGCGTAACCGATTGACTAA
AGCACGGGGCAGAGTCGATAGAAAAAATATACAGTTTTAAAGCGCTTAA
TTAGGTGTTTTCTAACGTTGGTACATCTCAACGGAGTGGATAACGAGAA
GAGTGAAGGGAGGAGAACCATTGGCAAGAACATACTCACCAAAATGGA
TAATTTCGATAAAATATTTAACAGTGAAAGTGAAAACGGTTGAAGTTTT
AAAATAAAAAGAAATAACTCGTACGCCAAAGAATGCAATATTAAGTGC
AGCCTTGGGTTAATGCCTATCAGGCATTTTACATTGACTGCCATTCGAGC
GTATTAATTGAAAATCTTATGAGGGAGCAAGGCTCTCGAGTAAGTTTAT
AAACTGTGTCCGAAGTAGCATTAAATATCAAAAAGTGAAAATACAAGAC
AAATATTTGAAAAGTGTGCCTGCATAAAACTGAATTAAAAGTAAGGGCC
GATTGCTCTTTAAAAAATAGGGTTAGTCTATGGCGTGCATTCACTAGAGA
AATATGGAAAAGCGGCTTAGCGACAGTCCCGGAGATTGTCGCGTAACGA
GATCCACCATGACGCCCACTCTGCGCCTGGATCAGACTTCCAGGCAAGA
GCCTCTGCCCCAGCAGCCGGATAATGGCCCAGCTGCAGCGCCTGGAAAG
TCTAAGTCCCCCACTCCGTTGCCCGGAAAATCACAGGCCGCCCAGCATC
ACCAGTTCCGCGCTCCGCAGCAGCAGCAGGGCCCGAAAAACCGGAACA
AGGCCTGGATCTACGGGCTCCTCGGTCATAGCCGCGACTCTCCTTCCCAC
TGCGGCGTCGGCTCACAAGGCGGATCTCGAGAACATCCAGAATATCCAT
AACAAAAATCTGACTGCCGGCGGTGGAGTCAACCATCATGGGAACGCCG
GAACAGCGCATCACGGCGGTGGCGGTGGTGCCGGCGCCCATCATGCCGC
AGCGGGTGGCCACCATCACCATCACAACACTAGGCTGGCGCAAAACGCT
GCCGCTGGTGGAGCCAGCGGAGGAGGAACCATTCAAATGCATAAGAAA
ATGTTGAGAGGTCACCATCACCACGTGCTGTGCGCCGGAAACAATGCTA
ACCACACGTGCTGCCTGGTGACGGGATGCAACGGCAGCTCTATCGGCGG
AGTAGGCGTGGCAGGAAGCGGAGGAGCTACCGCCTCAGCGGGGGGCGG
CGGAGCGTCGTGCAAGGAAGCGCAGAGCTGCAAGGACACCAGCTCGCT
GAGCGGCAACAGCAGCATTGCGGGCAGCGCTGGAGCGGGCAACGCAGT
CCACTATTGCTGCGGCCGCTCCAAGTTCTTTTTGCCGGAGAAGAGGTTAC
GCAAGGAGGTGATTGTACCGCCCACCAAGTTTCTGCTGGGCGGCAACAT
CTCCGATCCACTCAACCTTAATTCGCTGCAGAACGAGAACACCTCGAAT
GCCTCCTCCACCAACAACACGCCGGCGACCACGCCCCGCCAGTCGCCCA
TCACTACGCCTCCGAAAGTGGAGGTGATCATACCGCCTAACATCCACGA
TCCGCTCCACCTGCTGGACCCCGflGATTCCATGGAGTACGAGAAGCAG
CTGACGTCGCCGATGAAGCGCGCTGGGCCAGGAGGTGGGATGCTCCACC
ACCGGCAGCACCACTATCGCACGCGAAAGAACCGAAAGCGACGGCGCT
TTGACTCCAACAACACCTCGCATGCCGGCGATGAAGGAGGGGTCGGAAG
CGAGCTGACCGACGAACCGCCGCTGCCCGCAGCCACCTCTTCGCTGGCG
GCGTCGCCGGTGGCAGCGCCCCTTAACGTAGGCGGCAGCTTGCTGCTGA
GCGAATCCGCTGCCCCAGCCCCGGGCGAAACGGCGGAAATGGGACAAC
AGCAGGAGCAGGCGCATGTGCACTCTCCGCAGTCGGCATCAACGACGAC
GACCGCCGCTGAGATGCCCACCCCGACGCCAACCAGTGCAGCGGCAGCG
ACTGCGACCGCGGAGCACAAGGAGCAGTCGGCTCCAGCGCCGACTGCA
ACGTCGTCGCCACAGCGGCAGCAGCAACATGTGGCCGCTGCAGCCGAGG
AACTCCCCACTCCGGAAACTTCCGCTGCTGCTGAGACGCCGGCAGAGGA
GATGCTCCTTAGCTGTTCGGCCACGTCGGCCTCACTGGTGGCTTCGACGC
TGGCAGAGCGAAGGGCCAGCCGAGACCTGCGTCTGGACTTGTCGAGCAC
GTGCTACGGCGTTGGCGGCACGGGTCTGAGCTTCGGCGGCAGCATCTCA
TCCAGCGTCGGCAGTAGCTTTGGTGGCGGTGGGAGGAAGAGGAAAATCA
GCGAGAGCAGCACTTCGCAAAAGAGCAAGAAATTTCATCGTCACGATGC
CATGGACAAGATTGTCAGTCCAGTGGTTCCGCAGCCAGGAGCCTGGAAG
AGACCGCCACGCATCCTTCAGCCCAGCGGAGCTAGGAAGCCCAGCACCC
GCCGCTCTACGTCCGTCAGCGAATCGGAACTACTCAGTCCCGTGGAAGA
GCAGCCGCCCAAACAGCTGCCCCTCATCGGGGTGGAGATACCCCGTGAT
GACACGCCGGATTTGCCGGATCATGGGCTAGGCAGTCCGCTGAGCACTA
CTTCGGGGGCCACCTCGCACACGGCCGGCGAGCAGGATTCTCTGGCCGG
TGTGGACATCAGCATGGGGGATACATTGGGGTCTGGGGTCGTGGGCAAG
GCACCGCTGACTAGTAGTCTTATGCTGGAACCGGCTAAAATTCCACCAA
TTAAAATGCTGCCAAAGTTTCGGGCCGATGGATTAAAGTACCGGTACGG
AAACTTCGACCGCTACGTGGACTTTCGGCAGATGAACGAGTTTCGAGAC
GTGCGCTTGCAGGTTTTCCAGCGTCACGTGGAACTGTTCGAGAACAAGG
ACATTCTAGACATTGGCTGCAATGTTGGCCACATGACCATTACGGTGGC
CAGACATCTGGCACCAAAAACAATTGTCGGTATTGACATTGACCGGGAG
CTTGTTGCACGAGCAAGGAGAAATCTGTCGATCTTTGTGCGTATTCCCAA
GGAGGAAAAGCTGCTGGAGGTCAAAGCAGAGCCAACGGTTGATGCAAA
AGCGAATATCGCGGTGAAGGATGAGACATCTGGAGCAGCTCACAAGAA
AACGAGACGGGGCAAGAGGAGACGCAAGGTGCATCAAGGAATACATCA
TCATCACCATCATCATCATGATTTAGAACAGCTGCAACAGCAACAGAAG
CTGACCTACGGACGTATTCCCCGTATTTTATCATCGAGTAAATCGCCCAA
CATGCTCGGGAACAAGAATCAGTTTCCGGCAAACGTCTTCTTCAGACAC
ACCAACTATGTTCTCAAGGACGAGTCATTGATGGCCAGCGATACCCAGC
AATACGATCTCATACTGTGTCTCTCAGTTACTAAGTGGATCCATCTCAAC
TTTGGAGACAACGGCTTGAAGATGGCATTTAAGCGCATGTTCAACCAGC
TGCGACCCGGTGGAAAACTTATACTTGAAGCCCAAAACTGGGCCAGCTA
CAAGAAGAAAAAGAACCTAACGCCGGAAATATATAACAACTACAAGCA
GATCGAGTTCTTTCCGAACAAGTTTCACGAATACTTGCTTAGTTCGGAGG
TAGGATTCAGTCACAGCTATACGCTTGGCGTGCCTCGTCACATGAACAA
GGGCTTCTGCCGACCCATACAGTTGTATGCAAAGGGCGATTATACCCCG
AATCACGTTCGTTGGAGCGATGCATATTATCCCCAGACGCCATATGAAG
CATATCGGGGCATTTACGCCACCCTGCCCGTTCACCGGATGGGCGGCGG
TGGGAGCAGCGCGGGCGGTAGCAATAGTGGGCATGCTCAAATGCTGCAC
CTTAGCAGCTCCAGTCGGTCGCAGAACTATGATACGCCGCACTACGCAG
GTAGCGCATCGGGATCGGCCAGCTGCAGACAGACTCCAATGTACCAGCC
CACCTACAACCCGTTGGAAACGGACTCATACCAGCCAAGCTACGACATG
GAATATCTCAACCACATGTACGTGTTCGCCTCGCCGCTTTACCAGACCGT
CTGGTCACCTCCAGCCTCGCTGCGCAAAAGCAGCTCGCATACTCCGGTA
TTTGGAAGCGTGCGCGATGCAGAGCTGGACGGTGATGGCAGTGGTGGTG
GGGGCAGTGGTGGCGGAAGCTACCACCGCCACGTCTATCCGCCAAACGA
CGACACTTGTTCGCCCAACGCAAACGCTTGTAATGCGTTTAACTCGATTC
GGGACGCGGACACAGACGATTCTAACCAGCTGCCTGGGGGAAGTCGAC
GACATGTGTATGCAACCAACTGCGGAGAGAGCTCCTCATCGCCGCAGGT
AAATCACCACGATGCGGTTGGCGAATTTGTGGACGGTCTTATGGACGAC
GAACAGAAGTCTTCAACAGGCGGAGGAACTGGTGGCGCAGCTTATTGTG
ATCTGTCGGATGCCTAG
>bin3|FBgn0033073
MEKRLSDSPGDCRVTRSTMTPTLRLDQTSRQEPLPQQPDNGPAAAPGKSKS
PTPLPGKSQAAQHHQFRAPQQQQGPKNRNKAWIYGLLGHSRDSPSHCGVG
SQGGSREHPEYP
>>CG8330|FBgn0033074ICDNA sequence
ATGGGCAACGTAATGGCGTCCACCGCAGACGCTGAGTCCTCTCGTGGGC
GTGGACATCTGTCCGCCGGACTACGCTTGCCGGAGGCACCGCAGTATTC
CGGCGGAGTGCCGCCACAGATGGTGGAGGCCTTAAAGGCGGAAGCTAA
AAAGCCCGAATTGACAAATCCCGGAACTCTTGAGGAACTGCACAGTCGC
TGTCGCGACATCCAGGCCAACACCTTCGAAGGCGCCAAAATTATGGTGA
ACAAGGGTCTGAGCAACCACTTCCAAGTGACCCACACCATTAACATGAA
TTCGGCTGGTCCAAGTGGCTATCGTTTTGGAGCTACCTACGTGGGTACCA
AACAATACGGGCCGACTGAAGCCTTTCCGGTTCTTCTTGGCGAGATCGA
TCCGATGGGCAATCTTAATGCAAACGTTATCCATCAACTGACCTCTCGTT
TGAGGTGCAAGTTTGCCTCGCAGTTCCAGGACTCAAAGCTGGTGGGCAC
CCAGCTGACGGGGGACTATCGCGGCAGAGACTACACGCTGACCCTGACA
ATGGGCAATCCGGGGTTTTTTACGAGTTCCGGAGTATTGTGTGCCAGTA
CTTGCAGTCCGTCACCAAACGTCTGGCGCTAGGATCGGAGTTCGCCTATC
ACTACGGGCCGAATGTGCCCGGACGCCAAGTGGCTGTATTATCAGCGGT
GGGACGTTACGCCTTCGGTGATACCGTGTGGTCTTGCACTTTGGGACCCG
CCGGATTCCACCTFFAGTTACTACCAGAAGGCCAGTGATCAGCTACAGAT
CGGAGTCGAGGTGGAAACGAATATCCGCCAGCAAGAGTCGACGGCCAC
GGTGGCATACCAGATTGATCTGCCCAAGGCAGACCTAGTCTTCCGCGGC
AGTCTCGATTCCAATTGGCTTATTTCCGGAGTCCTTGAGAAAAGACTGCA
GCCGCTACCGTTCTCGTTGGCCATTAGCGGTCGTATGAATCACCAAAAA
AATAGTTTTCGGCTGGGATGTGGCCTCATGATAGGATGA
>CG8330|FBgn0033074
MGNVMASTADAESSRGRGHLSAGLRLPEAPQYSGGVPPQMVEALKAEAKK
PELTNPGTLEELHSRCRDIQANTFEGAKIMVNKGLSNHFQVTHTINMNSAGP
SGYRFGATYVGTKQYGPTEAFPVLLGEIDPMGNLNANVIHQLTSRLRCKFAS
QFQDSKLVGTQLTGDYRGRDYTLTLTMGNPGFFTSSGVFVCQYLQSVTKRL
ALGSEFAYHYGPNVPGRQVAVLSAVGRYAFGDTVWSCTLGPAGFHLSYYQ
KASDQLQIGVEVETNIRQQESTATVAYQIDLPKADLVFRGSLDSNWLISGVL
EKRLQPLPFSLAISGRMNHQKNSFRLGCGLMIG
>>BcDNA:LD21719|FBgn0027519|cDNA sequence
AAAGAAGAAGAGCGAAGAAAACAGATAACTCCAATGTTTGCAAAACAA
TTTGATTAGTCTTGAAGGAATCCGCTTTAGGCTCTTAGGAACCCGCTTTA
TAGCGAGCACAGGCACGCACTGGCACACACAGCAAGCAAAGAGTGACA
ACATTATCCGTGAAGTGGACATATGGCCAACAAGAACATGCGTAACACC
ACAACCAAAGTGGAGGCCCTGATTGAGAGCTGTCGCAGCGAGGGCAAG
TGGCACCGGGTCATCGAGCTAACGGATGAACTGAAGACCGGGTCCCCGC
ACAATGAGTGCCTGGCCAACTTTCTGGTGGGGGAAGCTCGTCTGGAGAG
TTACCTGGAGGAAAACGCTCTCGCGTCAGACTCAAATTTTGGTCGCGCC
AAGTCTGGATTGGCGGAGGCCCGGCGTTTCCTTCACTTGGCTTTGGGTGA
GAGTGGCCAGAAGGCGGGCATCGCCCTGGACGCCTATTTGCTGCTGGCC
AAGCTGTGCTTTGCCTGCGGCGAGTATGAGCAGAGTCTAGACAATTTCG
TCAAGGCAGAACTCAACACGCTTGCCGAGAAGGAGCTGACCCTTCGCAG
CCTGAAGATCCTTGCGGAATCGTATGCCATCAAGGGATTGTGTCTGGAG
CAGCAGACTACGAAGCCGTCATCTAAGTTTAAGAAGGCCGAAAAGGAC
ACGGAAATGATTAGCTGTTTTGAGCGCGCATCTGATCTGGGGCTGCTCTA
TCTGCAGGAATACGATCTCGTTAGTGGAAGCAGTGGCTCGTCCAACAAC
TCGACAGCTGGTTCTACGTTGAATGTAAACGCCTCTACTGTGCAGCCGTC
GAGCAGCAGTTTTGCAATCAGCAGTACAATACCGGCGAGTGGTCCAAGT
GGACTGGAAATGAACCGCAGGATGGGCGCCATCCTAGAGACCGCCCTGC
AACGGGCACCCATAGTGCTTATTAAGACGGAAAAGCTTCAAGAGGCCGT
TGAACGGTATCGAATCATGCTAAACGCCATCGAAACGAGGGCTACTCAA
TCGTTGCGCCTCACGCTGGCCCGCCAGCTAGCTGAGGTTCTTTTGAGAGG
GGTCTCGGGCACGATTTACTCGCCTCCTTTTACCGGAAAATCTGGAGGTG
GGACGCTGCGAGGAGGATCCTCCAAGAAACTTTGGAAACCGCGTAAATA
CGCAGCCCGCCAGCAGTTTAACCCTCGGAACCAGCAGGAAGAGGTAATT
CTGTTGCTACTCATAGCCGAGGCACTGGCTGTGAGGGATACAGTTTTGTC
GCAGAGTCCAGAGTTTAGACAGGCCCGTCAGCATGCTATGGGCAACGTT
ACGGCCGTCTATGACCTCCTGACGTTGGCTACTGTGCGCTGGGGGCTCGT
CCAGCTATTAAATGAGTCCTTTGAGAAGGCGCTAAAGTTTAGCTTTGGTG
AACAGCATGTGTGGCGGCAGTACGGCTTAAGTTTGATGGCAGCCGAAAA
GCACTCGCACGCTTTAAGGGTCTTGCAAGAATCAATGAAGTTGACTCCT
AGTGATCCTTTGCCATGTCTGTTGGCTTCTCGCCTTTGCTACGAGAGTCT
GGAGACGGTAAAGCAAGGTCTGGACTATGCTCAGCAAGCGCTGAAGCG
CGAAGTAAAGGGCTTGCGACCATCGCGAAGCCAACTCTTTGTGGGCATC
GGTCACCAACAGCTAGCCATCCAGTCAAATCTTAAAAGCGAGCGAGATG
CTTGTCACAAGCTGGCTTTGGACGCCCTGGAGCGCGCTGTGCAGTTTGAT
GGGAACGACCACCTGGCGGAATACTACTTGTCGTTGCAGTACGCACTTC
TGGGACAGCTGGCGGAGGCATTGGTTCATATCCGTTTCGCGCTGGCGTT
GCGTATGGAACATGCGCCATGTCTACACCTGTTCGCACTGTTGCTGACAT
CGTCGCGCCGACCTCGTGAAGCTTTGGGAGTTGTTGAGGATGCTTTACAC
GAGTTTCCCGATAACCTGCAGCTACTGCACGTTAAGGCACATCTTCAGCT
GCATCTAGAGGACGCGGAGACGGCGTTGGGCACTGTGCAGCACATGCTG
GCCGTGTGGCGGGACGTTTACGAGGCCCAGCTAGCGGGAGAGGAGGAA
AAGCACTCAGACACCAAGAGTGGTGTTCACTTGGCACATTCCTCACAGA
TGTCCGACAAGGATTCAAATTCTGTGTACGCGGCTTCATFITGGCTGCAGTC
TCCCGCGTTGAACAGGCTCTGAGTGAAGCAGCAAGCTCATTGAGCTCAT
TTACGCAGCGTCCTGGACCCCGACGACCCTGGATGCTACAGATTGAAAT
ATGGCTTCTGCTGGCTGATGTCTATCTGCGGATTGATCAGCCGAACGAG
GCACTCAACTGCATACACGAAGCCTCACAGATTTATCCGCTTTCGCATCA
GATTATGTTTATGCGTGGCCAGGTGCATGTCTATTTGGAGCAATGGTTTG
ACGCCAAGCAATGTTTCCTGAACGCCGTGGCCGCCAACCCAAATCACAC
AGAGGCTTTGCGTGCGCTTGGAGAGGCGCATTTGGTACTGGGCGAGCCG
AGGTTGGCTGAAAAAATGCTAAAAGATGCGGCCAAACTGGATCCGAGCT
GTCCAAAAATTTGGTTCGCACTGGGAAAGGTGATGGAGATCCTGGGCGA
TTTCCATGCCTCAGCCGATTGCTTCGCCACGTCGCTGCAGTTAGAGCCAT
CATGTCCGGTGCTACCTTTTACTTCTATACCTTTGGTGTTTGAATAGGAA
CACTTTCGTGTCTAATTCGAAGCTTGACAAACCTCAAAGTCAACAGCAA
TACTAGAATTATACTTCCTAATTCCTTCAGTGTAAAAATTGTTGTATCGC
AGTTTTGGAGCAACAAATGTTTAAATATTGTTTGTGTGTAAATTATTACC
AAGAATTGTTCGAGCTTGGCTGTATTATGTGAATGAACCATTCTGCTATC
TTCCTTAATCACCCACTTTTAAGGAGATGGTTCGGTTAAATTTATTACTC
TATTAGGTGTTGTTAATTACATACAAAATTGGTTATTATATAAATATACA
GTATTTCGTG
>BcDNA:LD21719|FBgn0027519
MANKNMRNTTTKVEALIESCRSEGKWHRVIELTDELKTGSPHNECLANFLV
GEARLESYLEENALASDSNFGRAKSGLAEARRFLIILALGESGQKAGIALDA
YLLLAKLCFACGEYEQSLDNFVKAELNTLAEKELTLRSLKILAESYAIKGLCL
EQQTTKPSSKFKKAEKDTEMISCFERASDLGLLYLQEYDLVSGSSGSSNNST
AGSTLNVNASTVQPSSSSFAISSTIPASGPSGLEMNRRMGAILETALQRAPIVL
IKTEKLQEAVERYRIMLNAIETRATQSLRLTLARQLAEVLLRGVSGTIYSPPFT
GKSGGGTLRGGSSKKLWKPRKYAARQQFNPRNQQEEVILLLLIAEALAVRD
TVLSQSPEFRQARQHAMGNVTAVYDLLTLATVRWGLVQLLNESFEKALKFS
FGEQHVWRQYGLSLMAAEKIHSHALRVLQESMKLTPSDPLPCLLASRLCYES
LETVKQGLDYAQQALRREVKGLRPSRSQLFVGIGHQQLAIQSNLKSERDAC
HKLALDALERAVQFDGNDHLAEYYLSLQYALLGQLAEALVHIRFALALRME
HAPCLHLFALLLTSSRRPREALGVVEDALHEFPDNLQLLHVKAHLQLHLEDA
ETALGTVQHMLAVWRDVYEAQLAGEEEKHSDTKSGVHLAHSSQMSDKDS
NSVYAASLAAVSRVEQALSEAASSLSSFTQRPGPRRPWMLQIEIWLLLADVY
LRIDQPNEALNCIHEASQIYPLSHQIMFMRGQVHVYLEQWEDAKQCFLNAV
AANPNHTEALRALGEAHLVLGEPRLAEKMLKDAAKLDPSCPKIWFALGKV
MEILGDFHASADCFATSLQLEPSCPVLPFTSIPLVFE
Scim21
AE003789 (insertion @11490), nearest ORE (CG9397 gene 1.28) @11901
>>1.28|FBgn0010347|cDNA sequence
CCCGTCATTGTTGTCAATAAACAAAAGCTGGCTCGCAGTCACAGCGACT
AGGAGGTTAACCTGACTFACCCGTTTCGGTTTTGAATTCGGTTTCATTGT
CGCTTTCTTGAACTTGCGAGCACCGCGTGGCCAATCTTGCAGAATGAATT
TCGGGTGTGCACCGGAAACAACCGACAGTGAAATAACTACTGGCCATTC
ACAAATCCATAAAGCACAGTGTAACACTTTGTAACGGATCGCAAATCCA
ACCACCCCACGCAAATCAAAGCTTAATGCCAAAAGTCATACAAGTGCAA
TTGATTTAAATGAATTTTTGTTTAATTTGTATAAAGTCGTAAAATGCAAG
TTCATGGCGATATATATATACCAATTTGTGCAAACTGATGGGGAAAATC
GGAATGGTAACACCATTAAGGGCAGTGCGGCTAAAAATTGCTGACGAG
CAACTTTCAGATTAATGCAATTCAAATTGGCTTTCCGTCGACTAACAATA
AGCCGTTGGGAATAGCAGATGTGTGCTAAAGCAGATCCAGAGTTCTGGC
AGCAGTTAACACCAATTAAAAGCGTCGTTATAGCAATAGTGCAGCAAAC
ATAGAGGGAAAATGTCAGTCACTCAGCCAAAGGATACGGCATTAAAGA
CCAAGGAGTCGGCAGCTGAAGTAGCAGCCCCTCTGGCCCCACTCTCTGT
CAAAACAGCAGGAGCAACGGGACGGAAAACTTTAACCTCCAGCGCGGC
TTTGTCACTGTTTGATCAGCTGAAAAATAGCGTCAATACAAACAGTTTGA
CAATTGGCGCCGGCGTCGGCAACAACAGTAGCCCAGAAGCCACACCACC
TATAACAGCACCAGCCAGTACAACCACCGCCTCCCCGATACTTACTCCC
AAAAGCCCACCACCCACACCACCCATCAACAAAAGCCCGTCTCTGTCCT
CCAATATTGAACTGAAGCCCCCGGCCAAACCCGCTCGGCCCTTTACCTC
GCCCAGCACTATTGGAATCGTGCAGGGTACCAAACGGGGGGTGGGCGG
AGTTTTTGGCGGGTTTGGAGCCCAAGCCGCCAAATTGGATATAAACGCA
TTGCGATCTCAGCTCTACCAGGGCGCCAGGAAGACTGCAACAACATCTC
GAGTGGGAGTCGGCAAAACAACAACGGTGGCAACAGCCCCAAGAACAA
CTGGAGGAGAACGTGGCACAAAGGGGTCCAAGAAGAGATGTCTAGATC
GGTACGACTCGTCGGAGTCATCAGACAGGTGAGTGCATGTCGTGGGCAC
ACTACTGAAGCATGCAGTGCGGCTGTCCCTGCATCTTGGCTACTGTTTAA
TGGCCTTCGGTGGGGCAGTGGTAGCTGGACGCACCCATTGTCAATATCA
ACAGGTAACGCCACGAAAAACCACTGAGGTATTTGCATTTCCCATTATT
ATTACTGCTGCTGCAGCTGTTGCACCTCGGTCCAACTCCTCGCCCTGGAG
TGCTAATGGCCATCAGTCTGAGCTGGTCAGAATATGCACCCTGGGAGCG
TGTGAAATTTTCTGTTGCCGTTTTCGGTTCCCTGGTCTTTGTTCTGTTGCG
GAGCAATTTGTTGCTGGCCACGTGAAACAAGGCCCGGCAAAAGGGTGTT
TTCAGGGAAATGAGCAGCGCAATGGCAATCGCAGAGGCAAAAGGCCAG
GGGCAAATTGGTAATTAAGTTGTCTCCCGAATGAGGAACTCCGAACGGG
GAGGAGCCACTCCTGGCCCGGTGGAAGCTTCGACTTCCCTAATGAAGTT
ATTTGGTCCAGGAAAATTTCTTTAATTGTAAAAGAATCACTATTTTAATA
AAAGAAACTGGTGATAGTGTTAATCTATATTATAA
>1.28|FBgn0010347
MSVTQPKDTALKTKESAAEVAAPLAPLSVKTAGATGRKTLTSSAALSLFDQ
LKNSVNTNSLTIGAGVGNNSSPEATPPITAPASTTTASPILTPKSPPPTPPINKS
PSLSSNIELKPPAKPARPFTSPSTIGIVQGTKRGVGGVFGGFGAQAAKLDINA
LRSQLYQGARKTATTSRVGVGKTTTVATAPRTTGGERGTKGSKKRCLDRY
DSSESSDR
Scim22
AE003789 (insertion @249000), nearest ORE (CG3268:phtf) @250208
>>phtf|FBgn0028579|cDNA sequence
ATTTGTGAGCACACACTTTAGTTTTTCGTTAGGAACGGGACGTTCGTTCT
GTTGCGCACCAAATTTTTTCGGACCCAATGCAAATGCAAACGCTTTTGCG
GCGTGTGTAGTGCATTCAAAATTACCAGATACCCAACGGGATCCAAAGT
TCCCAGAGCAGTGGCACCGGAATCGATGCGACCAGCAGTCAGCGGAAG
CGTAAGAAATTCGCGCCTAGGTGGACAAAAATCGATCTGTGACGCGGTT
TAAACCAAGGCTGCACGACACTTCGAGGACTTTTATGTGATTATTACTAT
GAAATTGGATGAAATAGTTGCATGGTACCAGAAGAGAATCGGCACCTAT
GACAAGCAAGAATGGGAAAAGACCGTCGAACAGAGGATATTGGACGGC
TTCAATAGTGTCAATTTAAAAAACACCAAGCTGAAGACGGAGCTAATCG
ATGTGGACTTGGTGCGAGGTTCCACGTTCCCTAAGGCCAAGCCCAAGCA
GTCGTTACTCACTGTGATACGCCTGGCCATTCTGCGCTATGTCCTGCTGC
CCCTCTATGCCCAGTGGTGGGTCAAGCAGACCACGCCAAACGCCTTCGG
CTTCATCCTTGTGCTTTACCTCACACAGTTAACCAACTGGGCTATCTACG
TGCTTCACAGCAGTCGCATAGTGCCCCTTGACTATGAGAAGCCGCCAAA
TGGAACCCTGCTTCAGGCAGAGGCAGATGGAGATGCCTCCGATAAGGAT
GCAGATAAGGAGTCCGAGGAACATGCCGCCCTCCTCAGTGCCCTGCTTA
TTCCGTGCGCCCTAAGCTTGCTGATCAGTCTCATCCACTCACAAATTGTA
GCCACTAACACCGCCTCGGGTGTCTCTGGCGGGAGTAGCAAGAACAAGC
TGCGTCGCATATCTGCAAGCTACTTAAGCGACAAAGCAGCAACCAGGGA
GAACCGGGTGCGACGTCGCAAGAAGATTGTGCGAGTTCGACAAGTGGA
GGCTGACTTGTCCCAGGCCAGCAGTAACATATCACTTCCAAACAGAAGA
ACCGCAACCAGCACAATCGAAGTTCTTCCCAGACCGGTCACGCCTTTGC
CTTCACCAACAGTTACCTGTGCCACGGTGCCAGACCCCACCACGCCGAC
TACGCCTTCGCCATCTGTTATCAGGCGGAGCACCAACGAGGAGACCTAT
TTGACAACGACTGCAATCAGCCCACTAACGCAACCGCTGGCAGCCATAG
ACGCATGCTACGATCTCAGCAGAAAGGCAGGGGGAGCTGCTCCCGAAA
GCCCCAAAAAGCGCAACGTCAACTGGCACACGCCTATTCAGATATACGC
TACCTACGAGCTGGGCGAAGAGCCGTGCTCCAGCAGAAAAGTCGCAGA
AGAAAGTGCGCCTGAGTCGGTTGGAGAAAGATTGTGTTCCGTCAAGCCA
GACTACCAGACGCGTCGAAACATCGGGGAGGACGATGGCTTCGAGAGT
CTGAATGGAAAGAGCTCAAGTGGAGAGGACAACAACCATTCGCCTTTGC
CAAACGCGGTGGCTGTTGCGGCTCCACCAGCTCCTGTTCAGACCAATCA
GTTGCGTCTGCGATTAAACACAACAAACGGTGTGACCGCCAGTGCTTCT
CCAACCGAGAAGAAACCCCAGTCGCGCGGCAATGAATCCTCAACGAGTT
GCGCCGAATCGGATGAGTGCGATGATGCCGACATTATGTCCAGTCCCGC
CTCGGGCTGTAACCAAGAGTGCACCACTTCTGCCACCGACTGGCTGGGG
GTGACGACAAATAGCGAAGACTGCAGTTACACCTCTGATCTGGATCACT
CTGACGGGGGCTTGAAGCACACGGCCTTTAGCGACGAAGATCCTGGAGA
GCTGGACATCACCCCTACCACTATACTAAATCCACATAGCAGCCTCGAC
CGTATTAGCTGCACCATTTGGGATCAGCGAGATGCCAAAAAGGCGCAGC
TTTCCGTGCTGGAGATCGCGTCTTGCATAATCGAACGCGTGGACTCAATG
GGCGAGGCCAACGACTACATCTACATAGGCGTGGTCTTCTCTTTCCTGCT
CACATTGATTCCCATCTTCTGCCGTCTCTGCGAGGTATGTTGCCGGGAAG
GTTTTGGTGGAGGAGACTACTTATTACTGGTCAAATGCACTCAGGTCACA
CTCGGGAGCGATGCAGAGAAGGCCAGTGAGATTAGCTACTTTAACATGC
CGCAGCTGCTGTGGGAGAAGTCATCGGCATCGCTCTTCACCCTGCTGGG
CCTTGCCTTCGGCGACAGCCAGTGGGAGCGCATGGTATTGGCTCTGGGC
TTTGTCCAACGCCTTTGCCTGACCCTCATACTGTTCATAATATTCGCCGTT
GCAGAGCGCACCTTCAAGCAACGCTTCCTTTACGCCAAACTCTTCTCCCA
CCTAACTTCATCACGTAGGGCTCGAAAGTCAAATCTTCCCCACTTCCGTT
TGAACAAGGTGCGTAACATCAAGACCTGGCTGAGCGTGAGGTCGTATTT
GAAGAAACGCGGACCCCAGCGATCGGTGGATATCATCGTTTCCGCCGCC
TTCATAGTAACCCTCCTGTTGCTGGCCTTCCTCAGCGTCGAGTGGCTGAA
GGATTCGGCTCATCTGCACACACACCTTACCTTGGAGGCCCTAATCTGGT
CCATAACAATCGGTATCTTTCTGCTGCGCTTCATGACCCTAGGTCAGAAG
ATACAGCACAAGTACCGCAGTGTGTCGGTGCTGATTACGGAGCAAATTA
ACTTGTATCTGCAGATCGAGCAGAAGCCAAAGAAAAAGGACGAGCTGA
TGGTGTCGAACAGCGTGCTCAAGCTGGCCGCCGATCTGCTAAAGGAACT
CGAAACGCCATTCAAGCTCTCTGGCCTTAGTGCCAATCCATATCTATTCA
CAACCATCAAGGTGGTAATCCTGTCGGCCCTATCGGGCGTGCTTAGCGA
AGTTTTAGGCTTTAAACTGAAGCTGCATAAAATCAAGATCAAGTAACCT
ATGCAAGGCGCAGACCCATCATATTTTTGTAGTACAACTTTTTAGAAACG
CTTTAAGAGAAATCTAACACTACACTCTAAATTAGTTAAGTGAATAAATT
TAAGCGAGCC
>phtf|FBgn0028579
MKLDEIVAWYQKRIGTYDKQEWEKTVEQRILDGFNSVNLKINTKLKTELIDV
DLVRGSTFPKAKPKQSLLTVIRLAILRYVLLPLYAQWWVKQTTPNAFGFILV
LYLTQLTNWAIYVLHSSRIVPLDYEKPPNGTLLQAEADGDASDKDADKESEE
HAALLSALLIPCALSLLISLIHSQIVATNTASGVSGGSSKNKLRRISASYLSDK
AATRENRVRRRKKIVRVRQVEADLSQASSNISLPNRRTATSTIEVLPRPVTPL
PSPTVTCATVPDPTTPTIPSPSVIRRSTNEETYLTTTAISPLTQPLAAIDACYDL
SRKAGGAAPESPKKRNVNWHTPIQIYATYELGEEPCSSRKVAEESAPESVGE
RLCSVKPDYQTRRNIGEDDGFESLNGKSSSGIEDNNHSPLPNAVAVAAPPAP
VQTNQLRLRLNTTh4GVTASASPTEKKPQSRGNESSTSCAFSDECDDADIMSS
PASGCNQECTTSATDWLGVTTNSEDCSYTSDLDHSDGGLKHTAFSDEDPGE
LDLTPTTILNPHSSLDRISCTIWDQRDAKKAQLSVLEIASCIIERVDSMGEAND
YIYIGVVFSFLLTLIPIFCRLCEVCCREGFGGGDYLLLVKCTQVTLGSDAEKAS
EISYFNMPQLLWEKSSASLFTLLGLAFGDSQWERMVLALGFVQRLCLTLILFI
IFAVAERTFKQRFLYAKLFSHLTSSRRARKSNLPHFRLNKVRNIKTWLSVRS
YLKKRGPQRSVDIIVSAAFIVTLLLLAFLSVEWLKDSAHLHTHLTLEALIWSIT
IGIFLLRFMTLGQK1QHKYRSVSVLITEQINLYLQIEQKPKKKDELMVSNSVL
KLAADLLKELETPFKLSGLSANPYLFTTIKVVILSALSGVLSEVLGFKLKLHM
KIK
Scim23
AE003838 (insertion @162500), nearest ORF (CG8709) @162503
>>CG8709|FBgn0033269|cDNA sequence
AGCAACAAGTGAACGGAAGAATCCGAGCAGTGAAGAATCAGAAAGACC
GAGGAAACACTCGAGAACTCTTTAATAACATTGTGAACCAAAAAACCAG
AAACAGCCACTGAAAATACACGGAAAGCAGAGTGATTCGCCATAGTTTT
GCTAGTGTTTTCAAGGGCACCCATCATACAGCTGTGCTGCAAATTTTGTG
CCAGTTGCCGTATCTCAGAAGCAGCGGGTCCAAAGTACCGCCAAATACG
CCGTAGAGCCGATTCCTTCGCCAATAAGCGGCGCATTTGACCGCCTGCC
CATAAACATGGCCGCCATATAACCACAAACGGTGAACGCAACCACACA
AAGTCGGAGCTTTGCGATTAGACAAGTAGATAGCAGCGGGGAGTTCAAG
GAGAGATCCCCGCCAGCAAGGAAATCCATTTTGAAGGGAGACCAGCCG
CAGCAGACCAAAGATGAATAGCCTGGCGCGGGTTTTCAGCAACTTCCGC
GACTTCTACAACGACATCAATGCCGCCACCCTCACGGGAGCCATCGATG
TGATCGTGGTGGAGCAGCGCGATGGCGAGTTCCAGTGCTCGCCCTTCCA
CGTCCGGTTCGGCAAACTGGGAGTGCTCAGGAGTCGGGAGAAGGTGGTG
GACATTGAGATCAATGGCGTACCGGTCGACATACAGATGAAGCTGGGCG
ATTCTGGCGAGGCCTTCTTTGTGGAGGAGTGCCTGGAGGATGAGGACGA
GGAGCTGCCAGCCAACCTGGCCACCTCGCCCATACCCAACAGCTTCTTG
GCGTCTCGGGACAAGGCCAACGACACCATGGAGGACATCAGTGGAGTG
GTGACAGATAAGCACACCGACAACACACTGGAGCGTCGCAACCTAAGC
GAAAAGCTCAAGGAGTTCACCACGCAGAAGATCCGGCAGGAGTGGGCC
GAGCACGAAGAGCTGTTTCAGGGCGAGAAGAAGCCGGCGGACTCGGAC
TCGCTGGACAACCAAAGCAAAGCTTCAAACGAAGCTGAGACGGAGAAG
GCAATTCCGGCGGTCATTGAAGACACGGAAAAAGAAAAGGATCAGATC
AAACCAGACGTTAACCTCACCACGGTCACAACCAGCGAAGCCACCAAG
GAGGTGTCCAAGAGCAAAACCAAGAAGCGGCGCAAGAAGTCGCAAATG
AGAAGAATGCCCAGCGCAAGAACTCTTCAAGCAGCTCATTGGGCAGCG
CCGGCGGCGGTGATTTGCCTTCGGCGGAGACGCCATCACTGGGAGTGAG
CAACATCGATGAAGGAGATGCCCCCATATCCAGTGCCACAAACAACAAC
AACACCTCGTCGTCGAACGATGAACAGCTATCCGCTCCCCTGGTGACAG
CTCGCACTGGGGACGATAGTCCGCTCAGCGAGATTCCCCACACCCCCAC
TAGCAATCCACGTCTGGATTTGGACATTCACTTCTTCAGCGACACGGAG
ATCACCACTCCCGTGGGTGGCGGTGGTGCTGGGTCAGGTCGTGCCGCCG
GCGGACGACCTTCGACTCCCATCCAAAGTGACAGTGAACTGGAAACCAC
CATGCGAGACAACCGTCACGTGGTGACTGAAGAAAGCACCGCATCGTGG
AAGTGGGGCGAGTTGCCCACACCGGAGCAGGCCAAGAATGAGGCCATG
AGCGCCGCCCAGGTGCAGCAAAGCGAGCACCAATCGATGCTCAGCAAC
ATGTTCAGCTTCATGAAGAGGGCAAATCGGCTACGCAAAGAGAAGGGC
GTCGGCGAAGTGGGTGACATCTACCTGTCTGATCTGGATGCCGGCAGCA
TGGACCCCGAGATGGCGGCCCTCTACTTCCCTAGTCCCCTGTCCAAGGCG
GCATCACCGCCGGAGGAGGATGGCGAAAGCGGCAATGGCACCAGTCTG
CCTCACTCGCCCAGCTCGCTGGAGGAAGGTCAGAAGAGTATTGACTCGG
ACTTTGACGAGACCAAGCAGCAGAGGGACAACAAGTACTTGGACTTTGT
GGCCATGTCCATGTGCGGAATGTCGGAGCAGGGAGCACCACCCTCGGAC
GAGGAGTTCGACCGCCACCTGGTCAACTATCCAGACGTGTGCAAAAGCC
CCAGCATTTTTTCATCGCCTAACCTAGTCGTACGGCTGAATGGCAAATAC
TACACCTGGATGGCTGCATGTCCCATTGTCATGACAATGATCACCTTCCA
GAAGCCACTAACCCATGATGCCATTGAGCAGCTGATGTCTCAGACAGTC
GACGGCAAGTGTCTGCCTGGCGACGAGAAGCAGGAGGCAGTTGCCCAG
GCCGACAATGGGGGTCAGACGAAGCGCTACTGGTGGAGCTGGCGACGC
TCGCAGGACGCTGCGCCCAACCACTTGAACAACACTCATGGTATGCCTT
TGGGCAAGGATGAGAAAGATGGTGATCAGGCAGCTGTGGCAACGCAAA
CTCGCGGCCTACCTCGCCCGACATCACCGATCCCACGCTGAGCAAGAG
CGACTCCCTGGTGAACGCGGAGAACACCTCGGCGTTGGTGGACAACCTG
GAGGAGCTAACCATGGCCTCCAACAAGAGCGACGAGCCCAAAGAGCGT
TACAAGAAGTCGCTGCGACTTAGCTCGGCGGCTATCAAAAAACTGAACC
TCAAGGAGGGCATGAATGAAATCGAGTTCAGCGTAACGACCGCTTATCA
AGGGACGACGCGCTGCAAGTGCTACTTGTTCCGCTGGAAGCACAACGAC
AAGGTGGTGATCTCGGACATTGACGGCACCATCACCAAGTCGGACGTGC
TGGGCCACATTTTACCCATGGTGGGCAAGGATTGGGCGCAACTCGGTGT
GGCGCAGCTCTTCGAAGATCGAGCAAAACGGCTACAAGCTGCTCTAT
CTGTCAGCCCGTGCCATCGGCCAAAGCAGGGTGACACGCGAGTACCTCC
GGTCGATCCGGCAGGGCAACGTGATGCTGCCGGACGGACCGCTGTTGCT
GAATCCCACGTCCCTGATATCGGCCTTCCACCGCGAGGTGATTGAGAAG
AAGCCGGAGCAGTTTAAGATCGCCTGTCTGTCGGACATCCGCGATCTGT
TTCCCGACAAGGAGCCCTTCTACGCCGGCTACGGCAACCGCATCAATGA
CGTGTGGGCATACCGAGCAGTGGGCATTCCCATCATGCGCATCTTTACG
ATCAACACCAAGGGCGAGTTGAAGCACGAGCTGACCCAAACATTCCAGT
CCTCTGGCTACATCAATCAGTCGCTAGAAGTCGACGAATACTTTCCCCTG
CTAACCAACCAAGATGAATTCGATTACCGGACGGACATCTTCGACGACG
AGGAGTCCGAGGAGGAGCTTCAGTTCAGCGACGACTACGACGTGGACGT
CGAGCACGGTTCGAGTGAGGAAAGCAGTGGGGATGAGGACGATGACGA
AGCCCTCTATAACGATGATTTTGCCAACGATGACAATGGCATCCAGGCA
GTCGTGGCCTCCGGCGACGAACGGACCGCCGATGTGGGCCTCATAATGC
GAGTCCGCCGCGTCTCCACCAAAAACGAAGTCATTATGGCTTCGCCTCC
CAAATACTGCAGCATGACGTACATCGTCGATCAACTGTTCCCGCCGGTG
AAACTCGACGAAGCCTCCGCCGAGTTCTCCAACTTCAACTACTGGCGCG
ACCCCATCCCCGACCTGGAGATCCCCGAGCTGGAGACGGCGCTGGTGCC
ACCGAGCACCAAGGTGGACATGGCCACCCTGCGCCCCATTCCCGAGAAG
TGA
>CG8709|FBgn0033269
MNSLARVFSNFRDFYNDINAATLTGAIDVIVVEQRDGEFQCSPFHVRFGKLG
VLRSREKVVDIEINGVPVDIQMKLGDSGEAFFVEECLEDEDEELPANLATSPI
PNSFLASRDKANDTMEDISGVVTDKHTDNTLERRNLSEKLKEFTTQKIRQE
WAEHEELFQGEKKPADSDSLDNQSKASNEAETEKAIPAVIEDTEKEKDQIKP
DVNLTTVTTSEATKEVSKSKTKKRRKKSQMKKNAQRKNSSSSSLGSAGGG
DLPSAETPSLGVSNIDEGDAPISSATNNNNTSSSNDEQLSAPLVTARTGDDSP
LSEIPHTPTSNPRLDLDIHFFSDTEITTPVGGGGAGSGRAAGGRPSTPIQSDSEL
ETTMRDNRHVVTEESTASWKWGELPTPEQAKNEAMSAAQVQQSEHQSML
SNMFSFMKRANRLRKEKGVGEVGDIYLSDLDAGSMDPEMAALYFPSPLSK
AASPPEEDGESGNGTSLPHSPSSLEEGQKSIDSDFDETKQQRDNKYLDFVAM
SMCGMSEQGAPPSDEEFDRHLVNYPDVCKSPSIFSSPNLVVRLNGKYYTWM
AACPIVMTMITFQKPLTHDAIEQLMSQTVDGKCLPGDEKQEAVAQADNGG
QTKRYWWSWRRSQDAAPNHLNNTHGMPLGKDEKDGDQAAVATQTSRPTS
PDITDPTLSKSDSLVNAENTSALVDNLEELTMASNKSDEPKERYKKSLRLSS
AAIKKLNLKEGMNEIEFSVTTAYQGTTRCKCYLFRWKHNDKVVISDIDGTIT
KSDVLGHILPMVGKDWAQLGVAQLFSKIEQNGYKLLYLSARAIGQSRVTRE
YLRSIRQGNVMLPDGPLLLNPTSLISAFHREVIEKKPEQFKIACLSDIRDLFPD
KEPFYAGYGNRINDVWAYRAVGIPIMRIFTINTKGELKHELTQTFQSSGYINQ
SLEVDEYFPLLTNQDEFDYRTDIFDDEESEEELQFSDDYDVDVEHGSSEESSG
DEDDDEALYNDDFANDDNGIQAVVASGDERTADVGLIMRVRRVSTKNEVI
MASPPKYCSMTYIVDQLFPPVKLDEASAEFSNFNYWRDPIPDLEIPIELETALV
PPSTKVDMATLRPIPEK
Scim24
AE003828 (insertion @25523), nearest ORF (CG6751) @23789

>>CG6751|FBgn0033562|cDNA sequence
TTTGAACTGCACGTGTTTATCAATTCGTTTGGTGTATCAAACTAAGTTGA
AAAATATAATCATAATGGCTGAGGAAGGACCACCGGAGCCGAGCATTG
ATTTTGTCCCAGCTCTTTGCTTTGTACCACGCGGCGTGGCTAAGGATCGT
CCCGACAAGATCGTGCTGACGCAGGCGGAGCTGGCCAGGATTATCGGTG
ATACGCAACAGGAATTGGACGAGGAGAGCGACGACGATGCAGAGGAGG
GCGAAAATGCCGAGGAAGACCAAAACGACATGGATGTGGACGACCACG
CGGATGCCAATAGTGAGAACCGCGATCCGCAGGACGAGTTCCAATTCCA
GGAGTATGACAACGAGGCGAATGCTAATGTCACCAGTCTGGCCAACATC
GTGGACGCTGGCGAGCAAATCCCCGATGAGGACGAAGACTCCGAGGCC
GAGGACGAGGTGATCAAGCCCAGCGACAACCTCATTCTAGTGGGTCACG
TTCAAGACGACGCCGCCTCCATGGAGGTGTGGGTTTTCAACCAGGAGGA
GGAGGCTCTCTACACCCACCACGACTTTCTGCTGCCAAGCTTTCCTCTGT
GCATCGAGTGGATGAATCACGACGCGGGCAGCGAAAAGGCGGGCAACA
TGTGCGCCATCGGCTGCATGGATCCGATAATCACAGTCTGGGATCTAGA
CATACAGGACGCTATCGAGCCCACATTTAAGCTGGGTTCCAAAGGCAGC
CGGAAGCAGAACAAAGAGCAGTATGGACACAAGGACGCCGTGCTGGAT
CTCTCTTGGAACACCAACTTTGAGCACATTCTGGCCAGCGGGTCCGTGG
ACCAAACTGTGATTCTGTGGGACATGGACGAGGGCCAGCCTCATACAAC
CATTACCGCTTTTGGCAAACAGATTCAGTCGCTGGAATTCCATCCGCAAG
AGGCTCAAAGCATTCTTACCGGCTGTGCCGATGGATACGTGCGACTCTTC
GATTGCCGCGACGCTGAGGGCGTCAACTCGTCCAGCATTGAGTGGAAAG
TTGACGGTGAAGTGGAGAAGGTCCTGTGGCATCCCACACAGACCGACTA
CTTCATCGTGGGCACCAACGATGGCACCTTGCATTACGCCGACAAACGT
TCTCCTGGACAACTGCTGTGGTCCGTAAAGGCCCACAACGAGGAAATCT
CCGGTGTGTGCTTCAACAACCAGAAGCCTAATCTGCTGACCTCCACCTCC
ACGGAGGGCACCCTAAAGGTGTGGAACTTTGATGGCACAGAGGCAAAG
CACGTCTACGAGCACGAGTTCAACATGGGTCGCTTGCAGTGCATGCGCC
AGTGCCCCGAGGATCCCTACACCCTGGCCTTCGGCGGAGAGAAGCCTCC
GCGCTGTGCGATCTTTAACATCAAGAACTCGATAGCCGTGCGCCGAACG
TTTGGAATCCCTGATGCAGAGTAGGCAAATCGTACAGCTACGTATTTATC
TGTGTATATGCTTTATATGACTTTTAAATAAATATGAATTATATATAAGA
ACCTTAATGATTGACTTTTATATTAATTAAAATTTTATTGATAACTTGCGC
ATATATGCACTTTACACTTTTATGCTTAAACAACTAATCGACATTTCAGG
GGGGATGGGTCACAAACGAAATACAAAACATTAATCCTAAACATTCCGA
GCATTCCTTAACACTACATTACGTATACCAAATAAGCTTATCTGTGCTCC
TAACTCTTGAATAGACCCACGCACATCAGGAGATTTCGGCGCGTAAAGT
GCAGGCTGACAAAT
>CG6751|FBgn0033562
MAEEGPPEPSIDFVPALCFVPRGVAKDRPDKIVLTQAELARIIGDTQQELDEE
SDDDAEBGENAEEDQNDMDVDDHADANSENRDPQDEFQFQEYDNEANAN
VTSLANIVDAGEQIPDEDEDSEAEDEVIKIPSDNLILVGHVQDDAASMEVWVF
NQEEEALYTHHDFLLPSFPLCIEWMNHDAGSEKAGNMCAIGCMDPIITVWD
LDIQDAIEPTFKLGSKGSRKQNKEQYGIIKDAVLDLSWNTNFEHILASGSVDQ
TVILWDMDEGQPHTTITAFGKQIQSLEFHPQEAQSILTGCADGYVRLFDCRD
AEGVNSSSIEWKVDGEVEKVLWTIPTQTDYFIVGTNDGTLHYADKRSPGQLL
WSVKAHNEEISGVCFNNQKPNLLTSTSTEGTLKVWNFDGTEAKHVYEHEFN
MGRLQCMRQCPEDPYTLAFGGEKPPRCAIFNIKNSIAVRRTFGIPDAE

Scim25
AE003815 (insertion @3170), two ORFs nearby: CG8151 @878 to 3125, CG13941
@3609 to 4190
ESTs in the clot C#3527—have 96% identity with CG8151 gene product
>>CG8151|FBgn0033929|cDNA sequence
GCAAATAACGTGGGATTGTGCGTTTTGCCGACCGCGAAATGGGGAAAAG
TATCGCCGGCGCAGGCGACATACGCAAACACCAGGCGGCACTTTTCCGC
CAGCCGAAGATGCTTCTTAGATCGCATTTCATGCTCTTTCAGGTTGCAGG
AATCTTCTGGCGTCCCCCTTCTCGTCCTTTCGAAGGCTCTCATGTGAGAC
GGCGAGCGTGGATCTGCGACTGGGACTTCGACTGCTGAGCGCCGGGCGT
AGAACAAGATGACCACCAGCAGTGAGGACGTGCTGCTCCAGATGGGCG
AGGTGCGGTACAAGAAGGGCGACGGCACGCTCTACGTAATGAATGAGC
GTGTGGCCTGGATGGCGGAACACCGGGACACGGTAACAGTCTCCCATCG
TTATGCGGATATCAAGACTCAAAAGATATCTCCTGAGGGCAAGCCCAAG
GTGCAGCTGCAAGTGGTTCTTCACGACGGCAACACATCGACCTTCCACTT
CGTCAACCGCCAGGGACAGGCCGCAATGCTTGCCGACAGGGACAAGGT
CAAGGAGCTATTGCAGCAACTGCTTCCCAACTTCAAGCGGAAGGTGGAC
AAAGACCTGGAAGACAAGAACCGCATCCTTGTTGAGAATCCCAACCTGC
TGCAACTCTACAAGGACCTTGTCATAACCAAAGTCCTAACCAGCGATGA
GTTCTGGGCTACGCATGCCAAGGATCACGCCCTTAAGAAAATGGGCAGA
TCCCAGGAGATCGGTATAGGTGTTTCTGGCGCCTTTCTGGCTGACATAAA
GCCGCAGACAGACGGCTGTAATGGCCTCAAGTACAACCTCACCTCTGAT
GTGATTCACTGCATTTTCAAGACCTATCCCGCCGTTAAACGCAAACATTT
TGAGAATGTGCCTGCCAAAATGTCCGAGGCCGAGTTTTGGACCAAGTTT
TTCCAATCACACTACTTTCATCGTGACAGACTGACAGCCGGCACAAAGG
ACATATTCACGGAGTGCGGCAAGATCGATGACCAAGCATTAAAAGCGGC
TGTTCAGCAGGGAGCTGGTGATCCTTTGCTAGACCTTAAAAAGTTTGAG
GATGTTCCTTTGGAAGAGGGCTTTGGCAGCGTAGCAGGGGACCGCAACG
TCGTGAACAGCGGGAATATTGTGCACCAAAACATGATCAAGCGATTCAA
TCAGCATTCCATCATGGTGCTTAAGACCTGTGCTAACGTGACCTCAGCGC
CGTCAACTATGACCAATGGTACCAATAATGCCAACGGGCCTGTTTCCCA
ATCCGCGTATACGAACGGGATGAATGGAAAGGGCCAGGCCACGGCCAC
CGCGACGAAGAGTTCCTCCGATCAGGTGGACAAAGACGAGCCGCAGAG
CAAAAAGCAACGACTGATGGAAAAGATTCACTATGTGGATCTCGGGGAC
CCTATATTGGAGGGAGATGATTCCGCCAACGGCGAGAAAGCCAAGTCTA
AGCACTTCGAACTGTCCAAAGTGGAGCGTTACCTCAATGGCCCTGTCCA
GAACAGCATGTACGACAACCACAACGATCCAATGAGTCTTGAAGAGGTG
CAGTACAAGCTGGTGCGGAATTCGGAGTCATGGCTAAACCGCAACGTGC
AACGAACGTTCATCTGTTCTAAGGCGGCAGTAAATGCTCTGGGTGAACT
AAGTCCTGGCGGTTCCATGATGCGCGGTTTCCAAGAGCAGTCAGCGGGA
CAACTTGTTCCGAACGACTTCCAACGAGAGCTGCGCCACTTATACCTTTC
GCTGTCCGAGCTGCTGAAACACTTTTGGAGCTGCTTTCCGCCCACCTCAG
AAGAGCTGGAGACAAAGTTACAGCGTATGCACGAGACGTTGCAGCGCTT
CAAAATGGCCAAACTAGTGCCTTTTGAGGTGAGTTFFTACAAACCGCGCT
ATGCACGAACTTTCGCCACTGCGATCCTCGCTGACGCAGCACTTGAATC
AGCTGCTGCGCACCGCCAACAGCAAGTTCGCAACTTGGAAGGAGCGAA
AACTGCGCAACACCAGGTAG
>CG8151|FBgn0033929
MTTSSEDVLLQMGEVRYKKGDGTLYVMNERVAWMAEHRDTVTVSHRYA
DIKTQKISPEGRPKVQLQVVLHDGNTSTFHFVNRQGQAAMLADRDKVKELL
QQLLPNFKRKVDKDLEDKNRILVENPNLLQLYKDLVITKVLTSDEFWATHA
KDHALKKMGRSQEIGIGVSGAFLADIKYQTDGCNGLKYNLTSDVIHCIFKTY
PAVKRKLHFENVPAKMSEAEFWTKFFQSHYFHRDRLTAGTKDIFTECGKIDD
QALKAAVQQGAGDPLLDLKRFEDVPLEEGFGSVAGDRNVVNSGNIVHQNM
IKRFNQHSIMVLKTCANVTSAPSTMTNGTNNANGPVSQSAYTNGMNGKGQ
ATATATKSSSDQVDKDEPQSKKQRLMEKIHYVDLGDPILEGDDSANGEKAK
SKIHFELSKVERYLNGPVQNSMYDNHNDPMSLEEVQYKIVRNSESWLNRNV
QRTFICSKAAVNALGELSPGGSMMRGFQEQSAGQLVPNDFQRELRHLYLSL
SELLKIIFWSCFPPTSEELETKLQRMHETLQRFKMAKLVPFEVSFTNRAMHEL
SPLRSSLTQHLNQLLRTANSKFATWKERKLRNTR
C>>G13941|FBgn0033928|cDNA sequence
ATGACGCAGATGTCCGACGAACAGTTTCGCATATTCATAGAAACCATTA
AATCGCTGGGGCCAATCAAAGAGGAACCGCCATCCAAGGGTAGCTTTAG
CAACTGCACGGTGAGATTCAGTGGCCAGCGGGATCACGATGCCGTGGAC
GAGTTCATCAATGCCGTGGAGACGTATAAAGAGGTGGAGGGCATCAGCG
ACAAGGATGCGCTAAAGGGTTTGCCGCTGCTCTTCAAGAGCATTGCCGT
GGTGTGGTGGAAGGGTGTGCGCCGGGATGCCAAGACCTGGTCGGATGCC
CTGCAGCTGCTGCGCGATCACTTCTCGCCCACTAAACCTTCCTACCAGAT
ATACATGGAGATCTTCGAGACGAAGCAGTCCTACGACGAAGTGATCGAC
TCATTCATCTGCAAGCAGCGAGCGCTCCTAGCCAAGTTGCCGGAGGGAC
GACACGACGAGGAGACGGAGCTGGACTTCATCTACGGGCTGATGCAGGC
CAAGTACCGGGAGAGCATACCCCGACACGAGGTCAAAACCTTCCGGGA
GCTACTCGATCGGGGGCGAACTGTGGAGCGCACAAGGCACTGA
>CG13941|FBgn0033928
MTQMSDEQFRIFIETIKSLGPIKEEPPSKGSFSNCTVRFSGQRDHDAVDEFINA
VETYKEVEGISDRDALKGLPLLFKSIAVVWWKGVRRDAKTWSDALQLLRD
HFSPTKPSYQIYMEIFBTKQSYDEVIDSFICKQRALLAKLPEGRLIDEETELDFI
YGLMQPKYRESIPRHEVKTFRELLDRGRTVERTRH
Scim26
AE003815 (insertion @33900), nearest ORF (CG13942) @36413
The EST GH23043 has 94% identity with CG8603 gene product: 3′end is at 17162
>>CG13942|FBgn0033922|cDNA sequence
ATGCAACATCGATTTCTCTTGCAGGATGACCTGCCGCACCACAACAGCA
GCAGCAGCCAGCTGGGCCAGCAACACGGCTCATCGTTGGACCAGTGCGG
ATTGACTCAGGCCGGCCTCGAGGAGTACAATAATAGATCGTCCTCGTAC
TACGACCAGACGGCCTTCCATCACCAGAAGCAGCCATCCTATGCCCAAT
CCGAGGGCTACCACAGCTATGTGTCAAGTTCGGATTCCACATCGGCCAC
GCCATTTCTGGATAAATTACGTCAGGAGAGCGATCTGCTGTCGCGCCAA
TCGCATCATTGGTCGGAGAACGATCTGTCCTCCGTTTGCAGCAACTCTGT
GGCGCCTTCGCCCATTCCGCTGTTGGCCCGTCAGTCTCACTCCCACTCTC
ATTCTCACGCGCATTCCCATTCGAACTCCCATGGCCATTCCCACGGTCAC
GCCCACTCAGCCTCCTCATCCTCATCCAGCAACAACAATAGCAACGGCA
GCGCCACCAACAACAACAACAACAACAGCTCGGAAAGCACTTCCTCCAC
GGAAACCCTCAAGTGGCTGGGCTCCATGAGCGATATATCCGAAGCCAGT
CATGCAACCGGCTACAGCGCCATCTCCGAATCGGTTTCCTCCTCGCAGCG
CATTGTCCACAGTTCCCGGGTGCCGACACCCAAGCGTCATCATAGCGAG
AGCGTGCTGTATCTGCACAACAACGAGGAGCAAGGCGACAGCTCGCCCA
CTGCGAGCAACTCCTCGCAGATGATGATCTCCGAGGAGGCGAATGGCGA
GGAATCGCCGCCGTCGGTGCAGCCACTTCGCATCCAGCACCGTCACAGT
CCCAGCTATCCGCCCGTGCACACCTCGATGGTGCTGCACCACTTTCAGCA
GCAGCAGCAGCAGCAGCAGGATTACCAGCACCCGAGTCGCCACCACAC
CAACCAGTCCACGTTGAGCACACAAAGTTCCCTGCTGGAGCTGGCCTCG
CCCACGGAGAAACCTCGCTCCCTCATGGGACAATCCCACTCCATGGGCG
ACCTGCAGCAAAAGAATCCGCATCAGAATCCGATGTTGGGACGATCGGC
TGGTCAGCAGCACAAGTCCAGCATTTCCGTGACCATTTCCAGCAGCGAG
GCCGTGGTCACCATTGCACCACAACCGCCAGCTGGTAAGCCCAGCAAGC
TGCAGTTGTCCCTGGGAAAGTCGGAGGCCCTCAGTTGCAGTACACCCAA
TATGGGGGAGCAGAGTCCCACGAACAGCATCGATTCCTACCGCAGCAAC
CATCGCCTGTTCCCGGTGAGCACCTACACGGAGCCGGTGCACAGCAACA
CCTCGCAGTACGTGCAGCATCCCAAGCCGCAGTTCAGCTCCGGGCTGCA
CAAGTCCGCCAAACTTCCTGTGATAACGCCAGCGGGGGCCACAGTGCAG
CCCACCTGGCACTCGGTGGCCGAGAGGATTAACGACTTTGAGCGCAGTC
AGTTGGGGGAGCCACCGAAGTTTGCCTACCTGGAGCCCACCAAGACGCA
CCGCCTCTCGAATCCGGCTCTAAAGGCTCTCCAGAAGAACGCAGTGCAG
TCCTATGTGGAACGACAGCAGCAGCAGCAGAAGGAGGAACAGCAGCTA
CTACGTCCGCACTCGCAATCCTACCAAGCGTGTCATGTGGAGCGCAAAT
CACTGCCGAACAACTTGAGTCCCATAATGGTGGGTCTGCCCACTGGGAG
TAACTCCGCATCGACTCGGGACTGCAGTTCACCCACTCCACCACCACCG
CCACGACGTTCGGGGAGTCTGCTGCCCAATCTGCTAAGGCGCTCCAGTT
CGGCCTCGGACTACGCGGAGTTCAGGGAGCTGCATCAGGCACAGGGTCA
GGTCAAGGGACCGAGCATTAGGAACATAAGCAATGCCGAGAAAATCTC
CTTCAATGACTGCGGAATGCCACCTCCGCCGCCGCCACCACGAGGACGT
TTGGCCGTGCCGACCAGACGCACATCCTCGGCAACGGAATACGCACCCA
TGCGGGACAAACTGCTGTTGCAGCAGGCCGCCGCCTTGGCCCACCAGCA
GCACCACCCGCAGCAGCATCGCCATGCCCAACCGCCCCATGTGCCGCCC
GAGCGTCCGCCCAAGCATCCCAATCTTCGGGTGCCGTCGCCTGAGCTGC
CACCGCCGCCGCAGAGTGAACTTGACATCAGTTATACCTTCGATGAGCC
ATTGCCGCCGCCACCGCCGCCGGAAGTGCTCCAGCCACGCCCACCGCCC
TCGCCCAACCGGCGGAATTGCTTCGCCGGAGCATCCACACGTCGCACCA
CCTACGAAGCACCACCGCCCACCGCAATTGTCGCCGCCAAGGTGCCACC
GCTGGTGCCCAAGAAGCCAACGAGCTTGCAGCACAAGCATCTCGCCAAC
GGAGGAGGCGGCAGTCGCAAGCGCCCGCACCACGCGACTCCACAGCCC
ATCCTCGAAAATGTGGCCAGTCCCGTGGCGCCACCGCCGCCCCTGTTGC
CGCGTGCCAGATCCACCGCCCATGACAATGTGATTGCCAGCAATCTGGA
GAGCAACCAGCAGAAACGGTGA
>CG13942|FBgn0033922
MQHRFLLQDDLPHHNSSSSQLGQQHGSSLDQCGLTQAGLEEYNNRSSSYYD
QTAFHHQKQPSYAQSEGYHSYVSSSDSTSATPFLDKLRQESDLLSRQSHHW
SENDLSSVCSNSVAPSPIPLLARQSHSHSHSHAHSHSNSHGHSHGHAHSASSS
SSSNNNSNGSATNNNNNNSSESTSSTETLKWLGSMSDISEASHATGYSAISES
VSSSQRIVHSSRVPTPKRHHSESVLYLHNNEEQGDSSPTASNSSQMMISEEA
NGEESPPSVQPLRIQHRHSPSYPPVHTSMVLHHFQQQQQQQQDYQHPSRHH
TNQSTLSTQSSLLELASPTEKPRSLMGQSHSMGDLQQKNPHQNPMLGRSAG
QQHKSSISVTISSSEAVVTIAPQPPAGKPSKLQLSLGKSEALSCSTPNMGBQSP
TNSIDSYRSNHRLFPVSTYTEPVHSNTSQYVQHPKPQFSSGLHKSAKLPVITP
AGATVQPTWHSVAERINDFERSQLGEPPKFAYLEPTKTHRLSNPALKALQK
NAVQSYVERQQQQQKEEQQLLRPHSQSYQACHVERKSLPNNLSPIMVGLPT
GSNSASTRDCSSPTPPPPPRRSGSLLPNLLRRSSSASDYAEFRELHQAQGQVK
GPSIRNISNAEKISFNDCGMPPPPPPPRGRLAVPTRRTSSATEYAPMRDKLLL
QQAAALAHQQHHPQQHRHAQPPHVPPERPPKHPNLRVPSPELPPPPQSELDI
SYTFDEPLPPPPPPEVLQPRPPPSPNRRNCFAGASTRRTTYEAPPPTAIVAAKV
PPLVPKKPTSLQHKHLANGGGGSRKRPHHATPQPILENVASPVAPPPPLLPR
ARSTAHDNVIASNLESNQQRR
>>CG8603|FBgn0033923|cDNA sequence
ATGAGACGTGCGATTCGGGCAATATTTTCGGTGCTTTTGGCTTTTGTCCT
CAAGTCGTGGCGGTTACTGCCGATGACCCCATCGAATTCCAAGGCCTCA
TACTTGCCGCGTCAGAGTCTGGAGAAGTTGAACAACACTGATCCCGACC
ATGGCATATACAAGCTCACCCTGACCTCCAACGAGGACTTGGTGGCCCA
CACGAAGCCCAGCTATGGGGTCACAGGAAAGCTGCCCAACAATCTGCCG
GATGTCCTGCCGCTGGGCGTTAAGCTCCACCAGCAGCCAAAGTTGCAGC
CAGGATCGCCGAACGGCGATGCGAATGTGACCCTGCGCTATGGCTCCAA
CAACAATCTGACTGGGAATTCCCCGACGGTTGCCCCGCCCCCCTACTATG
GGGGCGGCCAGCGGTATTCAACTCCTGTGCTGGGTCAAGGTTACGGCAA
AAGTTCGAAGCCCGTGACCCCGCAACAATATACGAGATCTCAGTCGTAC
GATGTGAAGCACACTAGTGCGGTGACTATGCCGACAATGTCCCAGTCCC
ACGTGGATCTCAAGCAGGCCGCCCATGACCTAGAGACGACGCTGGAGG
AGGTGCTGCCCACTGCCACGCCCACGCCGACGCCAACACCGACGCCCAC
ACCGCCACGCCTCTCGCCGGCTTCCTCGCACTCGGACTGCAGTCTGAGC
ACCAGTTCCTTGGAGTGCACAATCAATCCTATAGCGACACCGATTCCTA
AGCCTGAGGCGCACATCTTTCGCGCCGAGGTGATTAGCACCACCCTGAA
CACAAATCCGTTGACAACACCGCCCAAGCCCGCGATGAACCGCCAGGAA
TCCCTGAGGGAGAACATCGAAAAGATCACCCAACTACAGTCGGTGCTGA
TGTCGGCGCACCTGTGTGATGCGAGTCTACTAGGTGGTTACACCACTCCA
CTGATAACCAGTCCCACTGCCAGTTTCGCTAACGAACCACTAATGACAC
CACCACTGCCGCCCAGTCCGCCACCGCCACTAGAACCGGAGGAGGAGG
AGGAGCAGGAGGAGAACGATGTGCACGACAAGCAGCCAGAGATCGAGG
AACTGCAGCTGATGCAGCGCAGCGAATTGGTCCTAATGGTGAATCCCAA
GCCGAGCACAACGGATATGGCCTGCCAAACGGACGAGCTGGAGGACAG
GGACACGGACCTCGAAGCGGCACGCGAGGAGCACCAGACTAGAACGAC
TCTGCAGCCGCGACAGCGCCAGCCCATCGAGCTGGACTACGAGCAGATG
AGCCGGGAGCTGGTTAAGCTCCTACCGCCTGGTGACAAGATCGCCGACA
TCCTCACACCAAAGATCTGCAAGCCCACCTCGCAATACGTTAGCAATCT
GTACAATCCGGATGTGCCACTGCGCTTGGCCAAGCGCGATGTTGGCACC
TCTACGTTGATGCGAATGAAGTCCATCACGTCGTCTGCCGAGATCCGAG
TGGTCAGTGTGGAGCTGCAGCTGGCAGAGCCGAGCGAGGAGCCGACGA
ATTTAATCAAGCAAAAGATGGATGAGCTCATCAAGCATTTGAACCAAAA
AATTGTCTCCCTGAAACGCGAGCAGCAGACGATCAGCGAGGAGTGCTCG
GCCAATGACAGACTGGGCCAGGATCTATTCGCCAAGCTAGCGGAGAAG
GTTCGACCCAGCGAAGCCTCCAAGTTCCGTACCCATGTCGACGCCGTGG
GCAACATAACCAGTTTACTTCTGTCGCTTTCCGAGCGTTTGGCCCAAACC
GAAAGCAGCCTGGAAACGCGCCAGCAGGAAAGGGGCGCGCTGGAATCA
AAGCGGGATCTGCTGTACGAGCAGATGGAGGAGGCGCAGCGTCTCAAAT
CGGACATAGAACGACGTGGAGTCAGCATCGCCGGATTACTGGCCAAGA
ACCTCAGCGCGGACATGTGCGCCGACTACGACTACTTCATCAACATGAA
GGCCAAGCTGATCGCCGATGCACGCGACCTGGCCGTAAGGATCAAGGGC
AGCGAGGAGCAGCTTAGCTCCCTCAGCGATGCGCTAGTCCAAAGCGATT
GTTAG
>CG8603|FBgn0033923
MRRAIRAIFSVLLAFVLKSWRLLPMTPSNSKASYLPRQSLEKLNNTDPDH
GIYKLTLTSNEDLVAHTKPSYGVTGKLPNNLPDVLPLGVKLHQQPKLQPG
SPNGDANVTLRYGSNNNLTGNSPTVAPPPYYGGGQRYSTPVLGQGYGKSS
KIPVTPQQYTRSQSYDVKHTSAVTMPTMSQSHVDLKQAAHDLETTLEEVLP
TATPTPTPTPTPTPPRLSPASSHSDCSLSTSSLECTINPIATPIPKPEAH
IFRAEVISTTLNTNPLTTPPKPAMNRQESLRENIEKITQLQSVLMSAHLC
DASLLGGYTTPLITSPTASFANEPLMTPPLPPSPPPPLEPEEEBEQEEND
VHDKQPEIEELQLMQRSELVLMVNPKPSTTDMACQTDELEDRDTDLEAAR
EEHQTRTTLQPRQRQPIELDYEQMSRELVKLLPPGDKIADILTPKICKPT
SQYVSNLYNPDVPLRLAKRDVGTSTLMRKSITSSAEIRVVSVELQLAEP
SEEPTNLIKQKMDELIKHLNQKIVSLKREQQTISEECSANDRLGQDLFAK
LAEKVRPSEASKFRTHVDAVGNITSLLLSLSERLAQTESSLETRQQERGA
LESKRDLLYEQMEEAQRLKSDIERRGVSIAGLLAKNLSADMCADYDYFIN
MKAKLIADARDLAVRIKGSEEQLSSLSDALVQSDC
Scim27
AE003803 (insertion @144410), nearest ORF (CG10939) from 133835 to 144393
C#553: GH04176 98% identity with CG10939 gene product
>>CG10939|FBgn0034209|cDNA sequence
CGAAAGCGTTAACAACGTTTCAACGGATCTTCAGCGTGTGAGATAATAT
TACATACGTAGAAATAATATCAGGAAGGCAGCAGCAACAGCAGCAAAA
ACAACGCGAGTAGCCCTCTCTCTGCGCCTCTTTCGCCTGTCAACAGTTAT
TTTAGCCGATTGTTTTGTGTGACTTTTTCGTGTGCTGTTCGCTTTCGTTTC
GTTTAGCTGTTCGGCAACTCCTTCATTTCATTAAAAATAGTAAGGCCTTG
TAACAACAACAACAAGAACGACGACGTGTTTATGTGTGTGTATGTGACA
GCGTTTGCATACGGAAAAGAGTAGAGAGTGCAACAATAATAACTGCAAC
AAAAACAGAAAACTGAAAATCAACAGCAACATTTGAAAGGGAATCGTT
TCTACTTGTTTGTTTAAGCGAAGTCAAGATGTCCACGCCCACTTCCCCGA
AGACGCCCACACCGCCCACTTTGCCACCGGGCGTGACCAAAACATGTCA
CATTGTGAAAAGGCCCGATTTCGATGGCTATGGTTTCAATTTGCATTCGG
AGAAGGTGAAACCAGGACAGTTTATTGGCAAAAGTAGATGCGGMTTCTCC
GGCAGAGGCAGCCGGCCTGAAGGAGGGCGATCGCATCCTGGAGGTCAA
CGGGGTGTCCATTGGCAGCGAGACCCACAAGCAGGTGGTTGCCAGGATC
AAGGCCATTGCGAATGAAGTCCGCTTGCTGCTCATCGATGTGGATGGCA
AGGCCTTGGAGGTGAAACCGGCATCTCCGCCAGCCGCTGCGTGCAATGG
AAACGGTAGTGCCAGTCAGAATGGATACGAGGGCACCAAACAGGAGAT
GCCCGGAGCAAGTGCCAATATCAGTAGCATCAGTATGGTGAGCACCAG
CGATCCTCAAATGCCAGCAGCATTCAGAGCGGCAGTACCATGAATGCCT
CCGATTTGGATGTGGTCGATAGGGGAATACCGGCAGTCGCTGCTCCGGT
GGCTATCACCCCGCCTCCCGTTCAAAATGGAAGTAAACCCTCATCGCCG
ATTAATAATAACACTTTGATGAGCACACCGCCACCGCCGTCCGCTACTA
AGGCTGGCATCAACAACAATGGCAGTGTTTATAACACCAATGGAAATGG
TACAAATGGCATGACCACACCCACTACACCACCCCCACCGACCAGTGGC
TATAAGGCGGGCACCTTGCATTTACCAATGACGGCCGCCGAAATGCGCG
CCAAATTGGCATCCAAGAAGAAGTACGATCCCAAGAACGAGAGTGTGG
ACCTCAAGAAGAAGTTCGACATCATTCAGAAGCTCTGAGACGAAAAGGG
TAGCCCAACCAACTACTTGTTATAATGTCAGGATGAGGAGCTAGAGCTG
GTTTTGTCAGGCATACACCACACCACACAATATACAATATGTTTAGCTAT
TAGTACGAAGAGTCACTTATTAACTAAGCAAGTTTTTAATTATTACCCCC
TAAGAGAAAGAGCGACCAACGATGGTAGAGTAAACGGATATGATGGAG
CACCTACCCTTGGAATATCTATACATTGTACGACATACGCGTATTCTTCA
AATTCAAATATTGCAAACTCCGATTGGCAATGTTGCCCTGGTTCATTGAA
CAACTTTCATTGAATATGTACTTAGTTTTGCTTGTATTTTTGTAAAGTAAA
TAAAGCAAAAATATAAAAGAAATAC
>CG10939|FBgn0034209
MSTPTSPKTPTPPTLPPGVTKTCHIVKRPDFDGYGFNLHSEKVKPGQFIG
KVDADSPAEAAGLKEGDRILEVNGVSIGSETHKQVVARIKAIANEVRLLL
IDVDGKALEVKPASPPAAACNGNGSASQNGYEGTKQEMPGASANISSISM
VSTRRSSNASSIQSGSTMNASDLDVVDRGIPAVAAPVAITPPPVQNGSKY
SSPINNNTLMSTPPPPSATKAGINNNGSVYNTNGNGTNGMTTPTTPPPPT
SGYRAGTLHLPMTAAEMRAKLASKZKAQYDPKNESVDLKKKFDIIQKI
CG6568 is closeby on the opposite strand
>>CG6568|FBgn0034210|cDNA sequence
ATGAAGAGAGCAGCAACAACAAAGATGACTGGAGCCACGGCTGCGGGA
GCAACAACAACAACATCGTCAACAGGTGCAGTGGGATATCCCGTTCTCA
AAACACCCAAGTATGTGGTTCAGACTAGTCCGAGTGGATCCTCTGGCCA
TCAGCTCCAGATGCTGGCGAGGAAGGACACTCAAAGTCTGGGAGTGGCC
ATCAATTCACTGCCGCCCAACACAATCATCAAAGCAACCACAAGACCTT
CACAAACAGCGCCTTTGACACCAAACTCAGCGGCTGTCACGCCAAGCAC
GCCGAGCAGTAGCAGGAATTCCACTCAGTCCACACCAACTGTGGTACCT
GATGCGAGAGTTTCCTCCGCCGTGCGCCAAGCTGTGTTCATCAAGAGGG
AGCTACCCCAGCCGCAGAGGAGCATGCGAAATATGACACTTGGTTTGGT
GGAACAGGCGCCACTGCTTCATTTGGGTGTTGCGCCACAGCACCTGTCA
CTGCTGAAACGCCATATCTGCCGCAATGCTAATGTCACCCACTTGGACTG
TGCTTGACTCTAAGGAAACTCAAACAAAACGAGCACTTCGCCCTGTTG
GCCGAGCACTTGAGCTGAGCGAATCAGATGTCGAGGACACATTTAAGC
GCACCCTTATCAAGCTGGCCCGTTACCTCCGTCCACTGATTCGTTGGCCA
GATGCACGGCATCACAACGAGCGCTTCAAACATACCCCACTGAACTACC
GAGCCAACCTGTTGCATGTACGCTCGTTGATCGAGTGTGTGGAAACGGA
CGTGCCGATAGATCTGGGATTGGGCAGCGGCAGCTATAAGTTCATATTG
TGCATCAATACAAATGGCATCATCAGCTATGTGTCTAGCGCCTTTCCTGG
TAGTTGCGATGATCTTCAATTGTTTGAGGCCAGCAGATTTCGGGATGTCA
TTCCCAATTACCTAACACTATGCGCGGAACCAGGCKAAGCAGTACGCCG
TGCTCGCAGGTCGGGCTTCGGAGATCCTCACGACTCAGCGGATGAGGAT
GAGGCGGCGGCGGAACCAAAGCGATCACTTACCAAATTCGAGGCACAG
CGTTTGAGTGGCCAGCTAGCAAGCCAGCAATCCCTATCCGTTGTAGACG
GAGCACTGACTTCCAAGCGGGCTCCAGCGATTCAACTACCCACATTCAA
CGCACAAGAACCCGCCTGTAGAGCCCAAATGAGAGATATGATAGATTAT
TTAAGGGAATTCCGCATGCTGGATAATTCGGCTATTAAGCAAAAGTCAT
TGCTGGGTTATCTTGATGAAATGATCGTGGTGGCTGCGGGTCTATGCAAC
CTTAAGCGCCAAGAGTFfGGAATCTTAA
>CG6568|FBgn0034210
MKRAATTKMTGATAAGATTTTSSTGAVGYPVLKTPKYVVQTSPSGSSGHQ
LQMLARKDTQSLGVAINSLPPNTIIKATTRPSQTAPLTPNSAAVTPSTPS
SSRNSTQSTPTVVPDARVSSAVRQAVFIKRELPQPQRSMRNMTLGLVEQA
PLLHLGVAPQHLSLLKRHICRNANVTHLDCCLTLRKLKQNEHFALLAEHF
BLSESDVEDTFKRTLIKLARYLRPLIRWPDARHHNERFKHTPLNYRANLL
HVRSLIECVETDVPIDLGLGSGSYKEILCINTNGIISYVSSAFPGSCDDL
QLFEASRFRDVIPNYLTLCAEPGKAVRRARRSGFGDPHDSADEDEAAAEP
KRSLTKFEAQRLSGQLASQQSLSVVDGALTSKRAPAIQLPTFNAQEPACR
AQMRDMIDYLREFRMLDNSAIKQKSLLGYLDEMIVVAAGLCNLKRQELES
Scim28
AE003791 (insertion @81960), nearest ORF (CG13438) @86768 (5 kb away)
>>CG13438|FBgn0034545|cDNA sequence
ATGAAGTCGTTCGGGAACTTGACCTTTGGCCTACTCGTCATCCTTATAGC
AAGCTTTACTGTCGGCCTAGAGGCTCGTCGCCTGGCTTTGCGTCCATTGA
CAGGAAGGGAACTGAGAAGAGCTCTTAGGGAATCCGGATTCGATGAGGAT
TCTGCAGCTGGAAGATCAGTGGCGTCGGCGCTGTCCGGACTCAGTGGATT
CGCCCTGGGCATCACAAAGGGCATTGGTGGCTCACTGCTGTTCGATGTGG
TCACCTCGAATGTGACCATTGATTACATTACCAGTCTGCTGAACTCCACT
GCCTCATCGTCGACTTCAAGCAGCAGTGGAACTGCACAGGAGATCTGTTT
CAACAGTCGCAGTGCCGACGGTGAGGTGATTAACGGCAGGAGTAATGGCT
TCAATGACATGGATGATGGAGCAGATCTCGACGGCGAGTGGAGACAGACT
ACCAGTGGCACGGGCACGGGCTCTGTTACTGGCACTGGCACTGAGACAGG
AACTACCACCTCCTCGTCTTCCAACGGCCTCACCTGCATTGTCCTGAGCA
AGGAGGGTTCCCGTCGCAGGCGCCAGTTGCGAATCCAACCAGGAACGTTG
AGATCTGTTTATCCTAAAAGCCATCGGCAGACCCTGAAAAAGTACCGCCG
GCATAGGGTTTAG
>CG13438|FBgn0034545
MKSFGNLTFGLLVILIASFTVGLEARRLALRPLTGRELRRALRESGFDED
SAAGRSVASALSGLSGFALGITKGIGGSLLFDVVTSNVTIDYITSLLNST
ASSSTSSSSGTAQEICFNSRSADGEVINGRSNGFNDMDDGADLDGEWRQT
TSGTGTGSVTGTGTETGTTTSSSSNGLTCIVLSKEGSRRRRQLRIQPGTL
RSVYPKSHRQTLKKYRRHRV
Scim29
AE003458 (insertion @65550), CG2852: 64150 to 65533, CG13513 @65905
Cit#2921, Cit#5587—cyclophilin—this is very “busy”region with many loci.
>>CG2852|FBgn0034753|cDNA sequence
TGGCGACGTCGCTTGAGGAATAAACTGAAGCGCTGTGAATATTTAGAACG
ATGAAGCTGTTCTTATCCGTTTTCGTGGTAGCCCTGGTGGCCGGCGTCGT
TGTTGCCGACGATAGCAAGGGTCCCAAAGTGACCGAGAAGGTTTTCTTTG
ACATCACCATTGGCGGCGAGCCCGCTGGTCGCATCGAGATCGGTCTGTTC
GGCAAGACGGTGCCCAAGACGGTGGAGAACTTCAAGGAGCTGGCGCTGAA
GCCGCAGGGCGAGGGCTACAAGGGCAGCAAGTTCCACCGCATCATCAAGG
ACTTCATGATCCAGGGCGGTGACTTCACCAAGGGCGACGGCACCGGCGGT
CGCTCCATCTACGGCGAGCGCTTCGAGGATGAGAACTTCAAGCTGAAGCA
CTATGGCGCCGGCTGGCTGAGCATGGCCAACGCTGGCAAGGACACCAACG
GATCGCAGTTCTTCATCACCACCAAGCAGACCAGCTGGCTGGATGGACGC
CACGTCGTCTTCGGCAAGATCCTGTCGGGCATGAATGTGGTGCGCCAGAT
CGAGAACTCGGCCACTGATGCCCGCGACCGTCCCGTCAAGGATGTGGTCA
TCGCCAACAGCGGCACCCTGCCCGTTTCGGAGGCCTTCTCCGTGGCCAAG
GCCGATGCCACCGACTAAAGTGTTTGGGGAGCATGTCATCCATCAGCAAC
ATAACCGATTTGAACTAAGCATAAACGCATAATCGATTTTTCCAGACATT
TGCATTTACCATAGCTCGCCATGTTTATTACATTTCGTTCCGTAAGCAA
GTAATTGTGCTCAACTAAAAACAGAAATGGCATAAATAAAGAATGATTTT
TTGTGTGATAAA
>CG2852|FBgn0034753
MKLFLSVFVVALVAGVVVADDSKGPKVTEKVFFDITIGGEPAGRIEIGLF
GKTVPKTVENFKELALKPQGEGYKGSKFHRIIKDFMIQGGDFTKGDGTGG
RSIYGERFEDENFKIKHYGAGWLSMANAGKDTNGSQFFITTKQTSWLDGR
HVVFGKILSGMNVVRQIENSATDARDRPVKDVVIANSGTLPVSEAFSVAK
ADATD
>>CG13513|FBgn0034754|cDNA sequence
ATGACGACAACGCTGCCGGAGAAGGAAGCGGAAACGCAGCAGGAGATCAG
GGAGCGGGAGGCCAAGGCTCTGGAGGACCGAAAGGAGCGCAAGATCTACG
AGAACTTTGCCACGCCCCTGGCAGGCACTTTTCTCAACCTGCCACGCGAG
CCCGTGGAGATCGAGTGCCCCGCCTGCGGAATCAAGGATCTGAGTGTGGT
GCAAAATGATCTGAAGTGGTGGGCCAGTGAACTAAACCGCATTCCTCAGT
CTGCGTTTGTTTTTAAAGCAGTCAACATCTTTCGGTGCGAAATGGCTCAG
GATCCGAAACCACTTTACTTTGCCGTGGGCCCCGGCCCCAACGACATTAC
GTGTCCTTATTGCAGGACCAAGGCCAAGACCCGTGTGGTGCGTTCCTGGC
TGCGTTGCTGCACCAAGAGGCATCACTGCGGTGCCTGCGGGGAGTACCTG
GGCTCACCGATCGTTCTCGCTGGAACTCGCATCATGACCGTGGACGAACC
GCAGATTGTGGCCATCATTGTCAGCCACAAGCCACAAGTGGGATACCTGA
AGGAGGAGCCCACCTGGATCCGTTGTCCTTCGTGTGAGAAGTCTGGAACC
AGTTTGGTGCAACTGGAGTTGGTCACTTGCCTGCAGAGATTTCTGGGATT
CACAAAACTTTGTAAAAAATGGTCTGGCCGCCAGGACATCAATCACTATT
GTTCACACTGCGGTTGCTTCATTGGAAGATTTGTGCCCATCAGCTGCATG
GAACGATGCATTTCGAGATCAGCCCGTAAACAGGCGGCCGTGGATGATAT
GACCCTGAAGACACGACCCAAGGATTGCGCTGAAAGGGCCCAGAAATCCA
GGGAGAAAGTTCTGGCCAGCAGGGAGAAGAAGAGAGCAGAGAAGGCAGCC
AAGGATATGGACAAATCTCAGACGCAAATAGCAGTACACCAATAA
>CG13513|FBgn0034754
MTTTLPEKEAETQQEIREREAKALEDRKERKIYENFATPLAGTFLNLPRE
PVEIECPACGIKDLSVVQNDLKWWASELNRIPQSAFVFKAVNIFRCEMAQ
DPKPLYFAVGPGPNDITCPYCRTKAKTRVVRSWLRCCTKRHHCGACGEYL
GSPIVLAGTRIMTVDEPQIVAIIVSHKPQVGYLKEEPTWIRCPSCEKSGT
SLVQLELVTCLQRFLGFTKLCKKWSGRQDINHYCSHCGCFIGRFVPISCM
ERCISRSARKQAAVDDMTLKTRPKDCAERAQKSREKVLASREKKRAEKAA
RDMDKSQTQIAVHQ
Scim30
AE003676 (insertion @173210), CG17816 150150 to 173151, CG10092 @173697
C#3179: LP03266 98% identity with CG17816 gene product
>>CG17816|FBgn0037525|cDNA sequence
CCAGAAAAGAGCCATAGCATATTCTCACAGCTACATATACATATGAGCAG
GCAGCAGCAGCAGCAGTAGCAGCGGCAGAGAGAAAATCGGTTCAATCTTG
AAAAGTGTGTTTCCCAGTGCTTCACCTGAAGTTTTTTGGCACTACCTTGC
CTTACCAGAGTAAGCGGAAGTCAATTTGGCCTATGACAATACAAACGCAC
TTTCTTCGCTACGCATTGTCCAGGTGCGTGTGCGTATAGAGAGAGCGAGC
GAGAGGTGAAATATTTAGGTTTAAAGGCCAGGCGCGTGTGTGTGACCCAT
GAAAAGTTGTTAAACATAAGCAACGTCAATCGCCGCTGATCGAAAGAAGA
GAAACCCCTACGCGCGCGTGTTTAATTTGTATTTTTGGCACTTTGGTTGG
CAAACAGCAAAAGCATTTCCCTATGATTGGCTCATTCTGGAATCTGTGCA
GAGCGTGCCCATCAATGCCGGTAGACAAGCTCCACTCGGAGTATCGGAGC
ACTTATCGCTGGCATGAATTTACGGGCAACTCGCGGCCAGAGGTTGTGCG
ACGGGCGCCTGCCCCAAACCCAAGTCAATTTGTTGGAGCGACAAATGAGC
CGCCATTGCCACGCCGGAAAAAATGTCCAGAATTAGCATATAAATCGCAC
GAGTTTATTATAGGATCGGAGTATACAGATGGACGCCGAGATGCCAGTGC
ACATCGTTTGGCGAGATCGGAGGAGCGCGGTGGCACACCTTCGCGCCGCA
GCAAATCGGAGGGACCACCCGTTGTGCCCAATGGACGTGCGTATCCCATT
GCCACGGAGATCGACGGAACCACAAGAAAACAGGCGGGTGAGTCAAATGG
GCTATTGAAAAAGACCATCAATAAGTTGAGCACTGAGTACCGCCTGCAAT
TCGTTTGGCCCACCGTCCGACGCATAAAGGGCGGCGGCGAGGCGACGTCT
AGGGCCGCTGCCGGCGACTATCCGAGAAAGTCCATATCGCTGGGCGCCCT
TCGGTCCGGCGGCCAAGGTCACTGTCACAGTCACACCCAAAACCAGAATC
AGAGCCAGAGTCAGGGTCTGACCCAAGCCCAGAATGGTCACACACATCAC
ACGATGATGGGTGGCGGTGCGGGCTTGCCGACGGTGCATAAAAAACGAAC
AACAAATCAGAAAGAAGTGTTGCATGCTGCAGCCATCGAGAAGCACAGCA
GTTCCCACTTGAAGCTCAGAATCTCTCAGGAGCCAGCACTACTTCCCATT
GCTAATGACTCTCCCGATTCTTGCAGACAAGTGACCATTATGGAGCGCAA
GACCACCTCGCGTCCCTTCTCGCAGGCCATCGACCAGGAGCGCCTAAACC
ACTTCATCACGAAAAAGGAGAACTTTGGCTTCGCCGACGCCGCCGTGGCC
GCCGCGGCCCTCAAGGACGAGGTGGACAACCGGCAGGCCGGCGAATCCGG
CCAGGTGGTGGTCATGAACGGCTCTGCCCCGCCGCACTCGAAACCGAATT
TGGATTTGTGGTTCAAGGAGATGGTGGAGCTGCGCAAAAAAGCCGGCGAA
TACAAGTGTCGCGGTTGGGGCATAGAAATTGATCCGGAATTGTATAAGAA
ACAGAAGGATCTTTGGGATCAGGTTTCAAAGCGCAGCTCACTTTCGGCAC
TTTCCCTAGCCTCTTCAGTTCATAGACCTATTACAAAGGAGGAGAAGGAA
CAGGAGAACAATAAGAAGTCCACGCCATTGCAGAAGGCCCAGAAGCCGCG
TGTTCCTGGCCAAGCCTTTTTGATTGATAATAAGGATGAGATTTCAGCAC
TGCCAGCACGATTTAGCAATATACGCCATCACCTTGAACGCACCACAGGT
CCGGATGTGGAAGAGGGAGCTTTGTTGCCCTCGCCAACGCGCGAGAAGCT
GATGCCGGCTATTACCAAGCGGGAATCGGAATCTCAGCGAGGAAGTCCCA
AGAAGACCGCCTTGTCCAGGCACGGATCGCCTCAGAAGGGCAGTCCTCAA
AAGGGCAGCCCCAAGAAGGTCCTTAAAAGTGAGTAGTTCCCCGCTTTTTC
CCTCACCTTTGAGCAGAGGATACAAGGAAAGAATGCATACGCATATCCGA
TTTTAATTCCAAAGTTAACCATATCCGAATAGCAAATTTACTCTTTTGCA
ACAATGACACAAGTACACAAAATGCACTTACCAATAGAGTTACGAGTTTG
GGAACAGAACAAAACATTGTACACGCTCCAACAATAAGTATACGCCCCGT
TACCAATACCTTGATTTGGTTTCCTATGATTTTCGTTTTGCCATAGTTTG
CTCAACTGCTTCAACCGTTTGATTTGCATTTTCCCCGACAGTCGAAGGAG
TCGACTCGTTTTTCATCATTGTCAAGTGCACCGAAGACTTCTTGGGATAT
GAGATTTGTGCACAATCCTAATGTAGTTTCTATTTACTTACATTTGCCAG
TTTTTATCGAGGGTTTGTGTTCTGAATTCGGTTGAAAGTTGATTTTCATG
TTCTACGTTTAAGCTATGATTTGTAGAGAACCTTTTGAGAACATATGAGT
CAATCCCTTAAAACCACAACTACTTACATTTATATATTGAG
>CG17816|FBgn0037525
MIGSFWNLCRACPSMPVDKLHSEYRSTYRWHEFTGNSRPEVVRRAPAPNP
SQFVGATNEPPLPRRKKCPELAYKSHEFIIGSEYTDGRRDASAHRLARSE
ERGGTPSRRSKSEGPPVVPNGRAYPIATEIDGTTRKQAGESNGLLKKTIN
KLSTEYRLQFVWPTVRRIKGGGEATSRAAAGDYPRKSISLGALRSGGQGH
CHSHTQNQNQSQSQGLTQAQNGHTHHTMMGGGAGLPTVHKKRTTNQKEVL
HAAAIEKHSSSHLKLRISQEPALLPIANDSPDSCRQVTIMERKTTSRPFS
QAIDQERLNHFITKXENFGFADAAVAAAALRDEVDNRQAGESGQVVVMNG
SAPPHSKPNLDLWFKEMVELRKKAGEYKCRGWGIEIDPELYKKQKDLWDQ
VSKRSSLSALSLASSVHRPITKEEKEQENNKKSTPLQKAQRPRVPGQAFL
IDNKDEISALPARFSNIRHHLBRTTGPDVEEGALLPSPTREKIMPAITKR
ESESQRGSPKKTALSRHGSPQKGSPQKGSPKKVLKSE
>>CG10092|FBgn0037526|cDNA sequence
ATGATTCGCTTACGGCAAGCAATTTGTGAGCAGCTGCCGCATCTCAAGAA
TGCCTGCTATGCCCTGGAGGTGCCTGTCAAGAAACAACAGCTACAGAATA
GCCGACGTCCAACTGTGGAGTGGATTCTGCCCTCTGCGTTTGCGCAACAG
GAGGTGGAACTACTGGACTCATTGAAGAAGCGCAGATTCGAGGCCTATGT
GGAAAACGTTCGGATTGTACCTAGCGCTGGGCGCAGTGCAGCCAAAATCG
AGTTTCAGCTGCAGCCACAGGTCTTTGTAGAACAGCTCCTCCAAACTAAA
GAGATTGCTTTACATCCTTCGCCCTTTGCTGCGGAACACATAGTGGTTGA
GTACAGCTCGCCCAACATAGCCAAACCCTTCCACGTGGGCCACCTGCGCT
CCACAATCATCGGCAATGTTCTGGCCAACCTGCATGAGCATTTGGGCTAC
CGCACAACACGGTTGAACTATCTGGGGGATTGGGGCACGCAATTTGGACT
ACTGGTATTGGGAGTTCAACTGCTGAATGTAAGCGACAAAGAGATGCAAC
TATCCCCAATAGAAACGCTGTACAAATCCTACGTGGCCGCCAACAAGGCT
GCTGAACAAAGACCTGAAATCGCGCAACAGGCGAGGGACCTCTTCGCCGC
CTTGGAAGGAGGAACGGATAAATCAATGGCCAAGAAATGGCAGCAATACA
GAAACTACACTATAGAGGATCTATCCAAAGTCTATAACAGATTGGGCGTT
CACTTCGATAGCTACGAATGGGAATCCCAGTACTCCCAGCAGCAAATTCA
GGATGTTCTGGACAAACTGCGAAGCGCTGGACTCCTCCAGCCGGAGCACG
ATGGTCGTGAGATTGTCGTGGTGGACGGCCGACGCATTCCTGTGATCAAG
AGTAATGGATCCACTTTGTACCTGGCCAGGGACATAGCTGCCCTGCTGGA
GAGACTCTCCAGGTTCCAGTTCTCACGCTTGCTCTACGTCGTGGACAATG
GTCAAGCGGATCATTTTAATGCCCTTTTTAAAACAACGGCAGCCCTGGAT
GACCGCCTAAGTCTGGAACAGCTGCAACATGTGAAATTTGGACGCATTTA
TGGGATGAGCACTCGTCAGGGAAAGGCAATCTTTCTAAAAGATGTCCTAG
ATGAAGCACGAGACATAATGCGGGAAAAGCGAAACATAAGTGCCACTACC
AGAGAAAATTACAATCTGGATGATGAACATGTATGTGATATTTTGGGCGT
GTCAGCCGTCCTGGTCAATGTCCTTAAGCAGCGAAGGCAACGAGATCACG
AGTTCAGCTGGCAGCAGGCACTCCAAGTAAATGGTGACACAGGAATCAAG
CTTCAATACACACACTGCCGCCTGCACAGTTTGCTGGATAATTTCCGAGA
TGTAGATCTGGACGACATTAAGCCCGACTGGAAGCATTTCTCTACGGAGC
CTGCGGATGCTTTGGATCTGCTCTACGCACTGGCACGTTTCGATCAAAGC
GTTTGGCAATCGAAGGAACAACTGGAGGCTTGTGTCCTTGTCAACTATCT
CTTTGGATTGTGCAATGCCACCAGTCAAGCGCTGAAAAGATTGCCTGTGA
AACAAGAGTCCAGCCTAGAGAAGCAACTCCAACGCCTGCTTCTTTTTCAC
GCTGCCAAAAAAACACTGCGACACGGAATGGAGCTCCTTGGCCTGCGTCC
ACTGAACCAAATGTAG
>CG10092|FBgn0037526
MIRLRQAICEQLPHLKNACYALEVPVKKQQLQNSRRPTVEWILPSAFAQQ
EVELLDSLKKRRFEAYVENVRIVPSAGRSAAKIEFQLQPQVFVEQLLQTK
EIALHPSPFAAEHIVVEYSSPNIAKPFHVGHLRSTIIGNVLANLHEHLGY
RTTRLNYLGDWGTQFGLLVLGVQLLNVSDKEMQLSPIETLYKSYVAANKA
AEQRPEIAQQARDLFAALEGGTDKSMAKKWQQYRNYTIEDLSKVYNRLGV
HFDSYEWESQYSQQQIQDVLDKLRSAGLLQPEHDGREIVVVDGRRIPVIK
SNGSTLYLARDIAALLERLSRFQFSRLLYVVDNGQADHFNALFKTTAALD
DRLSLEQLQHVKFGRIYGMSTRQGKAIFLKDVLDEARDIMREKRNISATT
RENYNLDDEHVCDILGVSAVLVNVLKQRRQRDHEFSWQQALQVNGDTGIK
LQYTHCRLHSLLDNFRDVDLDDIKPDWKIIFSTEPADALDLLYALARFDQS
VWQSKEQLEACVLVNYLFGLCNATSQALKRLPVKQESSLEKQLQRLLLFH
AAKKTLRHGMELLGLRPLNQM
Scim31
AE003686 (insertion @193550), nearest ORF (CG4029: Dom) @187249 to 198668
>>Dom|FBgn0015660|cDNA sequence
ACCGGGCGGGTTTATTTTATCATTGCTCCGCGACTTCGAATACGAGACCG
GTGTTGTGCGCTCCTGATAACTGCGATATATTGAGCGCGAGCGCCATGTC
CTTTTGCTGGAAGTAGAATTTGAAAAGTGCAGAGATCACGGCTTGGATTG
CCAAGGAACAACGGTGTTGAGACTGAATATATTTTTTGTGCGCTGTTTCG
AAATAGAACCGTTAATTGGAATTGGCAGTAAGAAGCAGAGAGGCGGACGA
TATTCCGGTGAAATCTTCGCCAGGCGGAAACATCGATCAAAACAAAGTGC
ATGCTAAAAACATAAAAGATTCAACATGTTCGAACTAGAGGATTATTCGA
GCGGCATACATGAGGGATTCTTCAGCAAATATGCGGATGCGGCTGGACCC
TCGCTAGACTTTTATGTATCCGACTCGATGCAGGAGATGCTGAACGTGGA
CATCCGCGCAGAGATCGCCAATGTGGTGGGCAGTTCCAGCAGCGACTTGA
CCTCGTCCCTGGACCAAACACTGGAAGCTATATCCGCGATAAACAACAAC
CAGAGCAATGGAAACAGCAGCCAGTCAGCTTCTTACAATGCGAATGCGAA
TTTTCTGACCAGCAGCGGACTCCACGCCTCACCCACAGCGAAATGGATGG
GCTCGTCGGCCAATTTTTGGTCCAACAGCGATTACTATGCGGATCTGGGG
GCATGTGTGAACCCCATTTCCGTAATGCCACTGATAAATTCGACTTCTGC
AGGAATGTTCTCGCCAAAAAAAAACAAGACAGCCTCAAGTACGCAGGGAA
GATCGGGAGCGGTGCCCTCGTCGCCCAGCGCCGAAAGGGATCAGCACAAA
TCGCACCTGACATTTTCGCCGGCTCAGATGAAGGTCAGTGCAGGATCCAT
GCGGCGGGACCAGGTGATGGCACACATTCCCAAGCAAATATCCGTGGTCA
CGGGCACCGGAACCACAGCGCCCGCCACAATGGCCACCAATTCGGTGCTT
CAACGGCGTAATTCCTCGGCCGTGGATGCTGTACGTAAGGATTTGGTCAC
AGAGCTGCGTAAAGCACAATCCAGTCCAGTGCCCAATTCCTTGGAGGAGC
TGGGCAAGGGAAAAGGATCAACACTGCTAAATGCCAGTGTTGGGGCGACC
AACACCATTAAACTGGCGCCCGGTATCGGTGGGTTAACCTTTGCCAACAG
TGCAGCCTACCAGAAGCTGAAGCAAACATCCTTGGTTAAGTCACCAGGCG
GTATTTCGCCAGGAGCAGGATCCAATATGGGTCTCAAGCGGGAGGACTCA
AACAAGCGAGGACTGCAGGCCAGCACCACGCCAAAGAGCATTGCTTCGGC
GGCAAACTCGCCGCATCATCAAATGCAGAGCAACTACAGCCTGGGATCAC
CTTCATCACTGTCCTCCTCATCTGCATCCTCTCCCCTAGGGAATGTGAGC
AACCTGGTCAACATAGCGAATAACAATACAAGCGGAGCTGGATCCGGCTT
GGTGAAGCCTCTGCAGCAAAAGGTTAAACTGCCACCCGTGGGCAGTCCAT
TTCCCAAACCAGCATACTCGTACTCCTGCCTCATCGCTTTGGCCCTCAAG
AATTCGCGAGCAGGATCCCTTCCGGTCTCGGAAATATATAGTTTCCTATG
CCAGCATTTCCCTTACTTCGAGAATGCCCCCAGCGGCTGGAAGAACAGTG
TGCGTCACAACCTGTCTTTAAACAAATGCTTTGAGAAGATCGAAAGACCA
GCGACGAATGGCAACCAGAGAAAGGGCTGCCGTTGGGCCATGAATCCCGA
TCGTATCAACAAGATGGACGAGGAGGTGCAAAAGTGGTCGCGCAAGGATC
CGGCTGCCATACGTGGAGCCATGGTATATCCTCAGCATCTGGAGTCCCTG
GAAAGGGGAGAGATGAAGCACGGATCGGCAGACTCGGATGTAGAGCTGGA
CTCGCAATCGGAAATTGAGGAGTCTTCGGATCTGGAGGAACACGAATTCG
AGGACACTATGGTGGATGCAATGCTGGTAGAAGAGGAAGACGAGGAGGAG
GACGGGGATGATGATGAGCAAATAATCAACGATTTTGATGCGGAAGATGA
GCGTCATGCCAACGGAAACCAGGCAAACAACCTACCCATCAACCATCCAC
TACTTGGTCAGAAAAGTAACGACTTCGATATAGAGGTCGGGGATCTATAC
GACGCAATCGACATAGAGGATGATAAGGAGTCAGTGCGTCGAATTATCTC
GAATGACCAGCACATCATTGAGTTGAACCCTGCCGATCTGAATGCCACCG
ATGGCTACAACCAGCAGCCGGCATTGAAACGGGCTCGCGTCGACATTAAC
TATGCAATTGGTCCTGCTGGCGAGTTGGAACAGCAATACGGCCAGAAAGT
GAAGGTGCAGCAAGTCATACAGCCGCAGCAGCATCCGCCCACCTACAACA
GGCGCAAGATGCCGCTGGTCAACCGCGTCATCTAGAGCGGGGCACAGCCC
AAAAACCCATTACATTAATCAATTAGTTTTAGACCTTTGGCATTTAAGAA
ACCCATGCTACGCTTAAACGTAATCCTAGAAGCCCCATCATTCAATATCG
AATATCAGTTTTCAGTTTCGTGTGCAAAACCCAAATTGTTATTAAATCTC
CCTTCCTATTTGTAGTTCAGTTTGGCTGCTGTTTGATTTTAAATCTCGAT
TAGAGCCTGTGCCAACTGAAAAAGAGAGATAACTTGTGCCCTTTTGTTTT
TGCTTAATTTAAATCTTTCTATAGTCGCTTCTCGAATAAATCTGTATTAT
ATTGTTCGAAAAGAACTGAGATATTGCTCCACTTGACGATATTTCCGTTT
TAATTCGCCTGACGCTTGAGGAGAAAAACTCAATAGGTTCCACTGACGAC
GCATGAAGCATGTTAATACTTTTTACCAGACTCGAGCTGGTTTGAGTTGC
AACTTTTGATTTGATCTCCCTACTGACAAATAAATTTTCATCCTTCAATC
GATAAGAAACTTGACAATGCATTTATGACAAACGATTCCACGCTTAGTCG
TAGAATAATATTAATGTGCAACACAACGATTACTTTGACAACGAAATGTG
AACAGTAGGTTTATATTTCGAACTTTTGTTGATTATTTCACCACATGGTG
ACAATTTGCATTTGTTTCGAACATTTTCAGCTAACATTTAAGAAAATTGA
AAGAGAATTGATCACACATACTTGCCGGTCCAGTATTCGTAAGCGAGGTA
TATTGAGGATTTTTACAGAACTTTTATACGAATTGTACTATATATACATG
GAAAACCAACAATTAATGTCGGAAAGTTCAGTCAATTAATAATATGGATA
TTTTATAAGGCGGTTCCGTATGTAAATAGTTTACACGCAGAGAATAAATTT
GTATACTATGGCATAGATGTAAGTAATATGTATGTAAAATAATTTGTAAA
CGAAATCCGAATTCCAAATAATACAAGATACGAAAACCAATAAGACTTAA
AAGAAGCGTACCAGACTAATGAACATGATCAGGCCTCAGAAGTAAATAGT
ACAAAGAGCTAGACTTTTGGGTCCAAGCAGTTACAAAGCCAACTCAAGGA
TGGCTGATGGAATTAAACCGTTTTTGTTATTACCCTTTTCTTTTGTGATG
CTTCGACTTAGCTTGCGCATTTAACAATTCCATTTACGAACCAGGAACAT
TTATGCATTTTTTGTTGTAATATTAGCACCTAAATATTGTATTTAATCAT
TAAGTGAAGCTCTGTAAATCTTTAAGCTAAGAAAAACAATTTTTGTATAG
AGTTGTTAGAAAATCAATTGACAAAAACAAATTGAAACCAAAAAAAAAA
>Dom|FBgn0015660
MFELEDYSSGIHEGFFSKYADAAGPSLDFYVSDSMQEMLNVDIRAEIANV
VGSSSSDLTSSLDQTLEAISAINNNQSNGNSSQSASYNANANFLTSSGLH
ASPTAKWMGSSANFWSNSDYYADLGACVNPISVMPLINSTSAGMFSPKKN
KTASSTQGRSGAVPSSPSAERDQHKSHLTFSPAQMKVSAGSMRRDQVMAH
IPKQISVVTGTGTTAPATMATNSVLQRRNSSAVDAVRKDLVTELRKAQSS
PVPNSLEELGKGKGSTLLNASVGATNTIKLAPGIGGLTFANSAAYQKLKQ
TSLVKSPGGISPGAGSNMGLKREDSNRRGLQASTTPKSIASAANSPHHQM
QSNYSLGSPSSLSSSSASSPLGNVSNLVNIANNNTSGAGSGLVKPLQQKV
KLPPVGSPFPKPAYSYSCLIALALKNSRAGSLPVSEIYSFLCQHFPYFEN
APSGWKNSVRHNLSLNKCFEKIERPATNGNQRKGCRWAMNPDRINKMDEE
VQKWSRKDPAAIRGAMVYPQHLESLERGEMKHGSADSDVELDSQSEIEES
SDLEEHEFEDTMVDAMLVEEEDEEEDGDDDEQIINDFDAEDERHANGNQA
NNLPINHPLLGQKSNDFDIEVGDLYDAIDIEDDKESVRRIISNDQHIIEL
NPADLNATDGYNQQPALKRARVDINYAIGPAGELEQQYGQKVKVQQVIQP
QQHPPTYNRRKMPLVNRVI
Scim32¹
Scim32²
AE003697 (B198 insertion @25820), CG10120 19300 to 28025, (E587 insertion
somewhere between 29264 and 29813—CG10120 is still the nearest locus)
22 >CG10120|FBgn0038081|cDNA sequence
AGCCGCGATTTCAGCGCGAGTTCAGTTTTTGATTCAGTTTCAGGCGGTTC
GGAGTTGCTAAGTCAAGCGCAATAGCTCAAAATACACTTTTTTTAATTTT
TGTTAATAACTGTTTTTAATAATTCCGGTGAAACATCGCGTGGTCAAGCG
AACTGAGTTAATTTTCGCGTTAGAAAAGTTCACAAGTTTTGCGTTTACCA
AATAATTAACTATAACTATTTAACTGGAGCTAATTTAACTGAAATTTAGA
ACCCAAAATGGGTAATTCCAGTTCCATTTGCGCCGATCGCAATGTCATAA
CAAATTTCGATGAAAATGGCACGCCGGTTTATCCCACCGCCAACAATTCG
CAGAGTCCTTCCTCATATAGTCGCGGCAAAGAGCGCGAGCTCGGCTGTTA
CACGAAGAGAAACAGCAACAGCAACAACAACAATAGCCATGAGAGAGAGA
GTCAGAGCTGTTGTAGTAGTCGTGTGTGTAAAAATCATACGACCACAACG
ACAACCACACTCGAATACGAACTTTCCAATTTCGCAAAACTAACGACACG
AATCACAACGCAAAGTGCCGCCGAAGTGGACACATCGCCGCATACGGATA
CGGAAACGCATAGGGACAGAGATTCGAATCCGGGTAATATAGCCTTAGCC
ACCGATTTGGAACTGCCCAAGGGTCTGCCGTTATCGTTATCCTCGCGACA
CCACTGGAATCAGCTGCAGAGCAGTTTGCACGCCCTTCACCACCAGCAAC
AGCAACAACAACAGCAACTACGTTCATACAGCTCCACTAGCGAAACAAAT
TTGGAAGACAAGATGAGCAAACCCGATTCGAAACTAGATAAATACGCGCA
GCGCGATCGCCTGGGCCTTTGGGGCACTGGTGACAATGAGGTGGTCGGCA
GCCTCTCCGGATTCACCCGACTCTTGGACAAGCGCTACTCAAAGGGCCTG
GCCTTCACACACGAGGAGCGCCAGCAGTTGGGCATCCATGGCATGCTGCC
CTATGTGGTCCGTGAGCCCAGTGAGCAGGTGGAGCACTGCCGCGCTCTGC
TGGCGCGACTGGATCAGGATCTGGACAAGTACATGTACCTGATCAGCCTA
TCGGAGCGGAACGAGCGTCTGTTCTACAACGTGCTCAGCTCAGACATCGC
CTACATGATGCCACTGGTGTACACGCCCACCGTGGGATTGGCCTGCCAGC
GCTACAGTTTGATCCACCAGAACGCCAAGGGCATGTTCATATCCATCAAG
GACAAGGGACACATCTACGACGTGCTAAAGAACTGGCCGGAAACGGATGT
GCGTGCCATCGTTGTCACGGACGGCGAGCGCATCCTGGGACTGGGAGATC
TGGGCGCCAACGGAATGGGTATACCCGTGGGCAAACTGTCCCTGTATACG
GCCTTGGCGGGCATTAAGCCATCGCAGTGCCTGCCCATCACCTTGGATGT
GGGCACCAATACCGAATCCATCCTGGAGGATCCCCTGTACATCGGTCTGC
GCGAACGCAGGGCCACTGGAGATCTGTACGATGAGTTCATCGATGAGTTC
ATGCATGCCTGCGTTCGTCGCTTTGGTCAAAACTGCCTAATCCAGTTCGA
GGACTTTGCCAACGCCAATGCCTTCAGGCTGTTGTCCAAATACCGCGACT
CCTTCTGCACCTTCAACGACGATATTCAAGGAACCGCGTCGGTGGCCGTG
GCTGGTCTGCTGGCCTCGCTAAAGATCAAGAAGACCCAGCTGAAGGATAA
CACGCTGTTGTTCCTGGGCGCCGGAGAAGCGGCTCTTGGTATTGCCAACC
TGTGCCTGATGGCCATGAAGGTGGAGGGTCTCACCGAGGAGGAGGCCAAG
GCCCGCATCTGGATGGTGGATAGCCGTGGTGTCATCACCCGCGATCGTCC
AAAGGGCGGACTCACCGAACACAAGCTGCACTTTGCCCAGCTGCACGAAC
CCATCGATACTTTGGCAGAGGCGGTGCGAAAGGTGCGTCCCAATGTCCTG
ATTGGAGCGGCTGCGCAGGGCGGCGCCTTCAACCAGGAGATCCTTGAGCT
GATGGCCGATATTAATGAGACGCCGATCATCTTTGCACTGTCCAATCCGA
CCAGCAAGGCGGAGTGCACCGCCGAGGAGGCGTATACGTACACCAAGGGG
CGCTGCATCTTCGCCAGCGGTTCGCCTTTTGCTCCTGTGACGTACAACAA
CAAGAAGTTCTATCCGGGTCAGGGCAACAACTCGTACATTTTCCCTGGCG
TGGCACTGGGTGTTCTGTGTGCCGGCATGCTGAACATTCCCGAGCAGGTA
TTCTTGGTCGCCGCCGAGCGCTTGGCGGAGCTGGTCTCCAAGGACGACCT
GGCCAAGGGCAGCTTGTATCCACCACTCAGCTCCATTGTCAGCTGTTCGA
TGGCCATTGCCGAAAGGATTGTGGAGTACGCCTACAAAAACGGATTGGCC
ACTGTTCGTCCAGAGCCGGTCAATAAGCTGGCGTTCATCAAGGCCCAGAT
GTACGATCTGGACTATCCCCGATCCGTGCCCGCCACCTATAAGATGTAGA
TGATGGCCAGATGATGAGGATCCATTCCGCCTAACCCCAGAAACCAAAAG
GAGTGGCCATCAAAAGATATCCGGCAAGGGCGGCGAGCAGTAACCATGTT
ATTTATTTTATAATGTCGCACATTTGTCGTCTAGCATAATATCCAAATTT
GTATCGCGGCTAATTAACCACAACCACACACTATCCACCAACAACCATAT
TATAAAAAAAAACCAACTAAAATGGAATGTCATCAGCTTGCGGCGCGCGA
TTTTGTATAATGCTAACTATGTAAATGGGATATTGATCTAATAGATATGT
AAACAAATTTTATGTAATCTTGAACAACACAAACTATTCTAAGGATATAT
ACAAATAAAGAAATAAACAAAAAT
>>CG10120|FBgn0038081|cDNA sequence
AAAGCTGCAGCAACGCAGACAAAAGTCAAATACTCAGTGAGATAATTGTC
GGCAAATCAATATACTAATAAGTATATAATATAGAAGACTTTTAAGAGCA
CCGCCATGTACTCGATCCTACGACGCTGTTCTGGTATCAGAAAAACTTTT
GGACCCACGCCGGTTTATCCCACCGCCAACAATTCGCAGAGTCCTTCCTC
ATATAGTCGCGGCAAAGAGCGCGAGCTCGGCTGTTACACGAAGAGAAACA
GCAACAGCAACAACAACAATAGCCATGAGAGAGAGAGTCAGAGCTGTTGT
AGTAGTCGTGTGTGTAAAAATCATACGACCACAACGACAACCACACTCGA
ATACGAACTTTCCAATTTCGCAAAACTAACGACACGAATCACAACGCAAA
GTGCCGCCGAAGTGGACACATCGCCGCATACGGATACGGAAACGCATAGG
GACAGAGATTCGAATCCGGGTAATATAGCCTTAGCCACCGATTTGGAACT
GCCCAAGGGTCTGCCGTTATCGTTATCCTCGCGACACCACTGGAATCAGC
TGCAGAGCAGTTTGCACGCCCTTCACCACCAGCAACAGCAACAACAACAG
CAACTACGTTCATACAGCTCCACTAGCGAAACAAATTTGGAAGACAAGAT
GAGCAAACCCGATTCGAAACTAGATAAATACGCGCAGCGCGATCGCCTGG
GCCTTTGGGGCACTGGTGACAATGAGGTGGTCGGCAGCCTCTCCGGATTC
ACCCGACTCTTGGACAAGCGCTACTCAAAGGGCCTGGCCTTCACACACGA
GGAGCGCCAGCAGTTGGGCATCCATGGCATGCTGCCCTATGTGGTCCGTG
AGCCCAGTGAGCAGGTGGAGCACTGCCGCGCTCTGCTGGCGCGACTGGAT
CAGGATCTGGACAAGTACATGTACCTGATCAGCCTATCGGAGCGGAACGA
GCGTCTGTTCTACAACGTGCTCAGCTCAGACATCGCCTACATGATGCCAC
TGGTGTACACGCCCACCGTGGGATTGGCCTGCCAGCGCTACAGTTTGATC
CACCAGAACGCCAAGGGCATGTTCATATCCATCAAGGACAAGGGACACAT
CTACGACGTGCTAAAGAACTGGCCGGAAACGGATGTGCGTGCCATCGTTG
TCACGGACGGCGAGCGCATCCTGGGACTGGGAGATCTGGGCGCCAACGGA
ATGGGTATACCCGTGGGCAAACTGTCCCTGTATACGGCCTTGGCGGGCAT
TAAGCCATCGCAGTGCCTGCCCATCACCTTGGATGTGGGCACCAATACCG
AATCCATCCTGGAGGATCCCCTGTACATCGGTCTGCGCGAACGCAGGGCC
ACTGGAGATCTGTACGATGAGTTCATCGATGAGTTCATGCATGCCTGCGT
TCGTCGCTTTGGTCAAAACTGCCTAATCCAGTTCGAGGACTTTGCCAACG
CCAATGCCTTCAGGCTGTTGTCCAAATACCGCGACTCCTTCTGCACCTTC
AACGACGATATTCAAGGAACCGCGTCGGTGGCCGTGGCTGGTCTGCTGGC
CTCGCTAAAGATCAAGAAGACCCAGCTGAAGGATAACACGCTGTTGTTCC
TGGGCGCCGGAGAAGCGGCTCTTGGTATTGCCAACCTGTGCCTGATGGCC
ATGAAGGTGGAGGGTCTCACCGAGGAGGAGGCCAAGGCCCGCATCTGGAT
GGTGGATAGCCGTGGTGTCATCACCCGCGATCGTCCAAAGGGCGGACTCA
CCGAACACAAGCTGCACTTTGCCCAGCTGCACGAACCCATCGATACTTTG
GCAGAGGCGGTGCGAAAGGTGCGTCCCAATGTCCTGATTGGAGCGGCTGC
GCAGGGCGGCGCCTTCAACCAGGAGATCCTTGAGCTGATGGCCGATATTA
ATGAGACGCCGATCATCTTTGCACTGTCCAATCCGACCAGCAAGGCGGAG
TGCACCGCCGAGGAGGCGTATACGTACACCAAGGGGCGCTGCATCTTCGC
CAGCGGTTCGCCTTTTGCTCCTGTGACGTACAACAACAAGAAGTTCTATC
CGGGTCAGGGCAACAACTCGTACATTTTCCCTGGCGTGGCACTGGGTGTT
CTGTGTGCCGGCATGCTGAACATTCCCGAGCAGGTATTCTTGGTCGCCGC
CGAGCGCTTGGCGGAGCTGGTCTCCAAGGACGACCTGGCCAAGGGCAGCT
TGTATCCACCACTCAGCTCCATTGTCAGCTGTTCGATGGCCATTGCCGAA
AGGATTGTGGAGTACGCCTACAAAAACGGATTGGCCACTGTTCGTCCAGA
GCCGGTCAATAAGCTGGCGTTCATCAAGGCCCAGATGTACGATCTGGACT
ATCCCCGATCCGTGCCCGCCACCTATAAGATGTAGATGATGGCCAGATGA
TGAGGATCCATTCCGCCTAACCCCAGAAACCAAAAGGAGTGGCCATCAAA
AGATATCCGGCAAGGGCGGCGAGCAGTAACCATGTTATTTATTTTATAAT
GTCGCACATTTGTCGTCTAGCATAATATCCAAATTTGTATCGCGGCTAAT
TAACCACAACCACACACTATCCACCAACAACCATATTATAAAAAAAAACC
AACTAAAATGGAATGTCATCAGCTTGCGGCGCGCGATTTTGTATAATGCT
AACTATGTAAATGGGATATTGATCTAATAGATATGTAAACAAATTTTATG
TAATCTTGAACAACACAAACTATTCTAAGGATATATACAAATAAAGAAAT
AAACAAAAAT
>CG10120|FBgn0038081
MGNSSSICADRNVITNFDENGTPVYPTANNSQSPSSYSRGKERELGCYTK
RNSNSNNNNSHERESQSCCSSRVCKNHTTTTTTTLEYELSNFAKLTTRIT
TQSAAEVDTSPHTDTETHRDRDSNPGNIALATDLELPKGLPLSLSSRHHW
NQLQSSLHALHHQQQQQQQQLRSYSSTSETNLEDKMSKPDSKLDKYAQRD
RLGLWGTGDNEVVGSLSGFTRLLDKRYSKGLAFTHEERQQLGIHGMLPYV
VREPSEQVEHCRALLARLDQDLDKYMYLISLSERNERLFYNVLSSDIAYM
MPLVYTPTVGLACQRYSLIHQNAKGMFISIKDKGHIYDVLKNWPETDVRA
IVVTDGERILGLGDLGANGMGIPVGKLSLYTALAGIKPSQCLPITLDVGT
NTESILEDPLYIGLRERRATGDLYDEFIDEFMHACVRRFGQNCLIQFEDF
ANANAFRLLSKYRDSFCTFNDDIQGTASVAVAGLLASLKIKKTQLKDNTL
LFLGAGEAALGIANLCLMAMKVEGLTEEEAKARIWMVDSRGVITRDRPKG
GLTEHKLHFAQLHEPIDTLAEAVRKVRPNVLIGAAAQGGAFNQEILELMA
DINETPIIFALSNPTSKAECTAEEAYTYTKGRCIFASGSPFAPVTYNNKK
FYPGQGNNSYIFPGVALGVLCAGMLNIPEQVFLVAAERLAELVSKDDLAK
GSLYPPLSSIVSCSMAIAERIVEYAYKNGLATVRPEPVNKLAFIKAQMYD
LDYPRSVPATYKM
>CG10120|FBgn0038081
MYSILRRCSGIRKTFGPTPVYPTANNSQSPSSYSRGKERELGCYTKRNSN
SNNNNSHERESQSCCSSRVCKNHTTTTTTTLEYELSNFAKLTTRITTQSA
AEVDTSPHTDTETHRDRDSNPGNIALATDLELPKGLPLSLSSRHHWNQLQ
SSLHALHHQQQQQQQQLRSYSSTSETNLEDKMSKPDSKLDKYAQRDRLGL
WGTGDNEVVGSLSGFTRLLDKRYSKGLAFTHEERQQLGIHGMLPYVVREP
SEQVEHCRALLARLDQDLDKYMYLISLSERNERLFYNVLSSDIAYMMPLV
YTPTVGLACQRYSLIHQNAKGMFISIKDKGHIYDVLKNWPETDVRAIVVT
DGERILGLGDLGANGMGIPVGKLSLYTALAGIKPSQCLPITLDVGTNTES
ILEDPLYIGLRERRATGDLYDEFIDEFMHACVRRFGQNCLIQFEDFANAN
AFRLLSKYRDSFCTFNDDIQGTASVAVAGLLASLKIKKTQLKDNTLLFLG
AGEAALGIANLCLMAMKVEGLTEEEAKARIWMVDSRGVITRDRPKGGLTE
HKLHFAQLHEPIDTLAEAVRKVRPNVLIGAAAQGGAFNQEILELMADINE
TPIIFALSNPTSKAECTAEEAYTYTKGRCIFASGSPFAPVTYNNKKFYPG
QGNNSYIFPGVALGVLCAGMLNIPEQVFLVAAERLAELVSKDDLAKGSLY
PPLSSIVSCSMAIAERIVEYAYKNGLATVRPEPVNKLAFIKAQMYDLDYP
RSVPATYKM
Scim33
AE003722 (insertion @11670), nearest ORF (CG7682) @11602
CG18617 gene product
>>CG7682|FBgn0038614|cDNA sequence
CCGTTCGCTCTCTTCGCCTTCTCTTTTCTCTCCCGCTTTCTTCTCTCCAC
CTCTTCACTGCTTTGCTGGTCAATCTAGCCTTGTGTGCGTGAGTGTGGGT
GCGCCTATCACGATGTACAGGTAAACTTTTATTATTACTAAATGTCCAGA
AGTCTTTAATTAAAATAATTACTTATTGCGACAATAAGTAAGAAGGTGAA
ATGTAAGACTTCCCAAATTGCAATAAGAACAATAGCTGTGAGAGCAATAA
TAATCTTCAGCTTTTATCACTGAGCGGAGCGACATAAGCAAGCTGCACTG
TACTTCAGATGCAATGTTGTTGTTTCAGTTGTTGTAGCTGCTCCTTGGCT
TTCACACGCGAGTTGTGGGTGAGCCTTAATCACATTTATTCGATGGTGAG
TGGAGAAGGAGGTGCTGAGTGGGCGAGGGAGCGGCGCCGAGAGAGCGAAC
GAGCGGTTGACATTGACAACCCCCCTTTTTTGCTGTTGGGAGCGGGCGCA
CGCCCATGTCCAGCTGCCCAGCGGCAGCGACCAGGAGCGCAGGCAGGCGC
AGCTCGCGGCCAGCACCTACGACGTGCTACAGCGGACGACGGACAGTATC
CAGCGATCCAACCAGATTGCCATCGAAACGGAGAACATGGGGGCGGAGGT
ACTCGGCGAACTGGGCGAGCAGAGGGAGTCGCTGCTACGCACCACGCGCC
GCCTGGAGGACGCCGATCAGGATCTGTCCAAATCGAGGGTCATCATTCGG
AAGTTGAGCAGGGAGGTGCTCTACAACAAGATCATCCTAA
>CG7682|FBgn0038614
MSSCPAAATRSAGRRSSRPAPTTCYSGRRTVSSDPTRLPSRRRTWGRRYS
ANWASRGSRCYAPRAAWRTPIRICPNRGSSFGS
>>BcDNA:LD21735|FBgn0027516|cDNA sequence
CCTCTCAGCTGGCTCAGTGTTTTTTTAGTGTTCGAGCTGTGCGTGTGAAC
TGTGATATTGCGATATTGGGCTATCGCAATTGGAAACTGGACTTTTGGTT
GAATTCATTATAAACGAAAGTGCGCTCGTTGAATCATTAAACAACATTTG
AGCAGGCGAGTTACAATTCTATTACCGGTTTTTTTTTTAAAGCCAATCGA
GTTTTGGAGGTAATTCTCGTCGGCGGAGGGAACGTAAGCAGTCGCCAAGA
TGGGGGACATGTTCCGTAGTGAGGAGATGGCACTCTGCCAGATGTTCATT
CAGCCGGAGGCCGCGTATACCTCCGTATCTGAGCTGGGCGAAACCGGCTG
CGTGCAGTTCCGCGACTTGAATGTGAACGTGAACGCCTTCCAGCGCAAGT
TCGTCACCGAGGTGCGTCGCTGCGATGAGCTGGAGCGCAAGATCCGCTAC
ATCGAGACGGAGATCAAGAAGGACGGCATCGTCCTGCCCGACATCCAGGA
TGACATTCCGCGTGCGCCCAATCCACGCGAGATCATCGATCTGGAGGCGC
ATCTGGAGAAGACCGAGTCGGAGATGATCGAGCTGGCCCAGAACGAGGTG
AACATGAAGTCCAACTATCTGGAGCTGACCGAGCTGCGCAAGGTGCTGGA
GAACACGCAGGGCTTCTTCTCCGACCAGGAGGTTCTCAATCTGGACTCCT
CCAACCGAGCTGGAGGAGACAACGATGCTGCTGCTCAACACCGTGGCCGG
CTTGGATTCGTTGCCGGTGTAATTAACCGGGAGCGAGTGTTTGCCTTTGA
GCGTATGCTGTGGCGCATCTCCAGGGGCAATGTCTTCCTCAAGCGCTCCG
ATCTGGACGAGCCGCTGAACGATCCGGCCACCGGACATCCCATCTACAAG
ACCGTCTTCGTGGCCTTCTTCCAGGGCGAGCAACTGAAGAACCGTATCAA
GAAGGTGTGCACTGGCTTCCACGCCTCGCTGTATCCCTGTCCCAGCTCGC
ACAACGAGCGCGAGGAAATGGTTCGCAATGTGCGCACCCGCCTGGAGGAT
CTGAAGCTGGTCCTTAGCCAGACGGAGGATCATCGTAGCCGCGTCCTGGC
CACCGTGTCCAAGAATCTGCCCTCGTGGTCGATCATGGTCAAGAAGATGA
AGGCCATTTACCACACGCTGAATCTGTTCAACATGGACGTGACCAAGAAG
TGCCTGATTGGCGAGTGTTGGGTGCCCACCAATGATTTGCCCGTTGTCCA
AAAGGCTCTGTCCGATGGATCTGCTGCAGTGGGCAGCACCATACCCTCGT
TCCTGAACGTGATCGACACCAACGAGCAGCCGCCGACCTTTAACAGGACT
AACAAGTTTACCCGTGGCTTCCAGAATCTGATTGATGCCTACGGAGTGGC
CTCGTACAGAGAGTGCAATCCCGCCCTGTACACCTGCATCACCTTCCCCT
TCCTTTTCGCTGTGATGTTCGGCGATTTGGGTCACGGCCTTATTCTGGTT
TTGTTTGGAGCTTGGATGGTTTTGTGCGAGCGCAAGCTGGCTCGCATCCG
CAACGGTGGTGAGATCTGGAACATCTTCTTCGGCGGTCGCTATATCATTC
TGCTGATGGGTCTGTTTGCCATGTACACTGGTTTGGTTTACAACGATGTC
TTCTCCAAGTCGATGAACCTGTTTGGATCACGTTGGTTCAACAACTACAA
CACAACGACTGTCCTGACCAACCCGAATCTGCAGTTGCCGCCCAACAGCT
CCGCCGTGGGTGTCTATCCCTTCGGAATGGATCCCTTTCAAGATGAAGCT
CTCGATCATCTTCGGAGTGCTGCACATGGTCTTCGGCGTGTGCATGTCGG
TCGTTAACTTCACCCACTTCAAGCGTTATGCCTCCATTTTCCTGGAGTTC
GTGCCCCAAATTCTGTTCCTGCTACTGCTCTTCGGCTACATGGTGTTCAT
GATGTTCTTCAAGTGGTTCAGCTATAACGCTAGGACTAGCTTCCAGCCAG
AAACTCCTGGATGCGCTCCCTCCGTGTTGATCATGTTCATCAACATGATG
CTGTTCAAGAACACTGAGCCACCAAAGGGTTGCAACGAGTTCATGTTCGA
GTCACAGCCCCAGTTGCAGAAGGCCTTTGTGCTCATCGCCCTGTGCTGCA
TTCCTTGGATGCTTCTGGGCAAGCCCCTGTACATCAAGTTCACTCGCAAA
AACAAGGCTCATGCCAATCACAATGGTCAGTTGACCGGCAACATTGAACT
GGCCGAAGGCGAGACTCCTCTGCCCACAGGATTCTCTGGAAACGAGGAGA
ATGCCGGGGGTGCCCATGGCCATGACGATGAGCCCATGAGCGAAATCTAC
ATCCATCAAGCCATCCACACCATCGAATATGTGCTCAGTACCATCTCGCA
CACGGCGTCCTATCTGCGTCTCTGGGCTCTGTCCCTGGCTCACGCCCAGC
TCTCCGAGGTGCTGTGGCAAATGGTGTTGTCCCTGGGCCTTAAGATGTCC
GGCGTGGGCGGTGCCATTGGTCTGTTCATCATCTTCGGCGCCTGGTGCTT
GTTCACCCTGGCTATCCTGGTCCTCATGGAGGGTCTGTCCGCCTTCCTGC
ACACTCTGCGTCTGCACTGGGTGGAGTTCATGAGCAAGTTCTACGAGGGA
ATGGGCTACGCCTTCCAGCCGTTCAGCTTCAAGGCCATTCTCGATGGCGA
AGAGGAGGAGTAA
>>BcDNA:LD21735|FBgn0027516|cDNA sequence
ATCGCTGGCGGTGAGCAGACGTGTGCTGCGCTCCATTCAGATTCAGATTC
GAATAAAGATACTCTCGCTCGGCAACAAACAAGTTCTAAATTTATCGCTC
GCACTCGCGGGGAATCGAATCTGGTGTGTCTTTGACATAAAAAGCGTTTT
CGGAGCTGTGAAGGGAACGTAAGCAGTCGCCAAGATGGGGGACATGTTCC
GTAGTGAGGAGATGGCACTCTGCCAGATGTTCATTCAGCCGGAGGCCGCG
TATACCTCCGTATCTGAGCTGGGCGAAACCGGCTGCGTGCAGTTCCGCGA
CTTGAATGTGAACGTGAACGCCTTCCAGCGCAAGTTCGTCACCGAGGTGC
GTCGCTGCGATGAGCTGGAGCGCAAGATCCGCTACATCGAGACGGAGATC
AAGAAGGACGGCATCGTCCTGCCCGACATCCAGGATGACATTCCGCGTGC
GCCCAATCCACGCGAGATCATCGATCTGGAGGCGCATCTGGAGAAGACCG
AGTCGGAGATGATCGAGCTGGCCCAGAACGAGGTGAACATGAAGTCCAAC
TATCTGGAGCTGACCGAGCTGCGCAAGGTGCTGGAGAACACGCAGGGCTT
CTTCTCCGACCAGGAGGTTCTCAATCTGGACTCCTCCAACCGAGCTGGAG
GAGACAACGATGCTGCTGCTCAACACCGTGGCCGGCTTGGATTCGTTGCC
GGTGTAATTAACCGGGAGCGAGTGTTTGCCTTTGAGCGTATGCTGTGGCG
CATCTCCAGGGGCAATGTCTTCCTCAAGCGCTCCGATCTGGACGAGCCGC
TGAACGATCCGGCCACCGGACATCCCATCTACAAGACCGTCTTCGTGGCC
TTCTTCCAGGGCGAGCAACTGAAGAACCGTATCAAGAAGGTGTGCACTGG
CTTCCACGCCTCGCTGTATCCCTGTCCCAGCTCGCACAACGAGCGCGAGG
AAATGGTTCGCAATGTGCGCACCCGCCTGGAGGATCTGAAGCTGGTCCTT
AGCCAGACGGAGGATCATCGTAGCCGCGTCCTGGCCACCGTGTCCAAGAA
TCTGCCCTCGTGGTCGATCATGGTCAAGAAGATGAAGGCCATTTACCACA
CGCTGAATCTGTTCAACATGGACGTGACCAAGAAGTGCCTGATTGGCGAC
TGTTGGGTGCCCACCAATGATTTGCCCGTTGTCCAAAAGGCTCTGTCCGA
TGGATCTGCTGCAGTGGGCAGCACCATACCCTCGTTCCTGAACGTGATCG
ACACCAACGAGCAGCCGCCGACCTTTAACAGGACTAACAAGTTTACCCGT
GGCTTCCAGAATCTGATTGATGCCTACGGAGTGGCCTCGTACAGAGAGTG
CAATCCCGCCCTGTACACCTGCATCACCTTCCCCTTCCTTTTCGCTGTGA
TGTTCGGCGATTTGGGTCACGGCCTTATTCTGGTTTTGTTTGGAGCTTGG
ATGGTTTTGTGCGAGCGCAAGCTGGCTCGCATCCGCAACGGTGGTGAGAT
CTGGAACATCTTCTTCGGCGGTCGCTATATCATTCTGCTGATGGGTCTGT
TTGCCATGTACACTGGTTTGGTTTACAACGATGTCTTCTCCAAGTCGATG
AACCTGTTTGGATCACGTTGGTTCAACAACTACAACACAACGACTGTCCT
GACCAACCCGAATCTGCAGTTGCCGCCCAACAGCTCCGCCGTGGGTGTCT
ATCCCTTCGGAATGGATCCCTTTCAAGATGAAGCTCTCGATCATCTTCGG
AGTGCTGCACATGGTCTTCGGCGTGTGCATGTCGGTCGTTAACTTCACCC
ACTTCAAGCGTTATGCCTCCATTTTCCTGGAGTTCGTGCCCCAAATTCTG
TTCCTGCTACTGCTCTTCGGCTACATGGTGTTCATGATGTTCTTCAAGTG
GTTCAGCTATAACGCTAGGACTAGCTTCCAGCCAGAAACTCCTGGATGCC
CTCCCTCCGTGTTGATCATGTTCATCAACATGATGCTGTTCAAGAACACT
GAGCCACCAAAGGGTTGCAACGAGTTCATGTTCGAGTCACAGCCCCAGTT
GCAGAAGGCCTTTGTGCTCATCGCCCTGTGCTGCATTCCTTGGATGCTTC
TGGGCAAGCCCCTGTACATCAAGTTCACTCGCAAAAACAAGGCTCATGCC
AATCACAATGGTCAGTTGACCGGCAACATTGAACTGGCCGAAGGCGAGAC
TCCTCTGCCCACAGGATTCTCTGGAAACGAGGAGAATGCCGGGGGTGCCC
ATGGCCATGACGATGAGCCCATGAGCGAAATCTACATCCATCAAGCCATC
CACACCATCGAATATGTGCTCAGTACCATCTCGCACACGGCGTCCTATCT
GCGTCTCTGGGCTCTGTCCCTGGCTCACGCCCAGCTCTCCGAGGTGCTGT
GGCAAATGGTGTTGTCCCTGGGCCTTAAGATGTCCGGCGTGGGCGGTGCC
ATTGGTCTGTTCATCATCTTCGGCGCCTGGTGCTTGTTCACCCTGGCTAT
CCTGGTCCTCATGGAGGGTCTGTCCGCCTTCCTGCACACTCTGCGTCTGC
ACTGGGTGGAGTTCATGAGCAAGTTCTACGAGGGAATGGGCTACGCCTTC
CAGCCGTTCAGCTTCAAGGCCATTCTCGATGGCGAAGAGGAGGAGTAAAC
CCATCCAAATGTGCTCAAACTAG
>BcDNA:LD21735|FBgn0027516
MGDMFRSEEMALCQMFIQPEAAYTSVSELGETGCVQFRDLNVNVNAFQRK
FVTEVRRCDELERKIRYIETEIKKDGIVLPDIQDDIPRAPNPREIIDLEA
HLEKTESEMIELAQNEVNMKSNYLELTELRKVLENTQGFFSDQEVLNLDS
SNRAGGDNDAAAQHRGRLGFVAGVINRERVFAFERMLWRISRGNVFLKRS
DLDEPLNDPATGHPIYKTVFVAFFQGEQLKNRIKKVCTGFHASLYPCPSS
HNEREEMVRNVRTRLEDLKLVLSQTEDHRSRVLATVSKNLPSWSIMVKKM
KAIYHTLNLFNMDVTKKCLIGECWVPTNDLPVVQKALSDGSAAVGSTIPS
FLNVIDTNEQPPTFNRTNKFTRGFQNLIDAYGVASYRECNPALYTCITFP
FLFAVMFGDLGHGLILVLFGAWMVLCERKLARIRNGGEIWNIFFGGRYII
LLMGLFAMYTGLVYNDVFSKSMNLFGSRWFNNYNTTTVLTNPNLQLPPNS
SAVGVYPFGMDPFQDEALDHLRSAAHGLRRVHVGR
>BcDNA:LD21735|FBgn0027516
MGDMFRSEEMALCQMFIQPEAAYTSVSELGETGCVQFRDLNVNVNAFQRK
FVTEVRRCDELERKIRYIETEIKKDGIVLPDIQDDIPRAPNPREIIDLEA
HLEKTESEMIELAQNEVNMKSNYLELTELRKVLENTQGFFSDQEVLNLDS
SNRAGGDNDAAAQHRGRLGFVAGVINRERVFAFERMLWRISRGNVFLKRS
DLDEPLNDPATGHPIYKTVFVAFFQGEQLKNRIKKVCTGFHASLYPCPSS
HNEREEMVRNVRTRLEDLKLVLSQTEDHRSRVLATVSKNLPSWSIMVKKM
KAIYHTLNLFNMDVTKKCLIGECWVPTNDLPVVQKALSDGSAAVGSTIPS
FLNVIDTNEQPPTFNRTNKFTRGFQNLIDAYGVASYRECNPALYTCITFP
FLFAVMFGDLGHGLILVLFGAWMVLCERKLARIRNGGEIWNIFFGGRYII
LLMGLFAMYTGLVYNDVFSKSMNLFGSRWFNNYNTTTVLTNPNLQLPPNS
SAVGVYPFGMDPFQDEALDHLRSAAHGLRRVHVGR
Scim34
AE003725 (insertion @69750), nearest ORF (CG5557) @69916
The EST GH22029 has 73% identity with Zn finger transcription factor, 89%
identity with CG1004O gene product
>>1(3)02102|FBgn0010768|cDNA sequence
TGAACACGAACAGTTATTCTAGCTCCTCCTGTTCTGCTGCTTCTGTTGGT
GTTTTTTTGCGTTTGCCGCGACGTAAACAACCGAAGAGCGGAGCTTACGC
CCCCAACTGCTCCAGCTCCACGCCCCCCTCGCAACGTGTTCGTTGTAAAT
GCAATTAATCGTTGTTGGTCGCTGAAAAAACTAATATTTCGGGAGTCCGA
TACAAGAAGGGCAAAAAAGCACAAGTGCAGAAAAAGCAAAAAAAAACATA
AGCTTGCCGTGTGTGTGCGTTGGTGTGTGTGTGTATTTTTGGCGTGCGCG
GCCATTTTTTCTTGTTGCCACTCGAGAGTCTCTTCTGTGTGCGAAAGAGA
GGCCCGACGGCAAAGCAGAACGTAGCAAACTACGCAAATCCCCACTAAAT
TCGCTAATTTTCGCTAAATAAATCAATCGCAAGGGGTGCCCAAAAACAAA
CAAAAGTACGAAAATAGAGTGCCGCAGACCACAAACAAATCACCTGCGCC
AGTGTGTGTGAGTGTGTCAGTGTGCGAAACAGGCAAGCTCCCAGGCGGGC
TCCCACCCACAATCAGTCGGCGAACACGTGCTCCAAAATATCAACAAAAG
TTGGCCCACAAACATAAAAAAGGGGGCCGCGACTGCATCCGAGACCCGAA
CGCCGGAACACAAAAGGCACTTTCAAATTGCACAAAAACGCGCCGAGTTG
TGTAAGCCCGCCGCCAGTGAAGTGTCGACTTCCCGGCGACGAAGACCGCA
GCAATATTTCCACTAGCCAGTAACGCACAGGCTCGTTCCGCCGCTGACGA
CGACCCCAAACTGGACGCCCGCACCATCGCCTACCGCCCGCTGACCCTTC
GCGGCCACCAGACGCCGCTTATGGCCGAACTGCCGACGGCGCCGAACGGC
GTCCCCAGCGGCGATTATCTGCACCGCTCCATCGATCAGCTGCGTTCGCT
GGGTCATCTGACCACCGCCCAATTGGTTCACGACTACAAGCCCTTCAACA
TTAGCGAATTCCGGCAGAATGTCGCTGAGCGACTGGACTACTCGCTGAAG
AACGGCCTGGTGCAGCACCAACAGCAAATGGTCATGGAGCAGCAGCCACA
TCCCGATCAGCAGCAGCAGCAGCATCTGCATCACCCGCAACAGCAGCAGC
ACCCGCCGCAGCTGAAGGTCAGCTACAGTGCGCCCAACTCGCCGCCCACT
CCACACGAGCAGCAGGAACAGAAGTACGACCCGAATCGATCGCCGCCGCG
TCAGCAGATGAGCAGCGCTAGCGGCAGTGGCAGCAACGGCTCCTCGCCTG
AGGAGGAAAGCCGACGGGGAGACGGTGATCAGGCCAAGCCCTACAAGTGT
GGCTCGTGCAGCAAGTCCTTTGCCAACTCCTCGTACCTGTCGCAGCACAC
GCGTATCCACCTGGGGATCAAGCCGTACCGCTGCGAGATATGTCAGCGCA
AGTTCACGCAATTGTCGCATCTCCAGCAGCACATCCGTACGCACACGGGT
GACAAACCGTACAAATGCCGGCACGCCGGCTGCCCGAAGGCCTTCTCGCA
GCTATCCAATCTGCAGTCACACTCGCGTTGTCATCAGACGGACAAGCCGT
TCAAGTGCAACTCCTGCTACAAGTGCTTCAGCGACGAGATGACCCTGCTG
GAGCACATTCCCAAGCACAAGGACTCCAAGCACCTGAAGACGCACATCTG
CAATTTGTGTGGCAAATCGTACACGCAAGAGACCTACCTTCAGAAACATC
TGCAGAAGCACGCAGAGAAGGCGGAGAAGCAGCAGCATCGCCACACGGCC
CAGGTGGCTGCCCACCAGCAGCACGTACCGGCGAGCGGCATCGGCTTGAA
TTTGCAGCGCCAGGCCATGAACGATGTGAATGCCGCATATTGGGCCAAAA
TGGGCGCAGACAGTGCGGCGGCTTCGCTGGCGGAAGCCATTCAGCAGCAG
TTGCCGCAGGCCGGCGAATAAAGATTGTAACTATATATAAAAGT
>1(3)02102|FBgn0010768
MAELPTAPNGVPSGDYLHRSIDQLRSLGHLTTAQLVHDYKPFNISEFRQN
VAERLDYSLKNGLVQHQQQMVMEQQPHPDQQQQQHLHHPQQQQHPPQLKV
SYSAPNSPPTPHEQQEQKYDPNRSPPRQQMSSASGSGSNGSSPEEESRRG
DGDQAKPYKCGSCSKSFANSSYLSQHTRIHLGIKPYRCEICQRKFTQLSH
LQQHIRTHTGDKPYKCRHAGCPKAFSQLSNLQSHSRCHQTDKPFKCNSCY
KCFSDEMTLLEHIPKHKDSKHLKTHICNLCGKSYTQETYLQKHLQKHAEK
AEKQQHRHTAQVAAHQQHVPASGIGLNLQRQAMNDVNAAYWAKMGADS
AAASLAEAIQQQLPQAGE
Scim35
AE003732 (insertion @29310), no genes nearby. The nearest ORFs are CG15690
@5954 to 6364 and CG17838 @42621 to 55761
22 >CG15690|FBgn0038825|cDNA sequence
TGACTGATCTGTCGCCGACCACCTCTGGGTTTCTACATGGCCATGGCGG
CGCCAACGCACAGCATCACCAGCAACTTTTGCACCACCAACAGCAGCAGC
ATCAGCAGACCCACCAGCAGCACCTCCAGCAGCAGTACCACCACCGGCAG
CACTCGCTCCACCAGCAGCACCTGCAGCGCCAGCACTCGCACACCTCGCT
GACCAAGATCCACCGCCAGAGCAGCAGCCACAGCGTCCACGGTGGTGGCG
GGCGGGCGGATCACCACCGAACCACTACCGGAGCCACCGGACGGCTCTTC
TCCTACTTCGGAAACGAGCAGGATCACGAGCGCAAGAAGTCGGAGACTTC
GTTCTTTAATCTCGGATTTCGGAGGAAGTCCACTGTGGTCTACTACGCTC
CAGCGGATTGA

+UZ,/14 >CG15690|FBgn0038825
MTDLSPTTSGFLHGHGGANAQHHQQLLHHQQQQHQQTHQQHLQQQYHHR
QHSLHQQHLQRQHSHTSLTKIHRQSSSHSVHGGGGRADHHRTTTGATGRLF
SYFGNEQDHERKKSETSFFNLGFRRKSTVVYYAPAD
>>CG17838|FBgn0038826|cDNA sequence
ATGGCGGAAGGTAATGGCGAACTGTTGGATGACATTAACCAGAAAGCCGA
TGACCGTGGCGATGGCGAGCGTACAGAGGATTATCCCAAGCTGCTGGAAT
ACGGTCTGGACAAGAAGGTCGCCGGCAAACTGGATGAGATCTACAAAACC
GGCAAGTTGGCTCACGCCGAGCTGGACGAGCGCGCCCTGGACGCGCTCAA
GGAGTTTCCCGTCGATGGTGCCTTGAATGTGTTGGGACAGTTCCTGGAAT
CGAACCTGGAGCACGTGTCAAACAAGTCCGCCTACCTATGCGGCGTGATG
AAGACGTACCGACAGAAGAGTCGAGCCAGCCAACAGGGCGTGGCCGCGCC
CGCAACTGTCAAAGGTCCCGACGAGGACAAGATCAAGAAAATCCTCGAGC
GCACCGGCTACACATTAGATGTGACGACAGGTCAGCGTAAATACGGCGGA
CCGCCGCCGCATTGGGAGGGAAATGTGCCAGGCAACGGTTGCGAGGTTTT
CTGCGGCAAGATACCCAAGGACATGTACGAGGACGAACTGATTCCGCTAT
TCGAGAACTGCGGCATAATCTGGGACCTACGACTCATGATGGACCCGATG
ACGGGCACAAATCGTGGTTATGCATTTGTCACATTCACAAATCGCGAAGC
GGCCGTCAATGCAGTGCGACAGCTCGATAATCACGAAATAAAACCCGGCA
AGTGTCTAAAAATAAATATAAGCGTACCGAATCTGCGCCTTTTCGTAGGC
AATATTCCCAAGTCAAAGGGCAAAGATGAAATTTTAGAGGAATTTGGTAA
ACTTACAGGCAAAAAGATTGGTGTTACGATATCATTTAACAATCACCGGC
TATTTGTCGGCAATATACCTAAGAATAGAGATCGCGACGAATTAATTGAG
GAATTTTCAAAACATGCACCTGGCCTATACGAGGTAATCATATACAGTTC
GCCAGATGATAAGAAAAAGAATCGCGGCTTTTGCTTTCTTGAGTACGAGT
CACACAAGGCGGCGTCTTTGGCCAAACGAAGACTTGGCACAGGAACAATT
AAGGTTTGGGGATGTGATATAATAGTCGACTGGGCCGATCCACAGGAGGA
GCCGGATGAGCAAACAATGTCCAAGGTTAAAGTTCTTTATGTGCGAAATC
TTACCCAGGACGTCTCAGAGGATAAACTGAAGGAACAATTTGAGCAATAC
GGAAAAGTGGAACGCGTTAAGAAAATTAAAGACTATGCCTTTATACACTT
TGAGGATCGTGATAGCGCCGTCGAAGCTATGCGTGGCCTTAATGGCAAGG
AGATCGGCGCCTCGAATATTGAGGTCTCTCTAGCCAAACCCCCCTCGGAC
AAAAAGAAAAAGGAGGAGATTCTGCGTGCTCGTGAGCGCCGCATGATGCA
AATGATGCAAGCGCGTCCCGGGATCGTGGGAAACCTGTCGCCGACACATC
CTAGCATAATGTCCTTGACGCCCATGCGCCCAGGGGCGCGCATGCCGCTG
CGTACGCCGATACCCCGTGAATACGACTACTTTTACGACTTTTTCGGTTT
CTCGGACTATCGCCAAGGGGGGTCCTTTGGCAATAATGTGTCCTACTACG
ATGACATGTACCGCTGGATTGATGGGGATTACAACTACTATGATTACCCG
AACGGTGGCGGCGGGGGCAGCGGGGGAGGAGGAGGTAGTGTGTCCGGCGG
TACGGTGCTTCCGCTCTCGGCCGGCGGCTCCCAGAATTCACCGATGGCTA
GTGGACAGCGATCGGCCAGAGGATCGGCCAGTGGTCCCAGTGCTTCCCCG
AGCCTTATGCTGGAGCTGCATTCAAAACATTCATGGAGGGTAATTAAGCG
TAGCTCATCGGCCACCTCCTTGGACAGCGACAAGAGTCGCTCTCCGGGGA
AGAAGCGTAAAGTCCATAGGTCACATAACAAAAACAAGTCACACAAGTCT
AAGAAGCATGCCCACAAGTCCAAGTCGTCTCGGCCCGATAAAGAAAAGAA
ATCCAAGCGGAAGTCCGAATAA
>CG17838|FBgn0038826
MAEGNGELLDDINQKADDRGDGERTEDYPKLLEYGLDKKVAGKLDEIYKT
GKLAHAELDERALDALKEFPVDGALNVLGQFLESNLEHVSNKSAYLCGVM
KTYRQKSRASQQGVAAPATVKGPDEDKIKKILERTGYTLDVTTGQRKYGG
PPPHWEGNVPGNGCEVFCGKIPKDMYEDELIPLFENCGIIWDLRLMMDPM
TGTNRGYAFVTFTNREAAVNAVRQLDNHEIKPGKCLKINISVPNLRLFVG
NIPKSKGKDEILEEFGKLTGKKIGVTISFNNHRLFVGNIPKNRDRDEIIE
EFSKHAPGLYEVIIYSSPDDKKKNRGFCFLEYESHKAASLAKRRLGTGRI
KVWGCDIIVDWADPQEEPDEQTMSKVKVLYVRNLTQDVSEDKLKEQFEQY
GKVERVKKIKDYAFIHFEDRDSAVEAMRGLNGKEIGASNIEVSLAKPPSD
KKKKEEILRARERRMMQMMQARPGIVGNLSPTHPSIMSLTPMRPGARMPL
RTPIPREYDYFYDFFGFSDYRQGGSFGNNVSYYDDMYRWIDGDYNYYDYP
NGGGGGSGGGGGSVSGGTVLPLSAGGSQNSPMASGQRSARGSASGPSASP
SLMLELHSKHSWRVIKRSSSATSLDSDKSRSPGKKRKVHRSHNKNKSHKS
KKHAHKSKSSRPDKEKKSKRKSE

Scim36
AE003758 (insertion @217925), nearest ORF (CG6295) @217933
>>CG6295|FBgn0039471|cDNA sequence
ATGATGAAACTGTTCCTGGCCTTGGCCTTTTGTGTCCTGGCGGCTAATGC
CGTGGAGGTTCCTGTGAATGGTGAGAACGGATGGTATGTGCCCCAGGCCC
ATGGTACCATGGAGTGGATGGACCGCGAGTTCGCCGAGGCCTATTTGGAC
ACCAAGAACCGCATGGAAGGACGCAACGTCCTGAACCCCGTCACCTTCTA
CCTGTACACCAACTCGAACCGCAACTCTCCCCAGGAGATCAAGGCTACGT
CAGCATCGATCTCTGGCTCGCACTTCAACCCCAACCACCCCACCCGCTTC
ACTATCCACGGCTGGTCCTCCAGCAAGGATGAGTTCATCAACTACGGTGT
CCGCGATGCCTGGTTCACCCACGGCGACATGAACATGATTGCCGTCGACT
GGGGACGTGCTCGTTCCGTGGACTACGCCTCCTCCGTTCTGGCTGTTCCC
GGAGTCGGCGAGCAGGTGGCTACCCTGATCAACTTTATGCGCAGCAATCA
CGGCCTGAACCTGGACAACACCATGGTGATTGGTCACAGCCTGGGCGCCC
ATGTCTCGGGCTATGCTGGCAAGAATGTGAAGAACGGCCAGCTGCACACC
ATCATTGGTCTGGACCCCGCCCTGCCCCTTTTCAGCTACGATTCCCCCAA
CAAGCGCCTGAGCTCCACCGATGCTTACTACGTGGAGTCCATCCAGACCA
ACGGAGGAACCCTGGGATTCCTGAAGCCCATCGGCAAGGGAGCCTTCTAC
CCCAACGGAGGAAAGAGCCAGCCCGGATGTGGTGTTGATCTCACCGGATC
CTGCGCCCACAGCCGCTCAGTGATCTACTACGCCGAGTCCGTGACCGAGA
ACAACTTCCCCACCATGCGCTGCGGCGACTACGAGGAGGCTGTGGCCAAC
GAGTGCGGTAGCTCCTACAGCTCCGTCCGCATGGGAGCCACCACCAATGC
CTACATGGTCGCTGGAGATTACTATGTACCCGTCCGTAGCGATGCTCCCT
ACGGAATGGGCAACTAA
>CG6295|FBgn0039471
MMKLFLALAFCVLAANAVEVRVNGENGWYVPQADGTMEWMDREFAEAY
LETKNRMEGRNVLNPVTFYLYTNSNRNSPQEIKATSASISGSHFNPNHPTRF
TIHGWSSSKDEFINYGVRDAWFTHGDMNMIAVDWGRARSVDYASSVLAVP
GVGEQVATLINFMRSNHGLNLDNTMVIGHSLGAHVSGYAGKNVKNGQLHT
IIGLDPALPLFSYDSPNKRLSSTDAYYVESIQTNGGTLGFLKPIGKGAFY
PNGGKSQPGCGVDLTGSCAHSRSVIYYAESVTENNFPTMRCGDYEEAVAK
ECGSSYSSVRMGATTNAYMVAGDYYVPVRSDAPYGMGN
Scim37
AE003764 (insertion @56595), nearest ORF (CG12425) @48242 to 49549
>>CG12425|FBgn0039571|cDNA sequence
CAGCGCCTCCGAATTTTGGCCAATGCGTGTGCTCCTGGCGTTCAATGCTG
ACCATGTCGACCCTGATGATGCCAGCACCGACCATAGCCATGGGTGCCCC
GCAGATCACAATGGGTCCGCACAAGCCGCCGGAAACGAAGCTGTTGGCC
ATCCATCCCGCTGCAGCGGCCGCAGCAGCCGCCCAGCAGCAGCAGCAGTC
GGTGA
>CG12425|FBgn0039571
MLTMSTLMMPAPTIAMGAPQITMGPHKPPETKLLAIHPAAAAAAAAQQQQ
QSV

TABLE 5
DNA and AA sequences for known loci

Fim^{Scim}
>>Fim|FBgn0024238|cDNA sequence
CAACCTGCAATTTAGTTTTCTGTTTCTTGTTAAACATCTAACAAAAGCGC
TTCGCACGCACAGCAAACCGGTCGATTCCGAATTCAGACGTGCTAAATA
A
CGTTTGAAAACAAAATTTGCTCGGTTGACGTTGTAAATAACAACACGGA
G
GCTTTAAATCTGGCTTCATCTTTTTTTTTTTGGTAAAAACCAGCAACAAA
CAAAACCAGCAACGAGTGTGTATGGGTGAAAAGTTAAGAAAAAAGTAA
AA
GAAGTTTAAGAATATTAGCACAGAAACGCACAAATACATAGATACGTAT
A
GGAAAAGGAGCGCGCGGTAAAACAAAAAAGAAGAGGAGCAAAAAGAA
GAA
AATCGCCGACTGCGAAAACGGCGACAAAGTGAAATTGATTATAAACGG
AT
AATTTATTTAAAATAATTACCAATCAGCGGCGTCAGTTATAAATAATTAA
GAAAGTCAAGAAGAAAAGCAGCAGCAGCACACCACACACACATCGGCA
GC
GAAAATGGCAACACTTAACAAATTCACAAAAACGCTGTCCATTGATGAA
A
AGGCGGAGATCAAGGAGAAATTCATAGAGTTGGATGCCAACAAAGATG
GC
TTCATCGATCTGCACGAGCTAAAGGATGCCCTCAACCAGGTGGGCTTCA
A
GCTGGCCCGGCTACCAGGTGCGCGAAATGATTGACGAGTACAAGGGCAA
AC
AGCTGACTGCCTTCCAGGGCAAATTGAATCTGGAGGAGTTCGAGGCACT
G
TGCCTGGACCTGAAGAGCAAGGATGTGGCCAGCACATTCAAGACGGTCG
T
CTCCAAGAAGGAGAACTTGGAGACCCTGGGCGGCATGTCGAGCATTTCG
T
CGGAGGGCACCACCCATTCGGTGCGCCTCGAGGAGCAGCTGGCCTTCTC
C
GACTGGATTAACTCGAATCTGGGCCACGACAAGGATCTGCAACATCTGC
T
GCCCATCGATAGCGAGGGCAAGCGCTTGTATCTGAGCATCAAGGATGGT
A
TTCTGCTGTGCAAAATTATCAACCACTCCTGCCCGGACACAATCGATGA
G
CGTGCGATCAACAAGAAGAACCTCACCGTCTACCGGGAGTTTGAGAACT
T
GACCCTGGCCTTGGTTTCCTCCCAGGCGATTGGATGCAACATCGTAAC
A
TCGATGCCCACGATCTGGCCAAGGGCAAGCCACATCTGGTGCTGGGTCT
T
CTCTGGCAGATCATCCGCATCGGTCTGTTTAGCCACATCACCCTGGACAG
CTGTCCGGGATTGGCTGGCCTACTCTTCGACAACGAACGTCTCGAGGAT
C
TGATGAAGATGTCGCCGGAGGCCATCCTTTTGCGTTGGGTCAACCATCAT
TTGGAGCGAGCCGGCATCTCGAGGCGGTGCACCAACTTCCAGTCGGACA
T
CGTCGATTCTGAGATCTATTCGCATTTGCTGAAGCAAATTGCCGGCAATG
ATGCTGATGTCAATCTGGATGCCCTGCGGGAATCCGATCTGCAGTCGCG
C
GCGGAGATCATGTTGCAGCAGGCCGCCAAGCTCAACTGCCGCAGCTTCC
T
CACGCCACAGGATGTCGTCAACGGAGTCTACAAACTCAATCTAGCCTTC
G
TGGCTAATCTGTTCAACAACCATCCAGGATTGGACAAGCCCGAGCAGAT
C
GAGGGACTCGAGTCCATTGAGGAGACGCGCGAAGAGAAGACCTACCGC
AA
TTGGATGAACTCAATGGGCGTGGCACCGCACGTGAACTGGCTTTACTCC
G
ATTTGGCCGATGGTCTGGTCATTTTCCAGCTGTTCGACGTCATCAAGCCG
GGTATTGTCAACTGGAGCCGTGTGCACAAGCGTTTCAGCCCGCTGCGCA
A
GTTCATGGAAAAGCTGGAGAACTGCAACTATGCGGTGGATCTGGGCAAG
C
AGCTCAAATTCTCGCTGGTCGGAATCGCAGGCCAGGATCTAAACGATGG
C
AATGCCACGCTGACGTTGGCTCTCATCTGGCAGCTTATGCGTGCCTACAC
CCTGTCCATTCTGTCCCGCTTGGCCAACACTGGCAACCCCATTATCGAGA
AGGAGATCGTCCAGTGGGTGAATAACCGACTGTCGGAGGCAGGCAAC
AG
TCGCAGCTGCGTAACTTCAACGATCCGGCCATCGCCGATGGCAAGATCG
T
GATCGATCTGATCGATGCCATCAAGGAGGGCAGCATTAACTACGAGTTG
T
GATCGATCTGATCGATGCCATCAAGGAGGGCAGCATTAACTACGAGTTG
G
TGCGCACTAGCGGAACACAGGAGGATAACCTGGCCAATGCCAAGTATGC
C
ATCTCCATGGCCCGCAAGATCGGCGCCCGTGTCTACGCCCTGCCCGAGG
A
CATCACCGAGGTGAAGCCGAAAATGGTGATGACCGTTTTCGCCTGCATG
A
TGGCCCTCGACTACGTGCCCAACATGGACAGTGTGGACCAGAACAACCA
C
AACAGCTCCGCCAACAACAGCAATTAGGCGCCATAGCCACAACTCATTT
A
TACATAAACAAAAGGATGGGATTTTGAAAGGGAAGAACAGAGTGGGAA
CT
ACGTAAATTTGTCGTATTCGCTAATTACTAATAACTCTATTCGATTGCGT
TCCTTCCTGCGCTACCAATACATTTTTGTGTTTTTTTTTTATTTCAATTA
CTTTTTGTAATTTTTTTATTATATATTTATATATTATATAATTGAGTATG
AGTTTCCCCCTATTGTAATTAAGCAAACAGATTTTAGAGTGTCGCACATC
TGTTTTCGAGCCCGGCGGGGTGTTTCTTAATTAGTTTTGTTTAACAATTG
ATTAATTATACAACTGTAACCTTATTTTATAAATAAAGGTTTAGCTTGTT
CCGCTTAAAATCCTTAAAAGAAAAGAATGGTGAAACAAAAGAAAGAAA
AA
AAAAGCGACTTGTGCAGGATTAAGAAAGAATTATAATATTAACATTGCG
A
CG
>Fim|FBgn0024238
MATLNKFTKTLSIDEKAEIKEKFIELDANKDGRIDLHELKDALNQVGFKL
AGYQVREMIDEYKGKQLTAFQGKLNLEEFEALCLDLKSKDVASTFKTVVS
KKENLETLGGMSSISSEGTTHSVRLEEQLAFSDWINSNLGHDKDLQHLLP
IDSEGKRLYLSIKDGILLCKIINHSCPDTIDERAINKKNLTVYREFENLT
LALVSSQAIGCNIVNIDAHDLAKGKPHLVLGLLWQIIRIGLFSHITLDSC
PGLAGLLFDNERLEDLMKMSPEAILLRWVNHHLERAGISRRCTNFQSDIV
DSEIYSHLLKQIAGNDADVNLDALRESDLQSRAEIMLQQAAKLNCRSFLT
PQDVVNGVYKLNLAFVANLFNNHPGLDKPEQIEGLESIEETREEKTYRNW
MNSMGVAPHVNWLYSDLADGLVIFQLFDVIKPGIVNWSRVHKRFSPLRKF
MEKLENCNYAVDLGKQLKFSLVGIAGQDLNDGNATLTLALIWQLMRAYTL
SILSRLANTGNPIIEKEIVQWVNNRLSEAGKQSQLRNFNDPAIADGKIVI
DLIDAIKEGSINYELVRTSGTQEDNLANAKYAISMARKIGARVYALPEDI
TAVKPKMVMTVFACMMALDYVPNMDSVDQNNHNSSANNSN
bif^{Scim}
>>bif|FBgn0014133|cDNA sequence
TGTATTGGAAACGCGGCTTAACTTAATGCAGATTTTTCCGCTGATTCGGC
TGCGAAAGATGCACTTTTAAGGCGCAGCGAGTGCACCCACGCCCCGAGT
T
CGAGTGCAGTTGCAGTCGGGAAAGCTTGACAAGTGCGCGGAGCAAGGA
GA
GCGACGAGTTCGTTGAGCTACAGCGAAACGGAAAAGTGTAAAAGCGGG
GT
AAACAGGCGCAGCGGAGCGGATGATAAACGGAACTCGGGATCGGAAAA
TT
GGAAGCAAAACCAACCACCGATTATAAAAAATAGTCAGCATCTTAAAAA
C
TTGGTCTTTGGGTCGAGATGTAGAGCTGGATAGTGTGCCACACTATATAG
GGGATACTACTGCGATACAGACACCGCGGACACGGCTGACATGCATTAA
T
GCTGCGAATAATGCCTATTGCAGACGGGGATAATGGAGTCACAGAAGCG
G
CCTTCGTTGGACTTACACACGGATGTGCCAGCAGGATTAGCAGCTGGAG
G
ATCTGGCCTGGGAGCTGCAGCTGAGATGTCGCCCACTTCCGGTTTCCTGC
CGGACATGCCGCAGTGGAAGAAGGACCTCATCCAGCGCCGGAAAACGA
AC
GTGGCCCGCACCCAGGCGGCGTCCATCACCTCGCCCACCGATGGCAGTT
AC
GTGGCCCGCACCCAGGCGGCGTCCATCACCTCGCCCACCGATGGCAGTT
G
TGGGGCTTTGGCAGAAGCCAACGCAGCTCCAGGTGCAATTGCAGATTTC
A
CAGAACCGGCGACAATCAGTAGCACTAGTCAAAAGAGAAACATGATCG
GT
TCAGAGGAAAAGTCTGAGAAATCTTCTATTTCCAATACCAATTCCGATTC
CACTGGAGGTCATCACTCTGTTGTTGCCGTCTCCCTTTCGCCCGATGCGG
CAGCAACAACAAATGTAACAGTAACACCAATACCAAAGCAGCGATCGA
GT
TTACTCAACACAAGAAGTCAGGAGAGGGAGATGGTGCGATATATTCTAA
G
CGAGAGTGGAGAACGGGATGGAGAGCTTGAGAGCGGCGAACAGCCGGC
TG
GTGTGGTGAGTAACAGCCGGTGCGGTGAAGTTGAAACTGGCACAATTGG
A
TCGCCGTCGTCGTCAGCAAATCAAAATCCAAACCCAAATCATTTAAAAA
C
GAAATGCAAACCGGGACAGAGCGTTGCTGAGGGCAAGCCTTCAGCTAA
AG
AGACCATCGTCGATAACAGCAAAAGCTGCAGCAAAACCAAGAGTATTTC
C
GATAAATTGCAGAGCAACAAGTTTATAATTCAACAGCAGCAGCAACAAC
A
ACAACAACAGCAGCAACAACAGCAACTGTCGCCCACAAAGGTAACGGT
AA
AACCGACAATGGTCGCCATGCAAGAGATGAAGAAGACAACCAAACAAA
AT
GGCCAGCACCGACATCTAGCGGGCAAAATTGGCAGCGTAGCAGGAGGC
GA
TGTGGATCCACAACATCCACCGACGAATCCCATACCAGATTCATTGGAC
A
CCGGTGAGGATCTGAGCTACGCACCCGGAATTGTGTCCAAGCTGCGATG
G
CGCTACTTGAGCCTGGCCCTTCGCGAGTCCCGCCAGCAGAGCAGCAAAC
A
ACGCCTCCAACGCTCCACCAGTCTGAACACCCTGCTCGACCGCGACGAT
G
ACGAGGTGGAAGTGGAGGAGCCCGAAATGACCAACAGCCAGGTGCGTG
CC
AAATCAACACCGCCACCGATATTGGGGGCAAAACCGACACCGAAACCC
AG
CAGCCAGCATCAGCGTCCCGTCAGTCTGGGCGCCAATGGAGCTGGATCA
G
CTGTTAACCCGCCCTCGAACTCGGATCAGGCCCAAACGGGCGCCGTTGC
C
AACGGAGGCGCAGGATCTGGCCAACGTCGCGTCACTTTAAGCGCGGCA
A
CGAGGTGATGAAGCGGGCACGTTCCGTGGAGGCTCTGCTCTGCGAGAAA
T
CGCCATGGAACAGCCAGAGAATCAGCACTGCCGGCGCAGCTGCAGCAG
CA
GCACCTTCACCACCTGCTCCCAAGGCCACAGCCGCTGCTCCCTCACCTGT
TACATCGCCCACCTGCGTTACCATCGAGGACAAGATCCACAATGCCCGC
G
AACGACTGCACAGCGGGACGGATACGGCGCCACCCAAGCGTCTGACCT
C
ATCATCGATGACACCGAGCGGCCGCCACCGGATCTGGTCAAGCAGACGC
T
CAAGATGTTTGAGGCGAGTGCTAATCGACGACCGCGCGCAGCCCATCGT
T
CCAATGGTGTTGGCGGAGTGGCCAGCAAGGTGGCCAACTACAAATCGAT
C
ATTAAGGATCAGAAGCAACCATCATCAGCAGCGGCGACGCCACCGCCG
AC
CGGCATGGGTTTTGCCTCCTCAACGCCGCTAAGGCATGTCCATCCGGATA
TTATACCCCGTCAGGTGGATTCGCCCGTATCGGCGTTGAGTGTAATGATG
CGTCGCATGGAGCTGCAGGAGCCCGAGACGCCGGAAAGGGAGACACGG
GA
CGCGGAGGCCACACCGCAGAGCGAGGCGCTAAGTGAGACTGAGCGAAA
TG
ATCGCGATGAAGGCGATGGCGAAGTCGATGACGACAACCACAACAACA
AC
GACGACGATCACGACGACGGCGACGACGGCAGCGACAACAATGAGCAA
CG
CGATAAAATGGGCGCGGCGGCGGCAGGCGATAAGCCTAAGCCCCCAGC
GG
AATCGGCAGCGGGAGGCGCCCAACGATCATTCGCTGCCTCGACGGAGAA
C
GCGCATGTAGCCAGCGGTAGTAGCAGCAGTACCAGTGCCAGTGTACGGA
A
GCTCAGCAACAACAACGACTCCAGCAGCGGACCAGCGGTGACCAAACA
GA
TCGGTGTCATCCGTCCGCTATTCAATAGCCAGGGAGCCGGGAGCACGCC
G
CTAACGAGTCGCGAGATCGAAAAGAATCGCATCAATGAGATGAAGAAG
TC
GACGGCCACAGATGCCGGTGGTTCGGTATCCGGATCCGGAACGGTTGCT
G
GTGCTGGATCAGGAACCTCGCCCACCACTAGTCTCGACAGCGTGATAAA
C
ACCAAGGAAGCGGCCAGCAGCGAAACGGATGCCAGTGCCTCGCCACTG
TG
GACATTGCGCAAGCTGAGGAATCAGAGCCAGACGGCCGGCGGAGGATC
GG
GTCCCAGTTCGAGCCTTCATTCCACAGAGAACACCTCGATGGTGTTCAAC
TTTTCCAAGAGCACCAAGGAAGTGCCCGACTACATCGAAAGCGACGTTG
T
GATTTACAGGCGCAAGCGGGAGCTGCCAAAGCCAAATGAACCTGGCTTC
G
TGCTCCTGGGCGATCTCTCCGTGGAGACGTCGACGGACACGGACTACGA
C
GACTACTCCATGTGCCCGCCATCGCCGTGCGATGTGGAGTTTGAGAATG
C
CAACATTGTGATCGACGGCAAGTCCAGCATACGCCAGAAACCCAAAGA
GT
CTTCGTTCCGCGTGCAGTTCAACGACACGCTGACGTCGACGTTTGAATAC
CCCTCCGAGGCATCGATGACCATCGAGGATCCGCCGTACGCCGATCCCT
T
TGGCCATGTGAGCAAACACCATCAGATGCTGCTCGCCGAGCAGATGCAT
C
TGTTTGCAGCACCAGGAGCAGCTGGAGCTGGAGCAGATGCACCAGCTGG
GG
CTGGCGCCGGAGCAGCACCATCATGTGACCGTCGACGAGATCATCGAGC
T
GCCCACATCGACGGCGGGACATGGACATGGCATCGGACTTGGACACAG
GT
CGGGGGCGGCGGGGGGCGGTGGCACGATGCTGGGGAATTTACCGTTGG
AT
ATTATTTAAATGTAACAGAATACATAAGCAAAGCGTTGAAATAATATCT
A
TTGTATACCAAAACCAAAAATATCTATGATAACAAACAAGCAAAGGCAA
T
TAAACCGATCCGTAAACTGAATAACCGATTAATAACTGCAGCCGCTCAA
C
CCGAACCGCAATAAATAACTATCAAGTAAAGAATAGAACAACTTAACAA
T
GAAAAATGCAATATTCAATATGCGAAATGACAAATGCCTAATGCCAAAT
G
CCAATCCAAATAACCCAATAACCGATTCAAGTGCGAGAACAGAAATGCC
A
CACCTCTAACCCAGTGCACTTGCTCTGTCTGCTCCGCTGGGCAATCAAAC
TAATCGCCAATTCATATTATTATTAAGGCTCCACGGCTCTGGGCTTCTAC
ACGCCCATGAAGGGCACGGCGATGGACAACCTATTCCAGCTGGGTGTCA
C
CCGATACGCCCTGCCCGAGAGCAGCAGCAGTGGGAGCAGCAATGGCAG
CC
ACAGTCCAGGCAGCAACGGGAGCAGTCCAGCCGGAGCTGCGGGCAGCC
GG
TTGATGTTCAACGGGAATGGCAGCATTATGGGCATCGGCGAGGGACTGA
T
CAAGGAGGGCGACTTGGCGGCAACGGAAGCAGGACCTGGAACTGCAGA
TG
GAGCAGGTGCCTCCAAGAAGGGTTCAGCTGTGGACGAGGATGATTACCA
T
CTGACGGCGACGCCGGGCGCCGAAGTTGTGTACAGCGAGGGTACACAG
AA
AACGGATTTGCTATATTAGGTCGTCCTGCCAGCCAAAAAACCGGAGCGT
C
TAGCTCTTTTTCTACCGGTTTCCACCAAACTCCAAACCTTCCGAGCATAA
CAACCTACCACATCCTGATCGTTCCCCTAATTGAAAAGCGTGCACTTAGG
TGAAATATCAACTTCGGTTAGACGCGAAAACAGTAACGAAGGAAAGAA
AA
ATAAAAACAACAAGGCAACGTAAACCAAGTATGGCAAGAAATCCATAA
AG
ATACGTATATAAATAGATATTATTTGTAAGTTTGTAAGCCTAGAGATCCG
ATGTGTATGTGTACAACAGCAAGCGTGTGTGTCTGTCTTCTTTATAAATA
TATATATATGGTTTTTTTTTTTTGATAAATTAATAATTATATTATTGTAA
TGTATCTACATACGGCACTGAATGAACAACTTTGCTGCTGCAATTGTATT
TGTATTTGTATTGTGTATTCCATTTACGGCATTTTGATAGTAAATATACA
ACAACAACAAATCAACGTATTTCCGACTGCATCCCGAAGGTTACTGAAA
C
CTTAATCCACGAAGTACGCGACGAGCGGTTAGTCCCGCCCGAGTATTAA
C
TTAATGGATTCTAACGATCCGATCTTGTGACCCATCATCATCCCTTTTCG
CGACCCTTCCAAAACGAAAATGAAGAACATTCTAAGCTCCGCTTGCCCA
T
TCAGCGGAAGAAAGCAAAGGAAAATGTAACATTTGCCTCATAATTATTA
T
ATACATTTAGCGCTTATTGAATATTTTTTACAATTGTATTCCGT
>bif|FBgn0014133
MESQKRPSLDLHTDVPAGLAAGGSGLGAAAEMSPTSGFLPDMPQWKKDLI
QRRKTNVARTQAASITSPTDGSCGALAEANAAPGAIADFTEPATISSTSQ
KRNMIGSEEKSEKSSISNTNSDSTGGHHSVVAVSLSPDAAATTNVTVTPI
PKQRSSLLNTRSQEREMVRYILSESGERDGELESGEQPAGVVSNSRCGEV
ETGTIGSPSSSANQNPNPNHLKTKCKPGQSVAEGKPSAKETIVDNSKSCS
KTKSISDKLQSNKFIIQQQQQQQQQQQQQQQLSPTKVTVKPTMVAMQEMK
KTTKQNGQHRHLAGKIGSVAGGDVDPQHPPTNPIPDSLDTGEDLSYGPGI
VSKLRCRYLSLALRESRQQSSKQRLQRSTSLNTLLDRDDDEVEVEEPEMT
NSQVRAKSTPPPILGAKPTPKPSSQHQRPVSLGANGAGSAVNPPSNSDQA
QTGAVANGGAGSGQRSRHFKRGNEVMKRARSVEALLCEKSPWNSQRISTA
GAAAAAAPSPPAPKATAAAPSPVTSPTCVTIEDKIHNARERLHSGTDTAP
PKRLTSIIDTERPPPDLVKQTLKMFEASANRRPRAAHRSNGVGGVASKV
ANYKSIIKDQKQPSSAAATPPPTGMGFASSTPLRHVHPDIIPRQVDSPVS
ALSVMMRRMELQEPETPERETRDAEATPQSEALSETERNDRDEGDGEVDD
DNHNNNDDDHDDGDDGSDNNEQRDKMGAAAAGDKPKPPAESAAGGAQR
SF
AASTENAHVASGSSSSTSASVRKLSNNNDSSSGPAVTKQIGVIRPLFNSQ
GAGSTPLTSREIEKNRINEMKKSTATDAGGSVSGSGTVAGAGSGTSPTTS
LDSVINTKEAASSETDASASPLWTLRKLRNQSQTAGGGSGPSSSLHSTEN
TSMVFNFSKSTKEVPDYIESDVVIYRRKRELPKPNEPGFVLLGDLSVETS
TDTDYDDYSMCPPSPCDVEFENANIVIDGKSSIRQKPKESSFRVQFNDTL
TSTFEYPSEASMTIEDPPYADPFGHVSKHHQMLLAEQMHLLQHQEQLELE
QMHQLGLAPEQHHHVTVDEIIELPTSTAGHGHGIGLGHRSGAAGGGGTML
GNLPLDII
wap1^{Scim}
>>wap1|FBgn0004655|cDNA sequence
AATCGTTAGCCCAGCTCTAAAATAGTGTTTCTCTTCTCTCGTGTAGAAAA
ACAGAGAAATTAAACTTTTTCGCGCATTTCGCCATTGCAAAAATTGAAAT
TCGCGATCGCGTTCGTTTACCCTGCAAATTGCACAACTACGCGCTCTCGA
TTGGCGCCGATTTAGCGGAGAATCGCGAAATCAAAGAAATATTCGCAGC
A
AGAAGAAGAACGGAACGGCCAGTATAATCGTATTGTTGTTTGTGTTGGT
G
TTAGTTGTGTGTGTGTTTGTGGGTGTTGTTGTTGCTGCTGCTACTGCTTC
TGGGATGATGCTGTAAAGTAAAAGTGGTGGCAAAAGCACTAAGCCCGTG
G
TGCGACGCAAAAAACACGCTGCAGTTAGCGGCCAGGCGGAAAATGATA
AG
AGTGATGGTGGCTTGAGCCCCGAGTAATCGCTAAAATATCGAGCACACA
G
TTCGTGCAAATCAAATTGCGCCAATAAGCAAAAGCAAACGCAAACATCG
T
TTGTTTTATTGCATTCCACGCACACACACACCTATATATATGCACACGCA
CGCAGTCGCATACATATGCGCTGCAGCAGCAACAGAGATTTTTTTATTTG
ACCACACTCACTCACACACGCAACCACATGCATCCACGCAGCAAACGGA
T
AACAACAAAAACAAAGAGAGCAGCCAAATGTTTCGCCTGAGTGACCAC
AT
CACCAGGATTAGAGTCAGCAACACGGAGTTAGAGGATACCAGAAGCA
GC
AAAGGAACCACCGCTAGCGAAGATGTCGCGCTGGGGCAAGAACATCGT
GG
TGCCGCTGGACTCACTCTGCAAGGAGAAGGAGAACACCAACCGGCCCAC
T
GTCGCCCGTTCCGTGGGCACCGTTGGCAAGTGGGGCAAGATGGGCTTCA
C
CTCCACGCGCACCTACACCCTGCCCGCCATCCATCCTATGGCTGCGGCA
G
CGGCGGCTGCTGCAGCAGCCGCCAGTCCCTCCCAGAGTCCCGCCTCCAC
G
CAGGATCACGATCCCAACGACCTGTCCGTTTCGGTTCCGGAGCCGCCGA
A
GCCGAAAAAGTTCTTCAAATCGAGGAATACAGCTCCGCCGGAGGTCATA
G
CTCAGATCATTCAGCAGCTACCGCACTGTGGAGCCGGCGCATCACCCAT
G
CGAGACCATTTCTCGTCGGCGGGTGCGGGTGCTGGTGGTCTCACGCCTA
C
TTCCGGTGCCCAGGAGGCGGGCGGAGTGAAGCTGAAGCCGGGCAAGGG
CG
CCAGTTCCGCAGAGCGGAAGCGCAAATCGCCGAAAAAGAAGGCAGCAA
CA
ACGTCAGCCTCCACGCCCTCGACGCCTGGTGCGTTCTACGGCGCCTCCG
A
TCGAGATGGCGATGGGCTAAGCGATCCCGCCTCAGAGCAGCCGGAGCA
AC
CGTCCAGTGCCTCGGGCAAGCAGAAGCAGAAGAAGCCGAAGGAGGAGA
AG
AAGCTGAAGCCAGAGGCGCCGCCTTCGCGGGTACTGGGACGCGCCCGCA
A
GGCTGTCAACTACCGCGAAGTGGACGAGGACGAGCGCTATCCCACGCCC
A
CCAAGGATCTGATCATTCCCAAAGCGGGGCGCCAACCGGCTGAAGTGGC
G
GCTACGGCAACACTTGCCGCCGCTTCGTCGGAAGCCTTCATCAGTTCCAC
GTTTGGCAGCCCTGGATCGGAGCCGTCGTTACCCCCGCCTACGTCAGCG
C
CAAGTGCATCTGCGTCCACCTCGTCCCAACTGCCCTCCGCATCAGGCAG
C
GCGTCAAATCCTCCCAGCGCCTCCCGCACGCCAGAACATCCTCCTATCGT
GCTACGCATCTCCAAGGGCACTTCGCGGTTGGTCAGCACGGATAGCGAG
G
AGCCGCCGAGCAGCTCGCCCGCTCACCAGAACCAACTGAATCAACTTTC
G
GTCACGGAGGAGGAGCCAGCGGAGCGGTCCGGAGACGAAACAGTTCCT
GC
AAGTACGCCAAAAATCACAGTGAAGCCACTGAGGCCGCCCACAGCAGC
AG
ATTCCGTCGATGGATCATCTGCGGCAGTTGGAGGAGCATCAGCAGGCGA
T
TCTTTCGAGGAACGCAAGTCCCAGTCACTAGAACCCAACGAGGACGAGG
A
GGAGGAAGAAGAGGAGGAGGACGAGGAGGAGGAACCGCCGGAGATCA
ACT
ACTGCACGGTAAAGATATCCCCGGACAAACCGCCGAAGGAGCGGCTCA
AA
CTGATCATCAAGACGGACGTGATCCGCAACGCCATCGCCAAAGCCGCAG
C
AGCAGCCGAGTCCCGCAGTGAAAAGAAGTCGAGGAGCAAAAAGCACAA
GC
ACAAGCAGCTGCTTGCCGCGGGATCTGGTGCCGCTCCAGCTTCTGGAGC
C
ACCCCCGCCGAGATCAACTCCGAATTCAAGACACCCTCACCTCATTTGG
C
TCTCAGCGAGGCCAATAGTCAACAAGCACAGCATACACCATCTCATCTA
C
ATCAACTGCACCAGCTGCATCCGCAGCGAGGCTCCGCGGTCATATCACC
A
ACCACTCGATCCGATCACGACTTCGACTCGCAGTCCTCGGTGCTGGGCA
G
CATCTCCTCGAAGGGCAACAGCACGCCGCAACTGTTAGCGCAGGCTGTG
C
AGGAGGATAGTTGTGTGATTCGCAGCAGGGGATCTAGTGTGATCACCAG
T
GATCTAGAGACGAGTCAGCACTCCTCGCTGGTGGCTCCCCCCTCGGACA
T
TGAGTCACGACTGGAGTCCATGATGATGACCATCGACGGAGCGGGAACG
G
GAGCAGCATCTGCAGTGCCGGAGACACCACTGCAGGAGGACATACTGG
CT
GTGCTGCGAGGGGAAGTGCCACGGCTAAATGGCAATACGGACCCGGAG
CC
AACCGAGGAGGAGGATCAACAGCAACAGCCGAAGAGGGCCACGCGTGG
CA
GGGGCAGAAAGGCCAATAACAATGTGGATGTAACCCCACCCGCCACGG
AG
ACCAGAACCCGAGGTAGGGCCAAAGGAGCAGATGCGACCACGGCTGCC
AT
ATCGCCACCAACGGGCAAAAGAAACACGCGGGGCACCCGGGGCTCCAG
AA
AGGCCGAGCAGGAAGTCGACATGGAAGTGGACGAAACGGCGATGACGA
CG
GTGCCAGCGAACGAGGAACAGCTGGAACAGGCCACACTTCCACCGAGG
AG
AGGTCGCAATGCTGCTGCCCGAGCCAATAACAATAATTTGGCAAGCGTT
A
ACAATAACATTAATAAAATAGCCGCCAATTTGTCAGCCAAGGCCGAGGC
C
AGCCGACTAGCAGAAGGCGGAGTCGCGGGTGGAGCGGCACGAAGCTAT
GG
TCGGAAACGAAAGAACCAGCAGGTAACGCAGGTGCTACAGCAGGAGCC
AG
TGCCCGAAGAGCAGGAAACTCCTGATGCTGAGGAGGAGCAGCCCACAC
CC
GCCAAGATTCCGCACACAGATCACAGGGAACATTCGCCAGACCATGATC
C
GGATCCTGATCCGGATGAACTGTCGAACAACTCAAACAACTCCTCGTTG
C
AGCACGATGGGTCCTCCTCCTCGCCCCCACCCCGCGATTTCAAGTTTAAG
GATAAGTTTAAGCGAACATTGACACTGGACACACAGGGCGCGGCGAAC
GC
CGGAGCGGGAGGAGCAGCAGCGGCGGCGCCACCTGAGTCCTCTGGCGA
AC
AGCGGGGCGCTGTCAAGCTGGTCATCTCAAAGAAGAAAGGCAGCATCTT
C
AAAAGCCGCGCTCTGGTGCCATCCGATCAAGCGGAACAGGCTACAGTGG
C
CAAGCGACATCTGTACAAGCACAGTTGGGATGCTGCGCTAGAAGCGAAT
G
GCGGTGGAACCAACAGCGATGCCAGCAATGCCTCGGCATCTGGCGTGGG
C
GTCGCTGGGGCGAAGGATCATCTGCATCATTTAGCGGCGGGCAAGTCCG
A
TGGTGATTTCGGTGACAGTCCGTCGTCGAACAACAATGGCTCCTCCAGT
G
CGTGCAGCAGCGCATCCACGTTGCGCGGCGATAGCCCGGCCCTCGGAAA
G
ATCTCGCGACTGGCGGGAAAACAGGGAGTACCTGCCACCTCCACTAGTT
C
CGATGCCTTTGACCTGGATTTGGAACCAATTGCCGGAGAGCTTGACCTA
G
AGCGTAGTGCAGCTGGTGCTTCCGCTGGTGGAACAGGGGCAACGACAGG
C
GGAGTGGAGCGACGGGCGGTGGCGGCCCCGTTCGGGTCGACCGCAAA
AC
TAAGGACTACTATCCAGTGGTGCGTAACGTTAAGACGGCCCATCAAATT
C
AGGAGATCGGCGAGTACCAGGAAATGGACGACGACGTCGAGTACATCC
TG
GACGCACTTCAGCCGCACAATCCCCCGGCAACACGGTGTCTCTCCGCCC
T
TCAGCTAGCCGCCAAGTGTATGATGCCCGCCTTCCGGATGCACGTGCGT
G
CCCATGGGGTGGTCACCAAATTTTTCAAGGCATTATCCGACGCCAACAA
G
GACCTCAGCCTAGGCCTGTGCACCTCGGCCATCATGTACATTCTTTCCCA
GGAGGGCCTCAACATGGACCTGGATCGCGACTCCTTGGAGCTGATGATC
A
ACCTTCTGGAGGCGGACGGCGTGGGTGGCAGCACGGAGACTGGGCACC
CG
GATAGAGCGGGCTACGACCGCAACAAGCAGAAGGTGCGCGAGTTGTGC
GA
GGAGATCAAGGCGCAGGGCAAGGGAACGCATCTCAACGTTGATTCTCTG
A
CTGTGGGCACGCTGGCAATGGAAACGCTGCTATCGCTAACATCCAAGCG
C
GCGGGCGAGTGGTTTAAAGAGGATCTGCGCAAGCTGGGTGGCCTGGAGC
A
CATTATCAAGACCATCTCGGACTTCTGCAGACCGGTGATTGCCTGCGAC
A
CGGAGATTGACTGGCAGCCGACGCTGCTGGATAACATGCAAACGGTGGC
G
CGTTGTCTGCGAGTCCTCGAAAACGTGACGCAGCACAATGAGACGAACC
A
GCGCTACATGCTCACCTCTGGCCAGGGAAAAGCAGTGGAGACGCTTTGC
C
AACTGTACCGTCTTTGCAGCCGACAAATAATGCTGCATCCTTCGGATGGT
GGTGGCAGCAACAAGGAGCATCCGGGTGTGGCTATGCGCGAGCTGCTGG
T
GCCGGTGCTCAAGGTACTGATCAACCTGACGCACACGTTCAACGAGGCG
C
AGCCATCGTTGGGAGCCGAGCTGCTAGGTCAAAGGGGCGATGTGGTGGA
G
ACGAGCTTCCGATTGCTGCTGCTCTCGGCCAACTACATTCCCGACCAATG
TGTCTTTGAGCTAAGCATACTGGTTCTTACACTGCTAATCAATTTGTGCA
TGCATACTGTGCCCAACCGGGCTGCTCTAATGCAAGCTGCCGCTCCGGC
A
GAGTACGTAGCGGATAATCCACCAGCGCAGGGATCTGTGAGTGCACTGC
A
AGCTCTGCTTGAGTACTTCTACAAGTGCGAGGAGCTGGCTAGATTGGTG
G
AAAAGAACACGGACGCCTTCCTCGAGAGCAACGAGAAGGGAAAGAAGA
AA
CAAGAAGAAGTGGAGGAGACAGTCAACAATCTTGTGCAACGAGCCGGC
CA
CCACATGGAGCACACGCTAAAGGGAAGTTATGCGGCCATCCTGGTGGGA
A
ATCTGATAGCGGACAACGAGTTGTACGAGTCGGTGGTGCGCCGCCAGCT
G
CGAGGAAATAGCTTTAAGGAGATTATTGGAGTGCTGGAGAAGTACCACA
C
ATTCATGAACCTTACATCCAGCTTGGAGGCAGCCTTTGTGGCGCATATGA
AGTCCACGAAGCGCATCATCGACAACTTTAAGAAGCGCGACTACATCTA
C
GAGCACTCGGATGAGCACGACAACCCCCTGCCTCTGAATCTGGAAACGA
C
GGCGCAAGTCTTGGCCGTGGGAGCGGACGCGTCGCATGCTGCTACAAGC
T
CGACCACGGTCGGCTCTGGCTCCGCACCCTCATCCACATCGGCCACAGG
A
ACGACGAGGGCGCCGCGGGTCTATAAAACGTACAGCAGCCACAGGTAA
TC
GGACCTGGGATCTGAATCGGAATCGAAGAGCCATCGCAATTGTCGTTAA
C
CACACCGAAAGCCTGCCACCAAAATAAAATTCTGTTGTTTGTGATTATGT
AATTTGTTTATGTGTTGATATAACAATCACTACATGTATGTGTATAGCCT
GTAATGCACCTAGTTTCAGTCAGCTATATATATATAGATATATATATATA
TAGAAGTATATAGCTAAGAGCGATAGGATACAATGCGATCTGTTTTGC
G
TTTGTATTAACCCAAAGTGATAGCGAATTCCTGAGCATGGTCTGCGTTTT
AACTAAAGAAAATGATTAAGATTGACGAAACGAAAGCGACACCCCGAA
CA
ACACAAAGATCCAAATCCCCACTAATATGAGTACGTTATATAGTCATAA
G
CAATATTAGCAGCTAAATGTCGGCTTACCCTCGAAAGCACACACCCATG
T
AAATATTCTTTAATCAGCGATTTTATCGGACGCGTCTTAGGTTTATTTCG
AATGTGTTTTTTTTTTACCTATTCATACTTTATATATTAAACCTTTTTTT
TATTGATTGTAAAACAAGCGCTCTTAGCAAAAACGCTGCAGCAGAAAGA
G
CGAGAGCGATAGACATGTGTAAAGAGAGAGTTAGAGAGGAAAATTCTA
GA
TTAGATGTGGGAAATAAAGTTATTTTATTGCCTAAACGTAAGCTAAAGA
A
ACTCTACTTATAAAACTAACACTGATAAATAAATATATATATGTATATGG
A
<<wap1|FBgn0004655|cDNA sequence
GAATTGGAGTCGAGTCGCTCTCCCGAGTTGTTGGTGTTTTGTTTTTTGTT
CCGCATTTATCTGTCGGTTCTTGCCCATTTCGGTTCACTTGGAGTGCTAA
TTACCGGGAACGCGACATAGTTGTGCTTATTTTCATTTGCCAGATCAGCT
AGCCAAAAGGCACTTCGCGGTTGGTCAGCACGGATAGCGAGGAGCCGC
CG
AGCAGCTCGCCCGCTCACCAGAACCAACTGAATCAACTTTCGGTCACGG
A
GGAGGAGCCAGCGGAGCGGTCCGGAGACGAAACAGTTCCTGCAAGTAC
GC
CAAAAATCACAGTGAAGCCACTGAGGCCGCCCACAGCAGCAGATTCCGT
C
GATGGATCATCTGCGGCAGTTGGAGGAGCATCAGCAGGCGATTCTTTCG
A
GGAACGCAAGTCCCAGTCACTAGAACCCAACGAGGACGAGGAGGAGGA
AG
AAGAGGAGGAGGACGAGGAGGAGGAACCGCCGGAGATCAACTACTGCA
CG
GTAAAGATATCCCCGGACAAACCGCCGAAGGAGCGGCTCAAACTGATC
AT
CAAGACGGACGTGATCCGCAACGCCATCGCCAAAGCCGCAGCAGCAGC
CG
AGTCCCGCAGTGAAAAGAAGTCGAGGAGCAAAAAGCACAAGCACAAGC
AG
CTGCTTGCCGCGGGATCTGGTGCCGCTCCAGCTTCTGGAGCCACCCCCGC
CGAGATCAACTCCGAATTCAAGACACCCTCACCTCATTTGGCTCTCAGCG
AGGCCAATAGTCAACAAGCACAGCATACACCATCTCATCTACATCAACT
G
CACCAGCTGCATCCGCAGCGAGGCTCCGCGGTCATATCACCAACCACTC
G
ATCCGATCACGACTTCGACTCGCAGTCCTCGGTGCTGGGCAGCATCTCCT
CGAAGGGCAACAGCACGCCGCAACTGTTAGCGCAGGCTGTGCAGGAGG
AT
AGTTGTGTGATTCGCAGCAGGGGATCTAGTGTGATCACCAGTGATCTAG
A
GACGAGTCAGCACTCCTCGCTGGTGGCTCCCCCCTCGGACATTGAGTCA
C
GACTGGAGTCCATGATGAATGACCATCGACGGAGCGGGAACGGGAGCAG
CA
TCTGCAGTGCCGGAGACACCACTGCAGGAGGACATACTGGCTGTGCTGC
G
AGGGGAAGTGCCACGGCTAAATGGCAATACGGACCCGGAGCCAACCGA
GG
AGGAGGATCAACAGCAACAGCCGAAGAGGGCCACGCGTGGCAGGGGCA
GA
AAGGCCAATAACAATGTGGATGTAACCCCACCCGCCACGGAGACCAGA
AC
CCGAGGTAGGGCCAAAGGAGCAGATGCGACCACGGCTGCCATATCGCC
AC
CAACGGCAAAAGAAACACGCGGGGCACCCGGGGCTCCAGAAAGGCCG
AG
CAGGAAGTCGACATGGAAGTGGACGAAACGGCGATGACGACGGTGCCA
GC
GAACGAGGAACAGCTGGAACAGGCCACACTTCCACCGAGGAGAGGTCG
CA
ATGCTGCTGCCCGAGCCAATAACAATAATTTGGCAAGCGTTAACAATAA
C
ATTAATAAAATAGCCGCCAATTTGTCAGCCAAGGCCGAGGCCAGCCGAC
T
AGCAGAAGGCGGAGTCGCGGGTGGAGCGGCACGAAGCTATGGTCGGAA
AC
GAAAGAACCAGCAGGTAACGCAGGTGCTACAGCAGGAGCCAGTGCCCG
AA
GAGCAGGAAACTCCTGATGCTGAGGAGGAGCAGCCCACACCCGCCAAG
AT
TCCGCACACAGATCACAGGGAACATTCGCCAGACCATGATCCGGATCCT
G
ATCCGGATGAACTGTCGAACAACTCAAACAACTCCTCGTTGCAGCACGA
T
GGGTCCTCCTCCTCGCCCCCACCCCGCGATTTCAAGTTTAAGGATAAGTT
TAAGCGAACATTGACACTGGACACACAGGGCGCGGCGAACGCCGGAGC
GG
GAGGAGCAGCAGCGGCGGCGCCACCTGAGTCCTCTGGCGAACAGCGGG
GC
GCTGTCAAGCTGGTCATCTCAAAGAAGAAAGGCAGCATCTTCAAAAGCC
G
CGCTCTGGTGCCATCCGATCAAGCGGAACAGGCTACAGTGGCCAAGCGA
C
ATCTGTACAAGCACAGTTGGGATGCTGCGCTAGAAGCGAATGGCGGTGG
A
ACCAACAGCGATGCCAGCAATGCCTCGGCATCTGGCGTGGGCGTCGCTG
G
GGCGAAGGATCATCTGCATCATTTAGCGGCGGGCAAGTCCGATGGTGAT
T
TCGGTGACAGTCCGTCGTCGAACAACAATGGCTCCTCCAGTGCGTGCAG
C
AGCGCATCCACGTTGCGCGGCGATAGCCCGGCCCTCGGAAAGATCTCGC
G
ACTGGCGGGAAAACAGGGAGTACCTGCCACCTCCACTAGTTCCGATGCC
T
TTGACCTGGATTTGGAACCAATTGCCGGAGAGCTTGACCTAGAGCGTAG
T
GCAGCTGGTGCTTCCGCTGGTGGAACAGGGGCAACGACAGGCGGAGGT
GG
AGCGACGGGCGGTGGCGGCCCCGTTCGGGTCGACCGCAAAACTAAGGA
CT
ACTATCCAGTGGTGCGTAACGTTAAGACGGCCCATCAAATTCAGGAGAT
C
GGCGAGTACCAGGAAATGGACGACGACGTCGAGTACATCCTGGACGCA
CT
TCAGCCGCACAATCCCCCGGCAACACGGTGTCTCTCCGCCCTTCAGCTA
G
CCGCCAAGTGTATGATGCCCGCCTTCCGGATGCACGTGCGTGCCCATGG
G
GTGGTCACCAAATTTTTCAAGGCATTATCCGACGCCAACAAGGACCTCA
G
CCTAGGCCTGTGCACCTCGGCCATCATGTACATTCTTTCCCAGGAGGGCC
TCAACATGGACCTGGATCGCGACTCCTTGGAGCTGATGATCAACCTTCTG
GAGGCGGACGGCGTGGGTGGCAGCACGGAGACTGGGCACCCGGATAGA
GC
GGGCTACGACCGCAACAAGCAGAAGGTGCGCGAGTTGTGCGAGGAGAT
CA
AGGCGCAGGGCAAGGGAACGCATCTCAACGTTGATTCTCTGACTGTGGG
C
ACGCTGGCAATGGAAACGCTGCTATCGCTAACATCCAAGCGCGCGGGCG
A
GTGGTTTAAAGAGGATCTGCGCAAGCTGGGTGGCCTGGAGCACATTATC
A
AGACCATCTCGGACTTCTGCAGACCGGTGATTGCCTGCGACACGGAGAT
T
GACTGGCAGCCGACGCTGCTGGATAACATGCAAACGGTGGCGCGTTGTC
T
GCGAGTCCTCGAAAACGTGACGCAGCACAATGAGACGAACCAGCGCTA
CA
TGCTCACCTCTGGCCAGGGAAAAGCAGTGGAGACGCTTTGCCAACTGTA
C
CGTCTTTGCAGCCGACAAATAATGCTGCATCCTTCGGATGGTGGTGGCA
G
CAACAAGGAGCATCCGGGTGTGGCTATGCGCGAGCTGCTGGTGCCGGTG
C
TCAAGGTACTGATCAACCTGACGCACACGTTCAACGAGGCGCAGCCATC
G
TTGGAGCCGAGCTGCTAGGTCAAAGGGGCGATGTGGTGGAGACGAGCT
T
CCGATTGCTGCTGCTCTCGGCCAACTACATTCCCGACCAATGTGTCTTTG
AGCTAAGCATACTGGTTCTTACACTGCTAATCAATTTGTGCATGCATACT
GTGCCCAACCGGGCTGCTCTAATGCAAGCTGCCGCTCCGGCAGAGTACG
GTGCCCAACCGGGCTGCTCTAATGCAAGCTGCCGCTCCGGCAGAGTACG
T
AGCGGATAATCCACCAGCGCAGGGATCTGTGAGTGCACTGCAAGCTCTG
C
TTGAGTACTTCTACAAGTGCGAGGAGCTGGCTAGATTGGTGGAAAAGAA
C
ACGGACGCCTTCCTCGAGAGCAACGAGAAGGGAAAGAAGAAACAAGAA
GA
AGTGGAGGAGACAGTCAACAATCTTGTGCAACGAGCCGGCCACCACATG
G
AGCACACGCTAAAGGGAAGTTATGCGGCCATCCTGGTGGGAAATCTGAT
A
GCGGACAACGAGTTGTACGAGTCGGTGGTGCGCCGCCAGCTGCGAGGA
AA
TAGCTTTAAGGAGATTATTGGAGTGCTGGAGAAGTACCACACATTCATG
A
ACCTTACATCCAGCTTGGAGGCAGCCTTTGTGGCGCATATGAAGTCCAC
G
AAGCGCATCATCGACAACTTTAAGAAGCGCGACTACATCTACGAGCACT
C
GGATGAGCACGACAACCCCCTGCCTCTGAATCTGGAAACGACGGCGCAA
G
TCTTGGCCGTGGGAGCGGACGCGTCGCATGCTGCTACAAGCTCGACCAC
G
GTCGGCTCTGGCTCCGCACCCTCATCCACATCGGCCACAGGAACGACGA
G
GGCGCCGCGGGTCTATAAAACGTACAGCAGCCACAGGTAATCGGACCTG
G
GATCTGAATCGGAATCGAAGAGCCATCGCAATTGTCGTTAACCACACCG
A
AAGCCTGCCACCAAAATAAAATTCTGTTGTTTGTGATTATGTAATTTGTT
TATGTGTTGATATAACAATCACTACATGTATGTGTATAGCCTGTAATGCA
CCTAGTTTCAGTCAGCTATATATATATAGATATATATATATATAGAAGTA
TATAGCTAAGAGCGATAGGATACAATGCGATCTGTTTTGCGTTTGTATT
AACCCAAAGTGATAGCGAATTCCTGAGCATGGTCTGCGTTTTAACTAAA
G
AAAATGATTAAGATTGACGAAACGAAAGCGACACCCCGAACAACACAA
AG
ATCCAAATCCCCACTAATATGAGTACGTTATATAGTCATAAGCAATATTA
GCAGCTAAATGTCGGCTTACCCTCGAAAGCACACACCCATGTAAATATT
C
TTTAATCAGCGATTTTATCGGACGCGTCTTAGGTTTATTTCGAATGTGTT
TTTTTTTTACCTATTCATACTTTATATATTAAACCTTTTTTTTATTGATT
GTAAAACAAGCGCTCTTAGCAAAAACGCTGCAGCAGAAAGAGCGAGAG
CG
ATAGACATGTGTAAAGAGAGAGTTAGAGAGGAAAATTCTAGATTAGATG
T
GGGAAATAAAGTTATTTTATTGCCTAAACGTAAGCTAAAGAAACTCTAC
T
TATAAAACTAACACTGATAAATAAATATATATATGTATATGGA
>wap1|FBgn0004655
MSRWGKNIVVLDSLCKEKENTNRPTVARSVGTVGKWGKMGFTSTRTYTL
PAIHPMAAAAAAAAAAASPSQSPASTQDHDPNDLSVSVPEPPKPKKFFKS
RNTAPPEVIAQIIQQLPHCGAGASPMRDHFSSAGAGAGGLTPTSGAQEAG
GVKLKPGKGASSAERKRKSPKKKAATTSASTPSTPGAFYGASDRDGDGLS
DPASEQPEQPSSASGKQKQKKPKEEKKLKPEAPPSRVLGRARKAVNYREV
DEDERYPTPTKDLIIPKAGRQPAEVAATATLAAASSEAFISSTFGSPGSE
PSLPPPTSAPSASASTSSQLPSASGSASNPPSASRTPEHPPIVLRISKGT
SRLVSTDSEEPPSSSPAHQNQLNQLSVTEEEPAERSGDETVPASTPKITV
KPRPPTAADSVDGSSAAVGGASAGDSFEERKSQSLEPNEDEEEEEEEED
EEEEPPEINYCTVKISPDKPPKERLKLIIKTDVIRNAIAKAAAAAESRSE
KKSRSKKHKHKQLLAAGSGAAPASGATPAEINSEFKTPSPHLALSEANSQ
QAQHTPSHLHQLHQLHPQRGSAVISPTTRSDHDFDSQSSVLGSISSKGNS
TPQLLAQAVQEDSCVIRSRGSSVITSDLETSQHSSLVAPPSDIESRLESM
MMTIDGAGTGAASAVPETPLQEDILAVLRGEVPRLNGNTDPEPTEEEDQQ
QQPKRATRGRGRKANNNVDVTPPATETRTRGRAKGADATTAAISPPTGKR
NTRGTRGSRKAEQEVDMEVDETAMTTVPANEEQLEQATLPPRRGRNAAAR
ANNNNLASVNNNINKIAANLSAKAEASRLAEGGVAGGAARSYGRKRKNQQ
VTQVLQQEPVPEEQETPDAEEEQPTPAKIPHTDHREHSPDHDPDPDPDEL
SNNSNNSSLQHDGSSSSPPPRDFKFKDKFKRTLTLDTQGAANAGAGGAAA
AAPPESSGEQRGAVKLVISKKKGSIFKSRALVPSDQAEQATVAKRHLYKH
SWDAALEANGGGTNSDASNASASGVGVAGAKDHLHHLAAGKSDGDFGDS
P
SSNNNGSSSACSSASTLRGDSPALGKISRLAGKQGVPATSTSSDAFDLDL
EPIAGELDLERSAAGASAGGTGATTGGGGATGGGGPVRVDRKTKDYYPVV
RNVKTAHQIQEIGEYQEMDDDVEYILDALQPHNPPATRCLSALQLAAKCM
MPAFRMHVRAHGVVTKFFKALSDANKDLSLGLCTSAIMYILSQEGLNMDL
DRDSLELMINLLEADGVGGSTETGHPDRAGYDRNKQKVRELCEEIKAQGK
GTHLNVDSLTVGTLAMETLLSLTSKRAGEWFKEDLRKLGGLEHIIKTISD
FCRPVIACDTEIDWQPTLLDNMQTVARCLRVLENVTQHNETNQRYMLTSG
QGKAVETLCQLYRLCSRQIMLHPSDGGGSNKEHPGVAMRELLVPVLKVLI
NLTHTFNEAQPSLGAELLGQRGDVVETSFRLLLLSANYIPDQCVFELSIL
VLTLLINLCMHTVPNRALLMQAAAPEYVADNPPAQGSVSALQALLEYFY
KCEELARLVEKNTDAFLESNEKGKKKQEEVEETVNNLVQRAGHHMEHTLK
GSYAAILVGNLIADNELYESVVRRQLRGNSFKEIIGVLEKYHTFMNLTSS
LEAAFVAHMKSTKRIIDNFKKRDYIYEHSDEHDNPLPLNLETTAQVLAVG
ADASHAATSSTTVGSGSAPSSTSATGTTRAPRVYKTYSSHR
>wap1|FBgn0004655
MTIDGAGTGAASAVPETPLQEDILAVLRGEVPRLNGNTDPEPTEEEDQQQ
QPKRATRGRGRKANNNVDVTPPATETRTRGRAKGADATTAAISPPTGKRN
TRGTRGSRKAEQEVDMEVDETAMTTVPANEEQLEQATLPPRRGRNAAARA
NNNNLASVNNNINKIAANLSAKAEASRLAEGGVAGGAARSYGRKRKNQQV
TQVLQQEPVPEEQETPDAEEEQPTPAKIPHTDHREHSPDHDPDPDPDELS
NNSNNSSLQHDGSSSSPDDRDFKFKDKFKRTLTLDTQGAANAGAGGAAAA
APPESSGEQRGAVKLVISKKKGSIFKSRALVPSDQAEQATVAKRHLYKHS
WDAALEANGGGTNSDASNASASGVGVAGAKDHLHHLAAGKSDGDFGDSP
S
SNNNGSSSACSSASTLRGDSPALGKISRLAGKQGVPATSTSSDAFDLDLE
PIAGELDLERSAAGASAGGTGATTGGGGATGGGGPVRVDRKTKDYYPVVR
NVKTAHQIQEIGEYQEMDDDVEYILDALQPHNPPATRCLSALQLAAKCMM
PAFRMHVRAHGVVTKFFKALSDANKDLSLGLCTSAIMYILSQEGLNMDLD
RDSLELMINLLEADGVGGSTETGHPDRAGYDRNKQKVRELCEEIKAQGKG
THLNVDSLTVGTLAMETLLSLTSKRAGEWFKEDLRKLGGLEHIIKTISDF
CRPVIACDTEIDWQPTLLDNMQTVARCLRVLENVTQHNETNQRYMLTSGQ
GKAVETLCQLYRLCSRQIMLHPSDGGGSNKEHPGVAMRELLVPVLKVIIN
LTHTFNEAQPSLGAELLGQRGDVVETSFRLLLLSANYIPDQCVFELSILV
LTLLINLCMHTVPNRAALMQAAAPAEYVADNPPAQGSVSALQALLEYFYK
CEELARLVEKNTDAFLESNEKGKKKQEEVEETVNNLVQRAGHHMEHTLKG
SYAAILVGNLIADNELYESVVRRQLRGNSFKEIIGVLEKYHTFMNLTSSL
EAAFVAHMKSTKRIIDNFKKRDYIYEHSDEHDNPLPLNLETTAQVLAVGA
DASHAATSSTTVGSGSAPSSTSATGTTRAPRVYKTYSSHR
grp^{Scim}
>>grp⊕FBgn0011598|cDNA sequence
TATATTTTAGCAGCGTTTGCGGAATTTTTTGCATTTCAGTTACTCCAAGT
GTGAATAAGAGCACGGTCGCTGTTATAGTGTCTTGCAAAGCAGTGAAT
A
AAAAAGTTTAATAATCCAAATCGAGAATCCCAAATTTGTGTAGACGTAG
C
AACAACAGTAAAACGCGCTGGCCGGAAAAGACGCTGCTGGTAGTGATTC
C
CAATCAGCTTTGTTTACAAGGTCCACTCCAATTCCTACGCACTCGAGTTG
TGGCGAGAGTTTTTTATGCGACATCAACTGAAGCTGGAGCTGCCGAAAG
A
TATGCACGAGGTGGCGAAAGCTGAGCTAGAATCGGGCGGCAGATGACA
AG
GACAACGCCACGCACATGGCGGCAAGGCTACAACAAGGATACAGGATA
CG
GCCAACCGGATATTAAACCATCAACACTAGCCAGCACAGCAAAGCAAA
AT
TAAGGAACAACTGCAATCCCGGTACACCAGTACACCATGGCTGCAACGC
T
GACGGAAGCGGGAACAGGTCCTGCGGCCACCAGGGAGTTCGTCGAGGG
AT
GGACTTTGGCCCAAACTCTGGGCGAAGGTGCCTACGGCGAGGTAAAGCT
A
CTAATCAACCGGCAGACTGGCGAGGCTGTGGCCATGAAAATGGTGGATC
T
AAAAAAACATCCGGATGCAGCGAACTCGGTGCGAAAGGAGGTATGCAT
AC
AGAAGATGCTCCAGGATAAGCATATCCTCCGATTTTTCGGCAAACGTTC
G
CAAGGCAGTGTGGAGTACATATTCCTGGAATACGCCGCCGGCGGAGAGC
T
ATTCGATCGAATAGGTGAGGAACCAGACGTGGGAATGCCGCAGCATGA
GG
CTCAAAGGTATTTTACACAGCTCCTGTCCGGACTCAATTACCTGCATCAG
CGTGGGATCGCTCATCGGGATCTGAAGCCGGAAAATCTGCTGCTTGACG
A
GCATGACAACGTGAAAATATCGGACTTTGGCATGGCTACTATGTTTAGG
T
GCAAGGGCAAGGAGCGACTGCTGGACAAACGCTGCGGCACCTTGCCGT
AT
GTGGCGCCCGAGGTGCTACAGAAGGCATATCACGCCCAGCCGGCGGATC
T
CTGGTCGTGTGGCGTTATATTGGTGACAATGCTGGCGGGTGAGCTGCCCT
GGGATCAGCCGTCCACCAATTGCACGGAGTTCACCAACTGGAGGGATAA
C
GATCACTGGCAACTGCAGACTCCTTGGAGCAAACTGGACACCTTGGCTA
T
TTCGCTGCTCGCAAGCTGCTGGCCACCAGTCCTGGCACGCGTTTGACCC
TGGAGAAAACCCTGGATCACAAATGGTGCAACATGCAGTTTGCAGACAA
T
GAACGTTCCTATGACCTGGTGGACTCGGCGGCTGCCCTGGAGATATGCT
C
GCCAAAGGCTAAGAGGCAGCGTCTGCAGTCTAGTGCCCACTTGAGCAAT
G
GCCTGGATGACTCCATCTCCCGGAACTACTGCTCTAACCCATGCCCACA
ATGCGCAGCGACGATGACTTCAATGTCAGACTGGGCAGTGGCCGATCCA
A
GGAGGATGGAGGCGACCGCCAGACGTTGGCCCAGGAGGCTCGGCTCAG
TT
ACTCCTTCTCGCAACCAGCTTTGCTTGATGATCTCCTACTGGCTACCCAG
ATGAACCAAACGCAGAACGCTTCCCAGAACTATTTCCAGCGTTTGGTGA
G
GAGAATGACCCGATTCTTTGTGACCACACGATGGGATGACACTATCAAG
C
GATTGGTGGGAACCATCGAAAGACTGGGTGGTTATACGTGCAAATTTGG
T
GACGACGGAGTGGTCACCGTATCCACAGTCGATCGGAATAAGCTGCGAT
T
GGTTTTCAAGGCACACATTATAGAGATGGATGGCAAGATTCTTGTTGACT
GCCGGCTGTCAAAGGGTTGCGGCTTGGAGTTTAAACGACGATTTATCAA
G
ATCAAAAACGCCCTGGAGGATATCGTGCTTAAAGGACCCACCACCTGGC
C
GATTGCGATTGCTACAAATTCGGTGCCTTAGCTTTTAGTTTCATTCGAAC
TTAAAATCCTGTCCTGTCTTGTATTCGGTTATATTTACATTTGTCTAGCC
TGTAAGAGCAACTCATGCATCACCTTCGATCAGAATCATCTAAAATTCTC
AATGTGCTAACTTATTTTTAATTCATTGCATTGTTTACGAGTACGTTGTG
TATTGCTAAGCGGGCTCTCATTCGATTTTAATTTTGTTGAATTTTAAAGC
AAAAGTCGAGTATTGAAAACTAGTTTAAATGTTATCGAACAATAAATCT
T
ATGCGATTTTGAGTAGAAAA
>>grp|FBgn0011598|cDNA sequence
GTCCCACTGTGCACTGCTGAACTTGAGCGAGAGAGCAAGTTGAACTTGC
C
AAATGCTGTGCGGAAGAGGAAGAGCGAGAGGATTTCTCCAACCCCTTCC
C
GCCTATCGTTTTTGGCGCGCGCAAGCGTAAGCTCTCCCTGCTCTCGCGGC
CTCTCCGACGCTCTCTGGAAGTTTCGAGTTTCGGGCGAGGCTTTCTCGGT
TCGGCGAACCTAGGCGGCAGTTTGATTTCAACTCTCAGACAGGACGAAG
C
GCCTGCCACAGGATATGGCTGGGAAACTATAGTGGAAACCTACGCCGCT
A
TGTAATAGTAAATTAATAGTAGTTCTCTACTACTGCAGTAGTAGGCGGCG
CTTTTCAATCGTATTGAAAGGAATACAATGACGGGGGCAAGAGAGAAC
G
AACGGAGAAAGAGTGCACGGACCACGAGGCTGTTTCATTAAAATTGGA
T
ACAGACAAAACGTGGCTCGCAGGATACCGGATACGTCTGTGTGCGTGTG
T
GTGCCGCATCCATTCAGGAGGCTAAGAAAAAGGACGGCACACAGTTTTC
A
GTGGTGACTTTCTCGGCAAAATCCTAAAAAAGGACAAAACGGTTCGGTT
C
CTATTTGGTTTCTCGACTTTCGGTCTTCGGGGTAGTTCCTTAGTCTCTCA
GAATAGCAAGATGATAAACCTGAAAAAGAAGAAGAAGCCGTCCAAGAA
GC
AGCTTCGCAAAAATTCCGCGCTTTGGCTCTCAATCGCCAACAATTCGTCG
GAAATCAAGAAAAATATATGCACGAGGTGGCGAAAGCTGAGCTAGAAT
CG
GGCGGCAGATGACAAGGACAACGCCACGCACATGGCGGCAAGGCTACA
AC
AAGGATACAGGATACGGCCAACCGGATATTAAACCATCAACACTAGCCA
G
CACAGCAAAGCAAAATTAAGGAACAACTGCAATCCCGGTACACCAGT
CA
CCATGGCTGCAACGCTGACGGAAGCGGGAACAGGTCCTGCGGCCACCA
GG
GAGTTCGTCGAGGGATGGACTTTGGCCCAAACTCTGGGCGAAGGTGCCT
A
CGGCGAGGTAAAGCTACTAATCAACCGGCAGACTGGCGAGGCTGTGGCC
A
TGAAAATGGTGGATCTAAAAAAACATCCGGATGCAGCGAACTCGGTGCG
AAGGAGGTATGCATACAGAAGATGCTCCAGGATAAGCATATCCTCCGAT
T
TTTCGGCAAACGTTCGCAAGGCAGTGTGGAGTACATATTCCTGGAATAC
G
CCGCCGGCGGAGAGCTATTCGATCGAATAGGTGAAGAACCAGACGTGG
GA
ATGCCGCAGCATGAGGCTCAAAGGTATTTTACACAGCTCCTGTCCGGAC
T
CAATTACCTGCATCAGCGTGGGATCGCTCATCGGGATCTGAAGCCGGAA
A
ATCTGCTGCTTGACGAGCATGACAACGTGAAAATATCGGACTTTGGCAT
G
GCTACTATGTTTAGGTGCAAGGGCAAGGAGCGACTGCTGGACAAACGCT
G
CGGCACCTTGCCGTATGTGGCGCCCGAGGTGCTACAGAAGGCATATCAC
G
CCCAGCCGGCGGATCTCTGGTCGTGTGGCGTTATATTGGTGACAATGCTG
GCGGGTGAGCTGCCCTGGGATCAGCCGTCCACCATTGCACGGAGTTCA
C
CAACTGGAGGGATAACGATCACTGGCAACTGCAGACTCCTTGGAGCAAA
C
TGGACACCTTGGCTATTTCGCTGCTTCGCAAGCTGCTGGCCACCAGTCCT
GGCACGCGTTTGACCCTGGAGAAAACCCTGGATCACAAATGGTGCAACA
T
GCAGTTTGCAGACAATGAACGTTCCTATGACCTGGTGGACTCGGCGGCT
G
CCCTGGAGATATGCTCGCCAAAGGCTAAGAGGCAGCGTCTGCAGTCTAG
T
GCCCACTTGAGCAATGGCCTGGATGACTCCATCTCCCGGAACTACTGCTC
TCAACCCATGCCCACAATGCGCAGCGACGATGACTTCAATGTCAGACTG
G
GCAGTGGCCGATCCAAGGAGGATGGAGGCGACCGCCGACGTTGGCCC
AG
GAGGCTCGGCTCAGTTACTCCTTCTCGCAACCAGCTTTGCTTGATGATCT
CCTACTGGCTACCCAGATGAACCAAACGCAGAACGCTTCCCAGAACTAT
T
TCCAGCGTTTGGTGAGGAGAATGACCCGATTCTTTGTGACCACACGATG
G
GATGACACTATCAAGCGATTGGTGGGAACCATCGAAAGACTGGGTGGTT
A
TACGTGCAAATTTGGTGACGACGGAGTGGTCACCGTATCCACAGTCGAT
C
GGAATAAGCTGCGATTGGTTTTCAAGGCACACATTATAGAGATGGATGG
C
AAGATTCTTGTTGACTGCCGGCTGTCAAAGGGTTGCGGCTTGGAGTTTAA
ACGACGATTTATCAAGATCAAAAACGCCCTGGAGGATATCGTGCTTAAA
G
GACCCACCACCTGGCCGATTGCGATTGCTACAAATTCGGTGCCTTAGCTT
TTAGTTTCATTCGAACTTAAAATCCTGTCTTGTCTTGTATTCGGTTATAT
TTACATTTGTCTAGCCTGTAAGAGCAACTCATGCATCACCTTCGATCAGA
ATCATCTAAAATTCTCAATGTGCTAACTTATTTTTAATTCATTGCATTGT
TTACGAGTACGTTGTGTATTGCTAAGCGGGCTCTCATTCGATTTTAATTT
TGTTGAATTTTAAAGCAAAAGTCGAGTATTGAAAACTAGTTTAAATGTTA
TCGAACAATAAATCTTATGCGATTTTGAGTAGAAAA
>grp|FBgn0011598
MAATLTEAGTGPAATREFVEGWTLAQTLGEGAYGEVKLLINRQTGEAVAM
KMVDLKKHPDAANSVRKEVCIQKMLQDKHILRFFGKRSQGSVEYIFLEYA
AGGELFDRIGEEPDVGMPQHEAQRYFTQLLSGLNYLHQRGIAHRDLKPEN
LLLDEHDNVKISDFGMATMFRCKGKERLLDKRCGTLPYVAPELQKAYHA
QPADLWSCGVILVTMLAGELPWDQPSTNCTEFTNWRDNDHWQLQTPWSK
L
DTLAISLLRKLLATSPGTRLTLEKTLDHKWCNMQFADNERSYDLVDSAAA
LEICSPKAKRQRLQSSAHLSNGLDDSISRNYCSQPMPTMRSDDDFNVRLG
SGRSKEDGGDRQTLAQEARLSYSFSQPALLDDLLLATQMNQTQNASQNYF
QRLVRRMTRFFVTTRWDDTIKRLVGTIERLGGYTCKFGDDGVVTVSTVDR
NKLRLVFKAHIIEMDGKILVDCRLSKGCGLEFKRRFIKIKNALEDIVLKG
PTTWPIAITNSVP
>grp|FBgn|0011598
MAATLTEAGTGPAATREFVEGWTLAQTLGEGAYGEVKLLINRQTGEAVAM
KMVDLKKHPDAANSVRKEVCIQKMLQDKHILRFFGKRSQGSVEYIFLEYA
AGGELFDRIGEEPDVGMPQHEAQRYFTQLLSGLNYLHQRGIAHRDLKPEN
LLLDEHDVKISDFGMATMFRCKGKERLLDKRCGTLPYVAPEVLQKAYHA
QPADLWSCGVILVTMLAGELPWDQPSTNCTEFTNWRDNDHWQLQTPWSK
L
DTLAISLLRKLLATSPGTRLTLEKTLDHKWCNMQFADNERSYDLVDSAAA
LEICSPKAKRQRLQSSAHLSNGLDDSISRNYCSQPMPTMRSDDDFNVRLG
SGRSKEDGGDRQTLAQEARLSYSFSQPALLDDLLLATQMNQTQNASQNYF
QRLVRRMTRFFVTTRWDDTIKRLVGTIERLGGYTCKFGDDGVVTVSTVDR
NKLRLVFKAHIIEMDGKILVDCRLSKGCGLEFKRRFIKIKNALEDIVLKG
PTTWPIAIATNSVP
Rab5^{Scim}
>>Rab5|FBgn0014010|cDNA sequence
CCGTCGTCGGTCATAAAAACGAAAAGCATGTGAACGATACTTTGTGAAG
A
TTTGAAAACGACTTTAGCTTAGTTAATATATGCAACAATTCCGCATCCAC
ACTCAGCAGCAATCTTAGCAGAAAGACTTACTCAGCAGAAGAGGGAGTG
G
GAAGAGCGCATCCACATTCGCATCCGATCCAACCCGAACCGATCATGGC
A
ACCACTCCACGCAGCGGCGGTGCCAGCGGCACTGGAACGGCGCAGCGG
CC
CAATGGCACCTCGCAGAACAAAAGCTGCCAATTCAAGTTGGTGCTCCTC
G
GCGAGTCCGCTGTGGGCAAGTCCTCACTGGTGCTGCGCTTCGTCAAGGG
A
CAGTTCCACGAGTACCAGGAGAGCACGATAGGTGCGGCCTTTCTGACAC
A
GACTATTTGCATAGAGGACACTGTCGTTAAGTTCGAGATCTGGGACACG
G
CTGGCCAGGAGCGGTACCACAGCTTAGCTCCCATGTATTATCGAGGAGC
G
CAGGCCGCTATTGTCGTCTATGATATACAGAATCAGGACAGTTTTCAGC
G
TGCGAAGACCTGGGTCAAGGAACTGCATAAACAAGCCTCACCAAACATT
G
TCATTGCGCTGGCCGGCAACAAGGCAGATTTGTCAAACATTCGCGTCGT
A
GAGTTCGATGAAGCGAAGCAATATGCCGAGGAGAACGGGCTGCTGTTCA
T
GGAAACCTCCGCCAAGACGGGCATGAATGTGAACGACATCTTCTTGGCC
A
TTGCCAAGAAACTACCTAAGAACGATGGCGCCAACAATCAGGGAACCA
GC
ATAAGGCCGACTGGAACCGAAACAAATCGACCGACGAACAACTGCTGC
AA
GTGA
>Rab5|FBgn0014010
MATTPRSGGASGTGTAQRPNGTSQNKSCQFKLVLLGESAVGKSSLVLRFV
KGQFHEYQESTIGAAFLTQTICIEDTVVKFEIWDTAGQERYHSLAPMYYR
GAQAAIVVYDIQNQDSFQRAKTWVKELHKQASPNIVIALAGNKADLSNIR
VVEFDEAQYAEENGLLFMETSAKTGMNVNDIFLAIAKKLPKNDGANNQG
TSIRPTGTETNRPTNNCCK
oafScim
>>oaf|FBgn0011818|cDNA sequence
ATGATCTTAAAGGAGGAGCACCCACACCAGAGCATCGAAACTGCCGCA
AA
TGCGGCAAGGCAGGCGCAGGTCCGCTGGCGAATGGCGCATCTTAAGGCA
C
TCAGCCGCACTCGAACACCAGCGCACGGCAATTGCTGCGGTCGCGTCGT
C
AGTAAAAATCACTTTTTCAAGCACAGTCGCGCGTTTCTGTGGTTCCTGCT
GTGCAACTTAGTGATGAACGCGGACGCATTCGCCCACTCCCAGCTGCTC
A
TTAACGTCCAAAATCAGGGCGGCGAGGTGATCCAGGAGAGTATTACCTC
C
AACATTGGCGAGGACCTGATAACGCTGGAGTTTCAGAAGACCGACGGAA
C
GCTCATCACCCAGGTCATCGACTTTCGCAATGAGGTTCAAATCCTCAAG
G
CTCTGGTTCTCGGCGAGGAGGAGCGTGGCCAGAGCCAGTACCAGGTCAT
G
TGCTTCGCAACCAAGTTCAACAAGGGCGACTTCATCTCCTCGGCGGCAA
T
GGCCAAGCTGCGCCAGAAGAATCCGCACACCATCCGCACTCCCGAGGAG
G
ACAAGGGCCGTGAGACCTTCACCATGAGCAGCTGGGTACAGCTCAACCG
C
TCGCTGCCCATCACCAGACATCTGCAGGGACTCTGCGCCGAGGCCATGG
A
CGCCACCTATGTCCGGGATGTGGACCTTAAAGCTTGGGCGGAGCTACCA
G
GCTCCTCGATTTCCAGCCTGGAGGCCGCCACCGAAAAGTTCCCGGACAC
G
CTCTCGACGCGCTGCAACGAGGTGAGCAGCCTGTGGGCGCCCTGCCTGT
G
CAACCTGGAGACCTGCATCGGCTGGTATCCCTGCGGGCTCAAGTACTGC
A
AGGGCAAGGGAGTCGCCGGAGCGGACTCGTCGGGCGCCCAGCAGCAGG
CA
CAGCCGACGAATTATCGCTGCGGCATCAAGACCTGCCGCAAGTGCACAC
A
GTTCACCTATTATGTGCGGCAGAAACAACAGTGCCTCTGGGATGAATGA
C
GACGCGGCGAGCTGCAGCTGATGCAGATGCGCTGCGCGAGGCGGCGGA
AT
GGTAGCGAGTTTGGGGATGATGCCAGTGCCACCTGCCCGGGTGGCGAA
C
AAGAGCAGCAACCACGACCGCGACAATAACTGGCGGGGGAGCTGGGGG
AA
GTGGGAAGGATACAACGGCAGCGACAACAACGACAACCAACAAATTAC
GC
CAACTGCTTTTGTTGGTCCAGCAGCAGATGCCTTTTGCTCTGTGGAGTTT
TCCGGTCCATCACATTTCCCAGTCCCATCACCAGTCCCAGTCCCAACATA
AGCCCAGCCGGCAGCAGAAGCAGCATCAGCATCATTCTCAGGTTGCCCC
C
ACTTCGCATCACCAGTCATCATCATCAACACCACCAACACCGTCAACAT
C
ATCATCACCGCCATCATCATCATCGTCGTCGTCGTCGTCCGCAATGGCCG
CCATCGTTGCGT
>oaf|FBgn0011818
MILKEEHPHQSIETAANAARQAQVRWRMAHLKALSRTRTPAHGNCCGRVV
SKNHFFKHSRAFLWFLLCNLVMNADAFAHSQLLINVQNQGGEVIQESITS
NIGEDLITLEFQKTDGTLITQVIDFRNEVQILKALVLGEEERGQSQYQVM
CFATKFNKGDFISSAAMAKLRQKNPHTIRTPEEDKGRETFTMSSWVQLNR
SLPITRHLQGLCAEMDATYVRDVDLKAWAELPGSSISSLEAATEKFPDT
LSTRCNEVSSLWAPCLCNLETCIGWYPCGLKYCKGKGVAGADSSGAQQQA
QPTNYRCGIKTCRKCTQFTYYVRQKQQCLWDE
GliScim
>>Gli|FBgn0001987|cDNA sequence
GTTCGAAAGAGTCGGTTGCCTCTGTGTTTTGGCGGTAGTGGTTGTGCGCA
CAGGTGAGAGTTCGGTGCGAAGAGGCGGTTAAAGTTAAAGCTGCGCGC
G
CTCAATACAACGAAAGAATCGGTTGGCCAGGTGTATCTGTGTACGAATC
G
TTGGTAACAGCCGGCAACTGAGTATCATCATGATGCACAAATTGAAATA
T
CGCGATAAATTAAAATGGCTTTTAGCCCTTCTTGTGCTGATCGGCACTTG
TTTTATTCAGACAAGGGGACAAACAAGAGATCCCAGATTTTATTCTCGG
C
CAGGCGTTGACTACCATTGGCCAAATCCAGGCGATCCGGATTACAGAAC
C
TACACGTTCAACGATCGCCGATATGGTCATTATCAGCCAAATGGCTATG
G
AGCCAACTATCCAGGCAGAAATCCACCGGGACAATATCCACAGGGAAT
GC
CGAATGAAGATCGCTTTCGATTTGACCCGAACGATCCGAATGCGAGAAC
C
CAGTTTCCGGGAGTGCTGGCCGGATGGCGAGAGGATTTGCAGGGCAAGC
A
GCGGCGGGATTCGTTGACCCTGGAGCGGGATGTTTTCGTGACCACCAAC
T
ATGGCCAGGTGCAGGGCTTTAAGGTGTACATGTACGATAATCCAGATCC
G
AAGTCCTTCTATCGTCCCTACCACTCGACCGTGGATCGTGTGATGGGCGA
GTGCTCGGTCTTCCTGGGCATTCCCTACGCCCTGCCGCCCACCTTCGAGG
GCAGGTTCAAGCCACCACGCGTCCATCGAGGCTGGCAGCTGCTGCAGGC
C
GTCGACTTTGGACCCGCCTGTCCACAGCCTGTGCGATATACGGGTGCCA
C
GAAAGGAATCATGGACATGGACGAGGATTGCCTCTACTTGAACGTGTAT
T
CGCCGAAGACTGGTGCTGGTGTGGCTCAAAAATACCCGGTTATGGTGTA
C
ATCCATGGCGGCGAGTTCATTCGTGGAGCCTCCAACCTATTCCAGGGTC
A
TATTCTGGCCTCGTTCTACGACGTGGTCGTGGTGACCCTGAATTACCGCC
TTGGTGCCCTGGGATTCCTATCGACGGGTGATGAGAACTCGCCCGGAAA
C
TACGGAATCCTCGATCAAGCGATGGCGCTACGTTGGGTCTATGACAATA
T
TGAGTTCTTCAACGGCGATCGGAATTCCATCACTCTATTTGGTCCGGGAG
CAGGAGGCGCCTCCGCTGGACTCCTGATGGTGGCACCACAGACGCGGAA
C
ATTGTGCGTCGTGTGATCGCACAGTCCGGATCGGCTCTAGCGGATTGGG
C
GCTCATCCAGGACAAGTATCGCGCCCAGAACACGAGTCGCGTGCTGGGA
C
AGCTGCTGGGCTGCTCCATTGAATCGTCGTGGAAGTTGGTCAACTGCCTG
CGCACCGGACGCAGCTTCTATGAGCTGGGAAACGCTGAGTTCTCTCCCC
A
GGTGGGCAGCTTTCCATGGGGTCCAGTTCTGGACCACAACTTTACGTTGC
CCGGCGACGATTGGTACGAGGGATGGCGCGAAAAGGATTGGCGTTTCCT
C
ACCCAAACGCCGGAAACCCTCATCCGTGCCGGTAAATTCAACCGGAATA
T
TCAGTACATGACGGGCGTGACCACACAGGAAGCGCCTTTTTTGTGGCC
C
AAAACGAATCCCTAAGTCCGTACTATGAACTAGATGGACGTTTCTTCGA
T
CAGAAAATAAGGGAACACGTTTTCCGCTACAACTATACACTTAATCCGA
A
CGGAGTTTACGAGGCCATCAAGTACATATACACCTTCTGGCCGGATCCC
A
ATAATAACACCATAATCCGGGACCAGTACATAAACATGCTGAGTGATCT
C
TACTACCGAGCACCGGTGGATCAAATGGTCAAGCTAATGCTGGAGCAGA
A
GGTACCCGTTTATATGTACGTACTGAACACCACTGTGGAGGCACTGAAT
C
TGCCACAGTGGCGAAAGTATCCACACGACATCGAACGTTATTTCCTCAC
C
GGAGCTCCCTTCATGGACACCGAGTTCTTTCCCAAAAAGGAGCATCTGC
A
GCGCAATATGTGGACGGATAACGATCGCAATATGATCACTTTTTCATG
C
AGACCTACACGAATTTTGCTAGATATGGCAATCCGACGCCGCAACAGGT
G
CTAGGCATGCATTTCCAGCGCGCATACCAGGGCGAGATTCGGTACTTGA
A
CATCAATACCACGTACAACTCCTCCATTCTACTCAACTATCGGCAGACGG
AGTGCGCCTTCTGGACGCAATACTTGCCCACAGTTATTGGAGTGCTGGTG
CCCACTTATCCACCCACCACGGAGTATTGGTGGGAGCCCAAGGAGCCAC
T
ACAGATCGCCTTTTGGAGCATGTCGGTGGCCTGTTTCTTCCTCATAGTCC
TGGTGGTCATCTGCTGCATCATGTGGCGCAATGCCAAGCGCCAATCGGA
T
CGCTTCTATGACGAAGATGTCTTCATTAATGGTGAGGGCTTGGAACCGG
A
ACAGGATACGCGTGGAGTGGACAATGCCCACATGGTGACCAACCATCAT
G
CCCTGCGCTCCAGGGATAATATCTACGAGTACCCGCGACTCTCCATCCACC
AAAACCTTGGCCAGCAAAGCGCACACGGACACCACCTCGTTGCGCTCAC
C
CAGTTCGCTGGCCATGACCCAAAAGTCCAGCAGCCAGGCGTCCCTCAAG
T
CAGGGATCTCGCTCAAGGAAACCAATGGCCATTTGGTGAAGCAATCTGA
A
AGGGAGCCACGCCACGATCCCAACAAAATGGGTCCACCGCAAAGGTG
GC
GTCTCCTCCTGTGGAGGAGAAGCGTCTACTGCAGCCACTTTCCAGCACG
C
CCGTGACGCAGTTGCAGGCGGAGCCGGCCAAAAGAGTTCCCACCGCTGC
C
AGTGTCTCGGGCAGCAGTCGGAGCACCACTCCGGTGCCCTCTGCCCGCA
G
CACCACCACGCACACCACAACAGCCACCCTGAGTTCCCAGCCAGCGGCT
C
AGCCGAGGAGAACCCACCTGGTGGAGGGAGTGCCTCAGACATCCGTTT
C
TCCTGGAATTTTTCTTTAAAGTAAGTTAGTTATTTTCGAAAAAAGTAGTT
TATGTTTTGTATTATGATAGGTTTGTAAACAGATCTTTGAGATCTCAGT
TTTGAGTTGTAGTTTAGGCAAACCTGTATTTCTAATGCCTTACTTTAAGT
TAAATTGTTCGTAAGTTTGATAACCCAACGAATACAAATCCATATCAATG
ACATTCCATAGCCTTATCATCGGCCTTTACGTGTATATATAGAATTTAAA
TGTATAATAAGAATTATTTAAATCTAGGAAATTGCCTGCCTTAACTGAAG
GAAATTAGCTATATAATACAAGTATTTTTATAACAAATGCTGAGCTTAAA
TTTTTAAAACTCACTTTTAGTGGCAGTAATATCGAATGAAATTGTAAAGA
GAGTATATCTTTTAAATCAAATATTAAAATACAAAAATAAATTAACCTTA
TAAAAAATAATGCATTACATTATTTAAGACCTGCTATTATTTGATAACAA
ACTCATAACATTGTCACGTCCCGTATTATAAGAATATCATTAGAATACCA
TTAGATCGTAGCCACTCCCACACATCATCAAGAGCAATACACCTACAGT
G
CCTTAATAATATTTGGTAATTGTCCTCGATGGACCCACCCACCCCATCCA
TGATGTACCAACTAAGTGTGCCTTTCATACATACATAAATGTACTCCTAG
TTATATACTATATATGTTCTATATGTTCTGTGTGTCGCATATAAGCAAAA
TGACCATACAAAATCAGTGT
>Gli|FBgn0001987
MPNEDRFRFDPNDPNARTQFPGVLAGWREDLQGKQRRDSLTLERDVFVTT
NYGQVQGFKVYMYDNPDPKSFYRPYHSTVDRVMGECSVFLGIPYALPPTF
EGRFKPPRVHRGWQLLQAVDFGPACPQPVRYTGATKGIMDMDEDCLYLNV
YSPKTGAGVAQKYPVMVYIHGGEFIRGASNLFQGHILASFDVVVVTLNY
RLGALGFLSTGDENSPGNYGILDQAMALRWVYDNIEFFNGDRNSITLFGP
GAGGASAGLLMVAPQTRNIVRRVIAQSGSALADWALIQDKYRAQNTSRVL
GQLLGCSIESSWKLVNCLRTGRSFYELGNAEFSPQVGSFPWGPVLDHNFT
LPGDDWYEGWREKDWRFLTQTPETLIRAGKFNRNIQYMTGVTTQEAAFFV
AQNESLSPYYELDGRFFDQKIREHVFRYNYTLNPNGVYEAIKYIYTFWPD
PNNNTIIRDQYINMLSDLYYRAPVDQMVKLMLEQKVPVYMYVLNTTVEAL
NLPQWRKYPHDIERYFLTGAPFMDTEFFPKKEHLQRNMWTDNDRNMSHFF
MQTYTNFARYGNPTPQQVLGMHFQRAYQGEIRYLNINTTYNSSILLNYRQ
TECAFWTQYLPTVIGVLVPTYPPTTEYWWEPKEPLQIAFWSMSVACFFLI
VLVVICCIMWRNAKRQSDRFYDEDFINGEGLEPEQDTRGVDNAHMVTNH
HALRSRDNIYEYRDSPSTKTLASKAHTDTTSLRSPSSLAMTQKSSSQASL
KSGISLKETNGHLVKQSERAATPRSQQNGSTAKVASPPVEEKRLLQPLSS
TPVTQLQAEPAKRVPTAASVSGSSRSTTPVPSARSTTTHTTTATLSSQPA
AQPRRTHLVEGVPQTSVFSWNFSLK
Hr39Scim
>>Hr39|FBgn001229|cDNA sequence
GACTGTGTTGCGTCGTGTGATCGCTAGAGCGGTTGTGGAATCGGATTCG
A
GCGCAAAACACCGTTCATGCTGTGAAAAATCCGATATTTGTCGTGCAAT
A
ATTTCCTCGATTGGCATCAAGTGGCTTCCAGTCGGGTACATATTGCACAA
GAAATGTTATACGCATAATGTGCACGCAAATTAAACGAATTCTCTATGA
A
AATGTGACTAGAATGTGAGTCGAACAAAACGAGTAAAACGTGAAATCCC
A
ACTGGCTTTTGGGTAACAAATCTTATCAACACAGCAACGGAAATACATT
A
AAATCTTGATAGACTGAGAAAGGGACAATTGGAATACTTTTAGTTATTTT
TAAATGTTTTATTTTTCAAGTTTTACAACACAATGGAACTGCATCAACGA
CACCTCTCAAACTTTTACAAATTGCACAACTGAGAAATAGTCTTTGATAA
ATAAATAAAATATAAGAAATCGCTACTGAAACAAGATGCCAAACATGTC
C
AGCATCAAAGCGGAGCAGCAAAGCGGTCCTCTTGGAGGAAGTAGCGGC
TA
TCAAGTACCGGTCAACATGTGCACCACCACAGTCGCGAATACGACGACC
A
CTTTGGGAAGCTCCGCCGGGGGAGCCACTGGCTCCCGGCACAACGTCTC
C
GTGACAAACATCAAGTGCGAACTAGACGAACTACCGTCACCGAACGGC
AA
CATGGTGCCGGTTATCGCAAACTACGTTCACGGTAGCTTGCGCATTCCAC
TCAGTGGACATTCAAATCATAGGGAGTCCGATTCGGAGGAGGAGCTGGC
A
AGTATTGAGAACTTGAAGGTTCGGCGAAGGACGGCGGCGGACAAAAAT
GG
TCCTCGTCCAATGTCCTGGGAGGGCGAGCTGAGCGATACTGAGGTCAAC
G
GGGGCGAAGAGCTGATGGAAATGGAGCCAACAATTAAGAGTGAGGTGG
TC
CCTGCTGTTGCACCCCCACAACCCGTCTGCGCACTACAACCGATAAAAA
C
AGAGCTAGAGAACATTGCAGGCGAGATGCAGATTCAAGAGAAGTGTTA
CC
CCCAGTCCAACACACAACATCACGCTGCCACAAAATTAAAAGTGGCCCC
G
ACGCAAAGTGATCCGATCAATCTCAAGTTCGAACCGCCTCTGGGAGACA
A
TTCTCCGCTACTGGCTGCACGTAGCAAGTCCAGCAGTGGAGGCCACCTA
C
CACTGCCAACGAATCCCAGTCCCGACTCCGCCATACATTCCGTCTACAC
G
CACAGCTCCCCCTCGCAGTCGCCTCTGACGTCGCGCCACGCCCCCTACAC
TCCGTCTCTGAGCCGCAACAACAGCGACGCCTCGCACAGTAGCTGCTAC
A
GCTATAGCTCCGAATTCAGTCCCACACACTCGCCCATTCAAGCGCGTCAT
A
GCTATAGCTCCGAATTCAGTCCCACACACTCGCCCATTCAAGCGCGTCAT
GCCCCACCCGCCGGCACGCTCTATGGCAACCACCATGGTATTTACCGCC
A
GATGAAGGTGGAAGCCTCATCCACTGTGCCGTCCAGTGGGCAGGAGGCG
C
AGAACCTGAGTATGGACTCTGCCTCTAGCAATCTGGATACAGTGGGCTT
A
GGATCTTCGCACCCCGCATCTCCGGCGGGCATATCACGTCAGCAGTTGA
T
CAACTCGCCCTGCCCCATCTGCGGTGACAAGATCAGCGGATTTCATTAC
G
GGATTTTCTCCTGCGAGTCTTGCAAGGGCTTCTTCAAGCGCACCGTGCAA
AATCGCAAGAACTACGTGTGCGTGCGTGGTGGACCATGTCAGGTCAGCA
T
TTCCACGCGCAAGAAATGTCCAGCCTGCCGCTTCGAGAAGTGTCTGCAG
A
AGGGAATGAAACTAGAAGCGATTCGGGAGGACCGAACCCGTGGCGGCC
GC
TCCACATACCAGTGCTCCTACACGCTGCCCAACTCAATGCTTAGTCCGCT
GCTTAGTCCTGATCAAGCGGCAGCAGCTGCCGCCGCAGCAGCAGTGGCA
A
GTCAGCAGCAGCCGCACCAGCGACTACATCAACTAAATGGATTTGGAGG
T
GTACCCATTCCCTGCTCTACTTCTCTTCCAGCCAGCCCTAGTTTGGCAGG
AACTTCGGTCAAGTCGGAAGAGATGGCGGAGACGGGCAAGCAAAGCCT
CC
GAACGGGAAGCGTACCACCACTACTGCAGGAAATCATGGATGTAGAGC
AT
CTGTGGCAGTACACCGATGCAGAGCTGGCCCGCATCAACCAACCACTGT
C
CGCATTCGCCTCTGGCAGCTCTTCGTCGTCGTCATCGTCAGGTACATCCT
CAGGCGCCCATGCACAACTCACCAATCCACTACTGGCTAGTGCTGGTCT
C
TCGTCCAATGGCGAGAATGCCAATCCTGATCTTATCGCTCATCTCTGCAA
CGTGGCTGATCACCGTCTTTATAAAATCGTCAAATGGTGCAAGAGCTTG
C
CGCTTTTTAAGAACATTTCGATCGATGACCAAATCTGCTTGCTCATTAAC
TCGTGGTGCGAGCTGTTGCTCTTCTCCTGCTGTTTTAGATCAATTGATAC
TCCTGGAGAGATTAAAATGTCACAAGGCAGGAAGATAACCCTATCGCAG
G
CCAAATCAAATGGCTTGCAGACTTGCATTGAACGGATGCTCAACCTAAC
A
GATCACCTGAGGCGATTGCGCGTTGATCGCTACGAATATGTTGCCATGA
A
AGTTATTGTGCTGTTGCAGTCAGATACGACAGAGTTACAGGAAGCGGTA
A
AGGTGCGCGAGTGTCAGGAAAAAGCTTTGCAGAGCTTGCAAGCTTACAC
C
CTGGCGCATTATCCTGACACGCCATCCAAGTTTGGGGAGTTTTGCTACG
CATTCCTGATTTGCAGCGAACGTGCCAGCTTGGCAAGGAGATGTTGACG
A
TCAAGACTCGCGATGGAGCTGATTTCAATTTGCTAATGGAGCTTTTGCGC
GGAGAGCATTGACAATTGATAACTAAGACGAAATCTTTTACCATTGGC
A
AAACAAGTTTCACATATTTAGTATTAGATATATATATTCTATAGATAAGA
TCCTTACTGTAAGTTCTGAAAACATGTGCCTAAAAACCAAAGCCACGAT
A
GCAGTCACATCAGGCCCACTGGTCGAGATTAAATCCAAGAGCAAGATTG
C
CAAATTTTTACACCAATATATATTTTGATATGAGCCATGTGCAGGGCCTC
AGATCGCTGTTGTTGTCGGCTAAAGTTTCAGTAAGAAAAGTATATATTGA
TTTTGCTATTTATACATATTTGACTTATGTATAGTGTAAACTAAAGCACA
CATGGAAAATGAAAAGACTAAACAAATTTATTTAAAGATTACTTTTACT
A
TTATAGAAAAAGGGGAAAAATAAAAAACACAAAGGCAGAGAAGAAAAT
TT
AGTTACAACAGGTAGCGACATTTTTATATTTTCTTATATAAGGAAATATT
CAATGTATTTTAAATATAAAGCCAAACCCGATTTGGTTTGGGAAAGAGC
T
ACTGAAATTTTTGATATCTATATATTCATCACTAGAAGACGAATGAATGT
ATCCAATGTTTAAATGTTGTAGCGTTTAGTTTTAGTGCAATTTCACACAT
GTCTACATACATGAATATTCAGCGAGATATGTTTGCAAACTATTATAAAG
CAAAAGACCACTCGAAATCGCCATCACTGGGTTGGCTAAGACTATTCCA
G
TTATGCTGTTTGTTGCATAAAAAACCACAACTACGTACATCAATAAAATG
TATAATTTTTTATTGGAGTTTTAGATTTGTATTAACTTCTTCCTTATAAT
TACGATTATTATTATTATTACTAATTTTATGAATATTGTGTAACACTGAC
TTAAATAGCTGAAAAAATCCTGCAACAGGATTTAAAACACCTGAATACA
C
AAAACATTATAACATGAATACATTTTGCTTATGGCCTAGATAGTTTGATA
TGTACTTTGCATATGTATGCATGTGTCTATATGTGAGTACGTACCATACA
AATTCCTGTCCCACCAGAAAAATCACACGCAATAAAAAATTCCAAAATA
C
TAAGCTCGTATCTACAAAGAAAGATTAAAAGACAAATTGATGAATAGGA
A
TATGTTGCCGGAAGTCCAAGAGATTTGGCTGAAAGTATCGACAAATTTT
C
AACACTCGTTCATGGATATTGTGCTAACACTCTCAGTTTGAAAATCATT
TTCTGTTAAACTTTCTATATAATAAGTTCTCCATTCGATTTTGTATTTAC
AATTTGTTTCTTTAATTTTCCTTTATCAGTTGTATCTATGAAACATGAGG
ATCTCAGTTCATATTGATCGTGTTCTTCTGCCGTACACCGCTTCTGTCCG
TTAATGTAAACCATAAGTATAAATGAAATTAGTTAAATGTTTATTTATAA
ATAAAGCGCTATAATAAATTTCAATACATTTATCATAGTTAACTGATTAA
GACCACTGAAATCAAAAATATTTTATTTACTAAGCAAAGCACACGCAAA
C
AATTTATAATGTTTATTACGTTAACAACAAACTCATTTTAATAATTCTT
TATGAATACACAAAGTTACGCAATTTTCCCTCTAGGCGCATTGCTTAAAT
AGTTAAAGAAAAATAATAAACCCATAGCGCAATATTTAATGTAAAACAG
T
TTTCCTTGCGTGTGATGTTTGCTCTAGCTACGTACAAATTCATCATTTAT
TAAATTTAAAACTCAATTTTGCTTTTAAATAAATTTAATAAGTAAAATTC
AACAATAATTGATATACAATTGTCAATGCAATATTTTGTAATAAAAATGC
GAAAAATC
>Hr39|FBgn0010229
MPNMSSIKAEQQSGPLGGSSGYQVPVNMCTTTVANTTTTLGSSAGGATGS
RHNVSVTNIKCELDELPSPNGNMVPVIANYVHGSLRIPLSGHSNHRESDS
EEELASIENLKVRRRTAADKNGPRPMSWEGELSDTEVNGGEELMEMEPTI
KSEVVPAVAPPQPVCALQPIKTELENIAGEMQIQEKCYPQSNTQHHAATK
LKVAPTQSDPINLKFEPPLGDNSPLLAARSKSSSGGHLPLPTNPSPDSAI
HSVYTHSSPSQSPLTSRHAPYTPSLSRNNSDASHSSCYSYSSEFSPTHSP
IQARHAPPAGTLYGNHHGIYRQMKVEASSTVPSSGQEAQNLSMDSASSNL
DTVGLGSSHPASPAGISRQQLINSPCPICGDKISGFHYGIFSCESCKGFF
KRTVQNRKNYVCVRGGPCQVSISTRKKCPACRFEDCLQKGMKLEAIREDR
TRGGRSTYQCSYTLPNSMLSPLLSPDQAAAAAAAAAVASQQQPHQRLHQL
NGFGGVPIPCSTSLPASPSLAGTSVKSEEMAETGKQSLRTGSVPPLLQEI
MDVEHLWQYTDAELARINQPLSAFASGSSSSSSSSGTSSGAHAQLTNPLL
ASAGLSSNGENANPDLIAHLCNVADHRLYKIVKWCKSLPLFKNISIDDQI
CLLINSWCELLLFSCCFRSIDTPGEIKMSOGRKITLSOAKSNGLQTCIER
MLNLTDHLRRLRVDRYEYVAMKVIVLLQSDTTELQEAVKVRECQEKALQS
LQAYTLAHYPDTPSKFGELLLRIPDLQRTCQLGKEMLTIKTRDGADFNLL
MELLRGEH
ScaScim
>>sca|FBgn0003326|cDNA sequence
AAAACATCCTTCCATTGGAAGGTGCTCTAGTGAAATCGGTGATATACTTC
ATCCAGTGCTGCGAGCGAACTTTCCGTGGACCAAAAGTGAAAAGAACAG
C
TAAGCCAAGCAACAAGGATTAAGCCCGAGGAAACGCCTGGAATAATTGT
A
GCATTGTTTGCCAGCGCCTTTTTATAAGGTGTTTGACTGTCACGGCGCCG
GAAAGTCAAGGAGAATTGTTTGTGTGTCGGCATGCGTGCCAGTTATTTGC
ATAAATTGCCCATCTAACTCGAGTTCCCTTTTTTGTGCGGTGTGAATGAG
AGATTGGCAAACATTCCCGGACCTCCAAAAAAAGAAAGTTTCGCGTGAC
C
ATTTAAATTGCCCTGCAACAATGGCAGGTTCAAACGTTTTGTGGCCAATA
CTCCTGGCCGTGGTGCTGCTCCAAATATCCGTGGCATTCGTGAGTGGAGC
GGCCAGTGGTGGGGTAGTCCTTAGCGACGTGAACAACATGTTGCGCGAT
G
CCAAGGTGGTGACCTCGGAGAAACCCGTTGTGCACTCAAAACAGGAAAC
G
GAAGCGCCGGAATCCAGCGTGGAGCTGCTCCGCTTCGTCGATGATGACG
A
GGATAGCGAGGACATCAGCTCCATTGAACGGCAGGATGGCAGGACAAT
GG
AGAGCAAAAAAATGGCCGATCAGGTGCGCCTGTTGACCAAGCAGCTCA
AC
GCCCTGATGCTGCGGCGCCGCGAGGATTACGAGATGCTGGAGCACAATC
T
GCGCAAATCCCTGCGGCTCACCACGAATGCGAACAGCGTGGACGCCGAC
A
TGCGCAGCGAACTGAACCAACTCAGGGAGGAGCTGGCCGCGCTGCGCTC
C
TCGCAGAGTGGCAACAAGGAGCGCTTGACCGTCGAGTGGCTGCAGCAG
AC
GATCTCCGAGATCCGCAAACAGCTGGTGGATCTGCAAAGGACGGCCAGC
A
ACGTGGCCCAGGATGTCCAGCAGCGCAGCTCCACCTTTGAGGATCTGGC
C
ACCATTCGCAGTGACTATCAGCAGCTTAAGCTAGATCTGGCGCTCAGC
G
CGAGCGCCAGCAGCAGACGGAGGTCTACGTCCAGGAACTGCGCGAGGA
GA
TGCTCCAGCAGGAGCAGGACTTCCAGCATGCCCTCGTCAAGCTGCAGCA
G
AGGACTCGCAAGGACGGCTCATCCGCCAGTGTGGAGGAGGAGAGCGGT
AG
CCAGGAAGCCAACCAGGAGCAAACCGGACTTGAAACCACTGCTGATCA
CA
AGCGACGCCATTGCCGTTTTCAGAGCGAACAGATCCACCAGCTGCAACT
G
GCCCAGAGGAACCTGCGCCGACAGGTGAACGGATTGCGCTTCCACCACA
T
CGACGAGCGGGTTCGCAGCATCGAGGTGGAGCAGCACAGGATTGCCAA
TG
CCAACTTTAATTTGAGCAGCCAGATCGCTTCGCTGGACAAGCTGCATAC
C
TCGATGCTGGAGCTGCTGGAAGATGTGGAGGGCCTCCAGACCAAGATGG
A
CAAGAGCATACCGGAGCTGCGGCACGAGATCTCCAAGCTGGAGTTCGCC
A
ATGCTCAGATCACCTCGGAGCAGAGTCTGATCAGGGAGGAGGGCACTAA
T
GCGGCACGATCCCTGCAAGCCATGGCTGTAAGCGTCAGTGTCCTGCAGG
A
GGAGCGCGAAGGTATGCGGAAGCTGTCCGCCAATGTGGATCAGCTGAGA
A
CCAATGTGGATCGATTGCAGTCGCTGGTCAATGATGAAATGAAGAATAA
G
CTCACCCACCTGAACAAGCCGCACAAGCGACCACATCATCAGAATGTCC
A
GGCGCAGATGCCGCAGGATGATTCGCCCATTGACTCCGTCCTGGCCGAA
A
CTCTGGTCAGCGAGCTTGAGAACGTGGAGACCCAGTACGAGGCCATTAT
C
AACAAACTGCCGCACGACTGCAGCGAGGTGCACACTCAAACAGACGGA
CT
GCATCTGATTGCGCCCGCCGGCCAACGGCATCCGCTGATGACGCACTGC
A
CCGCCGATGGATGGACGACGGTGCAAAGGCGGTTCGATGGCAGTGCAG
AC
TTCAACCGCTCGTGGGCGGATTATGCCCAAGGATTTGGGGCGCCAGGCG
G
TGAATTCTGGATTGGCAACGAGCAGCTGCATCACCTGACCCTGGACAAC
T
GCAGTCGGCTGCAGGTGCAAATGCAGGACATCTACGACAACGTTTGGGT
G
GCCGAGTACAAGCGATTCTACATATCCTCGCGAGCCGATGGCTATCGGC
T
GCACATTGCCGAGTACTCCGGCAACGCTTCGGATGCACTGAACTACCAA
C
AGGGTATGCAGTTCTCGGCCATCGATGACGATCGGGACATCTCGCAGAC
G
CACTGTGCTGCTAACTATGAGGGTGGCTGGTGGTTCTCTCATTGCCAGCA
CGCCAATCTCAATGGGCGATACAATCTGGGCCTGACTTGGTTCGATGCC
G
CTCGCAATGAATGGATAGCGGTCAAGTCAAGCCGAATGCTGGTCAAGCG
C
CTGCCCGCCGTCGAGTGCCAGGCGAATGCCAGTGCCAGTGGCGCTTTTG
T
TTCCGTTTCCGGTTCGGCTGCTGATGCTGCGCCGTCGAGCGGTGCAACAA
CAACAACAACAACAGCAACAGCAGCGCCGGCGACGGTAACGACGCCGA
AA
ACCAACAACAGTGTGGTCCAGTTCGTGGCCGCCGGGCAGGCGTAA
>sca|FBgn0003326
MAGSNVLWPILLAVVLLQISVAFVSGAASGGVVLSDVNNMLRDAKVVTSE
KPVVHSKQETEAPESSVELLRFVDDDEDSEDISSIERQDGRTMESKKMAD
QVRLLTKQLNALMLRRREDYEMLEHNLRKSLRTTNANSVDADMRSELNQ
LREELAALRSSQSGNKERLTVEWLQQTISEIRKQLVDLQRTASNVAQDVQ
QRSSTFEDLATIRSDYQQLKLDLAAQRERQQQTEVYVQELREEMLQQEQD
FQHALVKLQQRTRKDGSSASVEEESGSQEANQEQTGLETTADHKRRHCRF
QSEQIHQLQLAQRNLRRQVNGLRFHHIDERVRSIEVEQHRIANANFNLSS
QIASLDKLHTSMLELLEDVEGLQTKMDKSIPELRHEISKLEFANAQITSE
QSLIREEGTNAARSLQAMAVSVSVLQEEREGMRKLSANVQLRTNVDRLQ
SLVNDEMKNKLTHLNKPHKRPHHQNVQAQMPQDDSPIDSVLAETLVSELE
NVETQYEAIINKLPHDCSEVHTQTDGLHLIAPAGQRHPLMTHCTADGWTT
VQRRFDGSADFNRSWADYAQGFGAPGGEFWIGNEQLHHLTLDNCSRLQVQ
MQDIYDNVWVAEYKRFYISSRADGYRLHIAEYSGNASDALNYQQGMQFSA
IDDDRDISQTHCAANYEGGWWFSHCQHANLNGRYNLGLTWFDAARNEWI
A
VKSSRMLVKRLPAVECQANASASGAFVSVSGSAADAAPSSGATTTTTTAT
AAPATVTTPKTNNSVVQFVAAGQA
cnnScim
>>cnn|FBgn0013765|cDNA sequence
CCAGAAACAGCTGTTCCAGCGCGCTTCATTTTCCAAACAGAAAAAAAGT
G
TAATTGTTAGCGTCCTTTGTGAAATTGTCAAGTGTTAGAATTATTGTGTG
CGAAAGTTAACTATTTGAGGACCTCCCATGGACCAGTCTAAACAGGTTT
T
GCGGGACTATTGCGGCGACGGCAATGGTACCTGTGCATCGTCCTTGAAG
G
AAATCACCTTAATTGAGACCGTGACCAGTTTCCTGGAGGAGAATGGCGC
C
GCCGAAATCGACAGAAGGGTCCTGCGCAAACTAGCCGAGGCACTGTCCA
A
AAGCATAGACGACACCAGTCCGGGAGCCCTGCAAGATGTCACCATGGA
GA
ACTCATATGCCAGTTTTGACGTTCCACGACCTCCAGGCGGCGGCAACTC
G
CCCTTGCCGTCACAGGGTCGCTCTGTACGCGAATTGGAGGAGCAGATGT
C
CGCGCTGCGCAAGGAGAACTTCAATCTAAAGCTGCGCATCTACTTCCTC
G
AGGAGGGTCAGCCGGGTGCCCGGGCAGACAGCTCCACAGAATCCTTAA
GC
AAACAGCTCATCGATGCCAAGATCGAAATCGCGACATTGAGAAAAACTG
T
CGATGTAAAGATGGAGCTGCTCAAGGATGCCGCTCGAGCCATTTCTCAT
C
ACGAGGAATTGCAGCGCAAAGCAGACATTGACAGCCAGGCAATAATCG
AC
GAGTTGCAAGAGCAAATACACGCCTATCAGATGGCGGAGTCTGGTGGTC
A
ACCTGTCGAAAATATTGCCAAAACCAGGAAAATGTTGCGCCTTGAATCG
G
AGGTGCAGAGATTGGAGGAGGAACTGGTGAATATCGAAGCTCGTAACGT
T
GCAGCCCGGAACGAGCTGGAATTCATGTTGGCCGAGCGCCTAGAATCCC
T
AACAGCCTGTGAGGGCAAGATTCAAGAGCTGGCCATCAAGAATTCCGAA
C
TGGTAGAGCGTCTTGAAAAGGAAACAGCATCCGCCGAGTCATCCAACGC
C
AATCGAGATCTGGGCGCCCAACTGGCGGATAAGATTTGCGAGCTGCAGG
A
AGCCCAGGAGAAGCTCAAGGAGCGCGAGCGCATCCACGAGCAGGCATG
CC
GCACCATTCAAAAGCTAATGCAAAAGCTAAGCAGCCAGGAGAAGGAGA
CC
GCACCATTCAAAAGCTAATGCAAAAGCTAAGCAGCCAGGAGAAGGAGA
TA
AAGAAGCTCAACCAGGAGAACGAACAGTCGGCAAACAAGGAGAACGAC
TG
CGCTAAGACGGTAATTTCGCCATCCTCCAGCGGCCGTTCCATGAGTGAC
A
ACGAGGCCAGCTCCCAGGAAATGTCCACCAACCTCAGGGTGCGCTACGA
A
CTAAAGATCAACGAGCAGGAGGAGAAGATCAAGCAGTTGCAGACGGAA
GT
AAAGAAGAAGACGGCGAATCTGCAAAATCTGGTCAACAAGGAGCTATG
GG
AGAAAAATCGTGAGGTGGAGCGCCTCACTAAGCTGCTGGCTAACCAACA
CA
GCAATCCTTCACGGAGGCGGAGTACATGAGGGCATTGGAGCGAAACAA
GC
TGCTGCAGCGAAAGGTGGATGTGCTCTTCCAGCGCCTGGCAGACGATCA
A
CAGAACAGCGCTGTGATTGGGCAGTTGCGTTTGGAACTTCAACAAGCTC
G
CACGGAAGTCGAGACGGCGGATAAGTGGCGTCTTGAATGCGTCGATGTC
T
GCAGTGTGCTGACAAACCGATTGGAAGAGCTGGCTGGTTTCCTCAACTC
T
CTGCTGAAGCACAAAGATGTTCTTGGCGTGTTGGCCGCTGATCGACGCA
A
TGCCATGCGTAAGGCGGTGGATCGCAGCTTGGATCTTTCCAAGAGTCTT
A
ATATGACTCTGAATATAACAGCTACATCCTTGGCTGATCAAAGCCTCGCT
CAGCTGTGCAATCTATCCGAGATCTTGTACACCGAAGGTGATGCAAGCC
A
CAAAACTTTCAATTCCCACGAAGAGCTGCACGCCGCTACTTCGATGGCT
C
CGACTGTAGAGAACTTAAAGGCCGAGAATAAGGCTCTTAAAAAGGAGTT
G
GAAAAGCGACGCAGCTCAGAAGGACAGAGGAAAGAGCGCCGCTCCTTA
CC
GCTGCCCTCCCAGCAGTTCGATAACCAGAGCGAGTCAGAGGCCTGGTCA
G
AGCCTGACCGCAAGGTTTCCTTGGCACGCATTGGCCTGGACGAAACCTC
C
AACAGTTTGGCAGCGCCTGAGCAGGCGATCAGCGAGTCGGAGAGCGAG
GG
AAGAACCTGTGCTACCCGTCAGGATCGCAATCGCAACAGTGAGCGTATT
G
CCCAGCTGGAGGAGCAGATTGCCCAGAAAGACGAACGTATGCTTAATGT
G
CAATGCCAAATGGTGGAGCTGGACAATAGATATAAGCAGGAGCAATTGC
G
CTGCCTCGATATTACTCAACAATTGGAGCAATTGCGTGCTATCAACGAA
G
CTCTGACTGCAGACCTGCATGCTATAGGATCACACGAAGAGGAACGCAT
G
GTCGAGTTGCAACGCCAGCTGGAGCTTAAGAACCAGCAGATTGATCAAC
T
AAAACTGGCCCACAGCACTCTGACGGCAGATTCGCAGATAACCGAGATG
G
AGCTGCAGGCGTTGCAGCAGCAAATGCAGGAAATAGAGCAGCTGCACG
CC
GATTCAGTAGAAACCCTGCAATCCCAGCTACAGAAACTCAAACTAGATG
C
CGTGCAGCAGCTAGAAGAGCACGAGCGCCTGCATCGCGAGGCTCTTGAA
C
GCGACTGGGTGGCACTGACCACTTACCAGGAGCAGGCTCAACAGTTGTT
G
GAACTGCAACGATCCCTGGACTATCACCAAGAAAATGAGAAGGAGCTG
AA
GCAGACGCTTGTCGAGAACGAGCTGGCCACGCGGGCCCTCAAAAAGCA
GC
TAGACGAAAGCACTCTGCAGGCCTCCAAGGCGGTGATGGAGCGCACAA
AG
GCCTACAACGACAAGCTGCAACTGGAGAAGCGTTCCGAGGAATTGAGG
CT
GCAACTGGAGGCGCTCAAGGAAGAGCATCAAAAGCTGCTGCAGAAGCG
CT
CCAACAGCAGCGACGTTTCCCAGTCCGGTTACACATCCGAAGAGGTGGC
G
GTGCCCATGGGGCCACCTTCGGGTCAGGCTACAACGTGCAAACAGGCTG
C
TGCCGCAGTTTTGGGCCAGAGGGTGAACACATCATCTCCCGATCTGGGC
A
TAGAAAGCGATGCCGGCAGGATATCTAGCGTAGAAGTATCCAACGCCCA
A
CGTGCGATGCTTAAGACTGTAGAGATGAAAACGGAGGGATCAGCGAGTC
C
AAAGGCAAAGTCAGAGGAATCTACATCACCGGACAGCAAGAGCAACGT
GG
CAACTGGTGCAGCCACAGTACACGACTGTGCCAAGGTAGATCTTGAAAA
C
GCCGAGTTGCGGCGCAAACTAATCCGCACCAAGCGCGCATTTGAAGACA
C
CTACGAAAAGTTGCGTATGGCTAACAAAGCAAAAGCACAAGTTGAGAA
AG
ACATCAAAAATCAAATACTAAAAACGCACAATGTGCTGCGAAACGTTCG
C
TCAAACATGGAGAATGAGTTATAACGATCGCGCCGACGTCCAGTATATC
A
GCATTAACCCGAGAGGCCATTCACCCAATGTTCAATTGCCATTTCTTGCC
ATTCTTATATTTTGTATGCATTGTATTTTGTTGTCTCGTTGCTAACTTGA
TATCAATTATTTATGTGTTTTCTTCTATTTTTTTTTTTTAAAAAGA
CCGTAAACAACGAAGCTATTAGTACGAAATACCCATGCATTTTCTAAAG
A
CTGTTGAGGGAATCGAACCCATTGAATGAACTATCACACACACACACAC
A
CACACCTGTTTTAAGCACAAAACGAAACTAACTTGAAACTTGAAGTGCT
G
AAAATATGTATCTAACCCGGCGCACTCATTCATATTGAATGACACATGT
A
TATTAGAATGTATATATATATATATCCCCTAGTACCATAACTTTAACGAA
TATTCTCTAGCATACTCGACCTGATTCTTGTCTCGCATAACCCTCCATTT
TCCAATCATTTGCGTGTAGTGACTGCCTGATTAATGTATTCTCCTGCTGC
TTAACTGCTCTTAATCCTTGATTTCAGTTAACCATCGAAACGGATATTGA
AATAATTGTTTGACTTGCCGGACAAAACAGTTTAGCTACGTTGCCATCCA
ATAACCCTTTTTGTTCGAAACATCCATTTGTACAATGTGTAAAAAGTAGT
GTGCGTTGTCCTGGAGAACAAGATCTAGAACTTAAGTCATTTGTACCCGT
CGTTTTATGGAATAAACAATTGCGTTAGAATTA
>cnn|FBgn0013765
MDQSKQVLRDYCGDGNGTCASSLKEITLIETVTSFLEENGAAEIDRRVLR
KLAEALSKSIDDTSPGALQDVTMENSYASFDVPRPPGGGNSPLPSQGRSV
RELEEQMSALRKENFNLKLRIYFLEEGQPGARADSSTESLSKQLIDAKIE
IATLRKTVDVKMELLKDAARAISHHEELQRKADIDSQAIIDELQEQIHAY
QMAESGGQPVENIAKTRKMLRLESEVQRLEEELVNIEARNVAARNELEFM
LAERLESLTACEGKIQELAIKNSELVERLEKETASAESSNANRDLGAQLA
DKICELQEAQEKLKERERIHEQACRTIQKLMQKLSSQEKEIKKLNQENEQ
SANKENDCAKTVISPSSSGRSMSDNEASSQEMSTNLRVRYELKINEQEEK
IKQLQTEVKKKTANLQNLVNKELWEKNREVERLTKLLANQQKTLPQISEE
SAGEADLQQSFTEAEYMRALERNKLLQRKVDVLFQRLADDQQNSAVIGQL
RLELQQARTEVETADKWRLECVDVCSVLTNRLEELAGFLNSLLKHKDVLG
VLAADRRNAMRKAVDRSLDLSKSLNMTLNITATSLADQSLAQLCNLSEIL
YTEGDASHKTFNSHEELHAATSMAPTVENLKAENKALKKELEKRRSSEGQ
RKERRSLPLPSQQFDNQSESEAWSEPDRKVSLARIGLDETSNSLAAPEQA
ISESESEGRTCATRQDRNRNSERIAQLEEQIAQKDERMLNVQCQMVELDN
RYKQEQLRCLDITQQLEQLRAINEALTADLHAIGSHEEERMVELQRQLEL
KNQQIDQLKLAHSTLTADSQITEMELQALQQQMQEIEQLHADSVETLQSQ
LQKLKLDAVQQLEEHERLHREALERDWVALTTYQEQAQQLLELQRSLDYH
QENEKELKQTLVENELATRALKKQLDESTLQASKAVMERTKAYNDKLQLE
KRSEELRLQLEALKEEHQKLLQKRSNSSDVSQSGYTSEEVAVPMGPPSGQ
ATTCKQAAAAVLGQRVNTSSPDLGIESDAGRISSVEVSNAQRAMLKTVEM
KTEGSASPKAKSEESTSPDSKSNVATGAATVHDCALVDLENAELRRKLIR
TKRAFEDTYEKLRMANKAKAQVEKDIKNQILKTHNVLRNVRSNMENEL
pavScim
>>pav|FBgn0011692|cDNA sequence
CTTGGCGCTGTAAATTTGAAGGTAACGAACTAAAAGAGAGATCGATTCC
A
GCAACAAAACAAGAAGAAACCCAACTCCCGTATATCAAATTAGTTGCAA
A
AACGAGCTGACAACATGAAGGCAGTACCCAGGACGCCGATGCGTGTCGT
C
CCGAAAACTCCGCGGGTGCATCAAACGGTGGAGAAACAGCGACGAGAC
AC
CTCCGACAAGGCCAGGGATCCAGTGAATGTGTTCTGTCGAGTGCGGCCA
C
TACAATCCGACGCAGATCTCACCTCCCTAAGGGTCAAGAACTCCACGAC
G
ATCGCACTGAATCCGCAGGATCAGCTGCTGCAGCATCACAAGCCACACA
A
CGGCGCCCAGCGCGAGGTTCAGTACATATTCAAGCACGTTTTCCAGCCG
G
ATGCCACGCAGCAGGATGTGTACGCAGCTGTGGCCCAGCCGTTGGTGGA
G
AACCTACTGAAAGGCCGAAACAGTCTGCTTTTCACCTACGGCGTGACTG
G
AAGTGGAAAAACGTACACCATGACCGGCAACCTGCGACACCGCGGCAT
CA
TGCCGCGCTGCCTGGACGTGCTCTTTCGCACCATTTCAGATTACCAGGCC
AAGAAGTTCGTCTTCAAGCCGGACAAGCTTAATGGCTTCGAGATCCTGT
C
CGAAGAGGATGCGCTTTTGGAGCGCCAACACGAGATGAACCAGCGTTTT
CTGGCTCAGGACGATTTGCCTTCAGGCACAAGGACTCGGATCCGGAAAT
T
GCATCCCAGGCCTCCGTGGAGCCAATTCCGCTGCTGGGTTTGGATGAGG
A
CAACATGTACTCGGTGTTTGTCACCTATATTGAAATTTACAATAACAGCG
TCTACGACCTCCTTGAGGACTCGGGTATACAGAAGACTTTGCAGAGCAA
A
ATCATTCGTGAAGATGCCAATCGACACATGTTCGTCCACGGCGTCACAG
A
GGTGGAGGTCAAGACCGTGGAGGACGCCCTCGAGGTCTTTCAAATGGGC
C
AGAAGCGCAAACGAATGGGACACACTGTTCTCAATGCAGAATCCAGTAG
G
AGTCACTCGGTGTTCAATATACGCTTGGTGCAGGCGCCCACTGATAGCC
A
GGGTGAGAATGTGGTTCAGGACCGGCAAAACATCACAGTGAGTCAACTG
T
CCCTGGTGGATCTGGCGGGCAGTGAGCGATCCTCAAGGACCAAGAACAC
C
GGTGTGCGACTCCGTGAGGCGGGCAACATCAATAATTCGCTGATGACGC
T
GCGCACTTGCCTGGAATATCTGCGCGAGAACCAACTCGCGGCTAGCAAT
G
GATTGGCACCCAAGAAGGTCCCCTATCGAGACTCCAAGATCACACACAT
G
TTTAAAAACTACTTCGATGGCGAAGGCCAGGTGTCCATGATTGTGTGCAT
CAACCCAAGAATTGAGGACTATGATGAGAATATGCAAGTGATGAAGTTC
G
CCGAGATGACCCAGGAGGTGCAAATTGCTCGGGCGACTCCTATGAAGCA
A
GATTTGGGTCTGACTCCCGGCCGTCGCAAGGCCAACAAGCTGTTCAAGA
T
TGCCGTTAATAATCTAAACGAGCTGGGCATTCCTGAGGCCAAGGATTTG
G
AGGTGGACGTGGGTTTGGTGTACAGTTTGGGACCCGATTTTCCCGCCTAC
GAAATGGACAGTCCCGAGGCACAGATCAAAATCCGCGAGCTAATGCATT
A
TCTGGAGCAGCGCATAGAAAAGCGAAAGAAGCTGCGTGCCAACTTGGA
CA
TTAAATGTGACAGCTTCCGGCAGATGCTGATGAACCTTGACCGCGACAA
T
CTTCAGCTGCGCACGGAGCTCGCCTCCTTAAAGGCTGTTTACAAGCAGG
A
GCGCGATCGCAGTGCGGCACTCGAGAAGAAGGTTCGCATCCACGAGAG
CT
CCATTGACGTGCCTAACAATACGCTAAGCAAGCAAGAACGGCAGATTGA
A
GAGCTGACTTTCAAGCTGAACGAGAAGGAGAATATGCTCACCCAAAAGG
A
GCACGAGAAGGAAAAGCAAAAGAAGAAGTTCAGCTCCAAGCTGGCCGT
TG
AGTCGGACAAAAATAAGCGAGAGTTTGAACTAAAGCTGCGTGAACAGC
GA
GTGAAATTGCAGGAACGCATGCGCATCAAGGACGAGAAGTTGCGATTGG
T
CTCCAACATTCTGCAGTCCGAAGATCTGCCCAGTTTGCCGCGGTCACAG
A
GCTCGGAAAACATACTTAATGAGAAAGATCGCGGTGCATATACGGCGCG
C
ACGGAGGAGTCCTCGGTGCCAGCTACACGAACAGATATATATGCAACGC
C
ACGTCATGGTGCAGCAGCGGCCAACAATCGGCATAGACGCTCTCGATCC
G
CAGGCGACAAGTGGCTGGAGCATCGGGCAGCTAATCCCGTTCCGCTTGG
C
ACTATTATGCAGCCGTATCTGAAGAATCGCAAATCCGTAACGAAACTAA
C
CGACTTGAAGGAGTTGACTGCTCACGGAACTACGAAGTACTGTCTCGTC
T
CGCAGGATGCGGATACGGATGGCGATGTGGAGACGAAGCTGTATAAGG
GC
AATGTAATTCCCACTTGTGGCGGTGGTGCCCAAGTTGTGTTCAACGATGT
GGAGTGCCTCAAGCAGAAGTCACCGGTTCACTCGCCCACGCGAAAGCGA
C
CCAGCAATGGCAACATTTCGGCTCTGGGCGGCGGTGCAGTGCCCAGCAC
G
GTCACCTCAGCCCAGGACGTAGCCTCCCGCTGCAATCTCGGCATTGAGG
G
ACACAGCAGCAAGAAGTCGAAAATCTAAAAATGA
>pav|FBgn0011692
MKAVPRTPMRVVPKTPRVHQTVEKQRRDTSDKARDPVNVFCRVRPLQSDA
DLTSLRVKNSTTIALNPQDQLLQHHKPHNGAQREVQYIFKHVFQPDATQQ
DVYAAVAQPLVENLLKGRNSLLFTYGVTGSGKTYTMTGNLRHRGIMPRCL
DVLFRTISDYQAKKFVFKPDKLNGFEILSEEDALLERQHEMNQRFAGSGR
FAFRHKDSDPEIASQASVEPIPLLGLDEDNMYSVFVTYIEIYNNSVYDLL
EDSGIQKTLQSKIIREDANRHMFVHGVTEVEVKTVEDALEVFQMGQKRKR
MGHTVLNAESSRSHSVFNIRLVQAPTDSQGENVVQDRQNITVSQLSLVDL
AGSERSSRTKNTGVRLREAGNINNSLMTLRTCLEYLRENQLAASNGLAPK
KVPYRDSKITHMFKNYFDGEGQVSMIVCINPRIEDYDENMQVMKFAEMTQ
EVQIARATPMKQDLGLTPGRRKANKLFKIAVNNLNELGIPEAKDLEVDVG
LVYSLGPDFPAYEMDSPEAQIKIRELMHYLEQRIEKRKKLRANLDIKCDS
FRQMLMNLRDNLQLRTELASLKAVYKQERDRSAALEKKVRIHESSIDVL
NNTLSKQERQIEELTFKLNEKENMLTQKEHEKEKQKKKFSSKLAVESDKN
KREFELKLREQRVKLQERMRIKDEKLRLVSNILQSEDLPSLPRSQSSENI
LNEKDRGAYTARTEESSVPATRTDIYATPRHGAAAANNRHRRSRSAGDKW
LEHRAANPVPLGTIMQPYLKNRKSVTKLTDLKELTAHGTTKYCLVSQDAD
TDGDVETKLYKGNVIPTCGGGAQVVFNDVECLKQKSPVHSPTRKRPSNGN
ISALGGGAVPSTVTSAQDVASRCNLGIEGHSSKKSKI
rfc4Scim
NOTE:need to use rfc40 to recover the DNA and
amino acid sequences from FlyBase
>>RfC40|FBgn0015287|cDNA sequence
ATGCCGGAAGAACCAGAGAAAACGGCGGACGACAAGCGCAGTCACTTG
CC
GTGGATTGAGAAGTACCGCCCCGTGAAGTTCAAGGAGATAGTGGGCAAT
G
AGGATACCGTAGCAAGACTATCCGTATTCGCCACCCAGGGAAATGCACC
C
AATATTATTATTGCTGGTCCTCCTGGCGTGGGCAAGACAACCACAATCCA
GTGTCTGGCCAGGATTCTCCTGGGAGACAGCTACAAGGAGGCTGTCCTC
G
AGCTGAATGCCTCCAATGAACGAGGAATCGATGTGGTCCGCAACAAGAT
C
AAGATGTTTGCTCAGCAGAAGGTGACACTGCCGCGGGGCAGGCACAAG
AT
TGTGATTCTGGACGAGGCGGACAGCATGACGGAGGGTGCCCAGCAGGC
AC
TGCGCCGGACCATGGAGATCTACAGCAGCACCACTCGTTTTGCCCTGGC
C
TGCAACACCAGCGAGAAGATCATAGAGCCTATCCAGTCCCGCTGCGCCA
T
GCTACGATTCACCAAGCTGTCCGATGCGCAAGTTCTGGCCAAGCTCATC
G
AGGTGGCCAAGTGGGAGAAGCTTAACTACACGGAGGACGGACTTGAGG
CC
ATCGTATTCACTGCCCAAGGTGATATGCGACAGGGACTGAACAACCTGC
A
GTCCACCGCTCAGGGATTTGGCGACATCACCGCTGAGAACGTCTTTAAG
G
TCTGCGACGAGCCGCACCCAAAGCTCCTAGAGGAGATGATCCACCATTG
C
GCTGCTAACGACATACACAAGGCCTACAAAATCCTGGCCAAGCTGTGGA
A
GCTGGGATACTCGCCGGAGGACATCATTGCGAACATATTCCGTGTCTGC
A
AGCGCATCAACATCGATGAGCATTTGAAGCTGGACTTTATCCGTGAGAT
C
GGAATCACCCACATGAAGATTATCGATGGTATCAACTCGCTGCTGCAGC
T
CACCGCCCTTCTGGCCAAACTCTGCATTGCCGCCGAGAAGCATTAG
>RfC40|FBgn0015287
MPEEPEKTADDKRSHLPWIEKYRPVKFKEIVGNEDTVARLSVFATQGNAP
NIIIAGPPGVGKTTTIQCLARILLGDSYKEAVLELNASNERGIDVVRNKI
KMFAQQKVTLPRGRHKIVILDEADSMTEGAQQALRRTMEIYSSTRFALA
CNTSEKIIEPIQSRCAMLRFTKLSDAQVLAKLIEVAKWEKLNYTEDGLEA
IVFTAQGDMRQGLNNLQSTAQGFGDITAENVFKVCDEPHPKLLEEMIHHC
AANDIHKAYKILAKLWKLGYSPEDILANIFRVCKRINIDEHLKLDFIREI
GITHMKIIDGINSLLQLTALLAKLCIAAEKH
LanAScim
>>LanA|FBgn0002526|cDNA sequence
AGCCATCCAGTGCGGTTGGATGAGCTCCACCAAAGGAATACGAAAAGA
AC
AGTGAAAAAAGTGAAATACAGCAGATAAGAAAGTGAAATACATACATA
AG
TGAACCGGGAAAGAAAGAGATAGAACCGGAAGTGGTCCGTGGAGAAG
GA
CTGTCGTTATCGCAGCGCGGGTGCTTGAACTCTCGGCACAATGGGGCAC
G
GAGTGGCCTCCATTGGGGCTCTGCTAGTAATCCTGGCCATCTCCTATTGC
CAGGCGGAGCTAACGCCACCATATTTCAATCTGGCCACGGGCAGGAAGA
T
TTATGCCACAGCTACGTGTGGCCAGGATACGGATGGACCAGAGCTCTAC
T
GCAAACTAGTGGGAGCCAATACGGAACACGATCATATCGACTATTCAGT
T
ATCCAGGGACAGGTCTGCGATTACTGCGATCCCACGGTGCCGGAGAGGA
A
TCATCCGCCAGAGAACGCGATCGATGGCACCGAGGCCTGGTGGCAGAGT
C
CTCCGTTGTCCAGGGGCATGAAATTCAATGAAGTCAATTTGACCATCAA
C
TTCGAACAGGAATTCCATGTGGCCTATTTGTTCATTCGCATGGGCAACTC
CCCTCGTCCGGGTCTCTGGACGCTGGAGAAGTCCACGGATTATGGCAAG
A
CCTGGACGCCTTGGCAGCACTTTTCCGATACCCCCGCCGATTGTGAGACG
TACTTTGGCAAGGACACCTACAAGCCAATTACCCGCGACGACGACGTCA
T
CTGCACCACTGAATACTCAAAGATTGTGCCGCTGGAGAACGGCGAGATT
C
CCGTGATGCTGCTCAATGAGCGACCCAGTTCACCAACTACTTCAACTCA
ACGGTGCTGCAGGAATGGACTCGTGCCACTAACGTCAGGATTCGTCTGC
T
TCGCACCAAGAATCTTTTGGGACATTTGATGTCTGTGGCTCGACAAGATC
CCACGGTGACGCGTCGCTACTTCTACTCGATCAAGGACATCTCGATCGG
A
GGAAGGTGCATGTGCAATGGACACGCCGACACCTGTGACGTCAAGGACC
C
GAAGAGCCCAGTCCGCATCCTCGCATGCCGTTGCCAGCATCACAGTGC
G
GCATCCAGTGCAACGAGTGCTGTCCTGGATTCGAGCAGAAAAAGTGGCG
C
CAAAACACCAATGCCCGACCATTCAACTGCGAACCCTGCAACTGTCATG
G
TCACAGCAACGAGTGCAAGTACGATGAGGAAGTGAACCGCAAGGGACT
GT
CCCTGGACATTCACGGACACTACGATGGTGGTGGTGTTTGCCAGAACTG
T
CAGCACAATACCGTGGGAATTAACTGCAACAAGTGCAAGCCGAAATACT
A
TAGACCCAAGGGCAAGCACTGGAACGAGACCGATGTCTGCAGCCCCTGC
C
AATGCGACTACTTCTTCTCCACCGGACACTGCGAGGAGGAGACCGGAAA
C
TGTGAGTGCCGTGCTGCCTTCCAACCGCCAAGTTGCGACTCCTGCGCGTA
TGGATACTATGGTTACCCCAATTGCCGGGAGTGCGAATGTAATCTGAAT
G
GAACCAATGGCTACCACTGCGAGGCTGAAAGCGGACAGCAGTGCCCGT
GC
AAGATCAATTTCGCGGGGGCCTACTGTAAGCAGTGCGCCGAAGGATACT
A
CGGATTCCCCGAGTGCAAGGCATGCGAGTGCAACAAAATCGGCTCGATT
A
CCAACGACTGCAACGTGACCACTGGAGAGTGCAAGTGCTTGACCAATTT
C
GGAGGCGACAACTGCGAGCGCTGCAAGCACGGATACTTCAACTACCCTA
C
TTGCTCATACTGCGACTGCGACAACCAGGGCACGAGTCGGAAATCTGC
A
ACAAGCAGTCTGGCCAGTGCATCTGTCGTGAAGGTTTTGGAGGCCCAAG
A
TGCGATCAGTGCTTGCCAGGATTTTACAACTATCCGGACTGCAAGCCAT
G
CAACTGCTCCAGCACGGGTAGTTCGGCGATCACCTGCGACAACACGGGC
A
AATGTACCTGCCTCAACAACTTTGCCGGGAAACAGTGCACGCTCTGTAC
T
GCCGGATACTACAGCTATCCGGATTGTCTACGGAGTGTCCTGTAATTCAG
ACGGACAGTGCTTGTGTCAGCCCAACTTTGACGGACGTCAGTGCGATTC
C
TGTAAGGAGGGCTTCTACAACTTCCCCAGCTGCGAGGACTGCAACTGTG
A
TCCTGCAGGAGTGATCGATAAGTTCGCCGGATGCGGATCGGTGCCCGTG
G
GTGAGCTCTGCAAGTGCAAGGAGCGAGTGACGGGCAGGATCTGCAATG
AG
TGCAAGCCATTGTACTGGAACCTGAACATCAGCAATACTGAGGGATGTG
A
GATTTGCGATTGTTGGACAGATGGTACTATTTCCGCACTGGACACCTGTA
CCTCGAAGTCCGGACAGTGTCCCTGCAAGCCGCATACTCAAGGCAGGCG
C
TGCCAGGAGTGTCGGGATGGTACCTTCGATCTGGACAGTGCCTCGCTGTT
TGGCTGCAAGGACTGCAGTTGCGATGTGGGCGGCTCGTGGCAAAGTGTT
T
GCGACAAGATCAGCGGACAGTGTAAGTGCCACCCGAGGATTACGGGTCT
G
GCATGTACCCAGCCACTGACCACCCACTTCTTCCCAACGCTGCATCAGTT
CCAGTACGAATACGAGGATGGATCACTACCATCGGGCACTCAGGTGCGC
T
ACGACTACGATGAGGCGGCATTCCCCGGATTTAGCAGCAAGGGCTATGT
G
GTGTTCAACGCCATTCAGAACGATGTACGGAACGAGGTCAATGTCTTTA
A
GTCATCTCTGTATCGTATTGTCCTGCGATATGTGAATCCAAATGCCGAGA
ATGTCACTGCCACCATTTCGGTTACTTCAGACAATCCTCTGGAGGTTGAT
CAGCACGTTAAGGTTCTACTGCAACCCACATCGGAACCTCAGTTCGTAA
C
TGTTGCGGGTCCCTTGGGAGTGAAACCCTCAGCCATTGTTTTGGATCCGG
GTCGTTATGTATTTACCACCAAGGCCAACAAGAATGTGATGCTTGATTAC
TTTGTTCTCCTACCCGCCGCTTACTACGAAGCCGGAATTCTAACGCGCCA
CATCTCAAATCCCTGTGAACTGGGCAACATGGAGCTGTGCCGTCACTAC
A
AGTACGCCTCTGTGGAAGTGTTCTCACCAGCGGCAACTCCATTCGTCATC
GGTGAAAACTCCAAGCGGACAATCCCGTCGAGACGTACACTGATCCGG
A
GCATCTGCAGATTGTGAGCCATGTCGGTGATATTCCCGTACTGAGCGGA
T
CCCAAAATGAGCTGCACTACATCGTGGATGTGCCACGAAGTGGCCGGTA
T
ATCTTCGTGATTGACTACATCAGCGATAGGAACTTCCCCGACAGTTACTA
TATCAACTTAAAACTAAAGGATAATCCAGATTCGGAAACATCCGTGCTG
T
TGTATCCATGCTTATACTCGACTATTTGCCGAACATCAGTTAACGAGGAT
GGTATGGAGAAGTCATTCTATATTAACAAAGAGGATTTGCAGCCAGTCA
T
CATCTCCGCTGACATCGAAGATGGTTCTCGTTTCCCGATTATCTCGGTGA
CAGCAATTCCCGTCGACCAATGGTCCATTGACTACATCAACCCAAGTCC
C
GTCTGTGTGATCCATGATCAGCAATGCGCCACTCCGAAATTCCGTTCCGT
TCCCGATTCCAAGAAGATCGAGTTCGAAACTGATCATGAGGACCGTATT
G
CCACCAACAAACCTCCATATGCCTCACTCGATGAACGAGTTAAGCTGGT
C
CACTTAGACAGTCAAAATGAAGCAACCATCGTGATTGAATCAAAGGTTG
A
TGCTACAAAACCGAATCTATTTGTGATCCTGGTGAAGTACTACCAACCG
A
GCCACCCCAAATACCAGGTGTACTACACGCTGACAGCGGGCAAGAACCA
G
TACGATGGCAAGTTCGACATTCAGCACTGTCCATCGAGTTCCGGATGCC
G
CGGTGTGATACGCCCCCGGAGAGGGTTCGTTCGAGATTGACGACGAG
T
TCAAGTTCACGATTACGACCGATCGATCCCAGAGCGTTTGGCTGGACTA
C
CTGGTGGTGGTTCCCTTGAAGCAGTACAATGATGACCTACTGGTGGAAG
A
GACCTTCGACCAGACCAAGGAGTTCATTCAGAACTGCGGCCATGACCAC
T
TCCACATAACCCACAATGCCAGTGATTTCTGCAAGAAATCCGTTTTCTCT
CTAACAGCCGATTACAATAGTGGAGCATTGCCTTGCAATTGCGACTACG
C
CGGATCCACGAGCTTCGAGTGTCATCCGTTCGGAGGTCAGTGCCAGTGC
A
AGCCGAATGTGATTGAGCGCACGTGCGGAGCCTGTCGCAGTAGATACTA
C
GGATTCCCCGACTGCAAGCCATGCAAGTGTCCCAATAGCGCCATGTGTG
A
GCCCACCACTGGTGAGTGCATGTGTCCGCCCAATGTCATTGGGGATTTGT
GCGAGAAGTGCGCCCCCAACACCTATGGATTCCATCAGGTCATTGGCTG
C
GAGGAGTGTGCATGCAATCCTATGGGCATTGCAATGGAAACTCCCAGT
G
CGATCTGTTCAATGGAACCTGCGAGTGTCGGCAAAATATCGAGGGCAGG
G
CCTGTGATGTTTGCTCCAATGGATACTTTAACTTCCCGCACTGCGAGCAG
TGCAGTTGCCACAAGCCCGGCACCGAACTGGAGGTGTGTGACAAGATCG
A
TGGCGCATGCTTCTGCAAGAAGAATGTGGTTGGAAGGGACTGTGACCAG
T
GTGTCGATGGAACATACAATCTGCAGGAGTCGAATCCGGATGGTTGCAC
C
ACTTGCTTCTGCTTTGGCAAGACTTCGCGATGCGACAGTGCCTATCTCAG
AGTTTACAATGTGAGCCTGCTTAAGCATGTGTCCATCACTACTCCGGAAT
TCCACGAGGAGAGCATCAAGTTTGATATGTGGCCCGTACCCGCGGATGA
A
ATTCTTCTGAATGAGACCACGCTGAAGGCAGACTTTACCTTACGGGAGG
T
CAACGATGAACGACCTGCGTACTTTGGGGTTCTGGACTATCTGCTTAACC
AGAATAATCACATCTCTGCTTATGGCGGAGACCTGGCTTATACTTTACAT
TTCACTTCTGGCTTCGACGGCAAATACATTGTGGCTCCGGATGTAATTTT
GTTCAGTGAACACAATGCCCTGGTCCATACAAGCTATGAGCAGCCCAGC
C
GAAATGAACCATTCACCAACCGCGTCAATATAGTGGAATCGAATTTCCA
G
ACGATTTCCGGAAAACCAGTTTCACGAGCTGATTTCATGATGGTTCTGCG
GGATCTAAAAGTAATCTTTATCAGAGCCAACTATTGGGAACAGACCTTG
G
TGACCCACCTGTCCGATGTGTATCTAACTCTAGCCGATGAGGATGCAGA
T
GGCACTGGCGAATACCAGTTCCTGGCCGTGGAGCGTTGCTCCTGCCCAC
C
TGGTTACTCCGGACACTCGTGCGAGGACTGTGCCCCGGGCTACTACCGA
G
ATCCCAGCGGACCTTATGGTGGTTACTGCATCCCTGCGAGTGCAATGG
A
CATTCGGAGACCTGTGATTGCGCTACCGGAATCTGTTCAAAGTGCCAGC
A
TGGAACAGAGGGTGATCACTGTGAGCGCTGTGTGTCCGGTTACTATGGA
A
ACGCCACGAATGGAACACCCGGAGATTGTATGATCTGCGCTTGCCCACT
G
CCATTTGACTCGAACAACTTTGCCACCAGTTGCGAAATCTCCGAGAGCG
G
AGACCAAATCCATTGTGAGTGCAAGCCAGGATATACAGGACCACGTTGC
G
AGTCTTGCGCCAATGGTTTCTATGGAGAGCCAGAGAGCATAGGACAGGT
G
TGCAAGCCGTGCGAGTGTTCCGGTAACATCAATCCAGAGGATCAGGGAT
C
TTGCGACACTAGGACCGGTGAATGTCTTCCGCTGCTTGAATAACACCTTC
GGTGCTGCCTGTAATCTGTGTGCACCTGGATTCTATGGCGATGCTATCAA
GCTAAAGAACTGCCAGAGCTGCGACTGCGATGATCTGGGCACCCAGACGT
G
TGATCCCTTTGTGGGCGTCTGCACCTGTCACGAGAACGTCATTGGCGACC
GTTGCGATCGCTGTAAGCCGGATCACTATGGATTTGAGTCAGGTGTTGG
G
TGCCGAGCCTGCGACTGCGGTGCTGCCTGCCAATTCAACGCAGTGTGACC
C
ACACACAGGTCACTGTGCCTGCAAGTCGGGAGTGACAGGACGACAGTGC
G
ATCGATGCGCCGTTGACCATTGGAAATACGAAAAGGACGGTTGCACTCC
G
TGCAATTGCAATCAGGGATACTCACGAGGATTCGGTTGCAATCCCAACA
C
CGGCAAGTGCCAATGTCTGCCAGGAGTAATTGGAGATAGATGCGACGCT
T
GTCCGAATCGATGGGTTCTGATCAAGGATGAAGGCTGTCAGGAGTGCAA
C
AACTGCCACCACGCCCTTTTGGATGTCACTGATCGAATGCGCTATCAGAT
CGATAGTGTCCTGGAGGACTTTAACAGTGTAACCCTGGCCTTCTTCACCA
GTCAAAAATTGAACTATTACGACCAGCTGGCCGATGAACTGGAGCCCAA
G
GTTAAGCTATTGGATCCCAACAGCGTGGACCTGAGTCCATCCAAAAAGG
C
CAACAGCGAATTGGAATCCGATGCCAAGTCCTATGCCAAACAGGTCAAC
C
AGACCCTGGCCAACGCCTTCGATATCCGTGAGCGATCGAGCACCACTTT
G
GGTAACATAACAGTTGCTTATGATGAGGCGGTCAAGAGTGCCGACCAGG
C
CAAAGAGGCCATCGCTTCGGTGGAAGCTCTTTCCAAGAATCTCGAGGCG
G
CAGCGAGCACAAAGATTGATGCTGCTCTGGAGCAAGCACAGCACATTTT
G
GGACAAATCAACGGAACCAGCGCAAAACAAGAGCCTCAATGCCCTCAA
GA
ATGATATTGGAGAATTCAGCGACCATCTGGAAGATCTTTTCAACTGGAG
C
GAAGCTTCACAAGCCAAGTCCGCTGACGTTGAGCGCCGCAATGTGGCCA
A
CCAGAAGGCCTTTGACAACTCTAAATTCGACACCGTTTCGGAGCAAAAG
C
TACAGGCAGAGAAGAACATCAAAGATGCTGGCAATTTCCTCATCAATGG
C
GATCTGACCCTGAATCAAATTAATCAGAAGTTGGATAACTTGAGAGATG
C
TCTAAACGAGCTCAATTCGTTCAATAAAAATGTGGACGAAGAATTACCC
G
TGAGGGAGGACCAGCATAAGGAGGCGGATGCCCTTACCGATCAAGCCG
AA
CAGAAGGCGGCCGAATTGGCCATTAAGGCCCAAGATCTGGCTGCCCAAT
A
CACGGATATGACCGCCAGTGCGGAGCCGGCCATCAAGGCCGCAACTGCC
T
ACTCGGGAATCGTTGAGGCCGTTGAAGCTGCCCAGAAACTCAGTCAGGA
T
GCAATCTCTGCGGCCGGAAATGCCACTGATAAGACAGATGGCATTGAAG
A
GAGAGCTCATCTCGCTGACACTGGATCTACTGATCTTCTCCAGAGAGCA
C
GCCAGTCTCTGCAGAAGGTACAAGACGACCTAGAGCCCCGTCTGAACGC
T
TCGGCCGGCAAGGTGCAGAAAATCTCAGCCGTGAACAATGCCACCGAAC
A
CCAGCTGAAGGATATTAACAAGCTGATTGACCAACTGCCAGCTGAGTCG
C
AGAGGGATATGTGGAAGAATTCAAATGCCAATGCCAGTGATGCTCTGGA
G
ATACTGAAGAACGTCCTAGAGATACTCGAACCTGTGAGTGTCCAAACGC
C
AAAGGAACTCGAGAAGGCACATGGCATCAATAGAGATTTGGATCTGACC
A
ATAAGGACGTTTCTCAAGCTAACAAACAATTGGATGACGTAGAAGGATC
C
GTTTCGAAGCTAAGCGAATTGGCAGAGGATATCGAGGAACAGCAGCACC
G
TGTGGGCAGTCAGAGCCGGCAGTTGGGCCAGGAAATCGAGAACTTGAA
GG
CCCAAGTGGAGGCTGCTCGCCAGTTGGCCAACAGCATCAAGGTGGGCGT
C
AATTTCAAGCCAAGCACGATCCTGGAACTGAAGACACCGGAAAAGACC
AA
GTTGCTAGCCACTCGCACCAATCTATCGACCTATTTCCGCACCACCGAGC
CATCCGGTTTCCTTTTATATCTGGGCAATGACAACAAGACCGCCCAGAA
G
AACAACGACTTTGTGGCCGTTGAGATTGTGAATGGCTATCCGATTCTTAC
CATAGATTTGGGCAATGGGCCGGAACGCATCACCAGCGATAAGTACGTG
G
CAGATGGTCGCTGGTATCAGGCTGTTGTGGATCGCATGGGTCCGAATGC
C
AAGCTAACAATTAGGGAAGAACTACCCAACGGTGACGTGGTTGAGCACA
G
CAAGTCTGGCTATCTTGAAGGATCCCAGAATATCCTTCATGTGGATAAG
A
ACAGTCGCCTATTTGTTGGTGGCTATCCGGGTATTTCGGACTTCAATGCT
CCGCCAGACTTGACCACTAACTCCTTCTCCGGCGACATTGAGGATCTAA
A
GATTGGCGATGAGAGCGTAGGATTGTGGAACTTTGTATATGGCGATGAT
A
ACGACCAGGGCGCCAGGGAGCGGGATGTGCTGCTCGAGAAGAAGAAGC
CG
GTAACTGGTCTGCGGTTCAAGGGTAATGGATACGTCCAACTGAACGCCA
C
ATCGAACTTGAAGAGCCGCTCCAGCATCCAGTTCAGCTTCAAGGCAGAC
A
AGGATACTTCCAATGGACTGCTGTTCTTCTACGGCAGGGATAAGCACTA
C
ATGAGCATTGAGATGATTGACGAGCTATCTTCTTCAACATTAGTTTGGG
TGAAGGTGGAGGCGTTCAATCCGGCAGCCAGGATCGCTACAATGACAAC
C
AATGGCACAAGGTCCAGGCGGAAAGGGAAAACCAAACGGCCTTCTCA
AG
GTCGACGATATAGTAATATCACGGACCAATGCTCCATTGGAAGCGGACT
T
GGAGTTGCCCAAGCTGAGGAGATTATACTTCGGTGGTCATCCGAGACGT
C
TGAATACCTCGATAAGTTTGCAACCCAACTTTGATGGCTGCATCGATAAT
GTTGTGATCAACCAGGGAGTGGTCGACCTTACAGAATACGTAACTGGCG
G
CGGAGTTGAGGAAGGATGCTCGGCGAAGTTCTCCACGGTGGTGTCATAT
G
CGCCGCATGAGTACGGATTCCTGAGGATGAACAACGTTTCCTCAGATAA
C
AATCTGCATGTGGTTCTGCACTTCAAGACCACGCAACCGAATGGCGTAC
T
CTTCTATGCCGCCAATCATGACCAGAGTTCCACCATTGGCCTCAGCCTAC
AAGATGGTTTGCTCAAGCTCAACAGCATGGGCAGTCAGTTGGTAATCGA
C
GATCGTATTCTGAACGATGGCGAGGATCATGTGGTTACAGTTCAGCACA
C
CCAAGGTGAACTTCGTCTTACGGTCGATGATGTAGATAACAAGAGACTT
G
GTTCTCCACAACCATTAATCCTGGAGGGTGGTGACATCTTCTTTGCTGGC
TTGCCGGACAACTATCGAACACCGAGGAACGCGTTGGCCTCTTTGGCCT
A
CTTTGTCGGTTGCATCAGCGATGTAACCGTGAACGAGGAGATCATTAAC
T
TTGCCAACAGTGCTGAGAAGAAGAATGGCAACATCAACGGTTGCCCACC
A
CATGTACTAGCCTACGAACCAAGTTTGGTGCCCAGTTACTACCCAAGTG
G
AGACAACGAGGTGGAGTCTCCGTGGTCAAACGCCGATACTCTGCCCCCA
C
TTAAGCCAGATATTGAAAGCACACTACCGCCCACAACGCCCACTACGAC
C
ACCACGACAACTACCACAACAACATCGACTACTACAACTTCAACAACGA
C
AACTACTACGACACCATCCCCGATAGTCATTGATGAGGAGAAGGAGATT
G
AGGCGAAAACACCGCAAAAGATCCTAACAACGCGGCCACCAGCCAAGC
TG
AACCTTCCCAGCGATGAGCGCTGCAAGTTGCCGGAGCAACCCAATTTCG
A
TGTTGATTTCACAGAGGCGGGTTACCGATTCTATGGACTGCGGGAGCAG
C
GCCTGCAGATTAACTCACTGCCGGTGAAGGTACGCCGTCACCATGACAT
T
GGCATCTCATTCCGTACTGAGCGACCAAACGGTCTGCTCATTTATGCCGG
AAGTAAGCAGCGCGATGACTTCATAGCCGTGTATCTTCTGGACGGTCGG
G
TGACATACGAGATACGGTGGGTGCACAGTTGCAGGCCAAGATCACTAC
C
GAGGCGGAGCTGAACGACGGCACCTGGCACACCGTGGAGGTGGTGCGG
AC
CCAACGCAAGGTATCCCTGCTCATCGACAAGTTGGAGCAGCCGGGTTCC
G
TGGATCTGAATGCGGAAAGATCCGCTCCGGTATTGGCTGTCGAGTTGCC
C
ATCTATCTGGGCGGAGTTAACAAGTTCCTGGAGTCGGAGGTTAAGAATC
T
CACCGATTTCAAGACTGAAGTACCGTACTTTAACGGCTGCCTCAAGAAC
A
TCAAGTTCGACGCTATGGATCTGGAAACACCACCGGAGGAGTTTGGAGT
G
GTTCCGTGCTCTGAGCAGGTGGAACGCGGTTTGTTCTTCAATAACCAGA
A
GGCGTTTGTAAAGATCTTTGATCACTTCGATGTGGGCACTGAGATGAAA
A
TCAGCTTCGATTTCCGACCGAGGGATCCCAACGGATTGCTCTTCTCTGTG
CACGGCAAGAACTCTTATGCCATACTCGAGCTAGTAGACAATACCTTGT
A
CTTTACCGTGAAGACCGATCTGAAGAACATTGTGTCCACCAACTATAAG
C
TGCCCAACAACGAAAGCTTCTGCGATGGCAAGACGCGCAATGTTCAGGC
C
ATCAAATCCAAGTTTGTGATCAACATAGCCGTGGATTTCATTAGCTCCAA
CCCCGGCGTGGGCAACGAAGGATCAGTGATCACAAGGACCAATCGTCCG
C
TGTTCTTGGGCGGACATGTGGCCTTCCAGAGGGCCCCGGGCATCAAGAC
C
AAGAAGTCCTTTAAGGGCTGCATCAGCAAGGTGGAGGTTAACCAAAGGA
T
GATCAACATCACGCCGAACATGGTCGTTGGCGATATTTGGCAGGGCTAT
T
GTCCCCTCAACTAATAAAGAACTTCGGAACGACTTAAGGACCTATGATT
T
ATTTGGACAACGACTACGAAAAACTGCCAGGAGAATCTTCTTTTGCCG
A
CAACTGCTAATCAAAGGACAACGAACGATCAAATTTCAAACGAACAAA
AT
CGACGGGTCATAAAAAGTCATAAGGTCTTCAAAGTAATAGATCCTTAG
C
AAAGATGCCAGTCGTATATATTCCTAGTACTATAAGCCAAACTGCGTGC
C
AATTCTATTAAAGCCATTTACATTTATATATCCTTTTTAAAACTCCTTAA
GAGAATCTTGCATTTTTGTAATTCCAATAAACCAATAAAATAAACATTTT
AATGAAAC
>LanA|FBgn0002526
MGHGVASIGALLVILAISYCQAELTPPYFNLATGRKIYATATCGQDTDGP
ELYCKLVGANTEHDHIDYSVIQGQVCDYCDPTVPERHNPPENAIDGTEAW
WQSPPLSRGMKFNEVNLTINFEQEFHVAYLFIRMGNSPRPGLWTLEKSTD
YGKTWTPWQHFSDTPADCETYFGKDTYKPITRDDDVICTEEYSKIVPLEN
GEIPVMLLNERPSSTNYFNSTVLQEWTRATNVRIRLLRTKNLLGHLMSVA
RQDPTVTRRYFYSIKDISIGGRCMCNGHADTCDVKDPKSPVRILACRCQH
HTCGIQCNECCPGFEQKKWRQNTNARPFNCEPCNCHGHSNECKYDEEVNR
KGLSLDIHGHYDGGGVCQNCQHNTVGINCKCKPKYYRPKGKHWNETDV
C
SPCQCDYFFSTGHCEEETGNCECRAAFQPPSCDSCAYGYYGYPNCRECEC
NLNGTNGYHCEAESGQQCPCKINFAGAYCKQCAEGYYGFPECKACECNKI
GSITNDCNVTTGECKCLTNFGGDNCERCKHGYFNYPTCSYCDCDNQGTES
EICNKOSGOCICREGFGGPRCDQCLPGFYNPDCKPCNCSSTGSSAITCD
NTGKCNCLNNFAGKQCTLCTAGYYSYPDCLRSVL
eIF-4Scim
>>eIF-4E|FBgn0015218|cDNA sequence
CGGCCACACTGTCTGGCCACCAAAATCCCAAACTTAATTAAAGAATTAA
A
TAATTCGAATAATAATTAAGCCCAGTAACCTACGCAGCTTGAGTGCGTA
A
CCGATATCTAGTATACATTTCGATACATCGAAATCATGGTAGTGTTGGAG
ACGGAGAAGTTTTTTGGTTAATAATACACAAGTGAAGAGCGAACTGCAG
G
GGAGCGAGATATCACGAAACAATCCAAAATCCACACACACTCAAACAG
AA
ATCAAAAGCTTCGCTCTCTCGCACACACACGCACCAACCAACTATCAAC
T
ATCACAAACACCGCGACAGAGAGAGAGCGGCAAGTGAATCACGGCGAA
TC
GAAACCGATCCGAACCCACTCCGGAGCCGAAAAAGAACTGATCCTACCA
T
CAAACGCATCCAATAAACACGGCCGCCAACATGCAGAGCGACTTTCACA
G
AATGAAGAACTTTGCCAATCCCAAGTCCATGTTCAAAACCAGCGCCCCC
A
GCACCGAGCAGGGTCGTCCGGAACCACCAACTTCGGCTGCAGCGCCCGC
C
GAGGCTAAGGATGTCAAGCCCAAGGAGGACCCACAGGAGACTGGTGAA
CC
AGCAGGCAACACTGCAACCACTACTGCTCCTGCCGGCGACGATGCTGTG
C
GCACCGAGCATTTATACAAACACCCGCTCATGAATGTCTGGACGCTGTG
G
TACCTTGAAAACGATCGGTCCAAGTCCTGGGAGGACATGCAAAACGAGA
T
CACCAGCTTCGATACCGTCGAGGACTTCTGGAGCCTATACAACCACATC
A
AGCCCCCATCAGAGATCAAGCTGGGTAGTGACTACTCGCTATTCAAGAA
G
AACATTCGTCCCATGTGGGAGGATGCAGCCAACAAACAGGGCGGTCGTT
G
GGTCATTACCCTTAACAAAAGCTCCAAGACCGATCTGGATAACCTATGG
C
TCGATGTGCTGCTCTGCCTGATTGGTGAGGCCTTCGATCACTCTGATCAG
ATCTGCGGCGCTGTTATAAACATTCGCGGCAAGAGCAACAAGATATCCA
T
CTGGACTGCCGACGGAAACAACGAGGAAGCTGCCCTTGAGATTGGTCAC
A
AGCTGCGCGATGCCCTGCGTCTGGGACGCAACAACTCGCTGCAGTATCA
G
TTGCACAAGGACACGATGGTCAAGCAGGGCTCCAACGTGAAATCGATCT
A
CACTTTGTAGGCGGCTAATAACTGGCCGCTCCTTATTCGGTCCGATCCCA
CACTGATTATTTTGTCTTTCATTTATTTATCGTTATAAGCAACAGTAGCG
ATTAATCGTGACTATTGTCTAAGACCCGCGTAACGAAACCGAAACGAA
C
CCCCTTTGTTATCAAAAATCGGCATAATATAAAATCTATCCGCTTTTTGT
AGTCACTGTCAATAATGGATTAGACGGAAAGTATATTAATAAAAACCT
A
CATTAAAACCGG
>>eIF-4E|FBgn0015218|cDNA sequence
GCCACACTGTCTGGCCACCAAAATCCCAAACTTAATTAAAGAATTAAAT
A
ATTCGAATAATAATTAAGCCCAGTAACCTACGCAGCTTGAGTGCGTAAC
C
GATATCTAGTATACATTTCGATACATCGAAATCATGGTAGTGTTGGAGAC
GGAGAAGACCAGCGCCCCCAGCACCGAGCAGGGTCGTCCGGAACCACC
AA
CTTCGGCTGCAGCGCCCGCCGAGGCTAAGGATGTCAAGCCCAAGGAGGA
C
CCACAGGAGACTGGTGAACCAGCAGGCAACACTGCAACCACTACTGCTC
C
TGCCGGCGACGATGCTGTGCGCACCGAGCATTTATACAAACACCCGCTC
A
TGAATGTCTGGACGCTGTGGTACCTTGAAAACGATCGGTCCAAGTCCTG
G
GAGGACATGCAAAACGAGATCACCAGCTTCGATACCGTCGAGGACTTCT
G
GAGCCTATACAACCACATCAAGCCCCCATCAGAGATCAAGCTGGGTAGT
G
ACTACTCGCTATTCAAGAAGAACATTCGTCCCATGTGGGAGGATGCAGC
C
AACAAACAGGGCGGTCGTTGGGTCATTACCCTTAACAAAAGCTCCAAGA
C
CGATCTGGATAACCTATGGCTCGATGTGCTGCTCTGCCTGATTGGTGAGG
CCTTCGATCACTCTGATCAGATCTGCGGCGCTGTTATAAACATTCGCGGC
AAGAGCAACAAGATATCCATCTGGACTGCCGACGGAAACAACGAGGAA
GC
TGCCCTTGAGATTGGTCACAAGCTGCGCGATGCCCTGCGTCTGGGACGC
A
ACAACTCGCTGCAGTATCAGTTGCACAAGGACACGATGGTCAAGCAGGG
C
TCCAACGTGAAATCGATCTACACTTTGTAGGCGGCTAATAACTGGCCGC
T
CCTTATTCGGTCCGATCCCACACTGATTATTTTGTCTTTCATTTATTTAT
CGTTATAAGCAACAGTAGCGATTAATCGTGACTATTGTCTAAGACCCGC
G
TAACGAAACCGAAACGGAACCCCCTTTGTTATCAAAAATCGGCATAATA
T
AAAATCTATCCGCTTTTTGTAGTCACTGTCAATAATGGATTAGACGGAAA
AGTATATTAATAAAAACCTACATTAAAACCGG
>>eIF-4E|FBgn0015218|cDNA sequence
GAACAACAAAAACAGGGTTGTTGCACAAGAGGGGAGGCGATAGTCGAG
CG
GAAAAGTGTGCAGTTGGCGTGGCTACATCATCATTGTGTTCACCGATTAT
TTTTTGCACAATTGCTTAATATTAATTTTTTTTGGTTAATAATACACAAG
TGAAGAGCGAACTGCAGGGGAGCGAGATATCACGAAACAATCCAAAAT
CC
ACACACACTCAAACAGAAATCAAAAGCTTCGCTCTCTCGCACACACACG
C
ACCAACCAACTATCAACTATCACAAACACCGCGACAGAGAGAGAGCGG
CA
AGTGAATCACGGCGAATCGAAACCGATCCGAACCCACTCCGGAGCCGA
AA
AAGAACTGATCCTACCATCAAACGCATCCAATAAACACGGCCGCCAACA
T
GCAGAGCGACTTTCACAGAATGAAGAACTTTGCCAATCCCAAGTCCATG
T
TCAAAACCAGCGCCCCCAGCACCGAGCAGGGTCGTCCGGAACCACCAAC
T
TCGGCTGCAGCGCCCGCCGAGGCTAAGGATGTCAAGCCCAAGGAGGAC
CC
ACAGGAGACTGGTGAACCAGCAGGCAACACTGCAACCACTACTGCTCCT
G
CCGGCGACGATGCTGTGCGCACCGAGCATTTATACAAACACCCGCTCAT
G
AATGTCTGGACGCTGTGGTACCTTGAAAACGATCGGTCCAAGTCCTGGG
A
GGACATGCAAAACGAGATCACCAGCTTCGATACCGTCGAGGACTTCTGG
A
GCCCTATACAACCACATCAAGCCCCCATCAGAGATCAAGCTGGGTAGTGA
C
TACTCGCTATTCAAGAAGAACATTCGTCCCATGTGGGAGGATGCAGCCA
A
CAAACAGGGCGGTCGTTGGGTCATTACCCTTAACAAAAGCTCCAAGACC
G
ATCTGGATAACCTATGGCTCGATGTGCTGCTCTGCCTGATTGGTGAGGCC
TTCGATCACTCTGATCAGATCTGCGGCGCTGTTATAAACATTCGCGGCAA
GAGCAACAAGATATCCATCTGGACTGCCGACGGAAACAACGAGGAAGC
TG
CCCTTGAGATTGGTCACAAGCTGCGCGATGCCCTGCGTCTGGGACGCAA
C
AACTCGCTGCAGTATCAGTTGCACAAGGACACGATGGTCAAGCAGGGCT
C
CAACGTGAAATCGATCTACACTTTGTAGGCGGCTAATAACTGGCGCTC
C
TTATTCGGTCCGATCCCACACTGATTATTTTGTCTTTCATTTATTTATCG
TTATAAGCAACAGTAGCGATTAATCGTGACTATTGTCTAAGACCCGCGT
A
ACGAAACCGAAACGGAACCCCCTTTGTTATCAAAAATCGGCATAATATA
A
AATCTATCCGCTTTTTGTAGTCACTGTCAATAATGGATTAGACGGAAAAG
TATATTAATAAAAACCTACATTAAAACCGG
>eIF-4E|FBgn0015218
MQSDFHRMKNFANPKSMFKTSAPSTEQGRPEPPTSAAAPAEAKDVKPKED
PQETGEPAGNTATTTAPAGDDAVRTEHLYKHPLMNVWTLWYLENDRSKS
W
EDMQNEITSFDTVEDFWSLYNHIKPPSEIKLGSDYSLFKKNIRPMWEDAA
NKQGGRWVITLNKSSKTDLDNLWLDVLLCLIGEAFDHSDQICGAVINIRG
KSNKISIWTADGNNEEAALEIGHKLRDALRLGRNNSLQYQLHKDTMVKQG
SNVKSIYTL
>eIF-4E|FBgn0015218
MVVLETEKTSAPSTEQGRPEPPTSAAAPAEAKDVKPKEDPQETGEPAGNT
ATTTAPAGDDAVRTEHLYKHPLMNVWTLWYLENDRSKSWEDMQNEITSF
D
TVEDFWSLYNHIKPPSEIKLGSDYSLFKKNIRPMWEDAANKQGGRWVITL
NKSSKTDLDNLWLDVLLCLIGEAFDHSDQICGAVINIRGKSNKISIWTAD
GNNEEAALEIGHLRDALRLGRNNSLQYQLHKDTMVKQGSNVKSIYTL
>eIF-4E|FBgn0015218
MQSDFHRMKNFANPKSMFKTSAPSTEQGRPEPPTSAAAPAEAKDVKPKED
PQETGEPAGNTATTTAPAGDDAVRTEHLYKHPLMNVWTLWYLENDRSKS
W
EDMQNEITSFDTVEDFWSLYNHIKPPSEIKLGSDYSLFKKNIRPMWEDAA
NKQGGRWVITLNKSSKTDLDNLWLDVLLCLIGEAFDHSDQICGAVINIRG
KSNKISIWTADGNNEEAALEIGHKLRDALRLGRNNSLQYQLHKDTMVKQG
SNVKSIYTL
Gap1Scim
>>Gap1|FBgn0004390|cDNA sequence
TCCGCTTCGAAAGGTTGTTCGCGGATGCGTGTAAACCCGCAAAGCAAAT
A
AACTCGGAGAACCAAGTTTTTGCGATTCTGATTCAAGCAACCGAATTAC
T
TTCCATACTTCGATAATATAATCCCACGTCTGCTAGTGCGTGCAAAAAAC
AGTTTGTAGTTAAAACGAAATACTAAAATCAGTCGAGCCCTGAAGCAAAC
C
AGCTCAATGCACAATGCAACGCTGCAGCGAGTGATGAAATTCAAATGCA
A
ATAGCCGGCTAATAAACCTGTTAAAGAAAAACAAATAATTTCGAGTTAT
T
AAAGTTTCGTGAAGATCGAGTGAAGGAGTTAGATAAATTTCCAATCGCG
T
GTTGCAAGTTCCCCAGTGGGTAGCTGACAAAAAGCTGGCTGCTTTAAGC
G
CACAATGCTGCTGAAGAAGAAGCGCTATATGTTCGACGAGCGAGACGAC
A
ATAATATTAACTCGCCCGTCGCCGTCGAGCCAAGCAGCAACAACAACAA
A
ATGGCCGATACACGCGAAGTGCGCATTGAAGAGCAATTAAAAGTGAAA
AT
TGGCGAGGCAAAGAATCTGAGCAGTCGCAATGCGGCCAACACTAGCTGC
A
GCACACAGGGCACTCGCGATGTCTACTGCACAATTGCGTTGGACCAGGA
G
GAGATCTGCCGCACACCCACCATCGAACGCACACTGACGCCGTTCTTCG
G
CGAGGAGCACCAGTTCAAGATTCCGCGCCGCTTCCGCTACCTGACCATC
T
ATCTGTGGGATCGTGATATGAAGCAGGATAAACCCATTGGCAAGATAGC
G
ATCAAGCGGGAGGAGCTGCACATGTACAATCACAAGGATCACTGGTTCT
C
ATTGAGGCCAGTCGACCAAGACTCCGAGGTTCAGGGCATGGTTAATGTG
G
AGGTGGCATTTACGGAAGCCCAACAGACCCAGTCGCTCTCCGAGGGCAT
A
GATCTGGGACAGCACACATTGCGGCATCATCAGAACCTGCCACACCACA
G
CCACCAGCAGCGGGCGCATCTAAACGATTACAAGGAGAACAGTGAACT
GA
GTAACATACAGCGGGCATCAGCAGCTGCAGCGAGTTCCTCATCGGCAGC
C
ATGACACTCAAGACACGAGCGGCCGGTCTATTTGGTCACGTGCATCATC
C
GCCATCGCAGACTCAGCACTTTCCGATCATCAATACCACATCCACGTCGT
CGGATCAGTTGTCCAATTGGAAATCGCATGGGAGATTCGTTGGTGTGAC
C
ATCAAAGTACCAGCCTGCGTGGACCTGGCCAAGAAGCAGGGCACGTGC
GC
TCCATTTGTGGTCTGTACCGCCCACTATAGCAACAAGCACCAGGTGACG
C
GACGAACTAAGCAGCGCAAAAAGACCGTGGATCCGGAGTTTGACGAGG
CC
ATGTACTTTGATCTTCACATCGATGCGGATGCGGGTAGCACAAATACCA
C
TGGAAGTAACAAGAGTGCCGGTTCCCTGGAATCATCAGCGAACAAGGGC
T
ATTCTATATACCCGGTGGGCGGTGCGGATCTGGTGGAGATTGTGGTGAG
C
GTGTGGCACGACGCACATGGAGCCATGTCCGATAAAGTGTTCCTTGGTG
A
AGTGCGGCTACCAATGCTCAATAAGCAGGAGCAGCAGGCAGTTAATCCA
T
CGGCTTGGTACTATCTACAGCCCCGCTCCATGACGCACAGTTCCCGCTCG
CTGAACGCCACGCCCCGCTCATGTGCGACGCCGCCGGGAACGAGATTAA
G
CGTGGACTCGACCATTGGATCACTGCGCTTAAATCTAAACTACACCGCC
G
ATCATGTCTTCCCGCTGGCCACATACGACGATCTTATGAATCTGCTCCTG
GAATCGGTTGATCAGCGACCCATCACCGTGTCGGCTGTGTCGATCCTAG
G
CGAGCTGGTCTCGGGCAAGACGGAGGTGGCCCAGCCGCTGGTGCGACTC
T
TTACGCACACGGAGCGCATTGCGCCGATCATCAAGGCGCTGGCTGACCA
C
GAGATCTCCCATCTGACAGATCCCACCACTATCTTCCGGGGTAACACCTT
GGTCTCTAAGATGATGGATGAGGCAATGCGATTGTCTGGACTGCACTAC
C
TACATCAGACATTGCGTCCGGTTTTGTCGCAGATCGTTGCCGAAAAGAA
A
CCATGTGAGATCGATCCCAGCAAGATTAAAGATCGCTCTGCGGTGGACA
C
GAATCTGCACAATCTGCAAGATTATGTGGAGCGGGTTTTTGAGGCAATC
A
CCAAGAGCGCTGATCGCTGTCCGAAAGTTCTGTGTCAGATCTTCCACGA
C
CTTCGCGAGTGTGCGGGTGAGCATTTCCCCAGCAATCGAGAAGTGCGCT
A
CTCCGTGGTGTCCGGCTTTATCTTCCTGCGCTTCTTTGCACCCGCTATCC
TGGGACCAAAGCTCTTCGATCTGACCACCGAGCGACTGGATGCCCAAAC
G
AGTCGCACGCTGACTCTCATCTCCAAGACGATCCAATCGCTGGGTAATCT
GGTCAGCTCCCGGTCGTCGCAACAGACTTGCAAGGAGGAGTTCACCGTC
G
AGCTGTACAAGAAGTTCTGCACGGAACAGCACGTGGATGCGGTTAAGCA
C
TTTCTGGAGGTGATCTCGACGCCCAGTCATGCGAGCAGCAGCGTTCATC
C
GGCGGCGGCGGCGGCAACGCCACTGGAACCAGTTTTACTTAAAGAGGGC
C
TGATGACCAAGTATCCGACATCCCGGAAGCGTTTTGGCCGGCAGTTCAA
A
CAACGCCACTTCCGCCTCACCACCCACTCGCTCAGCTATGCGAAGTCGA
A
GGGCAAACAACCCATCTGTGATATACCGCTCCAGGAGATCGCCAGCGTG
G
AGCAGCTGAAGGACAAGAGTTTCAAGATGCAGAACTGCTTTAAGATTGT
G
CACAACGATCGCTCGCTGATTGTGCAGACAACGAACTGCGTGGAGGAGC
G
CGAGTGGTTCGACCTGCTGCACAAGATATGTCTGATGAACTCCATCCGG
A
TGCAGTACTTCCATCCCTCGGCCTTTGTAAGTGGATTCTACAGCTGCTGC
GGCCGGTCGGATGAGAATTCACCGGGCTGTAAGAAGGTGTTGGACAAGA
G
ATCTGCAGCGCATCCACACGCTGATCATGTCTAACATGAGTGTGCTGGA
G
TCACTGCTAGATCCGCTCACCTATCACCAGTCTCTGTCGCAGACGCAACA
CCAACAGCACAATCCTCTGGTGCCGCTGGCCACCGATCTGCAGAAGCAT
T
CACCACAGGCCTTCGCTGAGTTCAAGCGAACGATTGAGAAGCTGAGAGA
G
AAGGCCTATGCCATCGATAGAGATCATCGGGACTACAAGAGGGCATTA
C
GCGGCAACTGAAATACGGCAGCAGACAGGCACCCATTGGCGATGATAA
CT
ATTGGCATATGATGCGAGCCGCCGGCCAGTTGAATCAGCAGCATCACCA
G
CAGCAGCAACATCAGCAGCAACAGCAACAGCAGCAGCAGCAGCAACTT
CA
GCAGTTCCAGCCGCAGCCAGTGCTGCCGCAATGCAGAACGTTCGGGCG
T
ATCCTTATCAGCCAGCTACGAGCAACATGAACGCCTACTGTCTGCACAA
C
ATGCAGTATCAGCAGCAGAGATTGCCGTTCCATCAGCAGCAACAGCAGC
A
CCATCAGCAGCTCCAGCAACAGCAGTCGCAGTTCCAGCCCCTGCGCAGC
C
ATCAACTGCAGCGCCACAACAACAATTTGAACAACAATAACTGCGGCAA
T
GGATCCTCCTCCAGTCCATCATCGACCACATCAAGTGTGGTGGCCGCAC
C
GCCCAGCACCACATCGTCATCGCAGCCAGCTCCACCGATTTATTAGCGTT
CAAGGCTGTGCGTATTCGCTTTGCCGCGGGCCATTATCACGACCAGGTA
A
AGGAATACGCATCGATGTGGCAAACACAACCCCCTACCGCTCCTACGCA
A
CCAGAGGATGCTGCTGTACAAAACGATTAATTGTGCTAATTTCTAGGCTC
TTGTTTTTTTTTATCATTTTTACGAGTTTAACGTTGTAAAGGTCTAGA
CTAAGTTTTTTTAATTAACAGTTTTTATACACCAGATTTTTACGGTACCG
CCTACACCAGCTACAACAACACGAGGAGAAAACAAGAGGAACGGAAAC
GA
AATCTAAATATTTATAAGCCTATTTATTTTTTTGTATGTGCTAACATTAT
TTTTACACGAAAACAAAATACCCAATATGGAAAGAGCATTTCTATACTT
G
TGTGAGCGCAAAGAGGGCAGAGAACCACTGAAAACTGATAACTTTATAA
G
CCTAAACTACTAAAAACTCAGAAACTGCTAAGATTTTTTAAAGCCTAGC
C
TAAGTTAAGTATCGGCGGGATGATGAGTAGCGAAATGAAAATGAAAAG
AT
GTAAATAGCAGATACACGAGTATATATATATCCCACAGTATGTATGTAC
A
TGTGTATGATTAACTTCATTATACCAGTTAATTTAGGCGTAGCAAAAATT
TCTAAAAAATCAACTAAACTAGGCGTTTTGCCCAGAGGCGACACAGCG
A
AAAGCAAATAATTCTAACGCAATCTGAACGCGAGAAAAATACTGCGAA
AA
GCATG
>Gap1|FBgn0004390
MLLKKKRYMFDERDDNNINSPVAVEPSSNNNKMADTREVRIEEQLKVKIG
EAKNLSSRNAANTSCSTQGTRDVYCTIALDQEEICRTPTIERTLTPFFGE
EHQFKIPRRFRYLTIYLWDRDMKQDKPIGKIAIKREELHMYNHKDHWFSL
RPVDQDSEVQGMVNVEVAFTEAQQTQSLSEGIDLGQHTLRHHQNLPHHSH
QQRAHLNDYKENSELSNIQRASAAAASSSSAAMTLKTRAAGLFGHVHHPP
SQTQHFPIINTTSTSSDQLSNWKSHGRFVGVTIKVPACVDLAKKQGTCDP
FVVCTAHYSNKHQVTRRTKQRKKTVDPEFDEAMYFDLHIDADAGSTNTTG
SNKSAGSLESSANKGYSIYPVGGADLVEIVVSVWHDAHGAMSDKVFLGEV
RLPMLNKQEQQAVNPSAWYYLQPRSMTHSSRSNATPRSCATPPGTRLSV
DSTIGSLRLNLNYTADHVFPLATYDDLMNLLLESVDQRPITVSAVSILGE
LVSGKTEVAQPLVRLFTHTERIAPIIKALADHEISHLTDPTTIFRGNTLV
SKMMDEAMRLSGLHYLHQTLRPVLSQIVAEKKPCEIDPSKIKDRSAVDTN
LHNLQDYVERVFEAITKSADRCPKVLCQIFHDLRECAGEHFPSNREVRYS
VVSGFIFLRFFAPAILGPKLFDLTTERLDAQTSRTLTLISKTIQSLGNLV
SSRSSQQTCKEEFTVELYKKFCTEQHVDAVKHFLEVISTPSHASSSVHPA
AAAATPLEPVLLKEGLMTKYPTSRKRFGRQFKQRHFRLTTHSLSYAKSKG
KQPICDIPLQEIASVEQLKDKSFKMQNCFKIVHNDSLIVQTTNCVEERE
WFDLLHKICLMNSIRMQYFHPSAFVGFYSCCGRSDENSPGCKKVLDKTM
DYFQMDLVTALDPALDLQRIHTLIMSNMSVLESLLDPLTYHQSLSQTQHQ
QHNPLVPLATDLQKHSPQAFAEFKRTIEKLREKAYAIDRDHRDYKQGITR
QLKYGSRQAPIGDDNYWHMMRAAGQLNQQHHQQQQHQQQQQQQQQQ
LQQ
FQPQPVLPQMQNVRAYPYQPATSNMNAYCLHNMQYQQQRLPFHQQQQQH
H
QQLQQQQSQFQPLRSHQLQRHNNNLNNNNCGNGSSSSPSSTSSVVAAP
STTSSSQPAPPIY
JIL-1Scim
>>JIL-1|FBgn0020412|cDNA sequence
CTACGTGCGGTTCGTGTGCTGTGTGTTGAAATTACAAATAGCCAATTATC
CGGCATTAATCGTTTTAGGGCTCTCTCGATTTTAATTGTATTATATTGTA
CTATAATACAGATATAAACCCGATTTGATCGAGTGTAACGGAAATCGAC
G
CGATTCCCATCAAGGTGGTCCGTACCTAATAAGTGTTCACGAATGAGAG
C
AAAGCAGATGGCGGAGAGCGTCCTGCGTTTCAATGTGTGTGTGCGTAAA
C
GAGTAGAAAGCGGCGGTAAAAACCTTCTCGTGTAACACAAAACAAATTA
T
ACAAAACATTCAATCTCAATATCAAAACGTTACTGTATTGTCTATTGAAA
AACAAGTACAAGCGTACGCAGGGAAATTGGAAAATCCATTGAAAATCG
AG
AGCAACAGTCGCCGGAGCGGAATCAGCGGACGAAAACAAAAACGCTTT
AA
AAACGCAGCCGATACAGAAAGTGCCAGCGTGTGAGTGTGCATATGTGTA
C
GTAGTTGTGGGCAAAGAGAACGGTAAAGAATAAGAAATCGAAAAAAAA
AA
ATAAAAAACTAACTGTGGAAGTGAAAAATGCGCGACTGATAAAGATTGT
G
ATAATCACGGCATAATATAACAAGCAAAAAAGAAGAATGAAAAGAAAC
CG
CAACCGAATTGGTTGAATAATATTTAAGCCAACGCCAGTGCTGCATAAA
T
CGGTGCACATTATCATTGGAACGTTCAGTGTTGCACATCGCTACCGCCCC
ACGCATAATCCAACACTCTTCCCCCTCCCCATCCAGATCCACCCACTCAC
ACACACACACACTCCGATTCGGATTTCTTCGTCTGCATTATAATATTGCG
CTTATAATCTTTCGCGAGATGAGTCGCTTGCAAAAGCAAAACTACGAGA
T
CTTAAGCGGTACAAGTACGTCGCGCCTCAAGAATCATCAACATCCCCGT
G
AATCGGAGTCACTAGCCTACGAGGAGCCAGATCAAATGGTGCGGAATCA
T
CTAAATGGCCAGCTGGTAGCCAATGGCAATGGCAAAACGCGAAAGAAT
AG
CAATTCCGAAACGATGACCAATGGGAAAAAGTCCAAACTGAATACGGA
GG
GATCCGGCTCTGGTTCTGGAAAGACCTTGAACTACAATAATAATAACAA
T
AACAACAACAGCATCAGTGCCACCAACGGTCAGTACACCAATAGCAGC
AG
CAAGACCACTTCAGCATCTGCAAGGGATTACACTTACCGCGAGACCATA
T
CACCGCCCACACCTCCATCCCCGCCCACCACAAATGTGGCGGATATTGT
T
TGCATATCGGATGCGGAGAGCGAGGACGGACGCGATCCCGAGCGGGAG
TA
CTACGACCAGGACATGGAGGAGGATGAGCCGAACGGGATAGAGATCGA
TG
AATCCAGCTCCAGCTTGTCCAAGGCCAAGTCCAACAATGCAGCTGCAGC
G
GCGGCGGCGGCGGCTGCAGCAGCGGCAGCAGCAGCCTCGAAGGCCTCC
TC
GTCGACCACGCCCTCCTACGCCATGCCCACCAGCAATAGTACACCCCTT
G
ATCTGGACAATGAGGCCCATCAACGGGACCTGGAGGCGGTCACCGATCT
G
AAATACTACGTAAAATTGTACAGTGATGAGGCCGTTAGTCTGAATGACT
T
CAAAATAATACGCGTTCTCGGAACAGGTGCCTATGGGCGGGTTTTCCTC
G
TTCGCAAGCTAACTAGACACGATGCCGGAAAATTGTATGCAATGAAGGT
T
CTCAATAAGATAACGGTGGTCCAGAAGCGAAAAACCGCGGAACACACC
AA
AACGGAGAGAGTGGTACTGGAGGCCATTCAACGTAATCCCTTCCTCGTG
A
GTCTACACTACGCCTTCCAATCCTCATCAAAGCTTTACCTCGTGCTAGAC
TTTGCCAATGGCGGTGAGCTCTTCACGCATCTCTACCACTCTGAGAACTT
TGAGGAGTCCCGGGTGCGTGTGTACATAGCCGAGGTGGTGCTGGCCCTG
G
AGCAACTGCATCAGTTGGGGATCATCTATCGAGATATTAAGTTGGAGAA
T
ATTTTGCTTGATGGCGAGGGGCACATTGTTCTGTCCGACTTTGGACTGTC
GAAGATCCTTACCGCGGAGAATGAATACCGTGCGCATAGCTTCTGCGGC
A
CTCTGGAGTATATGGCACCTGAGATAATCCGTACGGGGCGCCAGGACA
C
GACAGTGCCGTGGACTGGTGGTCGGTGGGTGTGCTCACTTTTGAGCTGCT
CACCGGCGCTTCGCCATCGCCACGTCCGACGGCCAGGTCCAGCAATCG
G
AAATCTCGCGGCGGATTCAGAAGGAGCAGCCAATGATTCCCTCATCGTT
C
AGTGCCAACGCCCGCGACTTTGTGCTAAAGATGTTGGAAAAGAATCCAA
A
GCGGCGCTTGGGTGGCAACCATCGCGACGCCAGCGAAATCAAGGAGCA
CC
CCTTCTTCAACGGCATTAACTGGCAGGAGTTGCGGACAAAGCGCCGCAA
A
GCCCCCTATAAGCCCACCCTCACCGCCGAGGACGATGTGCAGAACTTCA
G
CAACGAGTTTACCGACCAAGTGCCCGAGGATCCCGAGTGCGATGCTCCG
C
CCAGCCGGATACGACTATTTCGCGGTTACACATACGTGGCGCCAGAGCA
T
TTGGAGCAGATGCGAAGGGATAACCACTGTGAGATTCAATACTTTAATA
C
AGGACTTCAGAATATACCTTGTCGGCCAGATGATCTTGAATTGGGCACA
C
GCACTTCAAACGGAGCCTATGGCACATGCCATTTTGTGGTGGACAGCTC
T
ACGGATTTGGTGTTTTTGGCCAAAATAATACCGCTATCCAAGTTCCGCCC
CTCCGAGGTCGACGCGCTGATCTCGTGCGCTCTCGACACGACCAATCAC
A
AAAATATCGTAAGCTACCATGGAACGTTCAGGGAAAAGTGCGAGACATG
G
ATCGTTATGGAGTACCTGTCCGGCCCGGAGCTGACCGCATCCATTCGCAT
GGATGAGGATTCGTGTCGGGAGATCTTTCTGCAGCTGGTCATGGCTGTGC
GGCACATACACTCCAAGCACTTTATACACGGTGACCTGAAACCGGAGAA
C
ATAATGTTCGAGAACCGAGAGGATAGGACTGTCAAACTGATCGATTTTG
G
CAGCGCATGTTACAATAATCGCTTTAAGAGCTGGAAGGACAAGCCGCGC
T
ACACGCTGGACTATGCTCCGCCAGAAATGCTCGCGGATGCGAATCTCGT
T
ACTTATTCGCCAGCCGTGGATATATACGGTCTTGGGGCCACGCTGTATAC
CATGCTGGTGGGTCATCGGCCGTACAGACAGAACGAAGACGATGTGGAT
C
ATCCGCAGCGGCCCATCACGAGCTTCGAAAGCGAATGCGCCGGGGAAC
G
TTTAATCAGCGTTCTATGCGCTGGGAGAGTGCAAGTCCTGCGTTCCGACA
TTTGGTTAGCTGGTGTCTGCAGCGGGATCCGGCTGATCGGCCCACATTGT
CAGATATTCTCGACAGCGAGTGGCTGCAGTACGGCTCAAATGATCCGGA
C
GTGGATATCATTCTGCCACAACAGATGGTTGTGGACCTCAGCGAAGACA
C
TATGGAACAACCGACGGGAGGTATGTTCGACGATCAGCAACAACTGGAA
T
TCATGCACGACAAGTCGGCGGAGGATGAGGGCATAACCCTAGTCAGCG
AG
CCCATGGATACGACTGTCGCGACTCACGAGTCTAGGAGAAATGCAGCCG
C
ATTTTCGTCGGTAGTTGCTCCAACCACCGACGACGAGATAGTGCACGAA
C
GATTTGACCCGGCATTTGAGGTCCAAGCGGACTTCTACGTTTCGACGA
G
AATGCGCCGCCTCTGCCTCTGCCCGAGGAGTACTACTCCGAACTACCTCT
GCCAGAGGAGGACCGGCAGTACATACCACCTCCACCAGCACTGATTCCT
G
TTGAACCAGAAACGACCTTCCGACGGCCAAGAACACGCCAGCAGCGTCG
C
ACTGAAAGCCAGCTAGTACAACCTGTTTCCGTCGCTACGTATGAGGACA
G
CAAGGCGTCCCTGCGCGTACTAATGCAACAGCTGCCGCCGCCAGGTGAC
A
ATGTGGTAGCACGCATTCCGAAGAGAACACATCGAGTCGTACGTACATT
G
CCTCCGACCTTTGGAACCACCAAGCGAGAAGAGAATTTCTATGGATTTA
G
TAAGACAGCAATATCGTGGCGAAAGACTCGGGCCAGCTGGCGTCACTTC
T
GTCTGCTTATCAATGGTGTGCAGCAGGTGCTTAAGGTTCGGTTCAAAAA
A
GCGCGTCGCGTCTACTGCCTGCCCCATATAAAAGAGGAAAAACTTGATC
A
TGCGTACGAAAAACCGTTGACGTTCCCGCGGCCAAAGGCACAACTGAAG
C
GCACCAAGCGGGAGCCCAAGGTGCCGCGGCCACCGACTCGAGTTCAGCC
G
GAGAGGGCGCGGGCAATGCGTCAACTTTATCAGTTCCAATGACATGAGG
A
TGACGACGGCGATGATGTGGAGGTGGTGGAAATCAAGGAGGAGCCGCG
AA
AGGAGGGAGAGGAGCAACTGCGAGCGGTGCTGCAGTGGGAGGGCGACT
TC
AGGAGGCCACGGATGTACAGCTGACCACACTCATCGACGCAATGGCCCA
T
CCAGGACAATGACAACCAGACGCCGGCCAAAACAGAGGGTAAAGAACC
AG
AGGGTTTCAGTGACCCACAAGAAACCCACTAGCCCCCCTCATGAGGGGG
A
AGGATCCGCAAGACGCACCAAAGATCCTGTCAATACAGTTTTTCAAGTT
T
GTTATTCATTAGGTTAAGCTTCAGAACTCATTTCGCTTATCGTTTTTTTC
AATCACGAAAAACAAAAAGCAACTTAACGTTTACACCTTTAGAAGGACG
G
TCGGTGTATTCAATATGCAATTTGTTGTGCTAGTTAATTATTTGAATAGC
TTAAATGTAAAAAGTCAGACACAAAACGGGAATATAACAAAGGCAACT
TT
TGCATTGATCTTAGCGCGATTAAGACATACAAAAACAAATACCGACAAC
A
GAACACAACCAACTAATATGATAATAAGATTTGCAATTAGTTTAGTGCA
A
AATTATGATTAAGACAAAATTGAGAAAGATATTCTTAGGGCGCCTATAT
T
ATATACACTACACACACAGTTTGCTTTTAGTTGTGCTTTATGTTAAGTTA
TATACATTATTCTTAACACACTATCCCTGAACTAACTAAAAAAAAAAGA
A
ATACTTCCAAGCATGTATTGCCAAATGCAATATGAATTAGCTAACACATT
TAAATGTAATTGTGCTTGGCAATGTTATCTAATTGCATTCAATTAATTTC
AGTTAAAACTAAGGGGAAAAAGAAGCCTCGCCCGAAAAACCATTTCCA
CA
TGAAATTGCGAAGCTCTTCTTTGAGATCGTGTCGAAAATATAACGTTTTT
CGCACACAAAACTCAACTATTGGGGAAACGTGAATAGTCGTAGACATTT
G
TTGATGGCAAACAAAGATTTAATTGTTTTGTTAAGTATTTTTGATGTATA
TTTTTTATTTTTCTGAAAATTCTGTATCTAAATAAGATAAAGCAATGGAT
AGCGATGAAATCTAAATAAAACTTAAACAATTATATTATTTTAAACTCGC
TTGATTTGATTTGTATTGTTTTCAGTTTGTATGGCCCGCATTTGGTCAGT
TTTTAAGAGCACCCCACTTAAACCCTCCGCTGACATAAACTTTTAAACCC
TAACCCAAAATCCAATTCACATATCGAACTGTATAAAATTTGCGTTAGA
G
CTGGATAATAAAGAAGGAAACTGATTGTTGAGGTCATGGTGTAAATTTT
A
GTATAAAACAAATAACATTTATATTAAAAGTTGCCTTCTAACAAGTTGCC
TTAGAACACACACAAATGTAAAGCCCAATAACTCCAAAAGTCGCAATA
T
GAAAAGTTAAACATACCCTTTACATTTTATAAGCAAACTCATTCGGTATG
AGTTTAGTTTTTATGGGTGCGATCATTGGCAATCCGTCGTAGTTTAAGAT
CACTGGAACTATCAATGACCATAGAATCTGCCCAATCTACACTTACAAT
G
GACGCAACAACCGATGGGGGACAATTAGTTAAATGTAAGAATAAAGCA
AT
CAAATCGTATTGAACTCGATTTTTGCAGTTCGGCCAAAGCATAGTTATGT
TAACCAAACAATCGAAGGCGTTTGATTGATTAACATTGAAGATCAGTGG
A
GACGGAGGCATGGAGTTGAATATGAAACAAGTTGCTAGCGGTTAAGTCA
C
CAAAGTCTGTACATAAAAACAATATCAAAATGCAGTTAAAATAAAGCAT
T
TCAAATGATAAGCAAA
>JIL-1|FBgn0020412
MSRLQKQNYEILSGTSTSRLKNHQHPRESESLAYEEPDQMVRNHLNGQLV
ANGNGKTRKNSNSETMTNGKKSKLNTEGSGSGSGKTLNYNNNNNNNNSIS
ATNGQYTNSSSKTTSASARDYTYRETISPPTPPSPPTTNVADIVCISDAE
SEDGRDPEREYYDQDMEEDEPNGIEIDESSSSLSKAKSNNAAAAAAAAAA
AAAAAASKASSSTTPSYAMPTSNSTPLDLDNEAHQRDLEAVTDLKYYVKL
YSDEAVSLNDFKIIRVLGTGAYGRVFLVRKLTRHDAGKLYAMKVLNKITV
VQKRKTAEHTKTERVVLEAIQRNPFLVSLHYAFQSSSKLYLVLDFANGGE
LFTHLYHSENFEESRVRVYIAEVVLALEQLHQLGIIYRDIKLENILLDGE
GHIVLSDFGLSKILTAENEYRAHSFCGTLEYMAPEIIRTGPPGHDSAVDW
WSVGVLTFELLTGASPFATSDGQVQQSEISRRIQKEQPMIPSSFSANARD
FVLKMLEKNPKRRLGGNHRDASEIKEHPFFNGINWQELRTKRRKAPYKPT
LTAEDDVQNFSNEFTDQVPEDPECDAPPSRIRLFRGYTYVAPEHLEQMRR
DNHCEIQYFNTGLQNIPCRPDDLELGTRTSNGAYGTCHFVVDSSTDLVFL
AKIIPLSKFRPSEVDALISCALDTTNHKNIVSYHGTFREKCETWIVMEYL
SGPELTASIRMDEDSCREIFLQLVMAVRHIHSKHFIHGDLKPENIMFENR
EDRTVKLIDFGSACYNNRFKSWKDKPRYTLDYAPPEMLADANLVTYSPAV
DIYGLGATLYTMLVGHRPYRQNEDDVDHSAAAHHELRKRMRRGTFNQRS
M
RWESASPAFRHLVSWCLQRDPADRPTLSDILDSEWLQYGSNDPDVDIILP
QQMVVDLSEDTMEQPTGGMFDDQQQLEFMHDKSAEDEGITLVSEPMDTTV
ATHESRRNAAAFSSVVAPTTDDEIVHERFDPAFEVQADFYGFDENAPPLP
LPEEYYSELPLPEEDRQYIPPPPALIPVEPETTFRRPRTRQQRRTESQLV
QPVSVATYEDSKASLRVLMQQLPPPGDNVVARIPKRTHRVVRTLPPTFGT
TKREENFYGFSKTAISWRKTRASWRHFCLLINGVQQVLKVRFKKARRVYC
LPHIKEEKLDHAYEKPLTFPRPKAQLKRTKREPKVPRPPTRVQPERARAM
RQLYQFQ
nosScim
>>nos|FBgn0002962|cDNA sequence
CCGTACGCTTCGCAGTTGTTTCAAGTTGTCTAAGGGACATACGATTTTTT
TTGCCTCTGCGTCACGATTTTAACCCAAAAGCGAGTTTAGTTACATGTAC
ATTATTATTAGATAAAGAAGTATCGCGAATACTTCAGTTGAATAAACTGT
GCTTGGTTTTTGGGTGAGGATTTGTGGAAAGTAGAGTGCGCGATAACCG
T
AACTTTCGACCCGGATTTTCGCCATGTTCCGCAGCAACTTGGAGGGCAGT
GGCGCAGCAGCAGTAGGTGTTGCAAATCCCCCCTCGTTGGCTCAGTCTG
G
AAAGATTTTCCAATTGCAGGATAACTTTTCTGCTTTTCACGCCAGAGGAG
GGCTCAACATTCTGGGCCTGCAGGACATGTATTTGGATACCAGTGGGGC
C
AACTCGTCGGCCACTTTGAGTCCGCCCATTACGCCGGTGACCCCTGACCC
GTCGACGTCTGCGCAGTCGACGCACTTCCCTTTTCTGGCCGACAGCGCAG
CCACCGCCAATTCGCTCCTTATGCAGCGACAGTACCACTACCACTTGCTG
CTCCAGCAGCAGCAGCAACTGGCCATGGCGCAGCACCAATTGGCGCTGG
C
TGCATCAGCGGCAGCGGCTAGTGCGAGTCACCAGCAAACGGACGAGATT
G
CGCGATCCTTGAAAATCTTTGCGCAGGTGACGACGGGCGCAGCAGAAAA
T
GCGGCTGGCTCGATGCAGGATGTGATGCAGGAGTTCGCGACCAATGGCT
A
TGCCAGCGATGATCTCGGTCGCATGTCCTACGGGAGTGCTCCGCCACAG
G
TGCAAATGCCACCGCAGCAGCAGCATCAGCAACAGCAGGGGCTGCACCT
G
CCACTGGGCCGCAATCCTGCCCAGCTGCAGACCAATGGCGGCAACTTAA
T
GCCCATTCCACTCGCCACCCACTGGCTGAACAACTACCGGGAGCATCTG
A
ACAACGTGTGGCGAAACATGTCGTATATACCAGCCGCTCCCAATACAAT
G
GGTTTGCAGGCCCAAACAGCGGCCACTGTGTCCACCAATCTCGGCGTGG
G
AATGGGTCTGGGATTGCCCGTGCAGGGCGAACAGCTGCGCGGAGCTTCC
A
ATTCCAGTAACAATAATAACAACAACAACAAGGTGTACAAGCGTTACAA
C
AGCAAGGCCAAAGAGATCAGCCGCCACTGCGTCTTTTGTGAGAATAACA
A
CGAACCAGAGGCGGTTATCAATAGCCACTCAGTGCGAGATAACTTTAAC
C
GAGTGCTGTGCCCCAAACTACGCACCTACGTGTGCCCCATCTGCGGGGC
A
TCTGGGGACTCGGCGCACACGATTAAGTACTGCCCCAAGAAGCCGATCA
T
CACCATGGAGGATGCGATCAAGGCGGAATCGTTCCGCCTAGCCAAGAGC
A
GTTACTACAAGCAACAGATGAAGGTTTAG
>nos|FBgn0002962
MFRSNLEGSGAAAVGVANPPSLAQSGKIFQLQDNFSAFHARGGLNILGLQ
DMYLDTSGANSSATLSPPITPVTPDPSTSAQSTHFPFLADSAATANSLLM
QRQYHYHLLLQQQQQLAMAQHQLALAASAAAASASHQQTDEIARSLKIFA
QVTTGAAENAAGSMQDVMQEFATNGYASDDLGRMSYGSAPPQVQMPPQQ
Q
HQQQQGLHLPLGRNPAQLQTNGGNLMPIPLATHWLNNYREHLNNVWRNM
S
YIPAAPNTMGLQAQTAATVSTNLGVGMGLGLPVQGEQLRGASNSSNNNNN
NNKVYKRYNSKAKEISRHCVFCENNNEPEAVINSHSVRDNFNRVLCPKLR
TYVCPICGASGDSAHTIKYCPKKPIITMEDAIKAESFRLAKSSYYKQQMK
V

Publications

Adams et al., Requirement of yeast fimbrin for actin organization and morphogenesis in vivo. Nature, 354:404-408 (1991).

Adams et al., Pavarotti encodes a kinesin-like protein required to organize the central spindle and contractile ring for cytokinesis. Genes Dev., 12:1483-1494 (1998).

Adams et al., The genome sequence of Drosophila melanogaster. Science, 287:2185-2195 (2000).

Afshar et al., DNA binding and meiotic chromosomal localization of the Drosophila Nod kinesin-like protein. Cell, 81:129-138 (1995).

Ashburner, M., Drosophila: A laboratory handbook. Cold Spring Harbor Laboratory Press, Cold Spring Harbor, N.Y.: 421 (1989).

Ashburner, M., Drosophila: A laboratory manual. Cold Spring Harbor Laboratory Press, Cold Spring Harbor, N.Y.: 8-9 (1989).

Auld, V. J.et al., Gliotactin, a novel transmembrane protein on peripheral glia, is required to form the blood-nerve barrier in Drosophila. Cell, 81:757-767 (1995).

Bahri, S. M. et al., The Drosophila bifocal gene encodes a novel protein which colocalizes with actin and is necessary for photoreceptor morphogenesis. Mol. Cell Biol., 17:5521-5529 (1997).

Baker, B. S. et al., Genetic analysis of sex chromosomal meiotic mutants in Drosophila melanogaster. Genetics 71:255-286 (1972).

Bergstrom, D. E. et al., Regulatory autonomy and molecular characterization of the Drosophila out at first gene. Genetics, 139:1331-1346 (1995).

Bhat, M. A. et al., Chromatid segregation at anaphase requires the barren product, a novel chromosome-associated protein that interacts with topoisomerase II. Cell, 87:1103-1114 (1996).

Botstein and Shortle, Strategies and applications of in vitro mutagenesis, Science, 229:1193 (1985).

Buchowicz J. et al., Cytogenet. Cell. Genet., 86:194-203 (1999).

Cook, K. R. et al., Identification of trans-acting genes necessary for centromere function in Drosophila melanogaster using centromere-defective minichromosomes. Genetics, 145:737-747 (1997).

Creighton, T. E., Proteins-Structure and Molecular Properties, 2d Ed., W. H., Freeman and Company, New York, (1993).

Crooke E. et al., E. coli minichromosome replication: regulation of initiation at oriC, Res Microbiol., 142(2-3):127-30 (1991).

Dernburg, A. F. et al., Direct evidence of a role for heterochromatin in meiotic chromosome segregation. Cell, 86:135-146 (1996).

Deshpande, G. G. et al., Novel functions of nanos in downregulating mitosis and transcription during the development of the Drosophila germline. Cell, 99:271-281 (1999).

Dobie, K. W. et al., Centromere proteins and chromosome inheritance: a complex affair. Curr. Opin. Genet. Dev., 9:206-217 (1999).

Foe, V. E. et al., Mitosis and morphogenesis in the Drosophila embryo: Point and counterpoint. In The development of Drosophila melanogaster, edited by M. BATE and A. MARTINEZ-ARIAS. Cold Spring Harbor Laboratory Press, Cold Spring Harbor, N.Y. (1993).

Fogarty, P. et al., The Drosophila grapes gene is related to checkpoint gene chk1/rad27 and is required for late syncytial division fidelity. Curr. Biol., 7:418-426 (1997).

Garcia-Alonso, L. et al., Genetic analysis of Laminin A in Drosophila: extracellular matrix containing laminin A is required for ocellar axon pathfinding. Development, 122:2611-2621 (1996).

Gaul, U. G. et al., A putative Ras GTPase activating protein acts as a negative regulator of signaling by the Sevenless receptor tyrosine kinase. Cell, 68:1007-1019 (1992).

Gloor, G. B. et al., Type I repressors of P element mobility. Genetics, 135:81-95 (1993).

Greenfield et al., Conversion of closed circular DNA molecules to single-nicked molecules by digestion with DNAase I in the presence of ethidium bromide, Biochim. Biophys. Acta., 407:365 (1985).

Guiducci C. et al., Use of a human Invention minichromosome as a cloning and expression vector for mammalian cells, Hum Mol Genet., 8(8):1417-24 (1999).

Harrison, S. D. et al., A genetic analysis of the 63E-64A genomic region of Drosophila melanogaster: identification of mutations in a replication factor C subunit. Genetics, 139:1701-1709 (1995).

Hawley, R. S. and W. E. Theurkauf, Requiem for distributive segregation: achiasmate segregation in Drosophila females. Trends Genet., 9:310-317 (1993).

Hernandez, G. R. et al., Localization, structure and expression of the gene for translation initiation factor eIF-4E from Drosophila melanogaster. Mol. Gen. Genet., 253:624-633 (1997).

Hook, E. B., The impact of aneuploidy upon public health: mortality and morbidity associated with human chromosomal abnormalities, pp. 7-33 in Aneuploidy: Etiology & Mechanisms, edited by V. Dellarco, P. Voytek, and A. Hollaender, New York: Plenum press (1985).

Homer, M. A. et al., Ecdysteroid regulation and DNA binding properties of Drosophila nuclear hormone receptor superfamily members. Dev. Biol., 168:490-502 (1995).

Ilyin, Y. V. et al., Isolation and characterization of a new family of mobile dispersed genetic elements, mdg3, in Drosophila melanogaster. Chromosoma, 81:27-53 (1980).

Jin, Y. Y. et al., JIL-1: a novel chromosomal tandem kinase implicated in transcriptional regulation in Drosophila. Mol. Cell, 4129-135 (1999).

Kania, A. A. et al., P-element mutations affecting embryonic peripheral nervous system development in Drosophila melanogaster. Genetics, 139:1663-1678 (1995).

Kapler G M., Nuclear extrachromosomal DNA of higher plants, Acta Biochim Pol., 44(1):13-9 (1997).

Karpen, G. H. and S. A., Endow, Meiosis: Chromosome Behaviour and Spindle Dynamics, in Frontiers in Biology, edited by S. A. Endow and D. Glover. Oxford University Press (1998).

Karpen, G. H. et al., Centric heterochromatin and the efficiency of achiasmate disjunction in Drosophila female meiosis. Science, 273:118-122 (1996).

Kedes, L. H., Histone genes and histone messengers. Annu. Rev. Biochem., 48:837-870 (1979).

Le, M-H. et al., Islands of complex DNA are widespread in Drosophila centric heterochromatin. Genetics, 141:283-303 (1995).

Lee, E. C. et al., Functional analysis of the fibrinogen-related scabrous gene from Drosophila melanogaster identifies potential effector and stimulatory protein domains. Genetics, 150:663-673 (1998).

Liao, G-C. et al., Insertion site preferences of the P transposable element in Drosophila melanogaster. Proc. Natl. Acad. Sci. USA, 97:3347-3351 (2000).

Logarinho, E. and C. E. Sunkel, The Drosophila POLO kinase localises to multiple compartments of the mitotic apparatus and is required for the phosphorylation of MPM2 reactive epitopes. J. Cell Sci., 111:2897-2909 (1998).

Lopez, J. M. et al., The relationship between sister-chromatid cohesion and kinetochore formation at the centromere. in press.

Martino J. A. et al., Influence of nucleosome structure on the three-dimensional folding of idealized minichromosomes, Structure Fold Des., 7(8):1009-22 (1999).

Megraw, T. L. et al., The centrosomin protein is required for centrosome assembly and function during cleavage in Drosophila. Development, 126:2829-2839 (1999).

Meluh, P. B. et al., Cse4p is a component of the core centromere of Saccharomyces cerevisiae. Cell, 94:607-613 (1998).

Mitelman, F., Catalog of Chromosome Aberrations in Cancer, 5th Edition, New York:

Wiley (1994).

Moore, J. D. and S. A., Endow, Kinesin proteins: a phylum of motors for microtubule-based motility. Bioessays, 18:207-219 (1996).

Mossi, R. Z. et al., Replication factor C interacts with the C-terminal side of proliferating cell nuclear antigen. J. Biol. Chem., 272:1769-1776 (1997).

Murphy, T. D. and G. H., Karpen, Interactions between the nod⁺ kinesin-like gene and extracentromeric sequences are required for transmission of a Drosophila minichromosome. Cell, 81:139-148 (1995).

Murphy, T. D., and G. H. Karpen, Localization of centromere function in a Drosophila minichromosome. Cell, 82:599-609 (1995).

Nielsen, E. F. et al., Rab5 regulates motility of early endosomes on microtubules. Nature Cell Biol., 1:376-382 (1999).

Nüsslein-Volhard, C. E. et al., Mutations affecting the pattern of the larval cuticle in Drosophila melanogaster. I. Zygotic loci on the second chromosome. Roux's Arch. Dev. Biol., 193:267-282 (1984).

Pluta, A. F. et al., The centromere: hub of chromosomal activities. Science, 270:1591-1594 (1995).

Prokopenko, S. N. et al., A putative exchange factor for Rho1 GTPase is required for initiation of cytokinesis in Drosophila. Genes Dev., 13:2301-2314 (1999).

Rattan et al., Protein Synthesis: Posttranslational Modifications and Aging, Ann. N.Y. Acad. Sci., 663:48-62 (1992).

Review, Developmentally regulated processing and replication of the Tetrahymena rDNA minichromosome, Curr Opin Genet Dev., 3(5):730-5 (1993).

Rieder, C. L. et al., The vertibrate cell kinetochore and its roles during mitosis. Trends Cell Biol., 8:310-318 (1998).

Robertson, H. M. et al., A stable genomic source of P element transposase in Drosophila melanogaster. Genetics, 118:461-470 (1988).

Rørth, P., A modular misexpression screen in Drosophila detecting tissue-specific phenotypes. Proc. Natl. Acad. Sci. USA, 93:12418-12422 (1996).

Roseman, R. R. et al., A P element containing suppressor of hairy-wing binding regions has novel properties for mutagenesis in Drosophila melanogaster. Genetics, 141:1061-1074 (1995).

Salzberg, A. et al., P-element insertion alleles of essential genes on the third chromosome of Drosophila melanogaster: mutations affecting embryonic PNS development. Genetics, 147:1723-1741 (1997).

Sandler, L. D. et al., Mutants affecting meiosis in natural populations of Drosophila melanogaster. Genetics 60:525-558 (1968).

Seifter et al., Analysis for protein modifications and nonprotein cofactors, Methods in Enzymol, 182:626-646 (1990).

Sekelsky, J. J. et al., Identification of novel Drosophila meiotic genes recovered in a P-element screen. Genetics, 152:529-542 (1999).

Severin, F. F. et al., Kinetochores distinguish GTP from GDP forms of the microtubule lattice. Nature, 388:888-891 (1997).

Shirakata M. et al., Requirement of replication licensing for the dyad symmetry element-dependent replication of the Epstein-Barr virus oriP minichromosome, Virology., 263(l):42-54 (1999).

Sibon, O. C. et al., A novel histone H4 mutant defective in nuclear division and mitotic chromosome transmission. Mol. Cell Biol., 16:1017-1026 (1996).

Spradling, A. C. et al., The Berkeley Drosophila Genome Project gene disruption project: Single P-element insertions mutating 25% of vital Drosophila genes. Genetics, 153:135-177 (1999).

Spradling, A. C. et al., Gene disruptions using P transposable elements: an integral component of the Drosophila genome project. Proc. Natl. Acad. Sci. USA, 92:10824-10830 (1995).

Sun, X. J. et al., Molecular structure of a functional Drosophila centromere. Cell, 91:1007-1019 (1997).

Van den berg, D. J. and U. Francke, Roberts syndrome: a review of 100 cases and a new rating system for severity. Am. J. Med. Genet., 47:1104-1123 (1993).

Verni, F. R. et al., Genetic and molecular analysis of wings-apart-like (wap1), a gene controlling heterochromatin organization in Drosophila melanogaster. Genetics, 154:1693-1710 (2000).

Wakimoto, B. T., Beyond the nucleosome: epigenetic aspects of position-effect variegation in Drosophila. Cell, 93:321-324 (1998).

Wang, C. and R., Lehmann, Nanos is the localized posterior determinant in Drosophila. Cell, 66:637-647 (1991).

Williams, B. C. et al., Neocentromere activity of structurally acentric mini-chromosomes in Drosophila. Nature Genetics, 18:30-37 (1998).

Wold, F., Posttranslational Protein Modifications: Perspectives and Prospects, Posttranslational Covalent Modification of Proteins, 193, 1-12, B. C. Johnson, Ed., Academic Press, New York.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method to identify at least one candidate agent that modulates chromosomal inheritance in a cell comprising:
a) contacting a cell comprising a sensitized minichromosome with a candidate agent to produce a treated cell; and
b) determining if the inheritance pattern of the sensitized minichromosome in progeny of the treated cell is affected by the candidate agent.

2. The method of claim 1 comprising (c) determining if the inheritance pattern of the sensitized minichromosome in progeny of the treated cell differs from the inheritance pattern of the sensitized minichromosome in progeny of an untreated control cell.

3. The method of claim 1, wherein the sensitized minichromosome is Dp1187.

4. The method of claim 1, wherein the sensitized minichromosome is J21A.

5. The method of claim 1, wherein the sensitized minichromosome comprises a region of about 420 Kb of nucleic acid sequence from Dp1187 that confers chromosomal inheritance onto Dp1187.

6. The method of claim 1, wherein the sensitized minichromosome comprises a region of about 290 Kb of nucleic acid sequence from J21A that confers chromosomal inheritance onto J21A.

7. The method of claim 1, wherein the minichromosome comprises a detectable marker.

8. The method of claim 7, wherein the detectable marker is a gene that encodes a fluorescent polypeptide.

9. The method of claim 7, wherein the detectable marker is a gene that encodes drug resistance.

10. The method of claim 1, wherein fluorescence activated cell sorting, drug selection, fluorescent microscopy or growth rate is used to determine inheritance of the sensitized minichromosome.

11. A candidate agent identified according to claim 1.

12. A method to diagnose a patient having an indication associated with altered chromosome inheritance comprising:
a) obtaining nucleic acid from the patient; and
b) determining if the nucleic acid from the patient contains a mutation in a gene that encodes a polypeptide involved with chromosomal inheritance.

13. The method of claim 12, wherein the nucleic acid from the patient has at least 70% sequence identity to a nucleic acid sequence selected from SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 or 147-149.

14. The method of claim 12, wherein the nucleic acid from the patient encodes a polypeptide having at least 70% sequence identity to a polypeptide sequence selected from SEQ ID NO: 2, 4, 6, 8, 10, 12, 14, 16, 18, 20, 22, 24, 26, 28, 30, 32, 34, 36, 38, 40, 44-46, 48, 50, 52, 54, 56, 58, 60, 62, 64, 66, 68, 70, 72, 74, 76, 78, 80, 82, 84, 87, 88, 90, 93, 94, 96, 98, 100, 102, 104, 106, 108, 111, 112, 115, 116, 118, 120, 122, 124, 126, 128, 130, 132, 134, 138-140, 142, 144 or 146.

15. The method of claim 12, wherein the nucleic acid from the patient encodes a polypeptide having a substantially similar function to a polypeptide encoded by a nucleic acid sequence selected from SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 or 147-149.

16. The method of claim 12, wherein the nucleic acid from the patient encodes a polypeptide having a similar function to a polypeptide encoded by a nucleic acid sequence selected from SEQ ID NO: 1, 3, 5, 7, 9, 11, 13, 15, 17, 19, 21, 23, 25, 27,29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 or 147-149.

17. A method for treating a patient having an indication associated with altered chromosomal inheritance comprising, administering an agent that modulates chromosomal inheritance to the patient.

18. The method of claim 17, wherein the agent is identified according to the method of claim 1.

19. The method of claim 17, wherein the agent comprises a nucleic acid sequence selected from SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 or 147-149.

20. The method of claim 17, wherein the agent comprises a polypeptide sequence selected from SEQ ID NO: 2, 4, 6, 8, 10, 12, 14, 16, 18, 20, 22, 24, 26, 28, 30, 32, 34, 36, 38, 40, 44-46, 48, 50, 52, 54, 56, 58, 60, 62, 64, 66, 68, 70, 72, 74, 76, 78, 80, 82, 84, 87, 88, 90, 93, 94, 96, 98, 100, 102, 104, 106, 108, 111, 112, 115, 116, 118, 120, 122, 124, 126, 128, 130, 132, 134, 138-140, 142, 144 or 146.

21. The method of claim 17, wherein the agent comprises a nucleic acid sequence having at least 70% sequence identity to a nucleic acid sequence selected from SEQ ID NO: 1, 3, 5, 7, 9, 11, 13, 15, 17, 19, 21, 23, 25, 27, 29, 31, 33, 35, 37, 39, 41-43,47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 or 147-149.

22. The method of claim 17, wherein the agent comprises a polypeptide having at least 70% sequence identity to a polypeptide selected from SEQ ID NO: 2, 4, 6, 8, 10, 12, 14, 16, 18, 20, 22, 24, 26, 28, 30, 32, 34, 36, 38, 40, 44-46, 48, 50, 52, 54, 56, 58, 60, 62, 64, 66, 68, 70, 72, 74, 76, 78, 80, 82, 84, 87, 88, 90, 93, 94, 96, 98,100,102, 104, 106, 108, 111, 112, 115, 116, 118, 120, 122, 124, 126, 128, 130, 132, 134, 138-140, 142, 144 or 146.

23. The method of claim 17, wherein the agent comprises a nucleic acid sequence that encodes a polypeptide having a substantially similar function to a polypeptide encoded by a nucleic acid sequence selected from SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129, 131, 133, 135-137, 141, 143, 145 or 147-149.

24. The method of claim 17, wherein the indication associated with altered chromosomal inheritance is cancer, Down syndrome, cri du chat syndrome, Kleinfelter's syndrome, Tumer's syndrome or fragile X syndrome.

25. A method to identify a nucleic acid sequence involved with chromosomal inheritance comprising:
a) obtaining a test cell comprising a sensitized minichromosome, wherein the test cell has been mutagenized and has increased or decreased inheritance of the sensitized minichromosome; and
b) identifying the nucleic acid sequence that contains a mutation which produces increased or decreased inheritance.

26. The method of claim 25 comprising (c) comparing inheritance of the minichromosome in a cell that has been mutagenized to inheritance of the minichromosome in a cell that has not been mutagenized.

27. The method of claim 25, wherein the test cell is mutagenized with a chemical, ultraviolet light, radiation or a virus.

28. The method of claim 25, wherein the sensitized minichromosome is Dp1187.

29. The method of claim 25, wherein the sensitized minichromosome is J21A.

30. The method of claim 25, wherein the sensitized minichromosome is a nucleic acid construct comprising a minimal cis-acting nucleic acid sequence required for inheritance of the minichromosome.

31. A method to identify a polynucleotide involved with chromosomal inheritance comprising:
a) introducing a mutagenized polynucleotide into a cell comprising a sensitized minichromosome; and
b) determining if inheritance of the sensitized minichromosome is increased or decreased.

32. The method of claim 31 comprising (c) comparing inheritance of the sensitized minichromosome in a control cell that does not contain the mutagenized polynucleotide to inheritance of the minichromosome in a cell that contains the mutagenized polynucleotide.

33. A polynucleotide having at least 70% sequence identity to a nucleic acid sequence selected from SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110,113, 114, 117, 119, 121, 123, 125, 127, 129, 131, 133, 135-137, 141, 143, 145 or 147-149.

34. A polypeptide having at least 70% sequence identity to an amino acid sequence selected from SEQ ID NO: 2, 4, 6, 8, 10, 12, 14, 16, 18, 20, 22, 24, 26, 28, 30, 32, 34, 36, 38, 40, 44-46, 48, 50, 52, 54, 56, 58, 60, 62, 64, 66, 68, 70, 72, 74, 76, 78, 80, 82, 84, 87, 88, 90, 93, 94, 96, 98, 100, 102, 104, 106, 108, 111, 112, 115, 116, 118, 120, 122, 124, 126, 128, 130, 132, 134, 138-140, 142, 144 or 146.

35. A polynucleotide encoding a polypeptide having a substantially similar activity to a polypeptide encoded by a nucleic acid sequence selected from SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119,121, 123, 125, 127, 129, 131, 133, 135-137, 141, 143, 145 or 147-149.

36. A polynucleotide encoding a polypeptide having a substantially similar activity to an amino acid sequence selected from SEQ ID NO: 2, 4, 6, 8, 10, 12, 14, 16, 18, 20, 22, 24, 26, 28, 30, 32, 34, 36, 38, 40, 44-46, 48, 50, 52, 54, 56, 58, 60, 62, 64, 66, 68, 70, 72, 74, 76, 78, 80, 82, 84, 87, 88, 90, 93, 94, 96, 98, 100, 102, 104, 106, 108, 111, 112, 115, 116, 118, 120, 122, 124, 126, 128, 130, 132, 134, 138-140, 142, 144 or 146.

37. The polynucleotide of claim 33, wherein the nucleic acid sequence encodes a polypeptide involved with chromosomal inheritance.

38. The polypeptide of claim 34, wherein the polypeptide is involved with chromosomal inheritance.

39. An expression cassette comprising a nucleic acid sequence selected from SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119,121, 123, 125, 127, 129, 131, 133, 135-137, 141, 143, 145 or 147-149.

40. An expression cassette comprising a nucleic acid sequence having at least 70% sequence identity to a nucleic acid sequence selected from SEQ ID NO: 1, 3, 5, 7, 9, 11, 13, 15, 17, 19, 21, 23, 25, 27, 29, 31, 33, 35, 37, 39, 41-43, 47, 49, 51, 53, 55, 57, 59, 61, 63, 65, 67, 69, 71, 73, 75, 77, 79, 81, 83, 85, 86, 89, 91, 92, 95, 97, 99, 101, 103, 105, 107, 109, 110, 113, 114, 117, 119, 121, 123, 125, 127, 129,131, 133, 135-137, 141, 143, 145 or 147-149.

41. A construct comprising a vector and the expression cassette of claim 40.

42. The construct of claim 41, wherein the vector is a plasmid, phagemid or virus.

43. A cell comprising a polynucleotide of claims 33, 35-38, a polypeptide of claim 34, an expression cassette of claim 39 or 40, or a construct of claim 41.
